SEQUENCE LISTING (225 sequences)

SEQ ID NO: 1 - DNA, 42 bp, synthetic construct
  misc_feature 1..42: note = Description of Artificial Sequence: Synthetic primer
  source 1..42: mol_type = other DNA
gttaagcttgccaccatgggtctggagaagtccctcattctg

SEQ ID NO: 2 - DNA, 39 bp, synthetic construct
  misc_feature 1..39: note = Description of Artificial Sequence: Synthetic primer
  source 1..39: mol_type = other DNA
gataccaccggtagggaatctgcagcacagaagtttcag

SEQ ID NO: 3 - DNA, 40 bp, synthetic construct
  misc_feature 1..40: note = Description of Artificial Sequence: Synthetic primer
  source 1..40: mol_type = other DNA
ggctcgagcacagtagcatcaaagtggactggtacgtagg

SEQ ID NO: 4 - DNA, 48 bp, synthetic construct
  misc_feature 1..48: note = Description of Artificial Sequence: Synthetic oligonucleotide
  source 1..48: mol_type = other DNA
aaatctagacctcaaccaggtagggaatctgcagcacagaagtttcag

SEQ ID NO: 5 - DNA, 43 bp, synthetic construct
  misc_feature 1..43: note = Description of Artificial Sequence: Synthetic oligonucleotide
  source 1..43: mol_type = other DNA
tctagactatcacacagtagcatcaaagtggactggtacgtag

SEQ ID NO: 6 - DNA, 49 bp, synthetic construct
  misc_feature 1..49: note = Description of Artificial Sequence: Synthetic primer
  source 1..49: mol_type = other DNA
tgtccaccgtgtccagcacctgaactcctgggtggatcgtcagtcttcc

SEQ ID NO: 7 - DNA, 49 bp, synthetic construct
  misc_feature 1..49: note = Description of Artificial Sequence: Synthetic primer
  source 1..49: mol_type = other DNA
agatctcgagcccaaatcttctgacaaaactcacacatgtccaccgtgt

SEQ ID NO: 8 - DNA, 51 bp, synthetic construct
  misc_feature 1..51: note = Description of Artificial Sequence: Synthetic primer
  source 1..51: mol_type = other DNA
tctagattatcatttacccggagacagagagaggctcttctgcgtgtagtg

SEQ ID NO: 9 - DNA, 58 bp, synthetic construct
  misc_feature 1..58: note = Description of Artificial Sequence: Synthetic primer
  source 1..58: mol_type = other DNA
cctccatgcaaatgcccagcacctaacctcttgggtggatcatccgtcttcatcttcc

SEQ ID NO: 10 - DNA, 45 bp, synthetic construct
  misc_feature 1..45: note = Description of Artificial Sequence: Synthetic primer
  source 1..45: mol_type = other DNA
gaagatctcgagcccagaggtcccacaatcaagccctctcctcca

SEQ ID NO: 11 - DNA, 48 bp, synthetic construct
  misc_feature 1..48: note = Description of Artificial Sequence: Synthetic primer
  source 1..48: mol_type = other DNA
gtttctagattatcatttacccggagtccgagagaagctcttagtcgt

SEQ ID NO: 12 - DNA, 49 bp, synthetic construct; identical to SEQ ID NO: 7
  misc_feature 1..49: note = Description of Artificial Sequence: Synthetic primer
  source 1..49: mol_type = other DNA

SEQ ID NO: 13 - DNA, 39 bp, synthetic construct
  misc_feature 1..39: note = Description of Artificial Sequence: Synthetic primer
  source 1..39: mol_type = other DNA
gttttctcgatggaggctgggagggctttgttggagacc

SEQ ID NO: 14 - DNA, 48 bp, synthetic construct
  misc_feature 1..48: note = Description of Artificial Sequence: Synthetic primer
  source 1..48: mol_type = other DNA
aaggtctccaacaaagccctcccagcctccatcgagaaaacaatctcc

SEQ ID NO: 15 - DNA, 51 bp, synthetic construct; identical to SEQ ID NO: 8
  misc_feature 1..51: note = Description of Artificial Sequence: Synthetic primer
  source 1..51: mol_type = other DNA

SEQ ID NO: 16 - DNA, 31 bp, synthetic construct
  misc_feature 1..31: note = Description of Artificial Sequence: Synthetic primer
  source 1..31: mol_type = other DNA
accggtaaggaatcccgggccaagaaattcc

SEQ ID NO: 17 - DNA, 40 bp, synthetic construct
  misc_feature 1..40: note = Description of Artificial Sequence: Synthetic primer
  source 1..40: mol_type = other DNA
ctcgagatctgtagagtcctccacagaagcatcaaagtgg

SEQ ID NO: 18 - DNA, 33 bp, synthetic construct
  misc_feature 1..33: note = Description of Artificial Sequence: Synthetic primer
  source 1..33: mol_type = other DNA
agactgccgcctgacaaacgactccaggtaccc

SEQ ID NO: 19 - DNA, 33 bp, synthetic construct
  misc_feature 1..33: note = Description of Artificial Sequence: Synthetic primer
  source 1..33: mol_type = other DNA
gggtacctggagtcgtttgtcaggcggcagtct

SEQ ID NO: 20 - DNA, 37 bp, synthetic construct
  misc_feature 1..37: note = Description of Artificial Sequence: Synthetic primer
  source 1..37: mol_type = other DNA
accggtatgggctcacagaccctgccccatggtcaca

SEQ ID NO: 21 - DNA, 43 bp, synthetic construct
  misc_feature 1..43: note = Description of Artificial Sequence: Synthetic primer
  source 1..43: mol_type = other DNA
ctcgagatctgttgttccagtggtagccggagtgccgtacatg

SEQ ID NO: 22 - DNA, 45 bp, synthetic construct
  misc_feature 1..45: note = Description of Artificial Sequence: Synthetic primer
  source 1..45: mol_type = other DNA
gttaagcttgccaccatgtccctgcacccagcttccccacgcctg

SEQ ID NO: 23 - DNA, 44 bp, synthetic construct
  misc_feature 1..44: note = Description of Artificial Sequence: Synthetic primer
  source 1..44: mol_type = other DNA
ctcgagatctgaggagcgattgcctttttttctctttttgagag

SEQ ID NO: 24 - DNA, 42 bp, synthetic construct
  misc_feature 1..42: note = Description of Artificial Sequence: Synthetic primer
  source 1..42: mol_type = other DNA
accggtctaaggctctgctccttcaatgtgaggtcctttgga

SEQ ID NO: 25 - DNA, 44 bp, synthetic construct; identical to SEQ ID NO: 23
  misc_feature 1..44: note = Description of Artificial Sequence: Synthetic primer
  source 1..44: mol_type = other DNA

SEQ ID NO: 26 - DNA, 36 bp, synthetic construct
  misc_feature 1..36: note = Description of Artificial Sequence: Synthetic primer
  source 1..36: mol_type = other DNA
gttaccggtctgaagatcgcagccttcaacatccag

SEQ ID NO: 27 - DNA, 39 bp, synthetic construct
  misc_feature 1..39: note = Description of Artificial Sequence: Synthetic primer
  source 1..39: mol_type = other DNA
gttctcgagatctttcagcatcacctccactggatagtg

SEQ ID NO: 28 - DNA, 36 bp, synthetic construct
  misc_feature 1..36: note = Description of Artificial Sequence: Synthetic primer
  source 1..36: mol_type = other DNA
gttgatatcctgaagatcgcagccttcaacatccag

SEQ ID NO: 29 - DNA, 42 bp, synthetic construct
  misc_feature 1..42: note = Description of Artificial Sequence: Synthetic primer
  source 1..42: mol_type = other DNA
gtttctagattatcacttcagcatcacctccactggatagtg

SEQ ID NO: 30 - DNA, 49 bp, synthetic construct; identical to SEQ ID NO: 6
  misc_feature 1..49: note = Description of Artificial Sequence: Synthetic primer
  source 1..49: mol_type = other DNA

SEQ ID NO: 31 - DNA, 49 bp, synthetic construct; identical to SEQ ID NO: 7
  misc_feature 1..49: note = Description of Artificial Sequence: Synthetic primer
  source 1..49: mol_type = other DNA

SEQ ID NO: 32 - DNA, 42 bp, synthetic construct
  misc_feature 1..42: note = Description of Artificial Sequence: Synthetic primer
  source 1..42: mol_type = other DNA
gaagatctcgagcccaaatcttctgacaaaactcacacatgt

SEQ ID NO: 33 - DNA, 43 bp, synthetic construct
  misc_feature 1..43: note = Description of Artificial Sequence: Synthetic primer
  source 1..43: mol_type = other DNA
gttagatctcgagcccaaatcttctgacaaaactcacacatct

SEQ ID NO: 34 - DNA, 51 bp, synthetic construct; identical to SEQ ID NO: 8
  misc_feature 1..51: note = Description of Artificial Sequence: Synthetic primer
  source 1..51: mol_type = other DNA

SEQ ID NO: 35 - DNA, 48 bp, synthetic construct; identical to SEQ ID NO: 14
  misc_feature 1..48: note = Description of Artificial Sequence: Synthetic primer
  source 1..48: mol_type = other DNA

SEQ ID NO: 36 - DNA, 39 bp, synthetic construct; identical to SEQ ID NO: 13
  misc_feature 1..39: note = Description of Artificial Sequence: Synthetic primer
  source 1..39: mol_type = other DNA

SEQ ID NO: 37 - DNA, 43 bp, synthetic construct
  misc_feature 1..43: note = Description of Artificial Sequence: Synthetic primer
  source 1..43: mol_type = other DNA
aagcttgccaccatggctctggagaagtctcttgtccggctcc

SEQ ID NO: 38 - DNA, 40 bp, synthetic construct; identical to SEQ ID NO: 17
  misc_feature 1..40: note = Description of Artificial Sequence: Synthetic primer
  source 1..40: mol_type = other DNA

SEQ ID NO: 39 - DNA, 31 bp, synthetic construct; identical to SEQ ID NO: 16
  misc_feature 1..31: note = Description of Artificial Sequence: Synthetic primer
  source 1..31: mol_type = other DNA

SEQ ID NO: 40 - DNA, 45 bp, synthetic construct
  misc_feature 1..45: note = Description of Artificial Sequence: Synthetic primer
  source 1..45: mol_type = other DNA
gatatcccttccctgggcaaggaatcccgggccaagaaattccag

SEQ ID NO: 41 - DNA, 45 bp, synthetic construct
  misc_feature 1..45: note = Description of Artificial Sequence: Synthetic primer
  source 1..45: mol_type = other DNA
gtttctagattattaggtagagtcctccacagaagcatcaaagtg

SEQ ID NO: 42 - DNA, 45 bp, synthetic construct
  misc_feature 1..45: note = Description of Artificial Sequence: Synthetic primer
  source 1..45: mol_type = other DNA
ggtaagcttgccaccatgtcacgggagctggccccactgctgctt

SEQ ID NO: 43 - DNA, 42 bp, synthetic construct
  misc_feature 1..42: note = Description of Artificial Sequence: Synthetic primer
  source 1..42: mol_type = other DNA
ctcgagatctgaggagcgtttgctctttgttttcttccttag

SEQ ID NO: 44 - DNA, 41 bp, synthetic construct
  misc_feature 1..41: note = Description of Artificial Sequence: Synthetic primer
  source 1..41: mol_type = other DNA
accggtatgaggatctgctccttcaacgtcaggtcctttgg

SEQ ID NO: 45 - DNA, 36 bp, synthetic construct; identical to SEQ ID NO: 26
  misc_feature 1..36: note = Description of Artificial Sequence: Synthetic primer
  source 1..36: mol_type = other DNA

SEQ ID NO: 46 - DNA, 39 bp, synthetic construct; identical to SEQ ID NO: 27
  misc_feature 1..39: note = Description of Artificial Sequence: Synthetic primer
  source 1..39: mol_type = other DNA

SEQ ID NO: 47 - DNA, 36 bp, synthetic construct; identical to SEQ ID NO: 28
  misc_feature 1..36: note = Description of Artificial Sequence: Synthetic primer
  source 1..36: mol_type = other DNA

SEQ ID NO: 48 - DNA, 42 bp, synthetic construct; identical to SEQ ID NO: 29
  misc_feature 1..42: note = Description of Artificial Sequence: Synthetic primer
  source 1..42: mol_type = other DNA

SEQ ID NO: 49 - DNA, 60 bp, synthetic construct
  misc_feature 1..60: note = Description of Artificial Sequence: Synthetic primer
  source 1..60: mol_type = other DNA
gatggctgcgagccctgcaggaacgacaccttcaaccgagagccattcattgtcaggttc

SEQ ID NO: 50 - DNA, 60 bp, synthetic construct
  misc_feature 1..60: note = Description of Artificial Sequence: Synthetic primer
  source 1..60: mol_type = other DNA
gaacctgacaatgaatggctctcggttgaaggtgtcgttcctgcagggctcgcagccatc

SEQ ID NO: 51 - DNA, 39 bp, synthetic construct
  misc_feature 1..39: note = Description of Artificial Sequence: Synthetic primer
  source 1..39: mol_type = other DNA
ggagaagaacctgacaatgaatggctctcggttgaaggt

SEQ ID NO: 52 - DNA, 39 bp, synthetic construct
  misc_feature 1..39: note = Description of Artificial Sequence: Synthetic primer
  source 1..39: mol_type = other DNA
accttcaaccgagagccattcattgtcaggttcttctcc

SEQ ID NO: 53 - DNA, 35 bp, synthetic construct
  misc_feature 1..35: note = Description of Artificial Sequence: Synthetic primer
  source 1..35: mol_type = other DNA
accggtatgggccctggagctcgcagacagggcag

SEQ ID NO: 54 - DNA, 34 bp, synthetic construct
  misc_feature 1..34: note = Description of Artificial Sequence: Synthetic primer
  source 1..34: mol_type = other DNA
ctcgagatctttggtcctagcagaggctgtgacc

SEQ ID NO: 55 - DNA, 37 bp, synthetic construct
  misc_feature 1..37: note = Description of Artificial Sequence: Synthetic primer
  source 1..37: mol_type = other DNA
accggtctcgagatgggccctggagctcgcagacagg

SEQ ID NO: 56 - DNA, 31 bp, synthetic construct
  misc_feature 1..31: note = Description of Artificial Sequence: Synthetic primer
  source 1..31: mol_type = other DNA
ctcgagtttggtcctagcagaggctgtgacc

SEQ ID NO: 57 - DNA, 37 bp, synthetic construct; identical to SEQ ID NO: 20
  misc_feature 1..37: note = Description of Artificial Sequence: Synthetic primer
  source 1..37: mol_type = other DNA

SEQ ID NO: 58 - DNA, 43 bp, synthetic construct; identical to SEQ ID NO: 21
  misc_feature 1..43: note = Description of Artificial Sequence: Synthetic primer
  source 1..43: mol_type = other DNA

SEQ ID NO: 59 - DNA, 45 bp, synthetic construct; identical to SEQ ID NO: 22
  misc_feature 1..45: note = Description of Artificial Sequence: Synthetic primer
  source 1..45: mol_type = other DNA

SEQ ID NO: 60 - DNA, 44 bp, synthetic construct; identical to SEQ ID NO: 23
  misc_feature 1..44: note = Description of Artificial Sequence: Synthetic primer
  source 1..44: mol_type = other DNA

SEQ ID NO: 61 - DNA, 42 bp, synthetic construct; identical to SEQ ID NO: 1
  misc_feature 1..42: note = Description of Artificial Sequence: Synthetic primer
  source 1..42: mol_type = other DNA

SEQ ID NO: 62 - DNA, 40 bp, synthetic construct; identical to SEQ ID NO: 3
  misc_feature 1..40: note = Description of Artificial Sequence: Synthetic primer
  source 1..40: mol_type = other DNA

SEQ ID NO: 63 - DNA, 58 bp, synthetic construct; identical to SEQ ID NO: 9
  misc_feature 1..58: note = Description of Artificial Sequence: Synthetic primer
  source 1..58: mol_type = other DNA

SEQ ID NO: 64 - DNA, 53 bp, synthetic construct
  misc_feature 1..53: note = Description of Artificial Sequence: Synthetic primer
  source 1..53: mol_type = other DNA
agatctcgagcccagaggtcccacaatcaagccctctcctccatgcaaatgcc

SEQ ID NO: 65 - DNA, 45 bp, synthetic construct; identical to SEQ ID NO: 10
  misc_feature 1..45: note = Description of Artificial Sequence: Synthetic primer
  source 1..45: mol_type = other DNA

SEQ ID NO: 66 - DNA, 39 bp, synthetic construct
  misc_feature 1..39: note = Description of Artificial Sequence: Synthetic primer
  source 1..39: mol_type = other DNA
atcaagccctctcctccatctaaatccccagcacctaac

SEQ ID NO: 67 - DNA, 57 bp, synthetic construct
  misc_feature 1..57: note = Description of Artificial Sequence: Synthetic primer
  source 1..57: mol_type = other DNA
agtggcaaggagttcaaatgctcggtcaagaagaaagacctcccagcgtccatcgag

SEQ ID NO: 68 - DNA, 55 bp, synthetic construct
  misc_feature 1..55: note = Description of Artificial Sequence: Synthetic primer
  source 1..55: mol_type = other DNA
ggttctctcgatggacgctgggaggtctttgttgttgaccgagcatttgaactcc

SEQ ID NO: 69 - DNA, 48 bp, synthetic construct; identical to SEQ ID NO: 11
  misc_feature 1..48: note = Description of Artificial Sequence: Synthetic primer
  source 1..48: mol_type = other DNA

SEQ ID NO: 70 - DNA, 37 bp, synthetic construct
  misc_feature 1..37: note = Description of Artificial Sequence: Synthetic primer
  source 1..37: mol_type = other DNA
gctagctccgtcgactttacccggagacagagagagg

SEQ ID NO: 71 - DNA, 51 bp, synthetic construct
  misc_feature 1..51: note = Description of Artificial Sequence: Synthetic primer
  source 1..51: mol_type = other DNA
gactggctgaatggcaaggagtacaagtgctcggtctccaacaaagccctc

SEQ ID NO: 72 - DNA, 51 bp, synthetic construct
  misc_feature 1..51: note = Description of Artificial Sequence: Synthetic primer
  source 1..51: mol_type = other DNA
gagggctttgttggagaccgagcacttgtaagacttgccattcagccagtc

SEQ ID NO: 73 - DNA, 45 bp, synthetic construct
  misc_feature 1..45: note = Description of Artificial Sequence: Synthetic primer
  source 1..45: mol_type = other DNA
ccgcgggaggagcagtacagcagcacgtaccgtgtggtcagcgtc

SEQ ID NO: 74 - DNA, 45 bp, synthetic construct
  misc_feature 1..45: note = Description of Artificial Sequence: Synthetic primer
  source 1..45: mol_type = other DNA
gacgctgaccacacggtacgtgctgctgtactgctcctcccgcgg

SEQ ID NO: 75 - DNA, 45 bp, synthetic construct
  misc_feature 1..45: note = Description of Artificial Sequence: Synthetic primer
  source 1..45: mol_type = other DNA
gatatctctagatttacccggagtccgagagaagctcttagtcgt

SEQ ID NO: 76 - DNA, 47 bp, synthetic construct
  misc_feature 1..47: note = Description of Artificial Sequence: Synthetic primer
  source 1..47: mol_type = other DNA
gatatctccggagtcgactttacccggagtccgagagaagctcttag

SEQ ID NO: 77 - DNA, 44 bp, synthetic construct
  misc_feature 1..44: note = Description of Artificial Sequence: Synthetic primer
  source 1..44: mol_type = other DNA
cacaaacccatagagaggattacagcagtactctccgggtggtc

SEQ ID NO: 78 - DNA, 44 bp, synthetic construct
  misc_feature 1..44: note = Description of Artificial Sequence: Synthetic primer
  source 1..44: mol_type = other DNA
gaccacccggagagtactgctgtaatcctctctatgggtttgag

SEQ ID NO: 79 - skipped

SEQ ID NO: 80 - DNA, 51 bp, synthetic construct
  misc_feature 1..51: note = Description of Artificial Sequence: Synthetic primer
  source 1..51: mol_type = other DNA
gatatcaccggtagaaccacctccaccactcccacctcctccagtgcctcc

SEQ ID NO: 81 - DNA, 50 bp, synthetic construct
  misc_feature 1..50: note = Description of Artificial Sequence: Synthetic primer
  source 1..50: mol_type = other DNA
gtcgactccggaggaggtggctcaggtggtggaggcagtggaggaggtgg

SEQ ID NO: 82 - DNA, 48 bp, synthetic construct
  misc_feature 1..48: note = Description of Artificial Sequence: Synthetic primer
  source 1..48: mol_type = other DNA
aaagtcgacggagctagcagccccgtgaacgtgagcagccccagcgtg

SEQ ID NO: 83 - DNA, 30 bp, synthetic construct
  misc_feature 1..30: note = Description of Artificial Sequence: Synthetic primer
  source 1..30: mol_type = other DNA
cccatgatatcctgcacgctggggctgctc

SEQ ID NO: 84 - DNA, 41 bp, synthetic construct; identical to SEQ ID NO: 44
  misc_feature 1..41: note = Description of Artificial Sequence: Synthetic primer
  source 1..41: mol_type = other DNA

SEQ ID NO: 85 - DNA, 42 bp, synthetic construct
  misc_feature 1..42: note = Description of Artificial Sequence: Synthetic primer
  source 1..42: mol_type = other DNA
agatctttatcaggagcgtttgctctttgttttcttccttag

SEQ ID NO: 86 - DNA, 44 bp, synthetic construct
  misc_feature 1..44: note = Description of Artificial Sequence: Synthetic primer
  source 1..44: mol_type = other DNA
tctagattatcaggagcgattgcctttttttctctttttgagag

SEQ ID NO: 87 - DNA, 42 bp, synthetic construct; identical to SEQ ID NO: 24
  misc_feature 1..42: note = Description of Artificial Sequence: Synthetic primer
  source 1..42: mol_type = other DNA

SEQ ID NO: 88 - DNA, 39 bp, synthetic construct; identical to SEQ ID NO: 2
  misc_feature 1..39: note = Description of Artificial Sequence: Synthetic primer
  source 1..39: mol_type = other DNA

SEQ ID NO: 89 - DNA, 48 bp, synthetic construct; identical to SEQ ID NO: 4
  misc_feature 1..48: note = Description of Artificial Sequence: Synthetic primer
  source 1..48: mol_type = other DNA

SEQ ID NO: 90 - DNA, 42 bp, synthetic construct
  misc_feature 1..42: note = Description of Artificial Sequence: Synthetic primer
  source 1..42: mol_type = other DNA
tctagactatcacacagtagcatcaaagtggactggtacgta

SEQ ID NO: 91 - DNA, 33 bp, synthetic construct; identical to SEQ ID NO: 18
  misc_feature 1..33: note = Description of Artificial Sequence: Synthetic primer
  source 1..33: mol_type = other DNA

SEQ ID NO: 92 - DNA, 33 bp, synthetic construct; identical to SEQ ID NO: 19
  misc_feature 1..33: note = Description of Artificial Sequence: Synthetic primer
  source 1..33: mol_type = other DNA

SEQ ID NO: 93 - DNA, 52 bp, synthetic construct
  misc_feature 1..52: note = Description of Artificial Sequence: Synthetic primer
  source 1..52: mol_type = other DNA
ggctcaggtggtggaggatctggaggaggtggctcaggtggtggaggatctg

SEQ ID NO: 94 - DNA, 46 bp, synthetic construct
  misc_feature 1..46: note = Description of Artificial Sequence: Synthetic primer
  source 1..46: mol_type = other DNA
gttagatctctccggaggaggtggctcaggtggtggaggatctgga

SEQ ID NO: 95 - DNA, 46 bp, synthetic construct
  misc_feature 1..46: note = Description of Artificial Sequence: Synthetic primer
  source 1..46: mol_type = other DNA
ctcgagactcccacctcctccagatcctccaccacctgagccacct

SEQ ID NO: 96 - DNA, 50 bp, synthetic construct
  misc_feature 1..50: note = Description of Artificial Sequence: Synthetic primer
  source 1..50: mol_type = other DNA
aaagatctctccggaggaggtggctcaggtggtggaggatctggaggagg

SEQ ID NO: 97 - DNA, 50 bp, synthetic construct
  misc_feature 1..50: note = Description of Artificial Sequence: Synthetic primer
  source 1..50: mol_type = other DNA
ctcgagaccggtagaaccacctccaccactcccacctcctccagatcctc

SEQ ID NO: 98 - DNA, 28 bp, synthetic construct
  misc_feature 1..28: note = Description of Artificial Sequence: Synthetic primer
  source 1..28: mol_type = other DNA
gttagatctctccggaggaggtggctca

SEQ ID NO: 99 - DNA, 32 bp, synthetic construct
  misc_feature 1..32: note = Description of Artificial Sequence: Synthetic primer
  source 1..32: mol_type = other DNA
accggtctcgagactcccacctcctccagatc

SEQ ID NO: 100 - DNA, 82 bp, synthetic construct
  misc_feature 1..82: note = Description of Artificial Sequence: Synthetic oligonucleotide
  source 1..82: mol_type = other DNA
agatctctccggaggaggtggctcaggtggtggaggatctggaggaggtgggagtggtggaggtggttctaccggtctcgag

SEQ ID NO: 101 - DNA, 97 bp, synthetic construct
  misc_feature 1..97: note = Description of Artificial Sequence: Synthetic oligonucleotide
  source 1..97: mol_type = other DNA
agatctctccggaggaggtggctcaggtggtggaggatctggaggaggtggctcaggtggtggaggatctggaggaggtgggagtaccggtctcgag

SEQ ID NO: 102 - DNA, 91 bp, synthetic construct
  misc_feature 1..91: note = Description of Artificial Sequence: Synthetic oligonucleotide
  source 1..91: mol_type = other DNA
agatctctccggaggaggtggctcaggtggtggaggatctggaggaggtggctcaggtggtggaggatctggaggaggtgggagtctcgag

SEQ ID NO: 103 - DNA, 462 bp, synthetic construct
  misc_feature 1..462: note = Description of Artificial Sequence: Synthetic polynucleotide
  source 1..462: mol_type = other DNA
gtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacgactccaggtaccccaactgtgcataccggaccagcccgaaggagagacacatcattgtggcctgtgaagggagcccatatgtgccagtccactttgatgcttctgtggaggactctacctaataatctaga

SEQ ID NO: 104 - DNA, 798 bp, synthetic construct
  misc_feature 1..798: note = Description of Artificial Sequence: Synthetic polynucleotide
  source 1..798: mol_type = other DNA
gatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgataatctaga

SEQ ID NO: 105 - DNA, 798 bp, Homo sapiens
  source 1..798: mol_type = genomic DNA
gatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaatgataatctaga

SEQ ID NO: 106 - DNA, 798 bp, synthetic construct
  misc_feature 1..798: note = Description of Artificial Sequence: Synthetic polynucleotide
  source 1..798: mol_type = other DNA
gatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttagaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgataatctaga

SEQ ID NO: 107 - DNA, 795 bp, synthetic construct
  misc_feature 1..795: note = Description of Artificial Sequence: Synthetic polynucleotide
  source 1..795: mol_type = other DNA
accggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgag

SEQ ID NO: 108 - DNA, 795 bp, Homo sapiens
  source 1..795: mol_type = genomic DNA
accggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgag

SEQ ID NO: 109 - DNA, 795 bp, synthetic construct
  misc_feature 1..795: note = Description of Artificial Sequence: Synthetic polynucleotide
  source 1..795: mol_type = other DNA
accggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttagaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgag

SEQ ID NO: 110 - DNA, 715 bp, Homo sapiens
  source 1..715: mol_type = genomic DNA
agatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 111 - DNA, 858 bp, synthetic construct
  misc_feature 1..858: note = Description of Artificial Sequence: Synthetic polynucleotide
  source 1..858: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 112 - DNA, 918 bp, Homo sapiens
  source 1..918: mol_type = genomic DNA
atgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacatacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcctag

SEQ ID NO: 113 - DNA, 459 bp, Homo sapiens
  source 1..459: mol_type = genomic DNA
atgggtctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgctactgtgtag

SEQ ID NO: 114 - DNA, 1162 bp, synthetic construct
  misc_feature 1..1162: note = Description of Artificial Sequence: Synthetic polynucleotide
  source 1..1162: mol_type = other DNA
gttaagcttgccaccatggaaaccccagcgcagcttctcttcctcctgctactctggctcccagataccaccggtagggaatctgcagcacagaagtttcagcggcagcacatggatccagatggttcctccatcaacagccccacctactgcaaccaaatgatgaaacgccgggatatgacaaatgggtcatgcaagcccgtgaacaccttcgtgcatgagcccttggcagatgtccaggccgtctgctcccaggaaaatgtcacctgcaagaacaggaagagcaactgctacaagagcagctctgccctgcacatcactgactgccacctgaagggcaactccaagtatcccaactgtgactacaagaccactcaataccagaagcacatcattgtggcctgtgaagggaacccctacgtaccagtccactttgatgctactgtgctcgagcccagaggtctcacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctagaa

SEQ ID NO: 115 - DNA, 2004 bp, synthetic construct
  misc_feature 1..2004: note = Description of Artificial Sequence: Synthetic polynucleotide
  unsure 1921: note = a, c, t, g, unknown or other
  source 1..2004: mol_type = other DNA
aagcttgccgccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggctcggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccagnctgcctatggcctgagtgaccaactggcccaagccatcagtgaccactatccagtggaggtgatgctgaagtgataatctaga

SEQ ID NO: 116 - DNA, 2004 bp, synthetic construct
  misc_feature 1..2004: note = Description of Artificial Sequence: Synthetic polynucleotide
  source 1..2004: mol_type = other DNA
aagcttgccgccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttagaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgataatctaga

SEQ ID NO: 117 - DNA, 2004 bp, synthetic construct
  misc_feature 1..2004: note = Description of Artificial Sequence: Synthetic polynucleotide
  source 1..2004: mol_type = other DNA
aagcttgccgccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaatgataatctaga

SEQ ID NO: 118 - DNA, 1569 bp, synthetic construct
  misc_feature 1..1569: note = Description of Artificial Sequence: Synthetic polynucleotide
  source 1..1569: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 119 - DNA, 1569 bp, synthetic construct
  misc_feature 1..1569: note = Description of Artificial Sequence: Synthetic polynucleotide
  source 1..1569: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttagaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 120 - DNA, 1641 bp, synthetic construct
  misc_feature 1..1641: note = Description of Artificial Sequence: Synthetic polynucleotide
  source 1..1641: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 121 - DNA, 1656 bp, synthetic construct
  misc_feature 1..1656: note = Description of Artificial Sequence: Synthetic polynucleotide
  source 1..1656: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtggctcaggtggtggaggatctggaggaggtgggagtaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 122 - DNA, 1650 bp, synthetic construct
  misc_feature 1..1650: note = Description of Artificial Sequence: Synthetic polynucleotide
  source 1..1650: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtggctcaggtggtggaggatctggaggaggtgggagtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 123 - DNA, 1569 bp, synthetic construct
  misc_feature 1..1569: note = Description of Artificial Sequence: Synthetic polynucleotide
  source 1..1569: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 124 - DNA, 1173 bp, synthetic construct
  misc_feature 1..1173: note = Description of Artificial Sequence: Synthetic polynucleotide
  source 1..1173: mol_type = other DNA
gttaagcttgccaccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacgactccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 125 - DNA, 1245 bp, synthetic construct
  misc_feature 1..1245: note = Description of Artificial Sequence: Synthetic polynucleotide
  source 1..1245: mol_type = other DNA
gttaagcttgccaccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 126 - DNA, 1254 bp, synthetic construct
  misc_feature 1..1254: note = Description of Artificial Sequence: Synthetic polynucleotide
  source 1..1254: mol_type = other DNA
gttaagcttgccaccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtggctcaggtggtggaggatctggaggaggtgggagtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 127 - DNA, 1173 bp, synthetic construct
  misc_feature 1..1173: note = Description of Artificial Sequence: Synthetic polynucleotide
  source 1..1173: mol_type = other DNA
gttaagcttgccaccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 128 - DNA, 945 bp, synthetic construct
  misc_feature 1..945: note = Description of Artificial Sequence: Synthetic polynucleotide
  source 1..945: mol_type = other DNA
atgggctcacagaccctgccccatggtcacatgcagaccctcatcttcttagacctggaagccactggcctgccttcgtctcggcccgaagtcacagagctgtgcctgctggctgtccacagacgtgctctggagaacacttccatttctcagggacatccacctccagtgcccagaccgccccgtgtggtggacaagctctctctgtgcattgctccagggaaagcctgtagccctggggccagtgagatcacaggtctgagcaaagctgagctggaagtacaggggcgtcaacgcttcgatgacaacctggccatcctgctccgagccttcctgcagcgccagccacagccttgctgccttgtggcacacaacggtgaccgctatgactttcctctgctccagacagagcttgctaggctgagcactcccagtcccctagatggtaccttctgtgtggacagcatcgctgccctaaaggccttggaacaagctagcagcccctcagggaatggttcgaggaaaagctacagcctgggcagcatctacacccgcctgtactggcaagcaccgacagactcacatactgctgaaggtgatgttctaaccctgctcagcatctgtcagtggaagccacaggccctactgcagtgggtggacgaacatgcccggccctttagcaccgtcaagcccatgtacggcactccggctaccactggaacaaccaacctaaggccacatgctgccacagctactacacccctggccacagccaatggaagtcccagcaatggcaggagcaggcgacctaagagtcctcctccagagaaggtcccagaagccccatcacaggaggggctgctggccccactgagcctgctgaccctcctgaccttggcaatagccactctgtatggactcttcctggcctcacctgggcagtaa

SEQ ID NO: 129 - DNA, 735 bp, synthetic construct
  misc_feature 1..735: note = Description of Artificial Sequence: Synthetic polynucleotide
  source 1..735: mol_type = other DNA
atgggctcacagaccctgccccatggtcacatgcagaccctcatcttcttagacctggaagccactggcctgccttcgtctcggcccgaagtcacagagctgtgcctgctggctgtccacagacgtgctctggagaacacttccatttctcagggacatccacctccagtgcccagaccgccccgtgtggtggacaagctctctctgtgcattgctccagggaaagcctgtagccctggggccagtgagatcacaggtctgagcaaagctgagctggaagtacaggggcgtcaacgcttcgatgacaacctggccatcctgctccgagccttcctgcagcgccagccacagccttgctgccttgtggcacacaacggtgaccgctatgactttcctctgctccagacagagcttgctaggctgagcactcccagtcccctagatggtaccttctgtgtggacagcatcgctgccctaaaggccttggaacaagctagcagcccctcagggaatggttcgaggaaaagctacagcctgggcagcatctacacccgcctgtactggcaagcaccgacagactcacatactgctgaaggtgatgttctaaccctgctcagcatctgtcagtggaagccacaggccctactgcagtgggtggacgaacatgcccggccctttagcaccgtcaagcccatgtacggcactccggctaccactggaacaacagatctcgag

SEQ ID NO: 130 - DNA, 1593 bp, synthetic construct
  misc_feature 1..1593: note = Description of Artificial Sequence: Synthetic polynucleotide
  source 1..1593: mol_type = other DNA
aagcttgccaccatggaaaccccagcgcagcttctcttcctcctgctactctggctcccagataccaccggtatgggctcacagaccctgccccatggtcacatgcagaccctcatcttcttagacctggaagccactggcctgccttcgtctcggcccgaagtcacagagctgtgcctgctggctgtccacagacgtgctctggagaacacttccatttctcagggacatccacctccagtgcccagaccgccccgtgtggtggacaagctctctctgtgcattgctccagggaaagcctgtagccctggggccagtgagatcacaggtctgagcaaagctgagctggaagtacaggggcgtcaacgcttcgatgacaacctggccatcctgctccgagccttcctgcagcgccagccacagccttgctgccttgtggcacacaacggtgaccgctatgactttcctctgctccagacagagcttgctaggctgagcactcccagtcccctagatggtaccttctgtgtggacagcatcgctgccctaaaggccttggaacaagctagcagcccctcagggaatggttcgaggaaaagctacagcctgggcagcatctacacccgcctgtactggcaagcaccgacagactcacatactgctgaaggtgatgttctaaccctgctcagcatctgtcagtggaagccacaggccctactgcagtgggtggacgaacatgcccggccctttagcaccgtcaagcccatgtacggcactccggctaccactggaacaacagatctctccggaggaggtggctcaggtggtggaggatctggaggaggtggctcagggagtggtggaggtggttctaccggtctcgagcccagaggtcccacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctaga

SEQ ID NO: 131 - DNA, 1596 bp, synthetic construct
  misc_feature 1..1596: note = Description of Artificial Sequence: Synthetic polynucleotide
  source 1..1596: mol_type = other DNA
aagcttgccaccatggaaaccccagcgcagcttctcttcctcctgctactctggctcccagataccaccggtatgggctcacagaccctgccccatggtcacatgcagaccctcatcttcttagacctggaagccactggcctgccttcgtctcggcccgaagtcacagagctgtgcctgctggctgtccacagacgtgctctggagaacacttccatttctcagggacatccacctccagtgcccagaccgccccgtgtggtggacaagctctctctgtgcattgctccagggaaagcctgtagccctggggccagtgagatcacaggtctgagcaaagctgagctggaagtacaggggcgtcaacgcttcgatgacaacctggccatcctgctccgagccttcctgcagcgccagccacagccttgctgccttgtggcacacaacggtgaccgctatgactttcctctgctccagacagagcttgctaggctgagcactcccagtcccctagatggtaccttctgtgtggacagcatcgctgccctaaaggccttggaacaagctagcagcccctcagggaatggttcgaggaaaagctacagcctgggcagcatctacacccgcctgtactggcaagcaccgacagactcacatactgctgaaggtgatgttctaaccctgctcagcatctgtcagtggaagccacaggccctactgcagtgggtggacgaacatgcccggccctttagcaccgtcaagcccatgtacggcactccggctaccactggaacaacagatctctccggaggaggtggctcaggtggtggaggatctggaggaggtggctcaggtggtggaggatctggaggaggtgggagtctcgagcccagaggtcccacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctaga

SEQ ID NO: 132 - DNA, 57 bp, synthetic construct
  misc_feature 1..57: note = Description of Artificial Sequence: Synthetic oligonucleotide
  source 1..57: mol_type = other DNA
gtcgacggcgcggccgccagccccgtgaacgtgagcagccccagcgtgcaggatatc

SEQ ID NO: 133 - DNA, 2328 bp, synthetic construct
  misc_feature 1..2328: note = Description of Artificial Sequence: Synthetic polynucleotide
  source 1..2328: mol_type = other DNA
aagcttgccaccatggaaaccccagcgcagcttctcttcctcctgctactctggctcccagataccaccggtatgggctcacagaccctgccccatggtcacatgcagaccctcatcttcttagacctggaagccactggcctgccttcgtctcggcccgaagtcacagagctgtgcctgctggctgtccacagacgtgctctggagaacacttccatttctcagggacatccacctccagtgcccagaccgccccgtgtggtggacaagctctctctgtgcattgctccagggaaagcctgtagccctggggccagtgagatcacaggtctgagcaaagctgagctggaagtacaggggcgtcaacgcttcgatgacaacctggccatcctgctccgagccttcctgcagcgccagccacagccttgctgccttgtggcacacaacggtgaccgctatgactttcctctgctccagacagagcttgctaggctgagcactcccagtcccctagatggtaccttctgtgtggacagcatcgctgccctaaaggccttggaacaagctagcagcccctcagggaatggttcgaggaaaagctacagcctgggcagcatctacacccgcctgtactggcaagcaccgacagactcacatactgctgaaggtgatgttctaaccctgctcagcatctgtcagtggaagccacaggccctactgcagtgggtggacgaacatgcccggccctttagcaccgtcaagcccatgtacggcactccggctaccactggaacaacagatctcatgggctcacagaccctgccccatggtcacatgcagaccctcatcttcttagacctggaagccactggcctgccttcgtctcggcccgaagtcacagagctgtgcctgctggctgtccacagacgtgctctggagaacacttccatttctcagggacatccacctccagtgcccagaccgccccgtgtggtggacaagctctctctgtgcattgctccagggaaagcctgtagccctggggccagtgagatcacaggtctgagcaaagctgagctggaagtacaggggcgtcaacgcttcgatgacaacctggccatcctgctccgagccttcctgcagcgccagccacagccttgctgccttgtggcacacaacggtgaccgctatgactttcctctgctccagacagagcttgctaggctgagcactcccagtcccctagatggtaccttctgtgtggacagcatcgctgccctaaaggccttggaacaagctagcagcccctcagggaatggttcgaggaaaagctacagcctgggcagcatctacacccgcctgtactggcaagcaccgacagactcacatactgctgaaggtgatgttctaaccctgctcagcatctgtcagtggaagccacaggccctactgcagtgggtggacgaacatgcccggccctttagcaccgtcaagcccatgtacggcactccggctaccactggaacaacagatctctccggaggaggtggctcaggtggtggaggatctggaggaggtggctcaggtggtggaggatctggaggaggtgggagtctcgagcccagaggtcccacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctaga

SEQ ID NO: 134 - DNA, 1749 bp, synthetic construct
  misc_feature 1..1749: note = Description of Artificial Sequence: Synthetic polynucleotide
  source 1..1749: mol_type = other DNA
aagcttgccaccatggaaaccccagcgcagcttctcttcctcctgctactctggctcccagataccaccggtatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 135 - DNA, 1758 bp, synthetic construct
  misc_feature 1..1758: note = Description of Artificial Sequence: Synthetic polynucleotide
  source 1..1758: mol_type = other DNA
aagcttgccaccatggaaaccccagcgcagcttctcttcctcctgctactctggctcccagataccaccggtatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaagatctctccggaggaggtggctcaggtggtggaggatctggaggaggtggctcaggtggtggaggatctggaggaggtgggagtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 136 - protein, 20 aa, synthetic construct
  REGION 1..20: note = Description of Artificial Sequence: Synthetic peptide
GGGGSGGGGSGGGGSGGGGS

SEQ ID NO: 137 - protein, 25 aa, synthetic construct
  REGION 1..25: note = Description of Artificial Sequence: Synthetic peptide
GGGGSGGGGSGGGGSGGGGSGGGGS

SEQ ID NO: 138 - protein, 25 aa, synthetic construct
  REGION 1..25: note = Description of Artificial Sequence: Synthetic peptide
GGGGSGGGGSGGGGSGGGGSGGGGS

SEQ ID NO: 139 - protein, 260 aa, synthetic construct
  REGION 1..260: note = Description of Artificial Sequence: Synthetic polypeptide
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 140 - protein, 260 aa, Homo sapiens
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 141 - protein, 260 aa, synthetic construct
  REGION 1..260: note = Description of Artificial Sequence: Synthetic polypeptide
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 142 - protein, 260 aa, synthetic construct
  REGION 1..260: note = Description of Artificial Sequence: Synthetic polypeptide
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 143 - protein, 260 aa, Homo sapiens; identical to SEQ ID NO: 140

SEQ ID NO: 144 - protein, 260 aa, synthetic construct; identical to SEQ ID NO: 141
  REGION 1..260: note = Description of Artificial Sequence: Synthetic polypeptide

SEQ ID NO: 145 - protein, 233 aa, Homo sapiens
LEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 128 aa, synthetic construct
  REGION 1..128: note = Description of Artificial Sequence: Synthetic polypeptide
KESRAKKFQRQHMDSDSSPSSSSTYCNQMMRRRNMTQGRCKPVNTFVHEPLVDVQNVCFQEKVTCKNGQGNCYKSNSSMHITDCRLTNDSRYPNCAYRTSPKERHIIVACEGSPYVPVHFDASVEDST

SEQ ID NO: 147 - protein, 280 aa, synthetic construct
  REGION 1..280: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 148 - protein, 305 aa, synthetic construct
  REGION 1..305: note = Description of Artificial Sequence: Synthetic polypeptide
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 149 - protein, 156 aa, Homo sapiens
MGLEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 150 - protein, 378 aa, synthetic construct
  REGION 1..378: note = Description of Artificial Sequence: Synthetic polypeptide
METPAQLLFLLLLWLPDTTGRESAAQKFQRQHMDPDGSSINSPTYCNQMMKRRDMTNGSCKPVNTFVHEPLADVQAVCSQENVTCKNRKSNCYKSSSALHITDCHLKGNSKYPNCDYKTTQYQKHIIVACEGNPYVPVHFDATVLEPRGLTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 151 - protein, 659 aa, synthetic construct
  REGION 1..659: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGSEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 152 - protein, 659 aa, synthetic construct
  REGION 1..659: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 153 - protein, 659 aa, synthetic construct
  REGION 1..659: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 154 - protein, 513 aa, synthetic construct
  REGION 1..513: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 513 aa, synthetic construct
  REGION 1..513: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 533 aa, synthetic construct
  REGION 1..533: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 538 aa, synthetic construct
  REGION 1..538: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 538 aa, synthetic construct; identical to SEQ ID NO: 157
  REGION 1..538: note = Description of Artificial Sequence: Synthetic polypeptide

SEQ ID NO: 159 - protein, 513 aa, synthetic construct
  REGION 1..513: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 381 aa, synthetic construct
  REGION 1..381: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNDSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 401 aa, synthetic construct
  REGION 1..401: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 406 aa, synthetic construct
  REGION 1..406: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 381 aa, synthetic construct
  REGION 1..381: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 314 aa, synthetic construct
  REGION 1..314: note = Description of Artificial Sequence: Synthetic polypeptide
MGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTNLRPHAATATTPLATANGSPSNGRSRRPKSPPPEKVPEAPSQEGLLAPLSLLTLLTLAIATLYGLFLASPGQ

SEQ ID NO: 165 - protein, 245 aa, Mus sp.
MGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTDLE

SEQ ID NO: 166 - protein, 516 aa, synthetic construct
  REGION 1..516: note = Description of Artificial Sequence: Synthetic polypeptide
METPAQLLFLLLLWLPDTTGMGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTGGGGSGGGGSGGGGSGGGGSLEPRGPTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 167 - protein, 521 aa, synthetic construct
  REGION 1..521: note = Description of Artificial Sequence: Synthetic polypeptide
METPAQLLFLLLLWLPDTTGMGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTGGGGSGGGGSGGGGSGGGGSGGGGSLEPRGPTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 168 - protein, 18 aa, synthetic construct
  REGION 1..18: note = Description of Artificial Sequence: Synthetic peptide
VDGASSPVNVSSPSVQDI

SEQ ID NO: 169 - protein, 763 aa, synthetic construct
  REGION 1..763: note = Description of Artificial Sequence: Synthetic polypeptide
METPAQLLFLLLLWLPDTTGMGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTMGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTGGGGSGGGGSGGGGSGGGGSGGGGSLEPRGPTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 170 - protein, 570 aa, synthetic construct
  REGION 1..570: note = Description of Artificial Sequence: Synthetic polypeptide
METPAQLLFLLLLWLPDTTGMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 575 aa, synthetic construct
  REGION 1..575: note = Description of Artificial Sequence: Synthetic polypeptide
METPAQLLFLLLLWLPDTTGMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKGGGGSGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - DNA, 2091 bp, synthetic construct
  misc_feature 1..2091: note = Description of Artificial Sequence: Synthetic polynucleotide
  source 1..2091: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 173 - protein, 679 aa, synthetic construct
  REGION 1..679: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 174 - DNA, 1185 bp, synthetic construct
  misc_feature 1..1185: note = Description of Artificial Sequence: Synthetic polynucleotide
  source 1..1185: mol_type = other DNA
gttaagcttgccaccatggaaaccccagcgcagcttctcttcctcctgctactctggctcccagataccaccggtccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacgactccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgtccagcacctgaactcctgggtggatcgtcagtcttcctcttccccccaaaacccaaggacactctcatgatctcccggacccctgaggtcacgtgcgtggtggtggacgtgagccaggaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaagccctcccagcctccatcgagaaaacaatctccaaaaccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaacaccacgcctcccgtgctggactccgacggctccttctccctctacagcaagctcaccgtggacaagagcaggtggcagcaggggaacgtcttctcatgctccgtgatgcatgaggctctgcacaaccactacacgcagaagagcctctctctgtctccgggtaaatgataatctaga

SEQ ID NO: 175 - protein, 381 aa, synthetic construct
  REGION 1..381: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNDSRYPNCAYRTSPKERHIIVACEGSPYVPVHFDASVEDSTLEPKSSDKTHTCPPCPAPELLGGSSVFLFPPKPKDTLMISRTPEVTCVVVDVSQEDPEVQFNWYVDGMEVHNAKTKPREEQFNSTFRVVSVLTVVHQDWLNGKEYKCKVSNKALPASIEKTISKTKGQPREPQVYTLPPSREEMTKNQVSLTCLVKGFYPSDIAVEWESNGQPENNYNTTPPVLDSDGSFSLYSKLTVDKSRWQQGNVFSCSVMHEALHNHYTQKSLSLSPGK

SEQ ID NO: 176 - DNA, 2106 bp, synthetic construct
  misc_feature 1..2106: note = Description of Artificial Sequence: Synthetic polynucleotide
  source 1..2106: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtggctcaggtggtggaggatctggaggaggtgggagtaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 177 - protein, 684 aa, synthetic construct
  REGION 1..684: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 178 - DNA, 2100 bp, synthetic construct
  misc_feature 1..2100: note = Description of Artificial Sequence: Synthetic polynucleotide
  source 1..2100: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtggctcaggtggtggaggatctggaggaggtgggagtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 179 - protein, 684 aa, synthetic construct; identical to SEQ ID NO: 177
  REGION 1..684: note = Description of Artificial Sequence: Synthetic polypeptide

SEQ ID NO: 180 - DNA, 2019 bp, synthetic construct
  misc_feature 1..2019: note = Description of Artificial Sequence: Synthetic polynucleotide
  source 1..2019: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 181 - protein, 659 aa, synthetic construct
  REGION 1..659: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 182 - DNA, 1652 bp, synthetic construct
  misc_feature 1..1652: note = Description of Artificial Sequence: Synthetic polynucleotide
  source 1..1652: mol_type = other DNA
gaccaagcttgccaccatggaaaccccagcgcagcttctcttcctcctgctactctggctcccagataccaccggtctaaggctctgctccttcaatgtgaggtcctttggagcgagcaagaaggaaaaccatgaagccatggatatcattgtgaagatcatcaaacgctgtgaccttatactgttgatggaaatcaaggacagcagcaacaacatctgtcccatgctgatggagaagctgaatggaaattcacgaagaagcacaacatacaactatgtgattagttctcgacttggaagaaacacgtacaaagagcagtatgccttcgtctacaaggagaagctggtgtctgtgaagacaaaataccactaccatgactatcaggatggagacacagacgtgttttccagggagccctttgtggtttggttccattccccctttactgctgtcaaggacttcgtgattgtccccttgcacacaactcccgagacctccgttaaagagatagatgagctggtcgatgtctacacggatgtgagaagccagtggaagacagagaatttcatcttcatgggtgatttcaacgccggctgtagctatgtccccaagaaggcctggcagaacattcgtttgaggacggaccccaagtttgtttggctgattggggaccaagaggacactacggtcaagaagagtaccagctgtgcctatgacaggattgtgctttgtggacaagagatagtcaactccgtggttccccgttccagtggcgtctttgactttcagaaagcttatgacttgtctgaagaggaggccctggatgtcagtgatcactttccagttgagtttaagctacagtcttcaagggccttcaccaacaacagaaaatctgtttctctcaaaaagagaaaaaaaggcaatcgctcctcagatctcgagcccagaggtctcacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctagaa

SEQ ID NO: 183 - protein, 541 aa, synthetic construct
  REGION 1..541: note = Description of Artificial Sequence: Synthetic polypeptide
METPAQLLFLLLLWLPDTTGLRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSDLEPRGLTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 184 - DNA, 1667 bp, synthetic construct
  misc_feature 1..1667: note = Description of Artificial Sequence: Synthetic polynucleotide
  unsure 826: note = a, c, t, g, unknown or other
  unsure 832: note = a, c, t, g, unknown or other
  source 1..1667: mol_type = other DNA
gagaccagcttgccccatgtccctgcacccagcttccccacgcctggcctccctgctgctcttcatccttgccctccatgacaccctggccctaaggctctgctccttcaatgtgaggtcctttggagcgagcaagaaggaaaaccatgaagccatggatatcattgtgaagatcatcaaacgctgtgaccttatactgttgatggaaatcaaggacagcagcaacaacatctgtcccatgctgatggagaagctgaatggaaattcacgaagaagcacaacatacaactatgtgattagttctcgacttggaagaaacacgtacaaagagcagtatgccttcgtctacaaggagaagctggtgtctgtgaagacaaaataccactaccatgactatcaggatggagacacagacgtgttttccagggagccctttgtggtttggttccattccccctttactgctgtcaaggacttcgtgattgtccccttgcacacaactcccgagacctccgttaaagagatagatgagctggtcgatgtctacacggatgtgagaagccagtggaagacagagaatttcatcttcatgggtgatttcaacgccggctgtagctatgtccccaagaaggcctggcagaacattcgtttgaggacggaccccaagtttgtttggctgattggggaccaagaggacactacggtcaagaagagtaccagctgtgcctatgacaggattgtgctttgtggacaagagatagtcaactccgtggttccccgttccagtggcgtctttgactttcagaaagcttatgacttgtctgangaggangccctggatgtcagtgatcactttccagttgagtttaagctacagtcttcaagggccttcaccaacaacagaaaatctgtttctctcaaaaagagaaaaaaaggcaatcgctcctcagatctcgagcccagaggtctcacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctagaa

SEQ ID NO: 185 - protein, 546 aa, synthetic construct
  REGION 1..546: note = Description of Artificial Sequence: Synthetic polypeptide
  MOD_RES 270: note = Any amino acid
  MOD_RES 272: note = Any amino acid
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSXEXALDVSDHFPVEFKLQSSRAFTNNRKSVSLKKRKKGNRSSDLEPRGLTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 186 - DNA, 2094 bp, synthetic construct
  misc_feature 1..2094: note = Description of Artificial Sequence: Synthetic polynucleotide
  source 1..2094: mol_type = other DNA
gttaagcttgccaccatggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacatacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 187 - protein, 684 aa, synthetic construct
  REGION 1..684: note = Description of Artificial Sequence: Synthetic polypeptide
METPAQLLFLLLLWLPDTTGMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 188 - DNA, 1620 bp, synthetic construct
  misc_feature 1..1620: note = Description of Artificial Sequence: Synthetic polynucleotide
  source 1..1620: mol_type = other DNA
aagcttgccgccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 189 - protein, 527 aa, synthetic construct
  REGION 1..527: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSHVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 190 - DNA, 1692 bp, synthetic construct
  misc_feature 1..1692: note = Description of Artificial Sequence: Synthetic polynucleotide
  source 1..1692: mol_type = other DNA
aagcttgccgccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 191 - protein, 547 aa, synthetic construct
  REGION 1..547: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSHVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 192 - DNA, 2199 bp, synthetic construct
  misc_feature 1..2199: note = Description of Artificial Sequence: Synthetic polynucleotide
  source 1..2199: mol_type = other DNA
aagcttgccaccatggaaaccccagcgcagcttctcttcctcctgctactctggctcccagataccaccggtatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 193 - protein, 716 aa, synthetic construct
  REGION 1..716: note = Description of Artificial Sequence: Synthetic polypeptide
METPAQLLFLLLLWLPDTTGMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 194 - DNA, 2115 bp, synthetic construct
  misc_feature 1..2115: note = Description of Artificial Sequence: Synthetic polynucleotide
  source 1..2115: mol_type = other DNA
aagcttgccgccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaatgataatctaga

SEQ ID NO: 195 - protein, 696 aa, synthetic construct
  REGION 1..696: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSHVNVSSPSVQDIMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTK

SEQ ID NO: 196 - DNA, 2166 bp, synthetic construct
  misc_feature 1..2166: note = Description of Artificial Sequence: Synthetic polynucleotide
  source 1..2166: mol_type = other DNA
gttaagcttgccaccatggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacatacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctctctgtctccgggtaaagtcgacggtgctagcagccatgtgaatgtgagcagccctagcgtgcaggatatcccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaataatctaga

SEQ ID NO: 197 - protein, 704 aa, synthetic construct
  REGION 1..704: note = Description of Artificial Sequence: Synthetic polypeptide
METPAQLLFLLLLWLPDTTGMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKVDGASSHVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 198 - DNA, 2154 bp, synthetic construct
  misc_feature 1..2154: note = Description of Artificial Sequence: Synthetic polynucleotide
  source 1..2154: mol_type = other DNA
gttaagcttgccaccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacatacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcctaatgatctaga

SEQ ID NO: 199 - protein, 704 aa, synthetic construct
  REGION 1..704: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 200 - DNA, 2586 bp, synthetic construct
  misc_feature 1..2586: note = Description of Artificial Sequence: Synthetic polynucleotide
  source 1..2586: mol_type = other DNA
gttaagcttgccaccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaatgataatctaga

SEQ ID NO: 201 - protein, 848 aa, synthetic construct
  REGION 1..848: note = Description of Artificial Sequence: Synthetic polypeptide
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTK

SEQ ID NO: 202 - DNA, 2583 bp, synthetic construct
  misc_feature 1..2583: note = Description of Artificial Sequence: Synthetic polynucleotide
  unsure 2500: note = a, c, t, g, unknown or other
  source 1..2583: mol_type = other DNA
aagcttgccaccatggaaaccccagcgcagcttctcttcctcctgctactctggctcccagataccaccggtatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggctcggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccagnctgcctatggcctgagtgaccaactggcccaagccatcagtgaccactatccagtggaggtgatgctgaagtgataatctaga

SEQ ID NO: 203 - protein, 848 aa, synthetic construct
  REGION 1..848: note = Description of Artificial Sequence: Synthetic polypeptide
  MOD_RES 825: note = Any amino acid
METPAQLLFLLLLWLPDTTGMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGSEDVMLMGDFNAGCSYVRPSQWSSIRLWTSPTFQWLIPDSADTTATPTHCAYDRIVVAGMLLRGAVVPDSALPFNFQXAYGLSDQLAQAISDHYPVEVMLK

SEQ ID NO: 204 - DNA, 2190 bp, synthetic construct
  misc_feature 1..2190: note = Description of Artificial Sequence: Synthetic polynucleotide
  source 1..2190: mol_type = other DNA
gttaagcttgccaccatggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaatgataatctaga

SEQ ID NO: 205 - protein, 716 aa, synthetic construct
  REGION 1..716: note = Description of Artificial Sequence: Synthetic polypeptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIMGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTK

SEQ ID NO: 206 - DNA, 2589 bp, synthetic construct
  misc_feature 1..2589: note = Description of Artificial Sequence: Synthetic polynucleotide
  source 1..2589: mol_type = other DNA
gttaagcttgccaccatggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacatacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcagatctcgagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggagctagcagccccgtgaacgtgagcagccccagcgtgcaggatatcatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaaatgataatctaga

SEQ ID NO: 207 - protein, 854 aa, synthetic construct [sequence omitted]

SEQ ID NO: 208 - protein, 25 aa, synthetic construct
  REGION 1..25: note = Description of Artificial Sequence: Synthetic peptide
  REGION 1..25: note = misc_feature - This sequence may encompass 0 to 5 "GGGGS" repeating units
GGGGSGGGGSGGGGSGGGGSGGGGS

SEQ ID NO: 209 - protein, 25 aa, synthetic construct
  REGION 1..25: note = Description of Artificial Sequence: Synthetic peptide
GGGGSGGGGSGGGGSGGGGSGGGGS

SEQ ID NO: 210 - protein, 50 aa, synthetic construct
  REGION 1..50: note = Description of Artificial Sequence: Synthetic polypeptide
  REGION 1..50: note = misc_feature - This sequence may encompass 1 to 10 "GGGGS" repeating units
GGGGSGGGGSGGGGSGGGGSGGGGSGGGGSGGGGSGGGGSGGGGSGGGGS

SEQ ID NO: 211 - protein, 15 aa, synthetic construct
  REGION 1..15: note = Description of Artificial Sequence: Synthetic peptide
GGGGSGGGGSGGGGS

SEQ ID NO: 212 - protein, 20 aa, synthetic construct
  REGION 1..20: note = Description of Artificial Sequence: Synthetic peptide
GGGGSGGGGSGGGGSGGGGS

SEQ ID NO: 213 - protein, 5 aa, synthetic construct
  REGION 1..5: note = Description of Artificial Sequence: Synthetic peptide
GGGGS

SEQ ID NO: 214 - protein, 51 aa, synthetic construct
  REGION 1..51: note = Description of Artificial Sequence: Synthetic polypeptide
  REGION 2..51: note = misc_feature - This region may encompass 1 to 10 "GGGGS" repeating units
SGGGGSGGGGSGGGGSGGGGSGGGGSGGGGSGGGGSGGGGSGGGGSGGGGS

SEQ ID NO: 215 - protein, 16 aa, synthetic construct
  REGION 1..16: note = Description of Artificial Sequence: Synthetic peptide
SGGGGSGGGGSGGGGS

SEQ ID NO: 216 - protein, 21 aa, synthetic construct
  REGION 1..21: note = Description of Artificial Sequence: Synthetic peptide
SGGGGSGGGGSGGGGSGGGGS

SEQ ID NO: 217 - protein, 31 aa, synthetic construct
  REGION 1..31: note = Description of Artificial Sequence: Synthetic polypeptide
  REGION 2..31: note = misc_feature - This region may encompass 1 to 6 "GGGGS" repeating units
SGGGGSGGGGSGGGGSGGGGSGGGGSGGGGS

SEQ ID NO: 218 - protein, 25 aa, synthetic construct
  REGION 1..25: note = Description of Artificial Sequence: Synthetic peptide
  REGION 1..25: note = misc_feature - This sequence may encompass 3 to 5 "GGGGS" repeating units
GGGGSGGGGSGGGGSGGGGSGGGGS

SEQ ID NO: 219 - protein, 25 aa, synthetic construct
  REGION 1..25: note = Description of Artificial Sequence: Synthetic peptide
  REGION 1..25: note = misc_feature - This sequence may encompass 1 to 5 "GGGGS" repeating units
GGGGSGGGGSGGGGSGGGGSGGGGS

SEQ ID NO: 220 - DNA, 18 bp, synthetic construct
  misc_feature 1..18: note = Description of Artificial Sequence: Synthetic oligonucleotide
  misc_feature 1..18: note = This sequence may encompass 1 to 12 repeating "t" nucleotides
  source 1..18: mol_type = other DNA
tttttttttttttttttt

SEQ ID NO: 221 - protein, 5 aa, synthetic construct
  REGION 1..5: note = Description of Artificial Sequence: Synthetic peptide
GGGGG

SEQ ID NO: 222 - DNA, 1177 bp, synthetic construct
  misc_feature 1..1177: note = Description of Artificial Sequence: Synthetic polynucleotide
  source 1..1177: mol_type = other DNA
  CDS 16..1164
gttaagcttgccaccatgggtctggagaagtccctcattctgtttccattgtttttcctgctgcttggatgggtccagccttccccgggcagggaatctgcagcacagaagtttcagcggcagcacatggatccagatggttcctccatcaacagccccacctactgcaaccaaatgatgaaacgccgggatatgacaaatgggtcatgcaagcccgtgaacaccttcgtgcatgagcccttggcagatgtccaggccgtctgctcccaggaaaatgtcacctgcaagaacaggaagagcaactgctacaagagcagctctgccctgcacatcactgactgccacctgaagggcaactccaagtatcccaactgtgactacaagaccactcaataccagaagcacatcattgtggcctgtgaagggaacccctacgtaccagtccactttgatgctactgtgctcgagcccagaggtctcacaatcaagccctctcctccatgcaaatgcccagcacctaacctcttgggtggatcatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgctcggtcaacaacaaagacctcccagcgtccatcgagagaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagagcttctctcggactccgggtaaatgataatctagaa

SEQ ID NO: 223 - protein, 383 aa, synthetic construct
  REGION 1..383: note = Description of Artificial Sequence: Synthetic polypeptide
MGLEKSLILFPLFFLLLGWVQPSPGRESAAQKFQRQHMDPDGSSINSPTYCNQMMKRRDMTNGSCKPVNTFVHEPLADVQAVCSQENVTCKNRKSNCYKSSSALHITDCHLKGNSKYPNCDYKTTQYQKHIIVACEGNPYVPVHFDATVLEPRGLTIKPSPPCKCPAPNLLGGSSVFIFPPKIKDVLMISLSPMVTCVVVDVSEDDPDVQISWFVNNVEVHTAQTQTHREDYNSTLRVVSALPIQHQDWMSGKEFKCSVNNKDLPASIERTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKSFSRTPGK

SEQ ID NO: 224 - protein, 28 aa, synthetic construct
  REGION 1..28: note = Description of Artificial Sequence: Synthetic peptide
MGLEKSLVRLLLLVLILLVLGWVQPSLG

SEQ ID NO: 225 - protein, 20 aa, synthetic construct
  REGION 1..20: note = Description of Artificial Sequence: Synthetic peptide
METPAQLLFLLLLWLPDTTG

(19) United States
(12) Patent Application Publication
(10) Pub. No.: US 2026/0035680 A1
(43) Pub. Date: Feb. 5, 2026

(54) THERAPEUTIC NUCLEASE COMPOSITIONS AND METHODS

(71) Applicant: University of Washington, Seattle, WA (US)

(72) Inventors: Jeffrey A. Ledbetter, Seattle, WA (US); Martha Hayden-Ledbetter, Seattle, WA (US); Keith Elkon, Seattle, WA (US); Xizhang Sun, Seattle, WA (US)

(21) Appl. No.: 19/294,688

(22) Filed: Aug. 8, 2025

Related U.S. Application Data
(63) Continuation of application No. 17/696,364, filed on Mar. 16, 2022, now Pat. No. 12,435,322.
Application No. 17/696,364 is a continuation of application No. 15/679,746, filed on Aug. 17, 2017, now Pat. No. 11,306,297.
Application No. 15/679,746 is a continuation of application No. 14/516,161, filed on Oct. 16, 2014, now Pat. No. 9,790,479.
Application No. 14/516,161 is a division of application No. 13/799,843, filed on Mar. 13, 2013, now abandoned.
Application No. 13/799,843 is a division of application No. 13/197,731, filed on Aug. 3, 2011, now Pat. No. 8,841,416.
Application No. 13/197,731 is a continuation of application No. PCT/US2010/055131, filed on Nov. 2, 2010.
(60) Provisional application No. 61/257,458, filed on Nov. 2, 2009.
(60) Provisional application No. 61/370,752, filed on Aug. 4, 2010.

(51) Int. Cl. C12N 9/22; A61K 38/00; C07K 16/18; C12N 9/96; C12N 11/06

(52) U.S. Cl. CPC C12N 9/22; C07K 16/18; C12N 9/96; C12N 11/06; A61K 38/00; C07K 2319/30; C12Y 301/27005; Y02P 20/582

(57) ABSTRACT

Hybrid nuclease molecules and methods for treating an immune-related disease or disorder in a mammal, and a pharmaceutical composition for treating an immune-related disease in a mammal.

### STATEMENT REGARDING FEDERALLY SPONSORED RESEARCH OR DEVELOPMENT

This invention was made with support from the National Institutes of Health (Grants AI44257, NS065933, and AR048796), the Alliance for Lupus Research, and the Washington State Life Science Discovery Fund (2087750). The government has certain rights in the invention.

### REFERENCE TO A SEQUENCE LISTING

This application includes a Sequence Listing submitted electronically as a text file named 19-551-WO-USCDC3.xml, created on Aug. 6, 2025, with a size of 398,896 bytes. The sequence listing is incorporated by reference.

### BACKGROUND

Excessive release of (ribo) nucleoprotein particles from dead and dying cells can cause lupus pathology by two mechanisms: (i) Deposition or in situ formation of chromatin/anti-chromatin complexes causes nephritis and leads to loss of renal function; and (ii) nucleoproteins activate innate immunity through toll-like receptor (TLR) 7, 8, and 9 as well as TLR-independent pathway(s). Release of nucleoproteins can serve as a potent antigen for autoantibodies in SLE, providing amplification of B cell and DC activation through co-engagement of antigen receptors and TLRs. Thus, there exists a need for a means to remove inciting antigens and/or attenuate immune stimulation, immune amplification, and immune complex mediated disease in subjects in need thereof.

### SUMMARY

Disclosed herein is a hybrid nuclease molecule comprising a first nuclease domain and an Fc domain, wherein the first nuclease domain is operatively coupled to the Fc domain. In some embodiments, the hybrid nuclease molecule further includes a first linker domain, and the first nuclease domain is operatively coupled to the Fc domain by the first linker domain.

In some embodiments, a hybrid nuclease molecule is a polypeptide, wherein the amino acid sequence of the first nuclease domain comprises a human, wild-type RNase amino acid sequence, wherein the first linker domain is (Gly4Ser)n, where n is 0, 1, 2, 3, 4 or 5, wherein the amino acid sequence of the Fc domain comprises a human, wild-type IgG1 Fc domain amino acid sequence, and wherein the first linker domain is coupled to the C-terminus of the first nuclease domain and the N-terminus of the Fc domain. In some embodiments, a hybrid nuclease molecule is a polypeptide comprising or consisting of a sequence shown in Table 2. In some embodiments, a hybrid nuclease molecule is a polypeptide comprising SEQ ID NO:149. In some embodiments, a hybrid nuclease molecule is a polypeptide comprising SEQ ID NO:145. In some embodiments, a hybrid nuclease molecule is a polypeptide comprising SEQ ID NO:161. In some embodiments, a hybrid nuclease molecule is a polypeptide comprising SEQ ID NO:162. In some embodiments, a hybrid nuclease molecule is a polypeptide comprising SEQ ID NO:163.

In some embodiments, a hybrid nuclease molecule comprises wild-type, human DNase1 linked to wild-type, human IgG1. In some embodiments, a hybrid nuclease molecule comprises human DNase1 G105R A114F linked to a wild-type, human IgG1 Fc domain by a (gly4ser) n linker domain where n=0, 1, 2, 3, 4, or 5. In some embodiments, a hybrid nuclease molecule comprises wild-type, human RNase1 linked to wild-type, human IgG1 linked to wild-type, human DNase1. In some embodiments, a hybrid nuclease molecule comprises wild-type, human RNase1 linked to wild-type, human IgG1 linked to human DNase1 G105R A114F. In some embodiments, a hybrid nuclease molecule is a polypeptide, wherein the amino acid sequence of the first nuclease domain comprises a RNase amino acid sequence, wherein the first linker domain is between 5 and 32 amino acids in length, wherein the amino acid sequence of the Fc domain comprises a human, Fc domain amino acid sequence, and wherein the first linker domain is coupled to the C-terminus of the first nuclease domain and the N-terminus of the Fc domain. In some embodiments, the linker domain includes (gly4ser)5 and restriction sites BgIII, AgeI, and XhoI. In some embodiments, a hybrid nuclease molecule is a polypeptide, wherein the amino acid sequence of the first nuclease domain comprises a human RNase amino acid sequence, wherein the first linker domain is a NLG peptide between 5 and 32 amino acids in length, wherein the amino acid sequence of the Fc domain comprises a human, wild-type Fc domain amino acid sequence, and wherein the first linker domain is coupled to the C-terminus of the first nuclease domain and the N-terminus of the Fc domain.

In some embodiments, the Fc domain binds to an Fc receptor on a human cell. In some embodiments, the serum half-life of the molecule is significantly longer than the serum half-life of the first nuclease domain alone. In some embodiments, the nuclease activity of the first nuclease domain of the molecule is the same or greater than the nuclease domain alone. In some embodiments, administration of the molecule to a mouse increases the survival rate of the mouse as measured by a mouse Lupus model assay.

In some embodiments, a hybrid nuclease molecule includes a leader sequence. In some embodiments, the leader sequence is human VK3LP peptide from the human kappa light chain family, and the leader sequence is coupled to the N-terminus of the first nuclease domain.

In some embodiments, the molecule is a polypeptide. In some embodiments, the molecule is a polynucleotide.

In some embodiments, the first nuclease domain comprises an RNase. In some embodiments, the RNase is a human RNase. In some embodiments, the RNase is a polypeptide comprising an amino acid sequence at least 90% similar to an RNase amino acid sequence set forth in Table 2. In some embodiments, the RNase is a human RNase A family member. In some embodiments, the RNase is a human pancreatic RNase1.

In some embodiments, the first nuclease domain comprises a DNase. In some embodiments, the DNase is a human DNase. In some embodiments, the DNase is a polypeptide comprising an amino acid sequence at least 90% similar to a DNase amino acid sequence set forth in Table 2. In some embodiments, the DNase is selected from the group consisting of human DNase I, TREX1, and human DNase 1L3.

In some embodiments, the Fc domain is a human Fc domain. In some embodiments, the Fc domain is a wild-type Fc domain. In some embodiments, the Fc domain is a mutant Fc domain. In some embodiments, the Fc domain is a human IgG1 Fc domain. In some embodiments, the Fc domain is a polypeptide comprising an amino acid sequence at least 90% similar to an Fc domain amino acid sequence set forth in Table 2.

In some embodiments, the first linker domain has a length of about 1 to about 50 amino acids. In some embodiments, the first linker domain has a length of about 5 to about 31 amino acids. In some embodiments, the first linker domain has a length of about 15 to about 25 amino acids. In some embodiments, the first linker domain has a length of about 20 to about 32 amino acids. In some embodiments, the first linker domain has a length of about 20 amino acids. In some embodiments, the first linker domain has a length of about 25 amino acids. In some embodiments, the first linker domain has a length of about 18 amino acids. In some embodiments, the first linker domain comprises a gly/ser peptide. In some embodiments, the gly/ser peptide is of the formula (Gly₄Ser)n, wherein n is a positive integer selected from the group consisting of 1, 2, 3, 4, 5, 6, 7, 8, 9, and 10. In some embodiments, the gly/ser peptide includes (Gly₄Ser)3. In some embodiments, the gly/ser peptide includes (Gly₄Ser)4. In some embodiments, the gly/ser peptide includes (Gly₄Ser)5. In some embodiments, the first linker domain includes at least one restriction site. In some embodiments, the first linker domain includes about 12 or greater nucleotides including at least one restriction site. In some embodiments, the first linker domain includes two or more restriction sites. In some embodiments, the first linker domain includes a plurality of restriction sites. In some embodiments, the first linker domain comprises an NLG peptide. In some embodiments, the first linker domain comprises an N-linked glycosylation site.

In some embodiments, the first nuclease domain is linked to the N-terminus of the Fc domain. In some embodiments, the first nuclease domain is linked to the C-terminus of the Fc domain.

In some embodiments, the hybrid nuclease molecule further includes a second nuclease domain. In some embodiments, the first and second nuclease domains are distinct nuclease domains. In some embodiments, the first and second nuclease domains are the same nuclease domains. In some embodiments, the second nuclease domain is linked to the C-terminus of the Fc domain. In some embodiments, the second nuclease domain is linked to the N-terminus of the Fc domain. In some embodiments, the second nuclease domain is linked to the C-terminus of the first nuclease domain. In some embodiments, the second nuclease domain is linked to the N-terminus of the first nuclease domain.

Also disclosed herein is a dimeric polypeptide comprising a first polypeptide and a second polypeptide, wherein the first polypeptide comprises a first nuclease domain, and an Fc domain, wherein the first nuclease domain is operatively coupled to the Fc domain. In some embodiments, the second polypeptide is a second hybrid nuclease molecule comprising a second nuclease domain, and a second Fc domain, wherein the second nuclease domain is operatively coupled to the second Fc domain.

Also disclosed herein is a pharmaceutical composition comprising at least one hybrid nuclease molecule and/or at least one dimeric polypeptide as described herein, and a pharmaceutically acceptable excipient.

Also disclosed herein is a nucleic acid molecule encoding a hybrid nuclease molecule disclosed herein. Also disclosed herein is a recombinant expression vector comprising a nucleic acid molecule disclosed herein. Also disclosed herein is a host cell transformed with a recombinant expression vector disclosed herein.

Also disclosed herein is a method of making a hybrid nuclease disclosed herein, comprising: providing a host cell comprising a nucleic acid sequence that encodes the hybrid nuclease molecule; and maintaining the host cell under conditions in which the hybrid nuclease molecule is expressed.

Also disclosed herein is a method for treating or preventing a condition associated with an abnormal immune response, comprising administering to a patient in need thereof an effective amount of an isolated hybrid nuclease molecule disclosed herein. In some embodiments, the condition is an autoimmune disease. In some embodiments, the autoimmune disease is selected from the group consisting of insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbs-ve, cryptogenic cirrhosis, ulcerative colitis, Sjogren's syndrome, scleroderma, Wegener's granulomatosis, polymyositis, dermatomyositis, discoid LE, systemic lupus erythematosus (SLE), and connective tissue disease. In some embodiments, the autoimmune disease is SLE.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows the nucleotide and amino acid sequence of the mRNase-mIgG2a with mutations at P238S, K322S, and P331S. This sequence is listed in the sequence listing as huVK3LP+mrib1+mIgG2A-C-2S (SEQ ID NO:114).

FIG. 2 shows a schematic diagram of some embodiments of hybrid nuclease molecules described herein.

FIG. 3 shows the SDS-PAGE gel analysis of mRNase-mIgG2a-c under both reducing and non-reducing conditions.

FIG. 4 shows gel immunoprecipitation analysis of mRNasemIg2a-c.

FIG. 5 shows an anti-RNA antibody ELISA titer before and after intravenous injection of RNase-Ig hybrid nuclease molecule from mouse 410. The data show that injection of RNase-Ig caused a reduction in titer of anti-RNA antibody that persisted for over 3 weeks.

FIG. 6 shows that RNase-Ig addition abolished the induction of interferon-a from human peripheral blood mononuclear cells stimulated using immune complexes formed with serum from an SLE patient (J11) plus nuclear extract (NE). Titer of anti-RNA antibody was reduced after injection of RNase-Ig.

FIG. 7 shows that RNase-Ig addition abolished the induction of interferon-a from human peripheral blood mononuclear cells stimulated using immune complexes formed with serum from an SLE patient (J11) plus nuclear extract.

FIG. 8 shows single radial enzyme diffusion (SRED) analysis of serum from two RNase transgenic (Tg) mice compared to a normal B6 mouse.

FIG. 9 shows the concentration of RNaseA in Tg and double Tg (DTg) mice measured by ELISA. Each dot represents the concentration measured in an individual mouse.

FIG. 10 shows survival of TLR7.1 Tg versus TLR7.1×RNaseA DTg mice FIG. 11 shows quantitative PCR of IRGs in spleens of Tg versus DTg mice.

FIG. 12 shows a prototype structure for creating different embodiments of hybrid nuclease molecules.

FIG. 13 shows the enzyme kinetics for hRNase1-G88D-hIgG1 SCCH-P238S-K322S-P331S hybrid nuclease molecules as measured using RNase Alert Substrate™.

FIG. 14 shows the binding of hRNase1-WT-hIgG1-WT to human monocytic cell lines U937 and THP1. The peak on the left in both plots is control and the peak on the right in both plots is hRNase 1-WT-hIgG1-WT.

FIG. 15 shows the blocking activity of human IVIg for binding to U937 and THP-1 cells by hRNase1-WT-hIgG1-WT.

FIG. 16 shows the results of a DNA digestion assay by Trex 1-(g4s) n-mIgG alternative forms.

FIG. 17 shows the results of a Western Blot for trex1-(Gly4S)4-Ig and trex1-(Gly4S)5-Ig culture supernatants from COS-7 transient transfections.

FIG. 18 shows DNA digestion patterns by different stably transfected CHO DG44 clones designated as 2A3, 3A5, and 8H8, expressing DNAse 1L3-mIgG2a-c hybrid nuclease molecules.

FIG. 19 shows DNA digestion patterns of decreasing amounts of DNase 1L3-Ig hybrid nuclease molecules after various incubation times with and without heparin as an enzyme inhibitor.

FIG. 20 shows a Western blot of immunoprecipitated fusion proteins from transiently transfected COS cells expressing different embodiments of hRNase1-Ig-hDNase1 or hDNase1-Ig hybrid nuclease molecules.

FIG. 21 shows SRED analysis to assess the presence of RNase activity in the COS supernatants expressing different embodiments of hRNase1-Ig-hDNase1 or hDNase1-Ig hybrid nuclease molecules.

FIG. 22 shows a composite figure displaying results of DNase nuclease activity assays performed on COS supernatants from transfected cells. The description of the numbering (e.g., 090210-8 and 091210-8) from FIG. 21 applies to this figure as well.

FIG. 23 shows enzyme kinetics assayed using the Rnase Alert Substrate (Ambion/IDT) and fluorescence quantified with a Spectramax M2 microplate Reader. Data was analyzed using Softmax Pro software (Molecular Devices). Reaction rates at different substrate concentrations were measured and the data shown as a Lineweaver-Burk plot. The apparent Km, corrected for volume is 280 nM.

FIG. 24 shows the levels of anti-RNA antibodies in mouse sera from H564 and H564-RNaseA double transgenic mice at successive intervals as the transgenic mice aged.

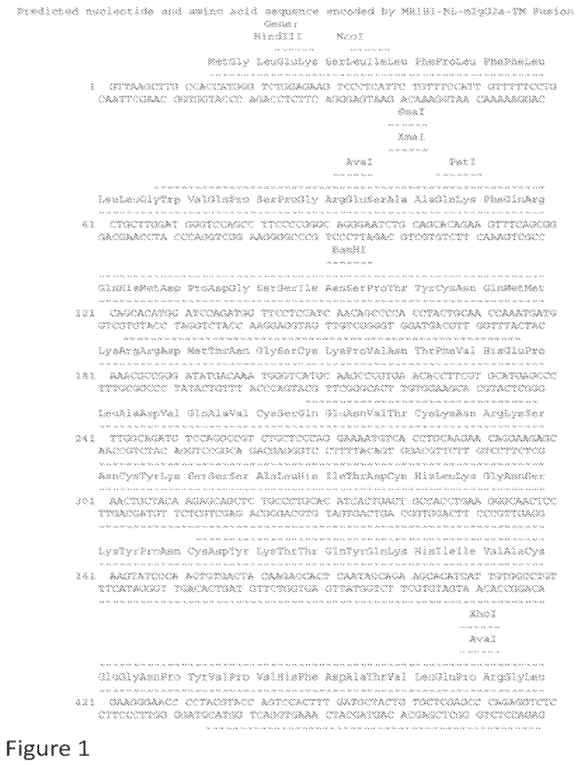

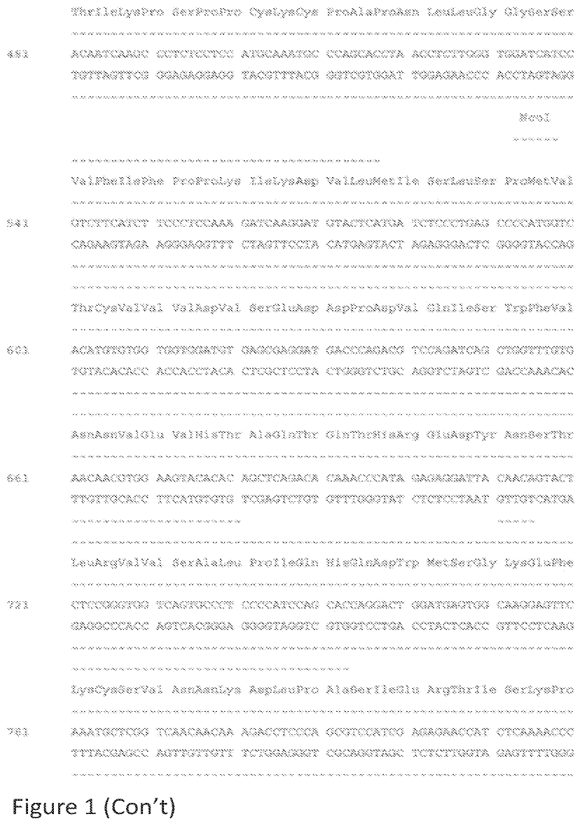

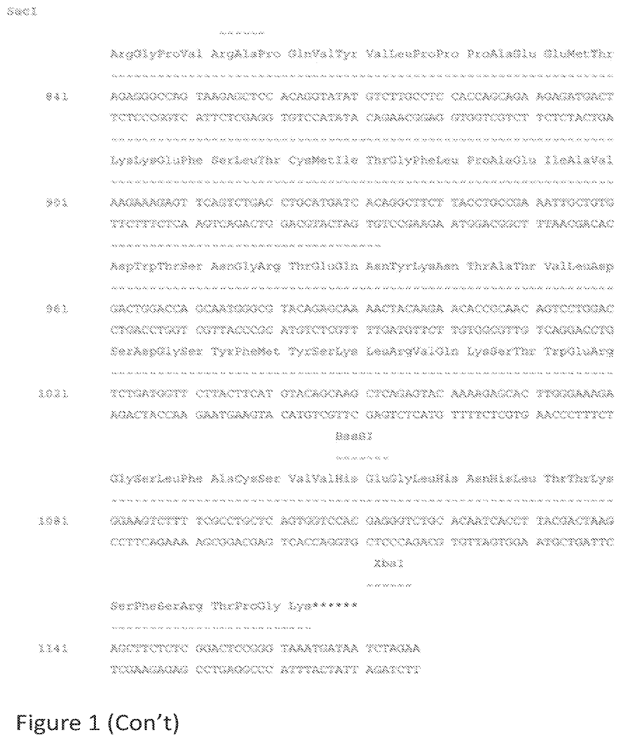

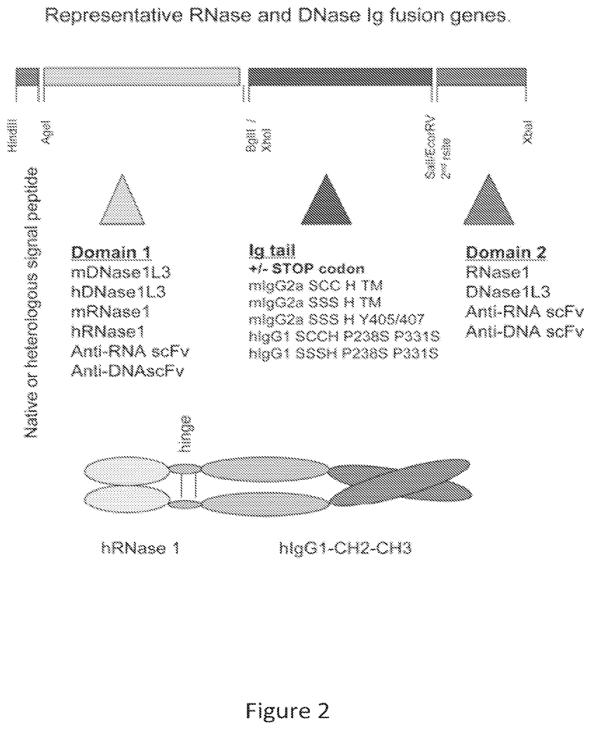

### DETAILED DESCRIPTION

Terms used in the claims and specification are defined as set forth below unless otherwise specified. In the case of direct conflict with a term used in a parent provisional patent application, the term used in the instant specification shall control.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “hybrid nuclease molecule” refers to polynucleotides or polypeptides that comprise at least one nuclease domain and at least one Fc domain. Hybrid nuclease molecules are also referred to as fusion protein(s) and fusion gene(s). For example, in one embodiment, a hybrid nuclease molecule can be a polypeptide comprising at least one Fc domain linked to a nuclease domain such as DNase and/or RNase. As another example, a hybrid nuclease molecule can include an RNase nuclease domain, a linker domain, and an Fc domain. SEQ ID NO:161 is an example of a hybrid nuclease molecule. Other examples are described in more detail below. In one embodiment a hybrid nuclease molecule of the invention can include additional modifications. In another embodiment, a hybrid nuclease molecule may be modified to add a functional moiety (e.g., PEG, a drug, or a label).

In certain aspects, the hybrid nuclease molecules of the invention can employ one or more “linker domains,” such as polypeptide linkers. As used herein, the term “linker domain” refers to a sequence which connects two or more domains in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect a nuclease domain to an Fc domain. Preferably, such polypeptide linkers can provide flexibility to the polypeptide molecule. In certain embodiments the polypeptide linker is used to connect (e.g., genetically fuse) one or more Fc domains and/or one or more nuclease domains. A hybrid nuclease molecule of the invention may comprise more than one linker domain or peptide linker.

As used herein, the term “gly-ser polypeptide linker” refers to a peptide that consists of glycine and serine residues. An exemplary gly/ser polypeptide linker comprises the amino acid sequence Ser(Gly₄Ser)n. In one embodiment, n=1. In one embodiment, n=2. In another embodiment, n=3, i.e., Ser(GlySer)3. In another embodiment, n=4, i.e., Ser(Gly₄Ser)4. In another embodiment, n=5. In yet another embodiment, n=6. In another embodiment, n=7. In yet another embodiment, n=8. In another embodiment, n=9. In yet another embodiment, n=10. Another exemplary gly/ser polypeptide linker comprises the amino acid sequence Ser(Gly₄Ser)n. In one embodiment, n=1. In one embodiment, n=2. In a preferred embodiment, n=3. In another embodiment, n=4. In another embodiment, n=5. In yet another embodiment, n=6.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, the term “Fc region” shall be defined as the portion of a native immunoglobulin formed by the respective Fc domains (or Fc moieties) of its two heavy chains.

As used herein, the term “Fc domain” refers to a portion of a single immunoglobulin (Ig) heavy chain. As such, Fc domain can also be referred to as “Ig” or “IgG.” In some embodiments, an Fc domain begins in the hinge region just upstream of the papain cleavage site and ending at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In other embodiments, an Fc domain comprises a complete Fc domain (i.e., a hinge domain, a CH2 domain, and a CH3 domain). In one embodiment, an Fc domain comprises a hinge domain (or portion thereof) fused to a CH3 domain (or portion thereof). In another embodiment, an Fc domain comprises a CH2 domain (or portion thereof) fused to a CH3 domain (or portion thereof). In another embodiment, an Fc domain consists of a CH3 domain or portion thereof. In another embodiment, an Fc domain consists of a hinge domain (or portion thereof) and a CH3 domain (or portion thereof). In another embodiment, an Fc domain consists of a CH2 domain (or portion thereof) and a CH3 domain. In another embodiment, an Fc domain consists of a hinge domain (or portion thereof) and a CH2 domain (or portion thereof). In one embodiment, an Fc domain lacks at least a portion of a CH2 domain (e.g., all or part of a CH2 domain). In one embodiment, an Fc domain of the invention comprises at least the portion of an Fc molecule known in the art to be required for FcRn binding. In another embodiment, an Fc domain of the invention comprises at least the portion of an Fc molecule known in the art to be required for FcγR binding. In one embodiment, an Fc domain of the invention comprises at least the portion of an Fc molecule known in the art to be required for Protein A binding. In one embodiment, an Fc domain of the invention comprises at least the portion of an Fc molecule known in the art to be required for protein G binding. An Fc domain herein generally refers to a polypeptide comprising all or part of the Fc domain of an immunoglobulin heavy-chain. This includes, but is not limited to, polypeptides comprising the entire CHI, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As with Fc variants and native Fc's, the term Fc domain includes molecules in monomeric or multimeric form, whether digested from whole antibody or produced by other means.

As set forth herein, it will be understood by one of ordinary skill in the art that any Fc domain may be modified such that it varies in amino acid sequence from the native Fc domain of a naturally occurring immunoglobulin molecule. In certain exemplary embodiments, the Fc domain retains an effector function (e.g., FcγR binding).

The Fc domains of a polypeptide of the invention may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting hybrid nuclease molecules. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e. same residue) with the starting amino acid residues, after aligning the sequences and introducing gaps, if necessary, to achieve the maximum percent sequence identity.

In one embodiment, a polypeptide of the invention consists of, consists essentially of, or comprises an amino acid sequence selected from Table 2 and functionally active variants thereof. In an embodiment, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in Table 2. In an embodiment, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence set forth in Table 2. In an embodiment, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence set forth in Table 2.

In an embodiment, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, siRNA design and generation (see, e.g., the Dharmacon siDesign website), and the like. In an embodiment, the nucleotide sequence of the invention comprises, consists of, or consists essentially of, a nucleotide sequence selected from Table 2. In an embodiment, a nucleotide sequence includes a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence set forth in Table 2. In an embodiment, a nucleotide sequence includes a contiguous nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous nucleotide sequence set forth in Table 2. In an embodiment, a nucleotide sequence includes a nucleotide sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous nucleotides of a nucleotide sequence set forth in Table 2.

Preferred hybrid nuclease molecules of the invention comprise a sequence (e.g., at least one Fc domain) derived from a human immunoglobulin sequence. However, sequences may comprise one or more sequences from another mammalian species. For example, a primate Fc domain or nuclease domain may be included in the subject sequence. Alternatively, one or more murine amino acids may be present in a polypeptide. In some embodiments, polypeptide sequences of the invention are not immunogenic and/or have reduced immunogenicity.

It will also be understood by one of ordinary skill in the art that the hybrid nuclease molecules of the invention may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. An isolated nucleic acid molecule encoding a non-natural variant of a hybrid nuclease molecule derived from an immunoglobulin (e.g., an Fc domain) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis.

The peptide hybrid nuclease molecules of the invention may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

The term “ameliorating” refers to any therapeutically beneficial result in the treatment of a disease state, e.g., an autoimmune disease state (e.g., SLE), including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” or “subject” or “patient” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### Compositions

### Hybrid Nuclease Molecules

In some embodiments, a composition of the invention includes a hybrid nuclease molecule. In some embodiments, a hybrid nuclease molecule includes a nuclease domain operatively linked to an Fc domain. In some embodiments, a hybrid nuclease molecule includes a nuclease domain linked to an Fc domain. In some embodiments the hybrid nuclease molecule is a nuclease protein. In some embodiments, the hybrid nuclease molecule is a nuclease polynucleotide.

In some embodiments, the nuclease domain is linked to the Fc domain via a linker domain. In some embodiments, the linker domain is a linker peptide. In some embodiments, the linker domain is a linker nucleotide. In some embodiments, the hybrid nuclease molecule includes a leader molecule, e.g., a leader peptide. In some embodiments, the leader molecule is a leader peptide positioned at the N-terminus of the nuclease domain. In some embodiments, the hybrid nuclease molecule will include a stop codon. In some embodiments, the stop codon will be at the C-terminus of the Fc domain.

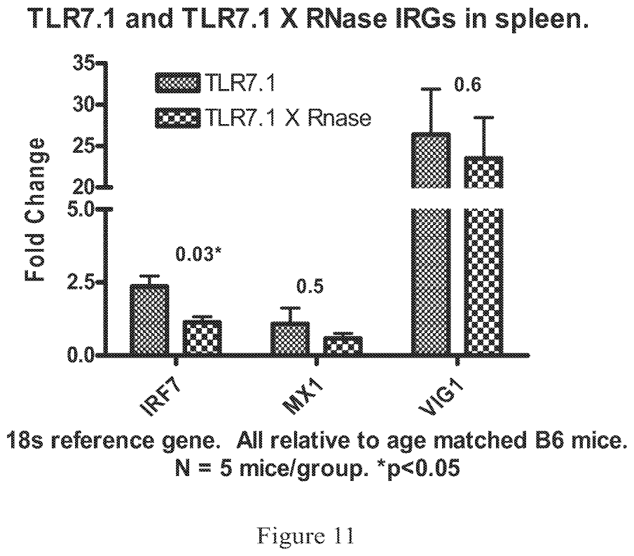

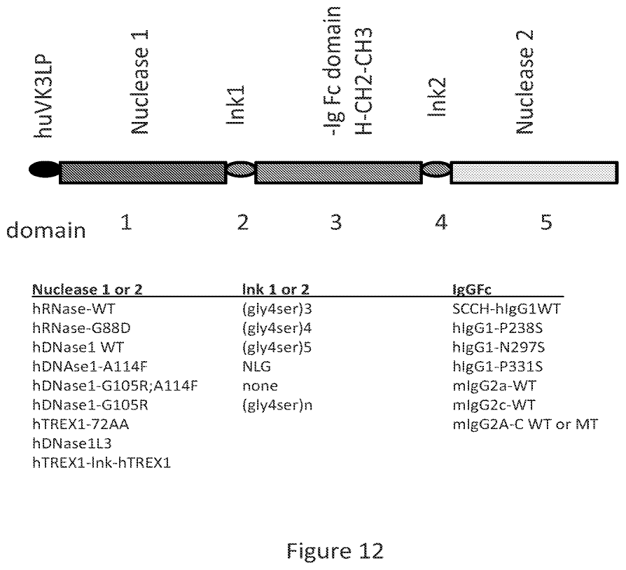

In some embodiments, the hybrid nuclease molecule further includes a second nuclease domain. In some embodiments, the second nuclease domain is linked to the Fc domain via a second linker domain. In some embodiments, the second linker domain will be at the C-terminus of the Fc domain. FIG. 12 shows at least one embodiment of a hybrid nuclease molecule. In some embodiments, a hybrid nuclease molecule includes a sequence shown in Table 2.

In some embodiments, a hybrid nuclease molecule is an RNase molecule or DNase molecule or a multi-enzyme molecule (e.g., both RNase and DNase or two RNA or DNA nucleases with different specificity for substrate) attached to an Fc domain that specifically binds to extracellular immune complexes. In some embodiments, the Fc domain does not effectively bind Fcγ receptors. In one aspect, the hybrid nuclease molecule does not effectively bind C1q. In other aspects, the hybrid nuclease molecule comprises an in frame Fc domain from IgG1. In other aspects, the hybrid nuclease molecule further comprises mutations in the hinge, CH2, and/or CH3 domains. In other aspects, the mutations are P238S, P331S or N297S, and may include mutations in one or more of the three hinge cysteines. In some such aspects, the mutations in one or more of three hinge cysteines can be SCC or SSS. In other aspects, the molecules contain the SCC hinge, but are otherwise wild type for human IgG1 Fc CH2 and CH3 domains, and bind efficiently to Fc receptors, facilitating uptake of the hybrid nuclease molecule into the endocytic compartment of cells to which they are bound. In other aspects, the molecule has activity against single and/or double-stranded RNA substrates.

In some aspects, the activity of the hybrid nuclease molecule is detectable in vitro and/or in vivo. In some aspects, the hybrid nuclease molecule binds to a cell, a malignant cell, or a cancer cell and interferes with its biologic activity.

In another aspect, a multifunctional RNase molecule is provided that is attached to another enzyme or antibody having binding specificity, such as an scFv targeted to RNA or a second nuclease domain with the same or different specificities as the first domain.

In another aspect, a multifunctional DNase molecule is provided that is attached to another enzyme or antibody having binding specificity, such as an scFv targeted to DNA or a second nuclease domain with the same or different specificities as the first domain.

In another aspect, a hybrid nuclease molecule is adapted for preventing or treating a disease or disorder in a mammal by administering an hybrid nuclease molecule attached to an Fc region, in a therapeutically effective amount to the mammal in need thereof, wherein the disease is prevented or treated. In other aspects, the disease or disorder is an autoimmune disease or cancer. In some such aspects, the autoimmune disease is insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbs-ve, cryptogenic cirrhosis, ulcerative colitis, Sjogren's syndrome, scleroderma, Wegener's granulomatosis, polymyositis, dermatomyositis, discoid LE, systemic lupus erythematosus, or connective tissue disease.

In some embodiments, the targets of the RNase enzyme activity of RNase hybrid nuclease molecules are primarily extracellular, consisting of, e.g., RNA contained in immune complexes with anti-RNP autoantibody and RNA expressed on the surface of cells undergoing apoptosis. In some embodiments, the RNase hybrid nuclease molecule is active in the acidic environment of the endocytic vesicles. In some embodiments, an RNase hybrid nuclease molecule includes a wild-type (wt) Fc domain in order to, e.g, allow the molecule to bind FcR and enter the endocytic compartment through the entry pathway used by immune complexes. In some embodiments, an RNase hybrid nuclease molecule including a wt Fc domain is adapted to be active both extracellularly and in the endocytic environment (where TLR7 can be expressed). In some aspects, this allows an RNase hybrid nuclease molecule including a wt Fc domain to stop TLR7 signaling through previously engulfed immune complexes or by RNAs that activate TLR7 after viral infection. In some embodiments, the wt RNase of an RNase hybrid nuclease molecule is not resistant to inhibition by an RNase cytoplasmic inhibitor. In some embodiments, the wt RNase of an RNase hybrid nuclease molecule is not active in the cytoplasm of a cell.

In some embodiments, a hybrid nuclease molecule including a wt Fc domain is used for therapy of an autoimmune disease, e.g., SLE.

In some embodiments, Fc domain binding to an Fc receptor (FcR) is increased, e.g., via alterations of glycosylation and/or changes in amino acid sequence. In some embodiments, a hybrid nuclease molecule has one or more Fc alterations that increase FcR binding.

Alternative ways to construct a hybrid nuclease molecule attached to an Fc domain are envisioned. In some embodiments, the domain orientation can be altered to construct an Ig-RNase molecule or an Ig-DNase molecule or an RNase-Ig molecule or an RNase-Ig molecule that retains FcR binding and has active nuclease domains.

In some embodiments, DNase hybrid nuclease molecules include a wt Fc domain that can allow, e.g., the molecules to undergo endocytosis after binding FcR. In some embodiments, the DNase hybrid nuclease molecules can be active towards extracellular immune complexes containing DNA, e.g., either in soluble form or deposited as insoluble complexes.

In some embodiments, hybrid nuclease molecules include both DNase and RNase. In some embodiments, these hybrid nuclease molecules can improve the therapy of SLE because they can, e.g., digest immune complexes containing RNA, DNA, or a combination of both RNA and DNA; and when they further include a wt Fc domain, they are active both extracellularly and in the endocytic compartment where TLR7 and TLR9 can be located.

In some embodiments, linker domains include (gly4ser) 3, 4 or 5 variants that alter the length of the linker by 5 amino acid progressions. In another embodiment, a linker domain is approximately 18 amino acids in length and includes an N-linked glycosylation site, which can be sensitive to protease cleavage in vivo. In some embodiments, an N-linked glycosylation site can protect the hybrid nuclease molecules from cleavage in the linker domain. In some embodiments, an N-linked glycosylation site can assist in separating the folding of independent functional domains separated by the linker domain.

In some embodiments, hybrid nuclease molecules can include both mutant and/or wild type human IgG1 Fc domains. In some embodiments, the hybrid nuclease molecules can be expressed from both COS transient and CHO stable transfections. In some embodiments, both the CD80/86 binding and the RNase activity are preserved in a hybrid nuclease molecule. In some embodiments, hybrid nuclease molecules include DNase 1L3-Ig-linker-RNase constructs. In some embodiments, a hybrid nuclease molecule includes a DNase 1-Ig-linker-RNase construct or an RNase-Ig-linker-DNase construct. In some embodiments, fusion junctions between enzyme domains and the other domains of the hybrid nuclease molecule is optimized.

In some embodiments, hybrid nuclease molecules include DNase-Ig hybrid nuclease molecules and/or hybrid DNase-RNase hybrid nuclease molecules.

In some embodiments, a hybrid nuclease molecule includes TREX1. In some embodiments, a TREX1 hybrid nuclease molecule can digest chromatin. In some embodiments, a TREX1 hybrid nuclease molecule is expressed by a cell. In some embodiments, the expressed hybrid nuclease molecule includes murine TREX-1 and a murine (wt or mutant) Fc domain. In some embodiments, a 20-25 amino acid (aa) linker domain between TREX1 and the IgG hinge can be required to allow DNase activity. In some embodiments, a hybrid nuclease molecule with a 15 aa linker domain is not active. In some embodiments, use of the 20 and 25 amino acid linker domains (plus 2 or more amino acids to incorporate restriction sites) results in functional activity as measured by chromatin digestion. In some embodiments, a hydrophobic region of approximately 72 aa can be removed from the COOH end of TREX-1 prior to fusion to the Fc domain via the linker domain. In some embodiments, a 20 amino acid linker domain version of the hybrid nuclease molecule exhibits high expression levels compared to controls and/or other hybrid nuclease molecules. In some embodiments, kinetic enzyme assays are used to compare the enzyme activity of hybrid nuclease molecules and controls in a quantitative manner.

In some embodiments, further optimization of the fusion junction chosen for truncation of a TREX1 enzyme can be used to improve expression of the hybrid nuclease molecules.

In some embodiments, the hybrid nuclease molecule includes a human TREX1-linker-Ig Fc domain hybrid nuclease molecule with 20 and/or 25 aa linker domains. In some embodiments, the linker domain(s) are variants of a (gly4ser)4 or (gly4ser)5 cassette with one or more restriction sites attached for incorporation into the hybrid nuclease molecules construct. In some embodiments, because of the head-to-tail dimerization useful for TREX1 enzyme activity; a flexible, longer linker domain can be used to facilitate proper folding.

In some embodiments, the hybrid nuclease molecule is a TREX1-tandem hybrid nuclease molecule. In some embodiments, an alternative method for facilitating head-to-tail folding of TREX1 is to generate a TREX1-TREX1-Ig hybrid hybrid nuclease molecule that incorporates two TREX1 domains in tandem, followed by a linker domain and an Ig Fc domain. In some embodiments, positioning of TREX1 cassettes in a head-to-tail manner can be corrected for head-to tail folding on either arm of the immunoenzyme and introduce a single TREX1 functional domain into each arm of the molecule. In some embodiments, each immunoenzyme of a hybrid nuclease molecule has two functional TREX1 enzymes attached to a single IgG Fc domain.

In some embodiments, the hybrid nuclease molecule includes TREX1-linker1-Ig-linker2-RNase.

In some embodiments, the hybrid nuclease molecule includes RNase-Ig-linker-TREX1. In some embodiments, cassettes are derived for both amino and carboxyl fusion of each enzyme for incorporation into hybrid nuclease molecules where the enzyme configuration is reversed. In some embodiments, the RNase enzyme exhibits comparable functional activity regardless of its position in the hybrid nuclease molecules. In some embodiments, alternative hybrid nuclease molecules can be designed to test whether a particular configuration demonstrates improved expression and/or function of the hybrid nuclease molecule components.

In some embodiments, the hybrid nuclease molecule includes 1L3-Ig. In some embodiments, the 1L3 DNase is constructed from a murine sequence and expressed. In some embodiments, the enzyme is active. In some embodiments, a murine 1L3 DNase-Ig-RNase hybrid nuclease is constructed and expressed. In some embodiments, the molecule includes human 1L3-Ig, human 1L3-Ig-RNase, and/or human RNase-Ig-1L3.

In some embodiments, the hybrid nuclease molecule includes DNase1-Ig. In some embodiments, a naturally occurring variant allele, A114F, which shows reduced sensitivity to actin is included in a DNase1-Ig hybrid nuclease molecule. In some embodiments, this mutation is introduced into a hybrid nuclease molecule to generate a more stable derivative of human DNase1. In some embodiments, a DNase1-linker-Ig containing a 20 or 25 aa linker domain is made. In some embodiments, hybrid nuclease molecules include RNase-Ig-linker-DNase1 where the DNase1 domain is located at the COOH side of the Ig Fc domain. In some embodiments, hybrid nuclease molecules are made that incorporate DNase1 and include: DNase 1-linker-Ig-linker2-RNase, and/or RNase-Ig-linker-DNase1.

Another aspect of the present invention is to use gene therapy methods for treating or preventing disorders, diseases, and conditions with one or more hybrid nuclease molecules. The gene therapy methods relate to the introduction of hybrid nuclease molecule nucleic acid (DNA, RNA and antisense DNA or RNA) sequences into an animal to achieve expression of the polypeptide or polypeptides of the present invention. This method can include introduction of one or more polynucleotides encoding a hybrid nuclease molecule polypeptide of the present invention operatively linked to a promoter and any other genetic elements necessary for the expression of the polypeptide by the target tissue.

In gene therapy applications, hybrid nuclease molecule genes are introduced into cells in order to achieve in vivo synthesis of a therapeutically effective genetic product. “Gene therapy” includes both conventional gene therapies where a lasting effect is achieved by a single treatment, and the administration of gene therapeutic agents, which involves the one time or repeated administration of a therapeutically effective DNA or mRNA. The oligonucleotides can be modified to enhance their uptake, e.g., by substituting their negatively charged phosphodiester groups by uncharged groups.

### Fc Domains

In some embodiments, a hybrid nuclease molecule includes an Fc domain. Fc domains useful for producing the hybrid nuclease molecules of the present invention may be obtained from a number of different sources. In preferred embodiments, an Fc domain of the hybrid nuclease molecule is derived from a human immunoglobulin. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species. Moreover, the hybrid nuclease molecule Fc domain or portion thereof may be derived from any immunoglobulin class, including IgM, IgG, IgD, IgA, and IgE, and any immunoglobulin isotype, including IgG1, IgG2, IgG3, and IgG4. In a preferred embodiment, the human isotype IgG1 is used.

A variety of Fc domain gene sequences (e.g. human constant region gene sequences) are available in the form of publicly accessible deposits. Constant region domains comprising an Fc domain sequence can be selected having a particular effector function (or lacking a particular effector function) or with a particular modification to reduce immunogenicity. Many sequences of antibodies and antibody-encoding genes have been published and suitable Fc domain sequences (e.g. hinge, CH2, and/or CH3 sequences, or portions thereof) can be derived from these sequences using art recognized techniques. The genetic material obtained using any of the foregoing methods may then be altered or synthesized to obtain polypeptides of the present invention. It will further be appreciated that the scope of this invention encompasses alleles, variants and mutations of constant region DNA sequences.

Fc domain sequences can be cloned, e.g., using the polymerase chain reaction and primers which are selected to amplify the domain of interest. To clone an Fc domain sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

The hybrid nuclease molecules of the invention may comprise one or more Fc domains (e.g., 2, 3, 4, 5, 6, 7, 8, 9, 10, or more Fc domains). In one embodiment, the Fc domains may be of different types. In one embodiment, at least one Fc domain present in the hybrid nuclease molecule comprises a hinge domain or portion thereof. In another embodiment, the hybrid nuclease molecule of the invention comprises at least one Fc domain which comprises at least one CH2 domain or portion thereof. In another embodiment, the hybrid nuclease molecule of the invention comprises at least one Fc domain which comprises at least one CH3 domain or portion thereof. In another embodiment, the hybrid nuclease molecule of the invention comprises at least one Fc domain which comprises at least one CH4 domain or portion thereof. In another embodiment, the hybrid nuclease molecule of the invention comprises at least one Fc domain which comprises at least one hinge domain or portion thereof and at least one CH2 domain or portion thereof (e.g, in the hinge-CH2 orientation). In another embodiment, the hybrid nuclease molecule of the invention comprises at least one Fc domain which comprises at least one CH2 domain or portion thereof and at least one CH3 domain or portion thereof (e.g, in the CH2-CH3 orientation). In another embodiment, the hybrid nuclease molecule of the invention comprises at least one Fc domain comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In certain embodiments, the hybrid nuclease molecule comprises at least one complete Fc region derived from one or more immunoglobulin heavy chains (e.g., an Fc domain including hinge, CH2, and CH3 domains, although these need not be derived from the same antibody). In other embodiments, the hybrid nuclease molecule comprises at least two complete Fc domains derived from one or more immunoglobulin heavy chains. In preferred embodiments, the complete Fc domain is derived from a human IgG immunoglobulin heavy chain (e.g., human IgG1).

In another embodiment, a hybrid nuclease molecule of the invention comprises at least one Fc domain comprising a complete CH3 domain. In another embodiment, a hybrid nuclease molecule of the invention comprises at least one Fc domain comprising a complete CH2 domain. In another embodiment, a hybrid nuclease molecule of the invention comprises at least one Fc domain comprising at least a CH3 domain, and at least one of a hinge region, and a CH2 domain. In one embodiment, a hybrid nuclease molecule of the invention comprises at least one Fc domain comprising a hinge and a CH3 domain. In another embodiment, a hybrid nuclease molecule of the invention comprises at least one Fc domain comprising a hinge, a CH2, and a CH3 domain. In preferred embodiments, the Fc domain is derived from a human IgG immunoglobulin heavy chain (e.g., human IgG1).

The constant region domains or portions thereof making up an Fc domain of a hybrid nuclease molecule of the invention may be derived from different immunoglobulin molecules. For example, a polypeptide of the invention may comprise a CH2 domain or portion thereof derived from an IgG1 molecule and a CH3 region or portion thereof derived from an IgG3 molecule. In another example, a hybrid nuclease molecule can comprise an Fc domain comprising a hinge domain derived, in part, from an IgG1 molecule and, in part, from an IgG3 molecule. As set forth herein, it will be understood by one of ordinary skill in the art that an Fc domain may be altered such that it varies in amino acid sequence from a naturally occurring antibody molecule.

In another embodiment, a hybrid nuclease molecule of the invention comprises one or more truncated Fc domains that are nonetheless sufficient to confer Fc receptor (FcR) binding properties to the Fc region. Thus, an Fc domain of a hybrid nuclease molecule of the invention may comprise or consist of an FcRn binding portion. FcRn binding portions may be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used.

In one embodiment, a hybrid nuclease molecule of the invention lacks one or more constant region domains of a complete Fc region, i.e., they are partially or entirely deleted. In a certain embodiments hybrid nuclease molecules of the invention will lack an entire CH2 domain (ΔCH2 constructs). Those skilled in the art will appreciate that such constructs may be preferred due to the regulatory properties of the CH2 domain on the catabolic rate of the antibody. In certain embodiments, hybrid nuclease molecules of the invention comprise CH2 domain-deleted Fc regions derived from a vector (e.g., from IDEC Pharmaceuticals, San Diego) encoding an IgG1 human constant region domain (see, e.g., WO 02/060955A2 and WO02/096948A2). This exemplary vector is engineered to delete the CH2 domain and provide a synthetic vector expressing a domain-deleted IgG1 constant region. It will be noted that these exemplary constructs are preferably engineered to fuse a binding CH3 domain directly to a hinge region of the respective Fc domain.

In other constructs it may be desirable to provide a peptide spacer between one or more constituent Fc domains. For example, a peptide spacer may be placed between a hinge region and a CH2 domain and/or between a CH2 and a CH3 domain. For example, compatible constructs could be expressed wherein the CH2 domain has been deleted and the remaining CH3 domain (synthetic or unsynthetic) is joined to the hinge region with a 1-20, 1-10, or 1-5 amino acid peptide spacer. Such a peptide spacer may be added, for instance, to ensure that the regulatory elements of the constant region domain remain free and accessible or that the hinge region remains flexible. Preferably, any linker peptide compatible with the instant invention will be relatively non-immunogenic and not prevent proper folding of the Fc.

### Changes to Fc Amino Acids

In certain embodiments, an Fc domain employed in a hybrid nuclease molecule of the invention is altered, e.g., by amino acid mutation (e.g., addition, deletion, or substitution). As used herein, the term “Fc domain variant” refers to an Fc domain having at least one amino acid substitution as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region.

The amino acid substitution(s) of an Fc variant may be located at a position within the Fc domain referred to as corresponding to the portion number that that residue would be given in an Fc region in an antibody.

In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

In certain embodiments, the hybrid nuclease molecules of the invention comprise an Fc variant comprising more than one amino acid substitution. The hybrid nuclease molecules of the invention may comprise, for example, 2, 3, 4, 5, 6, 7, 8, 9, 10 or more amino acid substitutions. Preferably, the amino acid substitutions are spatially positioned from each other by an interval of at least 1 amino acid position or more, for example, at least 2, 3, 4, 5, 6, 7, 8, 9, or 10 amino acid positions or more. More preferably, the engineered amino acids are spatially positioned apart from each other by an interval of at least 5, 10, 15, 20, or 25 amino acid positions or more.

In certain embodiments, the Fc variant confers an improvement in at least one effector function imparted by an Fc domain comprising said wild-type Fc domain (e.g., an improvement in the ability of the Fc domain to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In other embodiments, the Fc variant provides an engineered cysteine residue.

The hybrid nuclease molecules of the invention may employ art-recognized Fc variants which are known to impart an improvement in effector function and/or FcR binding. Specifically, a hybrid nuclease molecule of the invention may include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351 A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242, 195; 6,277,375; 6,528,624; 6,538,124; 6,737,056; 6,821,505; 6,998,253; 7,083,784; and 7,317,091, each of which is incorporated by reference herein. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

In certain embodiments, a hybrid nuclease molecule of the invention comprises an amino acid substitution to an Fc domain which alters the antigen-independent effector functions of the antibody, in particular the circulating half-life of the antibody. Such hybrid nuclease molecules exhibit either increased or decreased binding to FcRn when compared to hybrid nuclease molecules lacking these substitutions and, therefore, have an increased or decreased half-life in serum, respectively. Fc variants with improved affinity for FcRn are anticipated to have longer serum half-lives, and such molecules have useful applications in methods of treating mammals where long half-life of the administered polypeptide is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the hybrid nuclease molecules of the invention exhibit reduced transport across the epithelium of kidney glomeruli from the vasculature. In another embodiment, the hybrid nuclease molecules of the invention exhibit reduced transport across the blood brain barrier (BBB) from the brain, into the vascular space. In one embodiment, a hybrid nuclease molecule with altered FcRn binding comprises at least one Fc domain (e.g., one or two Fc domains) having one or more amino acid substitutions within the “FcRn binding loop” of an Fc domain. Exemplary amino acid substitutions which altered FcRn binding activity are disclosed in International PCT Publication No. WO05/047327 which is incorporated by reference herein.

In other embodiments, a hybrid nuclease molecule of the invention comprises an Fc variant comprising an amino acid substitution which alters the antigen-dependent effector functions of the polypeptide, in particular ADCC or complement activation, e.g., as compared to a wild type Fc region. In exemplary embodiment, said hybrid nuclease molecules exhibit altered binding to an Fc gamma receptor (e.g., CD16). Such hybrid nuclease molecules exhibit either increased or decreased binding to FcR gamma when compared to wild-type polypeptides and, therefore, mediate enhanced or reduced effector function, respectively. Fc variants with improved affinity for FcγRs are anticipated to enhance effector function, and such molecules have useful applications in methods of treating mammals where target molecule destruction is desired. In contrast, Fc variants with decreased FcγR binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation. In one embodiment, the polypeptide comprising an Fc exhibits at least one altered antigen-dependent effector function selected from the group consisting of opsonization, phagocytosis, complement dependent cytotoxicity, antigen-dependent cellular cytotoxicity (ADCC), or effector cell modulation as compared to a polypeptide comprising a wild type Fc region.

In one embodiment the hybrid nuclease molecule exhibits altered binding to an activating FcγR (e.g. FcγI, FcγIIa, or FcγRIIIa). In another embodiment, the hybrid nuclease molecule exhibits altered binding affinity to an inhibitory FcγR (e.g. FcγRIIb). Exemplary amino acid substitutions which altered FcR or complement binding activity are disclosed in International PCT Publication No. WO05/063815 which is incorporated by reference herein.

A hybrid nuclease molecule of the invention may also comprise an amino acid substitution which alters the glycosylation of the hybrid nuclease molecule. For example, the Fc domain of the hybrid nuclease molecule may comprise an Fc domain having a mutation leading to reduced glycosylation (e.g., N- or O-linked glycosylation) or may comprise an altered glycoform of the wild-type Fc domain (e.g., a low fucose or fucose-free glycan). In another embodiment, the hybrid nuclease molecule has an amino acid substitution near or within a glycosylation motif, for example, an N-linked glycosylation motif that contains the amino acid sequence NXT or NXS. Exemplary amino acid substitutions which reduce or alter glycosylation are disclosed in International PCT Publication No. WO05/018572 and US Patent Publication No. 2007/0111281, which are incorporated by reference herein.

In other embodiments, a hybrid nuclease molecule of the invention comprises at least one Fc domain having engineered cysteine residue or analog thereof which is located at the solvent-exposed surface. Preferably the engineered cysteine residue or analog thereof does not interfere with an effector function conferred by the Fc. More preferably, the alteration does not interfere with the ability of the Fc to bind to Fc receptors (e.g. FcγRI, FcγRII, or FcγRIII) or complement proteins (e.g. C1q), or to trigger immune effector function (e.g., antibody-dependent cytotoxicity (ADCC), phagocytosis, or complement-dependent cytotoxicity (CDCC)). In preferred embodiments, the hybrid nuclease molecules of the invention comprise an Fc domain comprising at least one engineered free cysteine residue or analog thereof that is substantially free of disulfide bonding with a second cysteine residue. Any of the above engineered cysteine residues or analogs thereof may subsequently be conjugated to a functional domain using art-recognized techniques (e.g., conjugated with a thiol-reactive heterobifunctional linker).

In one embodiment, the hybrid nuclease molecule of the invention may comprise a genetically fused Fc domain having two or more of its constituent Fc domains independently selected from the Fc domains described herein. In one embodiment, the Fc domains are the same. In another embodiment, at least two of the Fc domains are different. For example, the Fc domains of the hybrid nuclease molecules of the invention comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In yet other embodiments, the Fc domains of the hybrid nuclease molecules of the invention may differ in sequence at one or more amino acid positions. For example, at least two of the Fc domains may differ at about 5 amino acid positions (e.g., 1, 2, 3, 4, or 5 amino acid positions), about 10 positions, about 15 positions, about 20 positions, about 30 positions, about 40 positions, or about 50 positions).

### Linker Domains

In some embodiments, a hybrid nuclease molecule includes a linker domain. In some embodiments, a hybrid nuclease molecule includes a plurality of linker domains. In some embodiments, the linker domain is a polypeptide linker. In certain aspects, it is desirable to employ a polypeptide linker to fuse one or more Fc domains to one or more nuclease domains to form a hybrid nuclease molecule.

In one embodiment, the polypeptide linker is synthetic. As used herein the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) (e.g., an Fc domain sequence) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). The polypeptide linkers of the invention may be employed, for instance, to ensure that Fc domains are juxtaposed to ensure proper folding and formation of a functional Fc domain. Preferably, a polypeptide linker compatible with the instant invention will be relatively non-immunogenic and not inhibit any non-covalent association among monomer subunits of a binding protein.

In certain embodiments, the hybrid nuclease molecules of the invention employ a polypeptide linker to join any two or more domains in frame in a single polypeptide chain. In one embodiment, the two or more domains may be independently selected from any of the Fc domains or nuclease domains discussed herein. For example, in certain embodiments, a polypeptide linker can be used to fuse identical Fc domains, thereby forming a homomeric Fc region. In other embodiments, a polypeptide linker can be used to fuse different Fc domains (e.g. a wild-type Fc domain and a Fc domain variant), thereby forming a heteromeric Fc region. In other embodiments, a polypeptide linker of the invention can be used to genetically fuse the C-terminus of a first Fc domain (e.g. a hinge domain or portion thereof, a CH2 domain or portion thereof, a complete CH3 domain or portion thereof, a FcRn binding portion, an FcγR binding portion, a complement binding portion, or portion thereof) to the N-terminus of a second Fc domain (e.g., a complete Fc domain).

In one embodiment, a polypeptide linker comprises a portion of an Fc domain. For example, in one embodiment, a polypeptide linker can comprise an immunoglobulin hinge domain of an IgG1, IgG2, IgG3, and/or IgG4 antibody. In another embodiment, a polypeptide linker can comprise a CH2 domain of an IgG1, IgG2, IgG3, and/or IgG4 antibody. In other embodiments, a polypeptide linker can comprise a CH3 domain of an IgG1, IgG2, IgG3, and/or IgG4 antibody. Other portions of an immunoglobulin (e.g. a human immunoglobulin) can be used as well. For example, a polypeptide linker can comprise a CH1 domain or portion thereof, a CL domain or portion thereof, a VH domain or portion thereof, or a VL domain or portion thereof. Said portions can be derived from any immunoglobulin, including, for example, an IgG1, IgG2, IgG3, and/or IgG4 antibody.

In exemplary embodiments, a polypeptide linker can comprise at least a portion of an immunoglobulin hinge region. In one embodiment, a polypeptide linker comprises an upper hinge domain (e.g., an IgG1, an IgG2, an IgG3, or IgG4 upper hinge domain). In another embodiment, a polypeptide linker comprises a middle hinge domain (e.g., an IgG1, an IgG2, an IgG3, or an IgG4 middle hinge domain). In another embodiment, a polypeptide linker comprises a lower hinge domain (e.g., an IgG1, an IgG2, an IgG3, or an IgG4 lower hinge domain).

In other embodiments, polypeptide linkers can be constructed which combine hinge elements derived from the same or different antibody isotypes. In one embodiment, the polypeptide linker comprises a chimeric hinge comprising at least a portion of an IgG1 hinge region and at least a portion of an IgG2 hinge region. In one embodiment, the polypeptide linker comprises a chimeric hinge comprising at least a portion of an IgG1 hinge region and at least a portion of an IgG3 hinge region. In another embodiment, a polypeptide linker comprises a chimeric hinge comprising at least a portion of an IgG1 hinge region and at least a portion of an IgG4 hinge region. In one embodiment, the polypeptide linker comprises a chimeric hinge comprising at least a portion of an IgG2 hinge region and at least a portion of an IgG3 hinge region. In one embodiment, the polypeptide linker comprises a chimeric hinge comprising at least a portion of an IgG2 hinge region and at least a portion of an IgG4 hinge region. In one embodiment, the polypeptide linker comprises a chimeric hinge comprising at least a portion of an IgG1 hinge region, at least a portion of an IgG2 hinge region, and at least a portion of an IgG4 hinge region. In another embodiment, a polypeptide linker can comprise an IgG1 upper and middle hinge and a single IgG3 middle hinge repeat motif. In another embodiment, a polypeptide linker can comprise an IgG4 upper hinge, an IgG1 middle hinge and a IgG2 lower hinge.

In another embodiment, a polypeptide linker comprises or consists of a gly-ser linker. As used herein, the term “gly-ser linker” refers to a peptide that consists of glycine and serine residues. An exemplary gly/ser linker comprises an amino acid sequence of the formula (Gly₄Ser)n, wherein n is a positive integer (e.g., 1, 2, 3, 4, or 5). A preferred gly/ser linker is (Gly₄Ser)4. Another preferred gly/ser linker is (Gly₄Ser)3. Another preferred gly/ser linker is (Gly₄Ser)5. In certain embodiments, the gly-ser linker may be inserted between two other sequences of the polypeptide linker (e.g., any of the polypeptide linker sequences described herein). In other embodiments, a gly-ser linker is attached at one or both ends of another sequence of the polypeptide linker (e.g., any of the polypeptide linker sequences described herein). In yet other embodiments, two or more gly-ser linker are incorporated in series in a polypeptide linker. In one embodiment, a polypeptide linker of the invention comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of gly/ser amino acid residues (e.g., a gly/ser linker such as (Gly₄Ser)n).

In one embodiment, a polypeptide linker of the invention comprises a non-naturally occurring immunoglobulin hinge region domain, e.g., a hinge region domain that is not naturally found in the polypeptide comprising the hinge region domain and/or a hinge region domain that has been altered so that it differs in amino acid sequence from a naturally occurring immunoglobulin hinge region domain. In one embodiment, mutations can be made to hinge region domains to make a polypeptide linker of the invention. In one embodiment, a polypeptide linker of the invention comprises a hinge domain which does not comprise a naturally occurring number of cysteines, i.e., the polypeptide linker comprises either fewer cysteines or a greater number of cysteines than a naturally occurring hinge molecule.

In other embodiments, a polypeptide linker of the invention comprises a biologically relevant peptide sequence or a sequence portion thereof. For example, a biologically relevant peptide sequence may include, but is not limited to, sequences derived from an anti-rejection or anti-inflammatory peptide. Said anti-rejection or anti-inflammatory peptides may be selected from the group consisting of a cytokine inhibitory peptide, a cell adhesion inhibitory peptide, a thrombin inhibitory peptide, and a platelet inhibitory peptide. In a one preferred embodiment, a polypeptide linker comprises a peptide sequence selected from the group consisting of an IL-1 inhibitory or antagonist peptide sequence, an erythropoietin (EPO)-mimetic peptide sequence, a thrombopoietin (TPO)-mimetic peptide sequence, G-CSF mimetic peptide sequence, a TNF-antagonist peptide sequence, an integrin-binding peptide sequence, a selectin antagonist peptide sequence, an anti-pathogenic peptide sequence, a vasoactive intestinal peptide (VIP) mimetic peptide sequence, a calmodulin antagonist peptide sequence, a mast cell antagonist, a SH3 antagonist peptide sequence, an urokinase receptor (UKR) antagonist peptide sequence, a somatostatin or cortistatin mimetic peptide sequence, and a macrophage and/or T-cell inhibiting peptide sequence. Exemplary peptide sequences, any one of which may be employed as a polypeptide linker, are disclosed in U.S. Pat. No. 6,660,843, which is incorporated by reference herein.

It will be understood that variant forms of these exemplary polypeptide linkers can be created by introducing one or more nucleotide substitutions, additions or deletions into the nucleotide sequence encoding a polypeptide linker such that one or more amino acid substitutions, additions or deletions are introduced into the polypeptide linker. For example, mutations may be introduced by standard techniques, such as site-directed mutagenesis and PCR-mediated mutagenesis.

Polypeptide linkers of the invention are at least one amino acid in length and can be of varying lengths. In one embodiment, a polypeptide linker of the invention is from about 1 to about 50 amino acids in length. As used in this context, the term “about” indicates+/−two amino acid residues. Since linker length must be a positive integer, the length of from about 1 to about 50 amino acids in length, means a length of from 1 to 48-52 amino acids in length. In another embodiment, a polypeptide linker of the invention is from about 10-20 amino acids in length. In another embodiment, a polypeptide linker of the invention is from about 15 to about 50 amino acids in length.

In another embodiment, a polypeptide linker of the invention is from about 20 to about 45 amino acids in length. In another embodiment, a polypeptide linker of the invention is from about 15 to about 25 amino acids in length. In another embodiment, a polypeptide linker of the invention is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56,57, 58, 59, 60, or more amino acids in length.

Polypeptide linkers can be introduced into polypeptide sequences using techniques known in the art. Modifications can be confirmed by DNA sequence analysis. Plasmid DNA can be used to transform host cells for stable production of the polypeptides produced.

### Nuclease Domains

In certain aspects, a hybrid nuclease molecule includes a nuclease domain. Accordingly, the hybrid nuclease molecules of the invention typically comprise at least one nuclease domain and at least one linked Fc domain. In certain aspects, a hybrid nuclease molecule includes a plurality of nuclease domains.

In some embodiments, a nuclease domain is DNase. In some embodiments, the DNase is a Type I secreted DNase. In some embodiments, the DNase is DNase 1 and/or a DNase 1-like (DNaseL) enzyme, 1-3. In some embodiments, the DNase is TREX1.

In some embodiments, a nuclease domain is an RNase. In some embodiments, the RNase is an extracellular or secretory RNase of the RNase A superfamily, e.g., RNase A.

In one embodiment, the nuclease domain is operably linked (e.g., chemically conjugated or genetically fused (e.g., either directly or via a polypeptide linker)) to the N-terminus of an Fc domain. In another embodiment, the nuclease domain is operably linked (e.g., chemically conjugated or genetically fused (e.g., either directly or via a polypeptide linker)) to the C-terminus of an Fc domain. In other embodiments, a nuclease domain is operably linked (e.g., chemically conjugated or genetically fused (e.g., either directly or via a polypeptide linker)) via an amino acid side chain of an Fc domain. In certain exemplary embodiments, the nuclease domain is fused to an Fc domain via a human immunoglobulin hinge domain or portion thereof.

In certain embodiments, the hybrid nuclease molecules of the invention comprise two or more nuclease domains and at least one Fc domain. For example, nuclease domains may be operably linked to both the N-terminus and C-terminus of an Fc domain. In other exemplary embodiments, nuclease domains may be operably linked to both the N- and C-terminal ends of multiple Fc domains (e.g., two, three, four, five, or more Fc domains) which are linked together in series to form a tandem array of Fc domains.

In other embodiments, two or more nuclease domains are linked to each other (e.g., via a polypeptide linker) in series, and the tandem array of nuclease domains is operably linked (e.g., chemically conjugated or genetically fused (e.g., either directly or via a polypeptide linker)) to either the C-terminus or the N-terminus of a Fc domain or a tandem array of Fc domains. In other embodiments, the tandem array of nuclease domains is operably linked to both the C-terminus and the N-terminus of a Fc domain or a tandem array of Fc domains.

In other embodiments, one or more nuclease domains may be inserted between two Fc domains. For example, one or more nuclease domains may form all or part of a polypeptide linker of a hybrid nuclease molecule of the invention.

Preferred hybrid nuclease molecules of the invention comprise at least one nuclease domain (e.g., RNase or DNase), at least one linker domain, and at least one Fc domain.

In certain embodiments, the hybrid nuclease molecules of the invention have at least one nuclease domain specific for a target molecule which mediates a biological effect. In another embodiment, binding of the hybrid nuclease molecules of the invention to a target molecule (e.g. DNA or RNA) results in the reduction or elimination of the target molecule, e.g., from a cell, a tissue, or from circulation.

In certain embodiments, the hybrid nuclease molecules of the invention may comprise two or more nuclease domains. In one embodiment, the nuclease domains are identical, e.g., RNase and RNase, or TREX1 and TREX1. In another embodiment, the nuclease domains are different, e.g., DNase and RNase.

In other embodiments, the hybrid nuclease molecules of the invention may be assembled together or with other polypeptides to form binding proteins having two or more polypeptides (“multimers”), wherein at least one polypeptide of the multimer is a hybrid nuclease molecule of the invention. Exemplary multimeric forms include dimeric, trimeric, tetrameric, and hexameric altered binding proteins and the like. In one embodiment, the polypeptides of the multimer are the same (ie. homomeric altered binding proteins, e.g. homodimers, homotetramers). In another embodiment, the polypeptides of the multimer are different (e.g. heteromeric).

### Methods of Making Hybrid Nuclease Molecules

The hybrid nuclease molecules of this invention largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, case of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85:2149; Davis et al. (1985), Biochem. Intl. 10:394-414; Stewart and Young (1969), Solid Phase Peptide Synthesis; U.S. Pat. No. 3,941,763; Finn et al. (1976), The Proteins (3rd ed.) 2:105-253; and Erickson et al. (1976), The Proteins (3rd ed.) 2:257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

### Pharmaceutical Compositions and Therapeutic Methods of Use

In certain embodiments, a hybrid nuclease molecule is administered alone. In certain embodiments, a hybrid nuclease molecule is administered prior to the administration of at least one other therapeutic agent. In certain embodiments, a hybrid nuclease molecule is administered concurrent with the administration of at least one other therapeutic agent. In certain embodiments, a hybrid nuclease molecule is administered subsequent to the administration of at least one other therapeutic agent. In other embodiments, a hybrid nuclease molecule is administered prior to the administration of at least one other therapeutic agent. As will be appreciated by one of skill in the art, in some embodiments, the hybrid nuclease molecule is combined with the other agent/compound. In some embodiments, the hybrid nuclease molecule and other agent are administered concurrently. In some embodiments, the hybrid nuclease molecule and other agent are not administered simultaneously, with the hybrid nuclease molecule being administered before or after the agent is administered. In some embodiments, the subject receives both the hybrid nuclease molecule and the other agent during a same period of prevention, occurrence of a disorder, and/or period of treatment.

Pharmaceutical compositions of the invention can be administered in combination therapy, i.e., combined with other agents. In certain embodiments, the combination therapy comprises nuclease molecule, in combination with at least one other agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In certain embodiments, the invention provides for pharmaceutical compositions comprising a hybrid nuclease molecule together with a pharmaceutically acceptable diluent, carrier, solubilizer, emulsifier, preservative and/or adjuvant.

In certain embodiments, the invention provides for pharmaceutical compositions comprising a hybrid nuclease molecule and a therapeutically effective amount of at least one additional therapeutic agent, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, a hybrid nuclease molecule and/or a therapeutic molecule is linked to a half-life extending vehicle known in the art. Such vehicles include, but are not limited to, polyethylene glycol, glycogen (e.g., glycosylation of the hybrid nuclease molecule), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising a hybrid nuclease molecule, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a hybrid nuclease molecule, with or without at least one additional therapeutic agent, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired hybrid nuclease molecule, with or without additional therapeutic agents, in a pharmaceutically acceptable vehicle. In certain embodiments, a vehicle for parenteral injection is sterile distilled water in which a hybrid nuclease molecule, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, a hybrid nuclease molecule, with or without at least one additional therapeutic agent, can be formulated as a dry powder for inhalation. In certain embodiments, an inhalation solution comprising a hybrid nuclease molecule, with or without at least one additional therapeutic agent, can be formulated with a propellant for aerosol delivery. In certain embodiments, solutions can be nebulized. Pulmonary administration is further described in PCT application no. PCT/US94/001875, which describes pulmonary delivery of chemically modified proteins.

In certain embodiments, it is contemplated that formulations can be administered orally. In certain embodiments, a hybrid nuclease molecule, with or without at least one additional therapeutic agents, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of a hybrid nuclease molecule and/or any additional therapeutic agents. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of a hybrid nuclease molecule, with or without at least one additional therapeutic agents, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving a hybrid nuclease molecule, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising a hybrid nuclease molecule, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which a hybrid nuclease molecule, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 ug/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of a hybrid nuclease molecule and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, the composition can be administered locally via

implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising a hybrid nuclease molecule, with or without at least one additional therapeutic agent, in an ex vivo manner. In such instances, cells, tissues and/or organs that have been removed from the patient are exposed to a pharmaceutical composition comprising a hybrid nuclease molecule, with or without at least one additional therapeutic agent, after which the cells, tissues and/or organs are subsequently implanted back into the patient.

In certain embodiments, a hybrid nuclease molecule and/or any additional therapeutic agents can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

The hybrid nuclease molecules of the instant invention are particularly effective in the treatment of autoimmune disorders or abnormal immune responses. In this regard, it will be appreciated that the hybrid nuclease molecules of the present invention may be used to control, suppress, modulate, treat, or eliminate unwanted immune responses to both external and autoantigens. In yet other embodiments the polypeptides of the present invention may be used to treat immune disorders that include, but are not limited to, insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbs-ve, cryptogenic cirrhosis, ulcerative colitis, Sjogren's syndrome, scleroderma, Wegener's granulomatosis, polymyositis, dermatomyositis, discoid LE, systemic lupus erythematosus, or connective tissue disease.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. Sec, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Construction of RNase-Ig Fusion Genes

Murine RNase I was amplified as a full-length cDNA from an EST library (from Dr. C. Raine, Albert Einstein School of Medicine, Bronx, NY) who sent the clone to our laboratory without an MTA. Sequence specific 5′ and 3′ primers used were from the published sequences. The sequence of the clone was verified by sequencing analysis. The Genebank accession number is NCBI geneID 19752. Full length human RNase 1 was isolated from random primed and oligo dT primed cDNA derived from human pancreas total RNA (Ambion/Applied Biosystems, Austin, TX).

Once a full-length clone was isolated, primers were designed to create a fusion gene with the mouse IgG2a (SEQ ID NO: 114) or human IgG1 (SEQ ID NO:110) Fc domains. Two different primers were designed for the 5′ sequence fused at the amino terminus of the Fc tail; the first incorporated the native leader peptide from mouse (or human) RNase, while the second attached an AgeI site to the amino terminus of RNase at the predicted signal peptide cleavage site in order to fuse the RNase to a human VKIII leader peptide that we already had cloned and used for other expression studies. For the murine RNase, the sequence of the first primer is:

mribNL5′
30mer (RNase 5′ with native leader and
HindIII + Kozak)
(SEQ ID NO: 1)
gTT AAg CTT gCC ACC ATg ggT CTg gAg AAg TCC CTC
ATT CTg-3′

The second primer creates a gene fusion junction between an existing leader sequence and the mature sequence at the 5′ end of the RNase, at or near the predicted leader peptide cleavage site.

27mer (RNase 5′ mature sequence
(no leader, with AgeI site)
(SEQ ID NO: 2)
5′-gAT ACC ACC ggT Agg gAA TCT gCA gCA CAg AAg
TTT CAg-3′

The sequence of the 3′ primer for fusion to murine IgG2a at the carboxy end of RNase and the amino terminus of the Fc tail is as follows:

mrib3NH2
28mer (RNase 3′ end with XhoI site for
fusion to mIgG2a).
(SEQ ID NO: 3)
5′-ggC TCg AgC ACA gTA gCA TCA AAg tGG ACT ggT
ACg TAg g-3′

Two more oligos were designed to create an-Ig-RNase fusion gene, where the—Ig tail is amino terminal to the RNase enzyme domain.

mrib5X
36mer RNase 5′ end with linker aa and XbaI site for
fusion to carboxy end of Fc domain.
(SEQ ID NO: 4)
5′-AAA TCT AgA CCT CAA CCA ggT Agg gAA TCT gCA gCA
CAg AAg TTT CAg-3′
mrib3X
31mer RNase 3′ end with two stop codons and XbaI
site for fusion to carboxy end of Fc domain.
(SEQ ID NO: 5)
5′-TCT AgA CTA TCA CAC AgT AgC ATC AAA gTg gAC Tgg
TAC gTA g-3′

### Example 2: Isolation of Anti-RNA or Anti-DNA scFvs from Monoclonal Antibody Expressing Hybridomas

An anti-RNA hybridoma designated H564 was used to isolate V regions specific for RNA. Prior to harvesting, H564 anti-RNA hybridoma cells were kept in log phase growth for several days in RPMI 1640 media (Invitrogen/Life Technologies, Gaithersburg, Md.) supplemented with glutamine, pyruvate, DMEM non-essential amino acids, and penicillin-streptomycin. Cells were pelleted by centrifugation from the culture medium, and 2×10⁷ cells were used to prepare RNA. RNA was isolated from the hybridoma cells using the QIAGEN RNAeasy kit (Valencia, Calif.) total RNA isolation kit and QIAGEN QIAshredder according to the manufacturer's instructions accompanying the kit. Four microgram (4 μg) of total RNA was used as template to prepare cDNA by reverse transcription. The RNA, 300 ng random primers, and 500 ng Oligo dT (12-18), and 1 μl 25 mM dNTPs were combined and denatured at 80° C. for 5 minutes prior to addition of enzyme. Superscript III reverse transcriptase (Invitrogen, Life Technologies) was added to the RNA plus primer mixture in a total volume of 25 μl in the presence of 5.times.second strand buffer and 0.1 M DTT provided with the enzyme. The reverse transcription reaction was allowed to proceed at 50° C. for one hour.

The cDNA generated in the reverse transcriptase reaction was purified by QIAquick PCR purification kits (QIAGEN, Valencia CA) and tailed with a poly-G sequence using terminal transferase (Invitrogen, Carlsbad, CA) according to manufacturer's instructions. Tailed cDNA was again purified by QIAquick PCR purification and eluted in 30 ul elution buffer (EB buffer) provided with the kits. Two microliters of tailed cDNA was used as template along with an anchor-tail 5′ primer containing a poly-C domain, and constant region specific, degenerate 3′ primers to amplify by PCR the variable regions for the light and heavy chain of the H564 antibody. The two variable chains were designed with restriction enzyme sites so that a scFv could be assembled by three way ligation of the two V regions to a linker sequence after amplification and restriction enzyme digestion.

A (gly4ser)4 peptide linker to be inserted between the two V regions was incorporated by amplification of this linker sequence by overlap extension PCR using overlapping primers encoding the two halves of the molecule. PCR fragments were isolated by agarose gel electrophoresis, fragments isolated by cutting the appropriate bands from the gel and purifying the amplified DNA using QIAquick gel extraction kits (QIAGEN, Valencia, CA). scFv derivatives from the H564 hybridoma were assembled as VH-linker-VL fusion genes that could be attached at either end of a larger-Ig fusion gene. The V.sub.H domain was amplified without a leader peptide, but included a 5′ AgeI restriction site for fusion to the V.sub.L and a BglII restriction site at the 3′ end for fusion to the linker domain.

The scFv-Ig was assembled by inserting the scFv HindIII-XhoI fragment into pDG containing the human IgG1 hinge, CH2, and CH3 regions, which was digested with restriction enzymes, HindIII and XhoI. After ligation, the ligation products were transformed into DH5-alpha bacteria. The scFv-Ig cDNA was subjected to cycle sequencing on a PE 9700 thermocycler using a 25-cycle program by denaturing at 96° C. for 10 seconds, annealing at 50° C. for 30 seconds, and extending at 72° C. for 4 minutes. The sequencing primers were pDG forward and reverse primers and an internal primer that annealed to the CH2 domain human in the IgG constant region portion. Sequencing reactions were performed using the Big Dye Terminator Ready Sequencing Mix v3.1 (PE-Applied Biosystems, Foster City, Calif.) according to the manufacturer's instructions. Samples were subsequently purified using Autoseq G25 columns (GE Healthcare) and the eluates dried in a Savant vacuum dryer, denatured in Template Suppression Reagent (PE-ABI), and analyzed on an ABI 310 Genetic Analyzer (PE-Applied Biosystems). The sequence was edited, translated, and analyzed using Vector Nti version 10.0 (Informax/Invitrogen, North Bethesda, Md.).

### Construction of a Human RNase1-hIgG1 (SEQ ID NO:125-127) Fusion Gene

Human RNase1 (SEQ ID NO:113) was isolated by PCR amplification from human pancreas total RNA obtained from Ambion/Applied Biosystems (Austin, TX). Four microgram (4 μg) of total RNA was used as template to prepare cDNA by reverse transcription. The RNA, 300 ng random primers, and 500 ng Oligo dT (12-18), and 1 ul 25 mM dNTPs were combined and denatured at 80° C. for 5 minutes prior to addition of enzyme. Superscript III reverse transcriptase (Invitrogen, Life Technologies) was added to the RNA plus primer mixture in a total volume of 25 μl in the presence of second strand buffer and 0.1 M DTT provided with the enzyme. The reverse transcription reaction was allowed to proceed at 50° C. for one hour. Reactions were further purified by QIAquick PCR purification columns, and cDNA eluted in 40 microliters EB buffer prior to use in PCR reactions. Two microliters cDNA eluate were added to PCR reactions containing 50 pmol 5′ and 3′ primers specific for human RNase1, and 45 microliters of PCR high fidelity supermix (Invitrogen, Carlsbad, CA) was added to 0.2 ml PCR reaction tubes. PCR reactions were performed using a C1000 thermal cycler (BioRad, Hercules CA). Reactions included an initial denaturation step at 95 C for 2 minutes, followed by 34 cycles with a 94° C., 30 sec denaturation, 50° C., 30 sec annealing, and 68° C., 1 minute extension step, followed by a final 4 minute extension at 72° C. Once wild type tails were isolated, the fragments were TOPO cloned into pCR2.1 vectors; DNA prepared using the QIAGEN spin plasmid miniprep kits according to manufacturer's instructions. Plasmid DNA was sequenced using ABI Dye Terminator v3.1 ready reaction mix according to manufacturer's instructions.

### Example 3: Isolation of Human and Mouse-Fc Domains and Introduction of Mutations into the Coding Sequence

For isolation of mouse (SEQ ID NO: 114) and human-Fc domains (SEQ ID NO: 110), RNA was derived from mouse or human tissue as follows. A single cell suspension was generated from mouse spleen in RPMI culture media. Alternatively, human PBMCs were isolated from fresh, whole blood using Lymphocyte Separation Media (LSM) Organon Teknika (Durham, NC), buffy coats harvested according to manufacturer's directions, and cells washed three times in PBS prior to use. Cells were pelleted by centrifugation from the culture medium, and 2×10⁷ cells were used to prepare RNA. RNA was isolated from the cells using the QIAGEN RNAeasy kit (Valencia, Calif.) total RNA isolation kit and QIAGEN QIAshredder columns according to the manufacturer's instructions accompanying the kits. One microgram (4 μg) of total RNA was used as template to prepare cDNA by reverse transcription. The RNA, 300 ng random primers, and 500 ng Oligo dT (12-18), and 1 μl 25 mM dNTPs were combined and denatured at 80° C. for 5 minutes prior to addition of enzyme. Superscript III reverse transcriptase (Invitrogen, Life Technologies) was added to the RNA plus primer mixture in a total volume of 25 μl in the presence of second strand buffer and 0.1 M DTT provided with the enzyme. The reverse transcription reaction was allowed to proceed at 50° C. for one hour. cDNA was purified using QIAquick (QIAGEN) PCR purification columns according to manufacturer's directions, and eluted in 40 microliters EB buffer prior to use in PCR reactions.

Wild type mouse and human-Fc domains were isolated by PCR amplification using the cDNA described above as template. The following primers were used for initial amplification of wild type sequences, but incorporated the desired mutational changes in the hinge domain:

mahIgG1CH2M: 47 mer
(SEQ ID NO: 6)
5′-tgtccaccgtgtccagcacctgaactcctgggtggatcgtcagtctt
cc-3′
hIgG1-5scc: 49 mer
(SEQ ID NO: 7)
5′-agatctcgagcccaaatcttctgacaaaactcacacatgtccaccgt
gt-3′
mahIgG1S: 51 mer
(SEQ ID NO: 8)
5′-tctagattatcatttacccggagacagagagaggctcttctgcgtgt
agtg-3′
muIgG2aCH2: 58 mer
(SEQ ID NO: 9)
5′-cctccatgcaaatgcccagcacctaacctcttgggtggatcatccgt
cttcatcttcc-3′
mIgG2a-5scc: 47 mer
(SEQ ID NO: 10)
5′-gaagatctcgagcccagaggtcccacaatcaagccctctcctcc
a-3′
mIgG2a3S: 48 mer
(SEQ ID NO: 11)
5′-gtttctagattatcatttacccggagtccgagagaagctcttagtcg
t-3′

PCR reactions were performed using a C1000 thermal cycler (BioRad, Hercules CA) or an Eppendorf thermal cycler (ThermoFisher Scientific, Houston TX). Reactions included an initial denaturation step at 95° C. for 2 minutes, followed by 34 cycles with a 94° C., 30 sec denaturation, 50° C., 30 sec annealing, and 72° C., 1 minute extension step, followed by a final 4 minute extension at 72° C. Once wild type tails were isolated, the fragments were TOPO cloned into pCR2.1 vectors, DNA prepared using the QIAGEN spin plasmid miniprep kits according to manufacturer's instructions and clones sequenced using ABI Dye Terminator v3.1 sequencing reactions according to manufacturer's instructions.

DNA from the correct clones were used as templates in overlap extension PCRs to introduce mutations at the desired positions in the coding sequence for mouse IgG2a or human-IgG1. PCR reactions were set up using the full length wild type clones as template (1 microliter), 50 pmol 5′ and 3′ primers to PCR each portion of the -Fc domain up to and including the desired mutation site from each direction, and PCR hi fidelity Supermix (Invitrogen, Carlsbad CA), in 50 microliter reaction volumes using a short amplification cycle. As an example of the overlapping PCR mutagenesis, the primer combination used to introduce the P331S mutation into human-IgG1, was as follows:

A 5′ subfragment was amplified using the full-length wild type clone as template, and the 5′ primer was hIgG1-5scc: 5′-agatctcgagcccaaatcttctgacaaaactcacacatgtccaccgtgt-3′ (SEQ ID NO: 12), while the 3′ primer was P331AS: 5′-gttttctcgatggaggctgggagggctttgttggagacc-3′ (SEQ ID NO:13). A 3′ subfragments was amplified using the full-length wild type clone as template and the 5′ primer was P331S: 5′aaggtctccaacaaagccctcccagcctccatcgagaaaacaatctcc-3′ (SEQ ID NO:14), while the 3′ primer was mahIgG1S: 5′-tctagattatcatttacccggagacagagagaggctcttctgcgtgtagtg-3′ (SEQ ID NO: 15).

Once subfragments were amplified and isolated by agarose gel electrophoresis, they were purified by QIAquick gel purification columns and eluted in 30 microliters EB buffer according to manufacturer's instructions. Two rounds of PCR were then performed with the two subfragments as overlapping templates in new reactions. The cycler was paused and the 5′ (hIgG1-5scc, see above) and 3′ (mahIgG1S, see above) flanking primers were added to the reactions (50 pmol each). PCR amplifications were then carried out for 34 cycles at the conditions described for the wild type molecules above. Full length fragments were isolated by gel electrophoresis, and TOPO cloned into pCR2.1 vectors for sequence analysis. Fragments from clones with the correct sequence were then subcloned into expression vectors for creation of the different hybrid nuclease molecules described herein.

### Example 4: Expression of RNAse-Ig (SEQ ID NO 124, 125, 126,127, 174 (Nucleotide) or 160, 161, 162,163, 175 (Amino Acid)), DNAse-Ig (SEQ ID NO: 118, 119,120,121,122,123, 186 (Nucleotide) or SEQ ID NO 154, 155, 156, 157, 158, 159, 187 (Amino Acid)), Multi-Subunit Ig Fusion Constructs (SEQ ID NO: 115, 116, 117, 172, 176, 178, 180 (Nucleotide) or SEQ ID NO 151 152, 153, 173, 177, 179, 181 (Amino Acid)), and H564 scFv-Ig Fusion Proteins in Stable CHO Cell Lines

This example illustrates expression of the different-Ig fusion genes described herein in eukaryotic cell lines and characterization of the expressed fusion proteins by SDS-PAGE and by IgG sandwich ELISA.

The -Ig fusion gene fragments with correct sequence were inserted into the mammalian expression vector pDG, and DNA from positive clones was amplified using QIAGEN plasmid preparation kits (QIAGEN, Valencia, Calif.). The recombinant plasmid DNA (100 μg) was then linearized in a nonessential region by digestion with AscI, purified by phenol extraction, and resuspended in tissue culture media, Excell 302 (Catalog #14312-79P, JRH Biosciences, Lenexa, Kans./SAFC). Cells for transfection, CHO DG44 cells, were kept in logarithmic growth, and 10⁷ cells harvested for each transfection reaction. Linearized DNA was added to the CHO cells in a total volume of 0.8 ml for electroporation.

Stable production of the -Ig fusion protein was achieved by electroporation of a selectable, amplifiable plasmid, pDG, containing the RNase-Ig cDNA under the control of the CMV promoter, into Chinese Hamster Ovary (CHO) cells. The pDG vector is a modified version of pcDNA3 encoding the DHFR selectable marker with an attenuated promoter to increase selection pressure for the plasmid. Plasmid DNA was prepared using Qiagen maxiprep kits, and purified plasmid was linearized at a unique AscI site prior to phenol extraction and ethanol precipitation. Salmon sperm DNA (Sigma-Aldrich, St. Louis, Mo.) was added as carrier DNA, and 100 μg each of plasmid and carrier DNA was used to transfect 10⁷ CHO DG44 cells by electroporation. Cells were grown to logarithmic phase in Excell 302 media (JRH Biosciences) containing glutamine (4 mM), pyruvate, recombinant insulin, penicillin-streptomycin, and 2×DMEM nonessential amino acids (all from Life Technologies, Gaithersburg, Md.), hereafter referred to as “Excell 302 complete” media. Media for untransfected cells also contained HT (diluted from a 100× solution of hypoxanthine and thymidine) (Invitrogen/Life Technologies). Media for transfections under selection contained varying levels of methotrexate (Sigma-Aldrich) as selective agent, ranging from 50 nM to 1 μM. Electroporations were performed at 280 volts, 950 microFarads. Transfected cells were allowed to recover overnight in non-selective media prior to selective plating in 96 well flat bottom plates (Costar) at varying serial dilutions ranging from 125 cells/well to 2000 cells/well. Culture media for cell cloning was Excell 302 complete, containing 50 nM methotrexate. Once clonal outgrowth was sufficient, serial dilutions of culture supernatants from master wells were screened for expression of-Ig fusion protein by use of an-IgG sandwich ELISA. Briefly, NUNC immulon II plates were coated overnight at 4° C. with 7.5 microgram/ml F(ab′2) goat anti-mouse IgG (KPL Labs, Gaithersburg, MD) in PBS. Plates were blocked in PBS/3% BSA, and serial dilutions of culture supernatants incubated at room temperature for 2-3 hours. Plates were washed three times in PBS/0.05% Tween 20, and incubated with horseradish peroxidase conjugated F(ab′2) goat anti-mouse IgG2a (Southern Biotechnologies) and goat anti-mouse IgG (KPL) mixed together, each at 1:3500 in PBS/1.0% BSA, for 1-2 hours at room temperature. Plates were washed four times in PBS/0.05% Tween 20, and binding detected with SureBlue Reserve, TMB substrate (KPL Labs, Gaithersburg, MD). Reactions were stopped by addition of equal volume of IN HCl, and plates read at 450 nM on a Spectramax Pro plate reader (Microdevices, Sunnyvale CA). The clones with the highest production of the fusion protein were expanded into T25 and then T75 flasks to provide adequate numbers of cells for freezing and for scaling up production of the fusion protein. Production levels were further increased in cultures from the four best clones by progressive amplification in methotrexate containing culture media. At each successive passage of cells, the Excell 302 complete media contained an increased concentration of methotrexate, such that only the cells that amplified the DHFR plasmid could survive. The production level of the top four unamplified master wells from the RNase1g CHO transfectants ranged from 30-50 micrograms/ml culture. The amplified cultures are currently being assayed to determine production levels.

Supernatants were collected from CHO cells expressing the RNase-Ig, filtered through 0.2 μm PES express filters (Nalgene, Rochester, N.Y.) and were passed over a Protein A-agarose (IPA 300 crosslinked agarose) column (Repligen, Needham, Mass.). The column was washed with column wash buffer (90 mM Tris-Base, 150 mM NaCl, 0.05% sodium azide, pH 8.7), and bound protein was eluted using 0.1 M citrate buffer, pH 3.0. Fractions were collected and protein concentration was determined at 280 nM using a Nanodrop (Wilmington DE) microsample spectrophotometer, and blank determination using 0.1 M citrate buffer, pH 3.0. Fractions containing fusion protein were pooled, and buffer exchange performed by serial spins in PBS using centricon concentrators followed by filtration through 0.2 μm filter devices, to reduce the possibility of endotoxin contamination. An extinction coefficient of 1.05 was determined using the protein analysis tools in the Vector Nti Version 10.0 Software package (Informax, North Bethesda, Md.) and the predicted cleavage site from the online ExPasy protein analysis tools.

### Example 5: SDS-PAGE Analysis of RNaseIg Fusion Protein

Purified RNase-Ig (SEQ ID NO:115) was analyzed by electrophoresis on SDS-Polyacrylamide gels. Fusion protein samples were boiled in SDS sample buffer with and without reduction of disulfide bonds and applied to SDS 10% Tris-BIS gels (Catalog #NP0301, Novex, Carlsbad, Calif.). Five micrograms of each purified protein was loaded on the gels. The proteins were visualized after electrophoresis by Coomassie Blue staining (Pierce Gel Code Blue Stain Reagent, Catalog #24590, Pierce, Rockford, Ill.), and destaining in distilled water. Molecular weight markers were included on the same gel (Kaleidoscope Prestained Standards, Catalog #161-0324, Bio-Rad, Hercules, Calif). Other samples were run as follows: Rnase-Ig fusion protein in the sampling buffer (62.5 mM Tris-HCl, pH6.8, 2% SDS, 10% glycerol, 0.01% Bromophenol blue) with and without 5% 2-mercaptoethanol) was loaded onto the 4-12% pre-cast gel (Bio-RAD). The gel was running at 100 volts until the dye ran off the gel. The gel was stained in the GelCode Blue (Thermo scientific) at room temperature overnight and then washed with water.

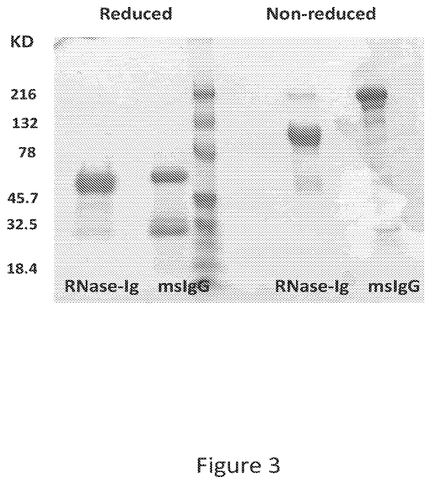

FIG. 3 shows the RNase-Ig fusion protein compared to mouse IgG. Rnase-Ig was purified from supernatant transfected CHO cells by binding and elution from Protein A sepharose. The SDS-PAGE gel shows that Rnase-Ig is approximately 50 kDa when reduced and approximately 110 kDa when non-reduced.

### Example 6: Detection of RNase-Ig in Mouse Sera

### SRED Assay

The 2% agarose gel was prepared with distilled water. Poly-IC (Sigma) was dissolved in distilled water at 3 mg/ml and the gel plate was prepared as follows: 1.5 ml reaction buffer (0.2M Tris-HCl pH 7.0, 40 mM EDTA and 0.1 mg/ml ethidium bromide), 1 ml Poly-IC and 0.5 ml water were place in the tube and maintained at 50° C. for 5 min. 3 ml of the agarose (kept at 50° C.) was added to the tube. The mixture was immediately poured onto a glass plate. Sampling wells were punched in the gel. 2 μl of each serum sample was loaded into wells and the gel was incubated at 37° C. for 4 hours in the moist chamber. Then the gel was incubated in a buffer (20 mM sodium acetate pH5.2, 20 mg/ml ethidium bromide) on ice for 30 min. and read under UV.

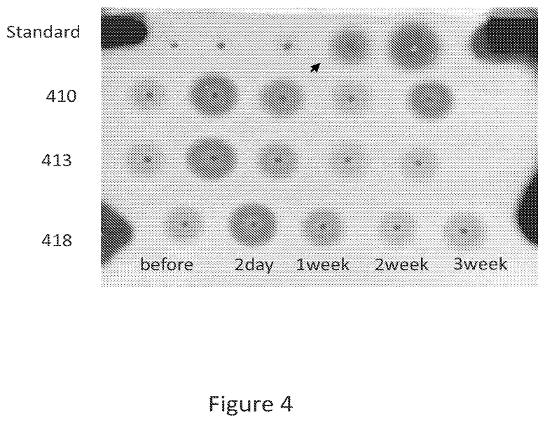

FIG. 4 shows RNase activity from three mice (410, 413, and 418) after intravenous injection of Rnase-Ig fusion protein (SEQ ID NO:150) (purified in this experiment from supernatant of transfected COS cells by binding and elution from protein A sepharose). A standard was used in the top row. Notice a second injection for mouse 410 (see arrow) after 2 weeks. 2 μl serum from each of three mice was loaded on 1% agarose gel containing 0.5 mg/ml poly-C. The gel was incubated for 4 hours in a moist chamber at 37° C., and then immersed in a buffer containing 20 mM sodium acetate and 20 ug/ml ethidium bromide for 30 min. The RNase activity is reflected by the size and intensity around the central well. This data shows that the RNase-Ig fusion protein has an extended half-life in mouse serum.

### Example 7: Use of an Anti-RNA ELISA to Measure RNA Specific Antibodies in Mouse Sera

A 96-well plate (Nunc, Thermal fisher scientific) was coated with 50 μg/ml of Poly-L-Lysine (Sigma) overnight. After washing five times with PBS containing 0.05% Tween, the plate was coated with 10 μg/ml of yeast RNA in PBS at 4° C. overnight. After washing five times, the plate was blocked with PBS containing 1% BSA at room temperature for 2 hours. Serum samples at 1:50 dilution were added to the plate and incubated at 4° C. overnight. Hybridoma H564 (anti-RNA) culture medium was used as standard, using two-fold serial dilutions starting at 1:300. Detection antibody was anti-mouse IgG conjugated with alkaline phosphatase (Jackson Lab), and was added to the plate at 1:5000 for 1 hour at room temperature. Phosphatase substrate (Sigma) was dissolved in developing buffer (ThermoFisher Scientific) and added to the plate at 50 μl/well. Samples were read at 405 nm using a Spectramax Plus plate reader (Microdevices, Sunnyvale, CA).

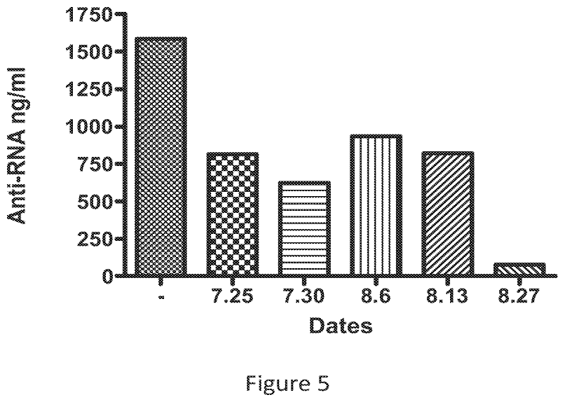

FIG. 5 shows the results from the anti-RNA Antibody ELISA titer before and after intravenous injection of RNase-Ig fusion protein (SEQ ID NO: 150) from mouse 410. The pre-coated Poly-L-lysine (50 μg/ml) plate was coated with 10 μg/ml yeast RNA. Serum (1:50) was loaded on the plate and incubated overnight at 4° C. Detection antibody was anti-mouse IgG-alkaline phosphatase (Jackson Labs) at 1:5000 for 1 hour at room temperature, and then phosphatase substrate was added and read at 405 nm. The data show that injection of Rnase-Ig caused a reduction in titer of anti-RNA antibody that persisted for over 3 weeks.

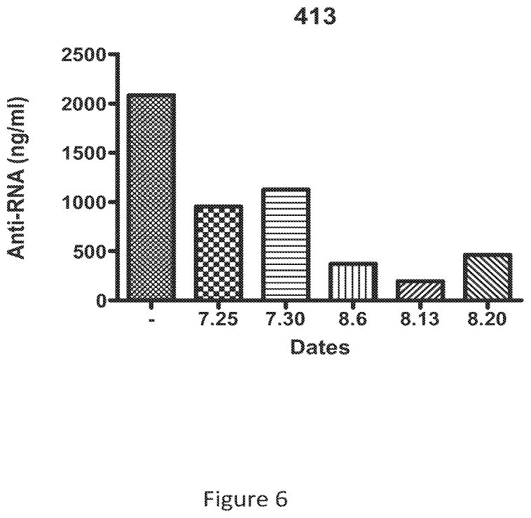

FIG. 6 shows the results from the anti-RNA Antibody ELISA titer before and after injection of RNase-Ig fusion protein (SEQ ID NO:150) within three weeks from mouse 413. The experiment was done as described for mouse 410. Titer of anti-RNA antibody was reduced after injection of Rnase-Ig.

### Example 8: IFN-Alpha Production by Human PBMCs is Inhibited by RNaseIg Addition to Cultures In Vitro

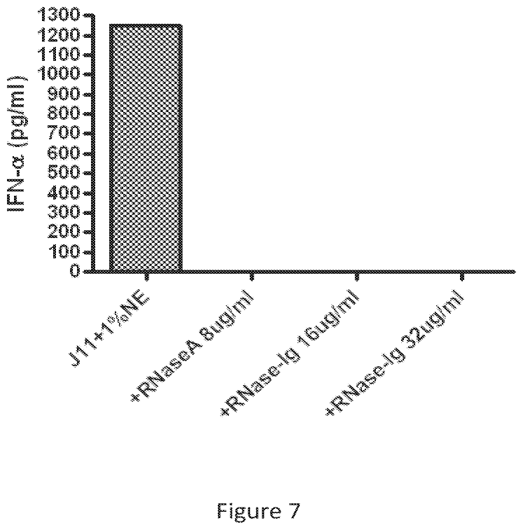

RNase-Ig (SEQ ID NO: 150) addition abolished the induction of interferon-a from human peripheral blood mononuclear cells stimulated using immune complexes formed with serum from an SLE patient (J11) plus nuclear extract (NE). Briefly, ELISA plates were coated with 50 microliters 1:2500 capture antibody (anti-IFN alpha, PBL 21112-1, Piscataway, NJ), and incubated overnight at 4° C. Plates were washed with PBS/0.05% Tween 20, blocked in PBS/1% BSA for 2 hours at room temperature, washed with PBS/0.05% Tween-20, and incubated with standard dilutions of IFN-alpha, or with serial dilutions of serum samples, and incubated 2 hours at room temperature. Plates were washed and incubated with 1:2000 detection antibody (PBL 31101-2, Piscataway, NJ) in PBS/1% BSA. Plates were washed in PBS/0.05% Tween-20, and incubated with 50 microliters donkey anti-rabbit HRP (Jackson Immunoresearch, Westgrove, PA) at 1:12,000 in PBS/1% BSA. Plates were washed five times prior to addition of TMB substrate. Reactions were stopped by addition of ½ volume 2N H2SO4, and samples read at 450 nm on a Spectramax Pro plate reader (MicroDevices, Sunnyvale, CA). The results are shown in FIG. 7, which shows RNase-Ig addition abolished the induction of interferon-a from human peripheral blood mononuclear cells stimulated using immune complexes formed with serum from an SLE patient (J11) plus nuclear extract.

### Example 9: Phenotype of TLR7.1×RNaseA Double Transgenic Mice

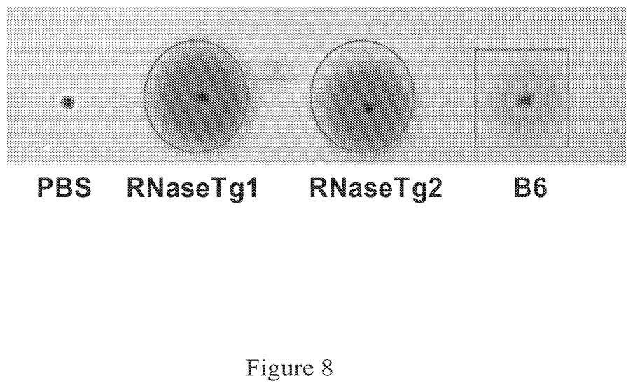

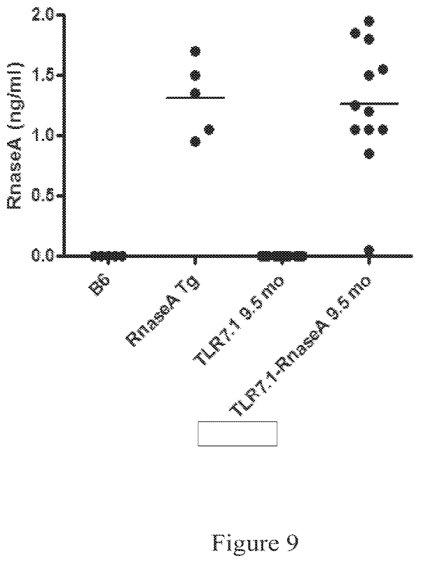

We have created mice that overexpress RNaseA (RNase Tg). This nuclease is expressed at high levels in RNase Tg mice (see FIG. 8). We have developed both a single radial diffusion (SRED) method (left panel) and a much more quantitative ELISA to quantify RNase in the serum (see FIG. 9). We crossed RNaseA Tg with TLR7.1 Tg mice to create the double Tg (DTg). TLR7.1 mice have 8-16 copies of TLR7 and develop a very aggressive, rapidly progressive lupus-like disease and start to die at 3 mo of age with a median survival of 6 mo. In a preliminary analysis, we bled DTg and littermate controls at 3 mo of age to see whether the DTg mice exhibited signs of improvement. As shown in FIG. 8, DTg mice had very high levels of RNase in their serum (equivalent to >13 U/ml RNase based on our standard with specific activity of 993 U/mg). RNaseA concentration in Tg and DTg mice was also measured by ELISA assay as shown in FIG. 9. The RNase A Tg and TLR7.1×RNaseA Dtg mice have RNase A serum concentrations between 1-2 ng/ml.

Detailed Method for Rnase A ELISA (Example 9, FIG. 9)
1. Coat plate with anti-RNaseA Abcam Ab (ab6610): 2.5-10 ug/ml O/N in 4 C.
2. Wash plate 3 times with 0.05% Tween/1×PBS
3. Block with 1% BSA in PBS for at least 1 hour
4. Wash plate 3 times with 0.05% Tween/1×PBS
5. Load samples. Sample dilutions at 1:50
6. Incubate Rm Temp for 2 hours
7. Wash plate 3 times with 0.05% Tween/1×PBS
8. Prepare dilution of biotin labeled Anti Rnase Ab at dilution of 1:4500 (2.2 ug/ml). Leave RT for 1 hour (Rockland 200-4688: 10 mg/ml).
9. Wash plate 3 times
10. Dilute StrepAV HRP (Biolegend 405210) 1:2500. Cover with foil and leave at RT for 25-30 min.
11. Wash 6 times, let the liquid sit in wells for at least 30 seconds in between washes.
12. Add BD OptEIA substrate A+B 1:1. Wait until color changes 5-10 min max. Don't let the top well standard go over 1.0. Add 80 ul. (CatNos: 51-2606KC; ReagentA, 51-2607KC; ReagentB)
13. Add 40 ul of 1M sulfuric acid to stop reaction

### Product/Reagent Information:

RNaseA Ab: ab6610 (90 mg/ml)
ELISA buffer: 1% BSA in PBS
ELISA wash buffer: 0.05% Tween/1×PBS
Anti RNaseA biotin conjugated Ab: Rockland: 200-4688 (10 mg/ml)
Strep AV HRP: Biolegend 405210
BD OptEIA reagent A and B: 51-2606KC and 51-2607KC

### Example 10. Survival Curves for TLR7.1 Transgenic Mouse Strains

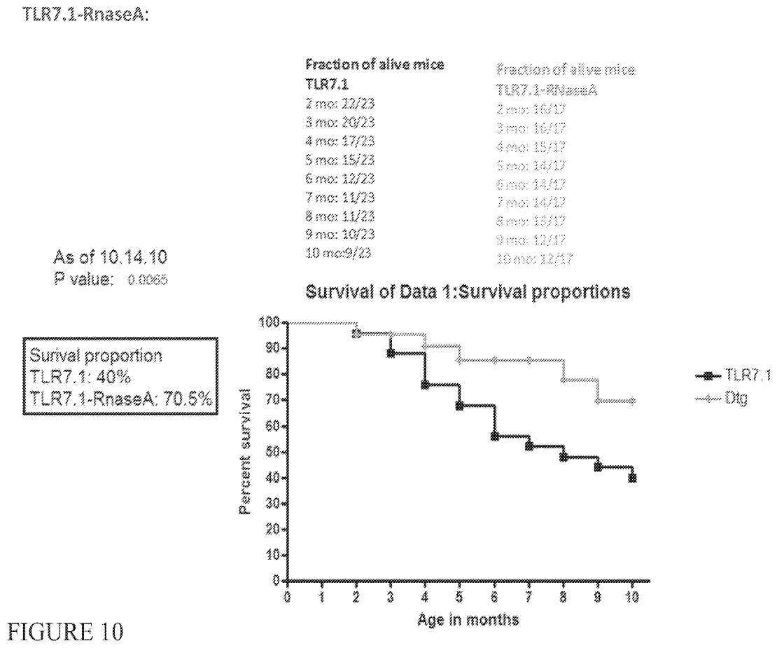

There was a highly significant difference between the DTg and the TLR7.1 littermate controls in survival. As shown in FIG. 10, at 10 months, 61% of TLR7.1 mice had died, whereas 31% of DTg mice had died. This data shows that overexpression of RNaseA exerted a strong therapeutic effect. The reasons why TLR7.1 mice die prematurely is not entirely clear, although severe anemia, thrombocytopenia, and glomerulonephritis could play a part. To determine whether red cell and platelet counts were positively impacted by RNaseA expression in the DTg mice, we performed blood counts but found no differences between the TLR7.1 and DTg mice. In contrast, there was a significant improvement in kidney histopathology in the DTg mice. We observed decreased deposition of IgG and C3 in DTg mice. PAS staining, which reflects inflammation in the mesangium was also reduced in DTg mice compared to TLR7.1 littermate controls. When we have now compared macrophage infiltration of the kidneys using anti-MAC-2 (galectin3) antibody (Lyoda et al. Nephrol Dial Transplat 22:3451, 2007), there were many fewer mac-2 positive cells in the glomeruli of the DTg mice. The results of counting 20 glomeruli per mouse in 5 mice in each group revelacd mean+/−SE of 3.8+/−1.1 and 1.4+/−0.2 for single versus DTg respectively, p=0.05. In addition, we quantified glomerular tuft size and observed a significant reduction in glomerular tuft size in the DTg mice (179+/−41 versus 128+/−16.8 um2 in single versus DTg respectively, p=0.037). In summary, TLR7.1×RNaseA DTg mice survive longer than their single Tg TLR7.1 littermates and have less inflammation and injury in their kidneys.

### Example 11. Analysis of IRGs in Spleens of TLR Tg Mice

Analysis of interferon response genes (IRGs) in the spleens of TLR7.1 Tg and TLR7.1×RNaseA DTg mice mice showed that expression of the IRF7 gene was significantly lower in the DTg mice (p=0.03). Some other IRGs including MX1 and VIG1 were lower in DTg mice compared to Tg mice, but the differences were not significant. See FIG. 11. Quantitative PCR was performed as follows: total RNA was isolated from mouse spleens using the RNeasy mini kit (Qiagen, Valencia, CA, USA), DNase treated using Turbo DNA-free (Applied Biosystems, Foster City, CA, USA) and first-strand cDNA was produced with the RNA-to-cDNA kit (Applied Biosystems) using random primers. The 260/280 was between 1.7 and 2.0 for isolated RNA measured with a NanoDrop (Thermo Scientific, Waltham, MA, USA). cDNA was diluted to an equivalent of Ing/ul total RNA and 8 ul were used per reaction. Primers for the reference gene (18s) and genes of interest (GOI) were synthesized (IDT, Coralville, Iowa, USA) and diluted to the appropriate concentrations for qPCR using molecular grade water. BLAST results of the primers show specific sequence homology only to the reference gene or GOI. Reactions in duplicate (20 ul) were run on an ABI Fast 7500 system using a 1:1 mix of template and primer to SensiMix SYBR low-ROX master mix (Bioline, London, UK). Relative quantification was calculated using the 2^{-ddCT} method with age matched wild type B6 mice as baseline to determine fold changes for each GOI. The dissociation curves for the reactions show a single melt peak for each gene. The standard curve showed similar amplification efficiencies for each gene and that template concentrations were within the linear dynamic range for each of primer set.

### Example 12. Structures for Generating Hybrid Nuclease Molecules

Hybrid nuclease molecules were designed to incorporate desired structures and functional activity of single enzyme or multi-enzyme structures as modular cassettes with compatible restriction enzyme sites for shuttling and domain exchange. The schematic structure of different embodiments of hybrid nuclease molecules is illustrated in FIG. 12. Primers are shown in Table 1. The nucleotide and amino acid sequences of representative hybrid nuclease molecules are shown in Table 2.

### General Approach for Generation of Hybrid Nuclease Molecules

Human cDNAs were isolated from human pancreas RNA (Ambion) or human PBMC RNA from normal human peripheral blood lymphocytes (approximately 5×10e6) using QIAgen RNAeasy kits (Valencia, CA) and QIAshredder kits to homogenize cell lysates (Qiagen, Valencia, CA). Human PBMCs were isolated from heparinized human blood diluted 1:1 in D-PBS and layered over LSM Lymphocyte Separation Medium (MP Biomedicals, Irvine, CA) Ficoll gradients.

Mouse spleen RNA was isolated using QIAgen RNAeasy kits (Valencia, CA) from approximately 5×10e6 splenocytes. Cells were pelleted by centrifugation from the culture medium, and 5×10e6 cells were used to prepare RNA. RNA was isolated from the cells using the QIAGEN RNAeasy kit (Valencia, Calif.) total RNA isolation kit and QIAGEN QIAshredder according to the manufacturer's instructions accompanying the kit. One to two microgram (1-2 μg) of total RNA was used as template to prepare cDNA by reverse transcription. The RNA, 300 ng random primers, and 500 ng Oligo dT (12-18), and 1 μl 25 mM dNTPs were combined and denatured at 80° C. for 5 minutes prior to addition of enzyme. Superscript III reverse transcriptase (Invitrogen, Life Technologies) was added to the RNA plus primer mixture in a total volume of 25 μl in the presence of 5 times second strand buffer and 0.1 M DTT provided with the enzyme. The reverse transcription reaction was allowed to proceed at 50° C. for one hour.

Between 10-100 ng cDNA was used in PCR amplification reactions using primers specific for the nuclease gene of interest (RNaseA, RNase1, DNase1, Trex1, DNase 1L3, etc.) For initial cloning reactions, primers were designed to isolate the full length cDNA or truncation products encoding the gene of interest. Full length or shortened PCR fragments were isolated by agarose gel electrophoresis, and purified using Qiagen QIAquick columns to remove nucleotides, primers, and unwanted amplified products. Purified fragments were cloned into pCR2.1 TOPO cloning vectors (Invitrogen, Carlsbad, CA) and transformed into TOP10 competent bacteria. Isolated colonies were picked into Luria Broth media containing 50 ug/ml carbenicillin, and grown overnight to isolate plasmids. TOPO clones were screened for inserts of the correct size by digestion with EcoRI (NEB, Ipswich, MA) restriction enzyme and agarose gel electrophoresis of digested fragments. DNA sequence analysis of positive clones was performed with ABI Ready Reaction Mix v 3.1 and analyzed using an ABI 3730 XL DNA sequencer. Once correct clones were obtained, further sequence modifications were designed and PCR reactions performed to generate the desired alleles or expression cassettes. Truncation products and alleles were generated by PCR mutagenesis using overlapping primers for introduction of mutations at specific positions in the genes. Linkers were synthesized by overlapping PCR using internal overlapping primers and successive rounds of PCR to attach additional sequence to each terminus. Hybrid nuclease molecules were assembled as a string of several interchangeable cassettes. Molecules of the preferred embodiment contain a fixed leader peptide, a nuclease cassette, an optional cassette encoding a choice of several different polypeptide linkers, an-Ig Fc domain cassette with either a STOP codon or a linker at the carboxyl end of the CH3 domain, and for resolvICase type molecules, a second linker cassette, followed by a second nuclease cassette. FIG. 12 illustrate the cassette type structure of these hybrid nuclease molecules and examples of potential sequences inserted at each position. Once hybrid nuclease molecules were assembled, they were transferred to a mammalian expression plasmid pDG appropriate for transient expression in COS7 or other cells and stable expression in CHO DG44 cells using selection for DHFR with methotrexate.

### Transient Expression of Hybrid Nuclease Molecules

COS-7 cells were transiently transfected with expression vector pDG containing hybrid nuclease molecule gene inserts. The day before transfection, cells were seeded at 4×10e5 cells per 60 mm dish in 4 ml DMEM (ThermoFisher/Mediatech cell gro)+10% FBS tissue culture media. DMEM basal media was supplemented with 4.5 g/L glucose, sodium pyruvate, L-glutamine 4 mM, and non-essential amino acids. Fetal bovine serum (Hyclone, Logan, UT ThermoFisher Scientific) was added to media at 10% final volume. Cells were incubated at 37° C., 5% CO2 overnight and were approximately 40-80% confluent on the day of transfection. Plasmid DNA was prepared using Qiagen (Valencia, CA) QIAprep miniprep kits according to manufacturer's instructions, and eluted in 50 ul EB buffer. DNA concentrations were measured using a Nanodrop 1000 (Thermo Fisher Scientific, Wilmington DE) spectrophotometer. Plasmid DNA was transfected using Polyfect (Qiagen, Valencia, CA) transfection reagent according to manufacturer's instructions, using 2.5 ug plasmid DNA per 60 mm dish and 15 ul polyfect reagent in 150 ul serum free DMEM transfection cocktails. After complex formation, reactions were diluted into 1 ml cell growth media containing serum and all supplements, and added drop-wise to the plates containing 3 ml fresh DMEM complete culture media. Transient transfections were incubated for 48-72 hours prior to harvesting culture supernatants for further analysis.

### Generation of Stable CHO DG44 Transfectants Expressing the Hybrid Nuclease Molecules of Interest

Stable production of the hybrid nuclease molecules was achieved by electroporation of a selectable, amplifiable plasmid, pDG, containing the nuclease-Ig cDNA under the control of the CMV promoter, into Chinese Hamster Ovary (CHO) cells. The PDG vector is a modified version of pcDNA3 encoding the DHFR selectable marker with an attenuated promoter to increase selection pressure for the plasmid. Plasmid DNA was prepared using Qiagen maxiprep kits, and purified plasmid was linearized at a unique AscI site prior to phenol extraction and ethanol precipitation. Salmon sperm DNA (Sigma-Aldrich, St. Louis, Mo.) was added as carrier DNA, and 100 μg each of plasmid and carrier DNA was used to transfect 10⁷ CHO DG44 cells by electroporation. Cells were grown to logarithmic phase in Excell 302 media (JRH Biosciences) containing glutamine (4 mM), pyruvate, recombinant insulin, penicillin-streptomycin, and 2×DMEM nonessential amino acids (all from Life Technologies, Gaithersburg, Md.), hereafter referred to as “Excell 302 complete” media. Media for untransfected cells also contained HT (diluted from a 100x solution of hypoxanthinc and thymidine) (Invitrogen/Life Technologies). Media for transfections under selection contained varying levels of methotrexate (Sigma-Aldrich) as selective agent, ranging from 50 nM to 1 μM. Electroporations were performed at 280 volts, 950 microFarads. Transfected cells were allowed to recover overnight in non-selective media prior to selective plating in 96 well flat bottom plates (Costar) at varying serial dilutions ranging from 125 cells/well to 2000 cells/well. Culture media for cell cloning was Excell 302 complete, containing 50 nM methotrexate. Once clonal outgrowth was sufficient, serial dilutions of culture supernatants from master wells were screened for expression of hybrid nuclease molecules by use of an-IgG sandwich ELISA. Briefly, NUNC immulon II plates were coated overnight at 4° C. with 7.5 microgram/ml F(ab′2) goat anti-mouse IgG (KPL Labs, Gaithersburg, MD) or 2 ug/ml goat anti-human or anti-mouse IgG (Jackson Immunoresearch, West Grove PA) in PBS. Plates were blocked in PBS/2-3% BSA, and serial dilutions of culture supernatants incubated at room temperature for 2-3 hours. Plates were washed three times in PBS/0.05% Tween 20, and incubated with horseradish peroxidase conjugated F(ab′2) goat anti-mouse IgG2a (Southern Biotechnologies) and goat anti-mouse IgG (KPL) mixed together, each at 1:3500 in PBS/1.0% BSA, or in horseradish peroxidase conjugated F(ab′) 2 goat anti-human IgG1 (Jackson Immunoresearch, West Grove, PA) at 1:2500 for 1-2 hours at room temperature. Plates were washed four times in PBS/0.05% Tween 20, and binding detected with SureBlue Reserve, TMB substrate (KPL Labs, Gaithersburg, MD). Reactions were stopped by addition of equal volume of 1N HCl, and plates read at 450 nM on a Spectramax Pro plate reader (Microdevices, Sunnyvale CA). The clones with the highest production of the hybrid nuclease molecule were expanded into T25 and then T75 flasks to provide adequate numbers of cells for freezing and for scaling up production of the fusion protein. Production levels were further increased in cultures from the four best clones by progressive amplification in methotrexate containing culture media. At each successive passage of cells, the Excell 302 complete media contained an increased concentration of methotrexate, such that only the cells that amplified the DHFR plasmid could survive.

Supernatants were collected from CHO cells expressing the hybrid nuclease molecule, filtered through 0.2 μm PES express filters (Nalgene, Rochester, N.Y.) and were passed over a Protein A-agarose (IPA 300 crosslinked agarose) column (Repligen, Needham, Mass.). The column was washed with column wash buffer (90 mM Tris-Base, 150 mM NaCl, 0.05% sodium azide, pH 8.7), and bound protein was eluted using 0.1 M citrate buffer, pH 3.0. Fractions were collected and protein concentration was determined at 280 nM using a Nanodrop (Wilmington DE) microsample spectrophotometer, and blank determination using 0.1 M citrate buffer, pH 3.0. Fractions containing hybrid nuclease molecules were pooled, and buffer exchange performed by serial spins in PBS using centricon concentrators followed by filtration through 0.2 μm filter devices, to reduce the possibility of endotoxin contamination.

### Example 13. Analysis of Enzyme Kinetics for hRNase1-G88D-hIgG1 [SCCH-P238S-K322S-P331S]

The human RNase1 sequence was isolated from human pancreas RNA by random primed cDNA reverse transcription and PCR amplification as described in Example 12 for nuclease molecules. The following primers at 50 pmol per reaction were used from the primer set listed in the PCR primer table.

hRNase5′age:
(SEQ ID NO: 16)
accggtaaggaatcccgggccaagaaattcc
hRNase3′bx:
(SEQ ID NO: 17)
ctcgagatctgtagagtcctccacagaagcatcaaagtgg

The mutant form of human RNase G88D was created by using the following two primers in PCR and overlap PCR reactions to introduce a mutation at position 88 that alters the resistance of the enzyme to the cytoplasmic inhibitor.

hRNaseG88D-S:
(SEQ ID NO: 18)
agactgccgcctgacaaacgactccaggtaccc
hRNAseG88D-AS:
(SEQ ID NO: 19)
gggtacctggagtcgtttgtcaggcggcagtct

Both wild type and mutant versions of human RNase1 were isolated and cloned as described for hybrid nuclease molecules above. The wild type sequence was cloned using the first two primers listed above. Once the RNase fragments were TOPO cloned and sequenced, the AgeI-XhoI cassettes were transferred to the pDG expression vector already containing the human VK3LP insert and the human IgG1-WT cassette. Constructs were verified by digestion, and plasmid DNA prepared for transient transfections. Once function was confirmed from small scale transient transfections, the molecules were stably transfected into CHO DG44 in order to express sufficient quantities for further in vitro analysis. The wild type human RNase1 fusion protein is shown in Table 2, hVK3LP-hRNase1-WT-hIgG1-WT (SEQ ID NO:163). Similarly, wild type human RNase1 was also expressed as a fusion gene with a (gly4ser)4 (SEQ ID NO:125 or SEQ ID NO:161) or (gly4ser)5 (SEQ ID NO:126 or SEQ ID NO: 162) linker domain inserted between the hRNase cassette and the hIgG1 Fc domain. The G88D mutant of human RNase1 was also expressed as a fusion gene designated hVK3LP-hRNase-G88D-hIgG1-WT (SEQ ID NO:124 or 160) or hIgG1-SCCH-P238S-K322S-P331S (SEQ ID NO:174 or 175), listed in Table 2.

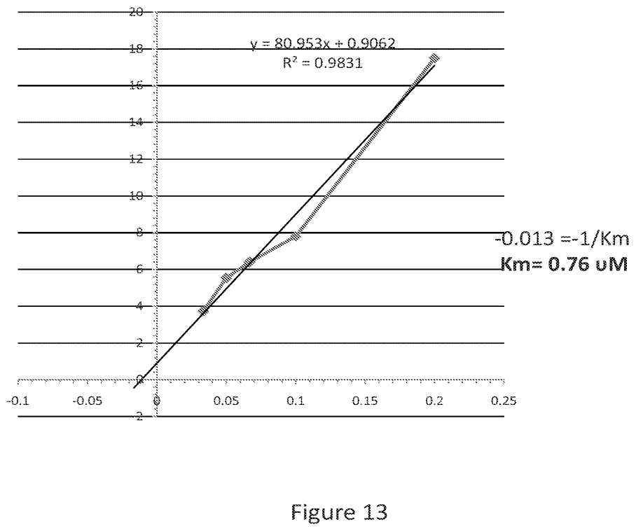

The Lineweaver Burk plot of enzyme kinetics for the mutant hRNase1-G88D-hIgG1 [SCCH-P238S-K322S-P331S] (SEQ ID NO:175) is shown in FIG. 13. To further define the functional characteristics of the bivalent RNase-Ig fusion protein, we performed preliminary determinations of the Michaelis constant, Km. Enzyme kinetics of purified human RNase1-Ig fusion protein was assayed using the RNase Alert Substrate (Ambion/IDT, San Diego, CA.) according to manufacturer's instructions and fluorescence assayed using a Spectramax M2 microplate reader (Molecular Devices, Sunnyvale, CA). Fluorescence data was collected at 30 second intervals over the course of a 30 minute incubation, and analyzed using SoftmaxPro Software (Molecular Devices) Reaction rates at different substrate concentrations were measured and the data is shown in the form of a Lineweaver Burke plot.

### Example 14. Analysis of Binding of hRNase1-hIgG to Human Monocytic Lines

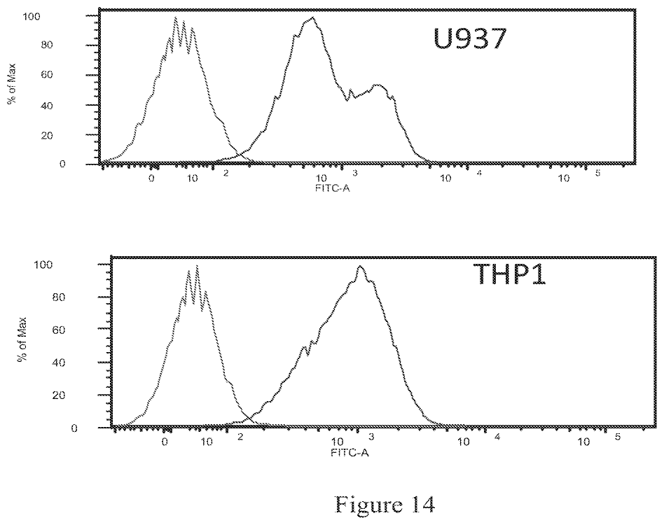

Protein A purified hybrid nuclease molecules hRNase1-hIgG1-WT were incubated with human monocytic cell lines THP-1 or U937 to assess FcR mediated binding of the wild type or mutant Fc containing molecules. FIG. 14 shows the binding pattern of hRNase1-WT-hIgG1-WT (SEQ ID NO:161) to these two cell lines. Cells were incubated with 5 ug/ml purified fusion protein in PBS/2% FBS for 45 minutes on ice, washed three times in PBS/2% FBS, and incubated with FITC-goat anti-human IgG (Fc specific) (Jackson Immunoresearch, West Grove, PA) at 1:200 for 45 minutes on ice. Cells were washed two times in PBS/2% FBS and analyzed by flow cytometry using a FACS Canto (BD, Franklin Lakes, NJ) flow cytometer, and FlowJo software (TreeStar, Ashland, OR).

### Example 15. IVIg Blocking of hRNase1-hIgG1 Binding to Human Monocytic Lines

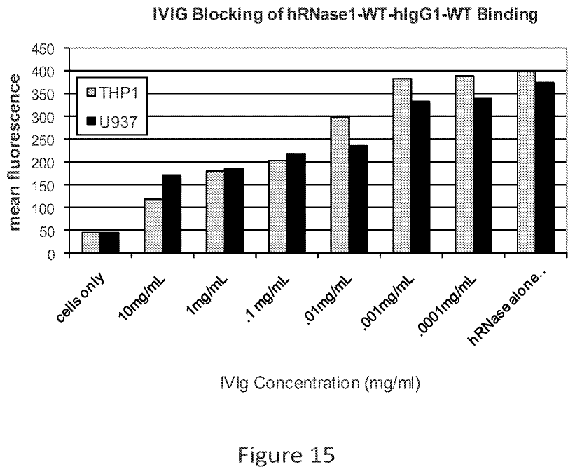

THP-1 or U937 cells were pre-incubated with IVIg starting at 10 mg/ml and performing 10-fold serial dilutions across the wells of a 96 well plate. Cells (approximately 1×10e6 per well) were incubated on ice for 45 minutes. Pre-bound cells were washed twice and AF750 conjugated hRNase1-WT-hIgG1-WT (SEQ ID NO:161) at approximately 5 ug/ml was added to each well. Binding reactions were incubated 45 minutes on ice, washed twice in PBS/2% FBS, and analyzed by flow cytometry as described above. IVIg was able to partially block the binding of the labeled nuclease fusion protein, but even at 10 mg/ml, there was still residual binding detectable above background. FIG. 15 shows the blocking activity of human IVIg for binding to U937 and THP-1 cells by hRNase1-WT-hIgG1-WT (SEQ ID NO: 161).

### Example 16. Trex1-Ig Activity Assay

Murine Trex 1 was cloned from mouse cDNA using the primers listed below:

mTrex1-5′age:
(SEQ ID NO: 20)
accggtatgggctcacagaccctgccccatggtcaca
mTrex1-3′bx:
(SEQ ID NO: 21)
ctcgagatctgttgttccagtggtagccggagtgccgtacatg

PCR reactions were performed using 50 pmol each primer in a total volume of 50 ul, under an amplification profile of 94 C 30 sec; 50 C 60 sec; 68 C 90 sec for 35 cycles of amplification. PCR products were cloned into the pCR2.1 vector and TOPO clones screened as previously described for prototype nuclease fusion gene cloning. Once sequence was verified, the cassettes were subcloned into the pDG expression vector fused to the mIgG tail or co-cloned with one of the (g4s) n linkers to construct Trex 1-Ink molecules with different length linkers. Plasmid isolates were transiently transfected into COS cells as described and stable CHO transfectants generated as described for prototype nuclease fusion genes.

Fusion genes were constructed encoding Trex1Ig as follows: the genes incorporate the human VK3 leader peptide fused to murine Trex 1 truncated at the COOH terminus by 72 amino acids (to remove the intracellular nuclear targeting sequences) fused to a (gly4ser)4 (SEQ ID NO: 130) or (gly4ser)5 linker (SEQ ID NO:131), fused to the murine IgG2a/c allele that incorporates some changes from the IgGe sequence of the Balb/c IgG2a allele.

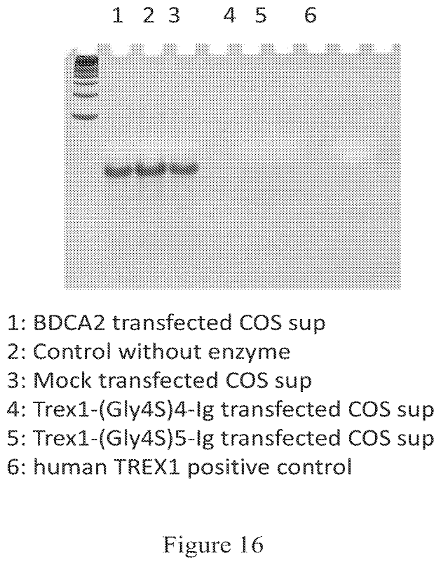

The exonuclease activities of Trex1-Ig were measured in 30 ul reactions containing 20 mM Tris (pH7.5), 5 mM MgCl₂, 2 mM DTT, using a 36-mer oligonucleotide as substrate. Incubation reactions were allowed to proceed for 20-30 min at 37° C. Samples were subjected to electrophoresis on 23% polyacrylamide DNA gels overnight. Gels were incubated in TBE buffer containing 0.5 ug/ml ethidium bromide. The DNA was visualized by UV transilluminator, and photographed using a Kodak EDAS 290 digital camera equipped with ethidium bromide filters and analyzed using Kodak Molecular Imaging Software. The trex1 activity assay results for COS produced mTrex1-(g4s)4-mIgG2a-c (SEQ ID NO:166) and mTrex1-(g4s)5-mIgG2a-c (SEQ ID NO:167) are shown in FIG. 16.

### Example 17. Western Blot of mTrex1-Ig Single Hybrid Nuclease Molecules Produced by COS-7 Transient Transfection

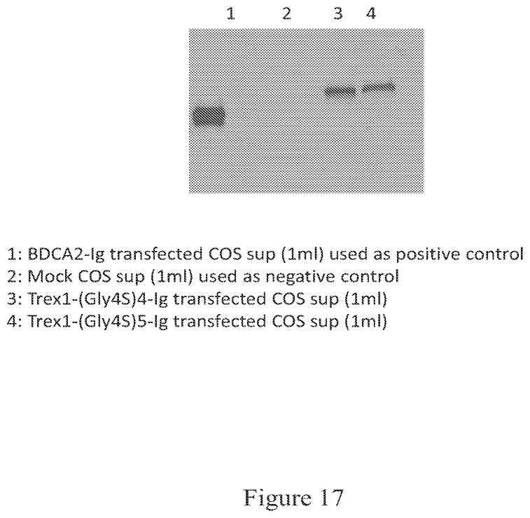

COS-7 cells were transiently transfected with plasmids containing hybrid nuclease molecules encoding Trex 1-Ig as follows: the genes incorporate the human VK3 leader peptide fused to murine Trex 1 truncated at the COOH terminus by 72 amino acids (to remove the nuclear envelope targeting sequences) fused to a (gly4ser)4 or (gly4ser)5 linker, fused to the murine IgG2a/c allele that incorporates some changes from the IgGe sequence from the Balb/c IgG2a allele. COS supernatants were harvested after 72 hours and 0.5-1.0 ml samples (depending on the experiment) were immunoprecipitated overnight at 4° C. with 100 ul protein A-agarose beads. Protein A beads were centrifuged and washed twice in PBS prior to resuspending in reducing SDS-PAGE loading buffer. Samples were heat treated at 100 C for 5 minutes, protein A beads centrifuged to pellet, and sample buffer loaded onto 10% SDS-PAGE gels. Samples were electrophoresed at 150 volts for 1.5-2 hours, and gels blotted to nitrocellulose membranes at 30 mAmp for 1 hour. Western blots were blocked in TBS/5% non-fat milk overnight. Blots were incubated with 1:2500 HRP (horseradish peroxidase) conjugated goat anti-mouse IgG2a/c (Fc specific, KPL) for 1.5 hours at room temperature, washed in PBS/0.5% Tween20 five or more times, and blots developed using ECL reagent. FIG. 17 shows a Western blot of immunoprecipitates from COS7 culture supernatants expressing mTrex 1-(g4s)4 (SEQ ID NO:166) or (g4s)5-mIgG2a-c (SEQ ID NO:167) fusion proteins.

### Example 18. Exonuclease Activity of DNase1L3Ig CHO Derived Fusion Protein

DNase 1L3 was cloned from mouse spleen cDNA using the following primer pair to clone the mDNase1L3 including its native leader peptide sequence.

mdnase1L3-NL:
(SEQ ID NO: 22)
GTT AAG CTT GCC ACC ATG TCC CTG CAC CCA GCT TCC
CCA CGC CTG
Mdnase1L3-3bx:
(SEQ ID NO: 23)
CTC GAG ATC TGA GGA GCG ATT GCC TTT TTT TCT CTT
TTT GAG AG

Alternatively, PCR reactions were set up using the following primer pair to attach to the human VK3 leader peptide instead of the native leader.

mdnase1L3-age:
(SEQ ID NO: 24)
ACC GGT CTA AGG CTC TGC TCC TTC AAT GTG AGG TCC TTT
GGA
Mdnase1L3-3bx:
(SEQ ID NO: 25)
CTC GAG ATC TGA GGA GCG ATT GCC TTT TTT TCT CTT TTT
GAG AG

PCR reactions were performed using 50 pmol each primer in a total volume of 50 ul, under an amplification profile of 94 C 30 sec; 50 C 60 sec; 68 C 90 sec for 35 cycles of amplification. PCR products were cloned into the pCR2.1 vector and TOPO clones screened as previously described for prototype nuclease fusion gene cloning. Once sequence was verified, the cassettes were subcloned into the pDG expression vector fused to the mIgG tail. Plasmid isolates were transiently transfected into COS cells as described and stable CHO transfectants generated as described for prototype nuclease fusion genes.

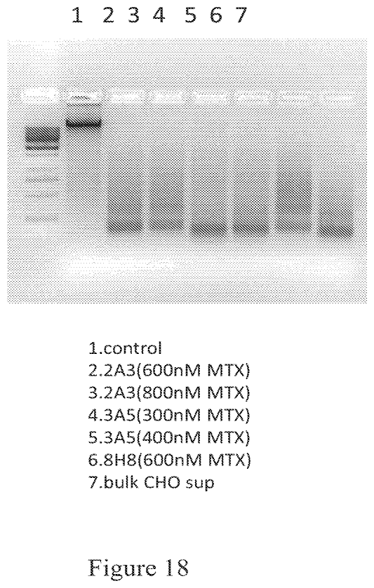

The exonuclease activity in protein extracts from DNase 1L3Ig (SEQ ID NO: 185) CHO clones was measured in 30 ul reactions containing 20 mM Tris (pH7.5), 5 mM MgCl₂. 2 mM DTT, and a substrate. Incubation was 20-30 min at 37° C. Samples were then run on agarose DNA gel overnight. The gel was incubated in TBE buffer containing Ethidium bromide. The DNA was visualized under UV. The results of chromatin digestion analysis are shown in FIG. 18.

### Example 19. Dose Titration of Increasing Volumes of CHO Supernatant for Exonuclease Activity

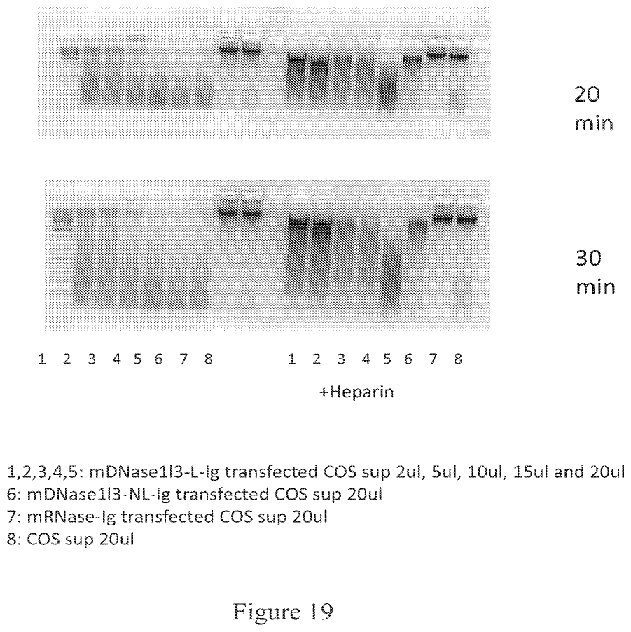

FIG. 19 shows titration analysis of the exonuclease digestion patterns obtained from COS supernatants expressing DNase 1 L3Ig fusion proteins (SEQ ID NO:183 or 185). Nuclear DNA Degradation assays were performed as follows: HeLa cells were cultured in DMEM media and nuclei from 10e5 cells were isolated using NP-40 lysis. Nuclei were diluted into 200 ul reaction buffer containing 10 mM Hepes (pH 7.0), 50 mM NaCl, 2 mM MgCl₂, 2 mM CaCl₂), and 40 mM b-glycerophosphate. Nuclei were incubated for 3 hours at 37° C. in the volumes of culture supernatant indicated on the figure from DNase1L3 transfected COS cells. Nuclear DNA was isolated using QiAmp blood DNA minikit. DNA was analyzed by 1.5% agarose gel electrophoresis. For control reactions, heparin was used at 250 i.u./ml, to inhibit nuclease activity.

### Example 20: Construction and Expression of DNase1-Ig Single and Dual Enzyme Hybrid Nuclease Molecules

Naturally occurring alleles of human DNase1 or DNase1 like molecules have been reported. The A114F mutation has been previously reported to occur in natural variants of human DNAse1 like enzymes, and to result in actin resistance of the enzymes containing this sequence change. See Pan, C Q, Dodge T H, Baker D L, Prince W E, Sinicropi D V, and Lazarus R A. J Biol Chem 273:18374-18381, (1998); Zhen A, Parmelee D, Hyaw H, Coleman T A, Su K, Zhang J, Gentz R, Ruben S, Rosen C, and Li Y. Biochem and Biophys Res Comm 231:499-504 (1997); and Rodriguez A M, Rodin D, Nomura H, Morton C C, Weremowicz S, and Schneider M C. Genomics 42:507-513 (1997), all of which are herein incorporated by reference.

Similarly, the G105R mutation has been reported recently as a single nucleotide polymorphism in the gene encoding human DNAse 1 that is polymorphic in some or all populations, and that is relevant to autoimmunity. (See Yasuda T, Ueki M, Takeshita H, Fujihara J, Kimura-Kataoka K, Lida R, Tsubota E, Soejima M, Koda Y, Dato H, Panduro A. Int J Biochem Cell Biol 42 (7): 1216-1225 (2010), herein incorporated by reference). Allelic variants at this position resulted in high activity harboring DNase 1 isoforms relative to wild type. Another naturally occurring, polymorphic mutation (R21S) has also been reported to confer higher activity. (See Yasuda, supra)

SLE patients have been reported to have significantly decreased levels of DNase1 activity (See Martinez-Valle F, Balada E, Ordi-Ros J, Bujan-Rivas S, Sellas-Fernandez A, Vilardell-Tarres M. Lupus 18 (5): 418-423 (2009), herein incorporated by reference).

Naturally occurring enzyme variants may thus be less immunogenic when administered to patients, since these isoforms occur in the human population. We reasoned that the combination of the actin resistant properties of alleles similar to A114F with the increased enzymatic activity of alleles like G105R would generate novel allelic variants of human DNase1 that might show improved clinical activity in vitro and in vivo. To our knowledge, ours is the first report of this new mutant form of DNase1 generated from a combination of two naturally occurring variants G105R and A114F.

Human DNase I was isolated as described previously from human pancreas RNA (Ambion), by random primed cDNA and PCR using the following primer sets:

5′hDNase1-age:
(SEQ ID NO: 26)
GTT ACC GGT CTG AAG ATC GCA GCC TTC AAC ATC CAG
5′hDNase1-bx:
(SEQ ID NO: 27)
GTT CTC GAG ATC TTT CAG CAT CAC CTC CAC TGG ATA
GTG

Alternatively, the 3′ DNase cassettes were amplified by PCR using the following primer pair.

3′hDNase1-RV:
(SEQ ID NO: 28)
GTT GAT ATC CTG AAG ATC GCA GCC TTC AAC ATC CAG
3′hDNase1-stop:
(SEQ ID NO: 29)
GTT TCT AGA TTA TCA CTT CAG CAT CAC CTC CAC TGG ATA
GTG

PCR reactions were performed using 50 pmol each primer, 2 ul cDNA, in a total volume of 50 ul using Platinum PCR Supermix as previously described. The amplification profile was 94 C 30 sec; 55 C 30 sec; 68 C 90 sec for 35 cycles.

Once the wild type gene was amplified by PCR, the fragments were subjected to gel electrophoresis and 850 bp fragments purified by QIAquick column purification. Fragments were cloned into pCR2.1, transformed by TOPO cloning according to manufacturer's instructions as described for the other constructs. Once sequence was verified, PCR primers were used to generate subfragments containing naturally occurring alleles for DNase1 that have been reported to improve specific activity and improve resistance to the inhibitory activity of actin. These subfragments contained overlapping sequence, permitting amplification of complete DNase1 subclones containing the desired allelic variations. COS 7 cells were transiently transfected in 60 mm dishes using Polyfect (Qiagen, Valencia, CA) transfection reagent. Plasmid DNA was prepared using the Qiagen QIAprep miniprep kits according to manufacturer's instructions. Plasmids were eluted in 50 ul EB buffer. DNA concentration was measured using the Nanodrop and an aliquot equivalent to 2.5 ug plasmid DNA used for each transfection reaction. Each DNase1g (SEQ ID NOS.: 118, 119, 120, 121, 122 or 123) or RNase-Ig-DNase (SEQ ID NOS.: 115, 116, 117) expression cassette was inserted into the mammalian expression vector pDG, a derivative of pcDNA3.1. Transfected cells were incubated for 72 hours at 37° C., 5% CO2 prior to harvest of culture supernatants for further analysis. Culture supernatants were harvested, residual cells centrifuged from the solution, and the liquid transferred to new tubes.

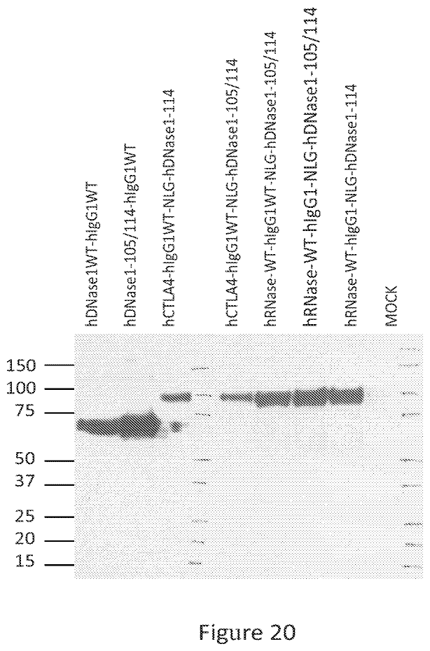

COS-7 cells were transiently transfected with plasmids containing human DNase1 wild type (SEQ ID NO: 118) or naturally occurring DNase 1 mutant alleles (G105R and/or A114F) (SEQ ID NO:115, 116, or 117) fused to the wild type human IgG1 Fc domain. This hinge-CH2-CH3 cassette contains a single C→S mutation in the hinge region to eliminate the first cysteine in this domain since it is unpaired due to absence of its pairing partner present in the light chain of the antibody. In addition, more complex multi-nuclease fusion proteins were also expressed from COS cell transient transfections. Western blot analysis was performed on supernatants from transient transfectants. The molecules shown in FIG. 20 contain human DNase1 fused to the human IgG1 wild type Fc domain (SEQ ID NO:154, 155, 156, or 159) or include human RNase1 (wild type) fused to the SCC hinge-CH2-CH3 Fc domain of human IgG1, followed by a novel linker containing an N-linked glycosylation site to protect the linker domain from protease cleavage, and the wild type (SEQ ID NO:153) or mutant allele (SEQ ID NO:151 or 152) forms of human DNase1 at the carboxy terminus of the molecule. COS supernatants were harvested after 72 hours and 0.5-1.0 ml samples (depending on the experiment) were immunoprecipitated overnight at 4° C. with 100 ul protein A-agarose beads. Protein A beads were centrifuged and washed twice in PBS prior to resuspending in SDS-PAGE loading buffer, for NuPAGE gels—reducing or nonreducing LDS sample buffer. Samples were heated according to manufacturer's instructions, protein A beads centrifuged to pellet, and sample buffer loaded onto 5-12% NuPAGE gradient gels. Samples were electrophoresed at 150 volts for 1.5-2 hours, and gels blotted to nitrocellulose membranes at 30 mAmp for 1 hour. Western blots were blocked in TBS/5% non-fat milk overnight. Blots were incubated with 1:2500 HRP (horseradish peroxidase) conjugated goat anti-human IgG (Fc specific, Jackson Immunoresearch) or goat anti-mouse IgG for 1.5 hours at room temperature, washed in PBS/0.5% Tween20 five or more times, and blots developed using ECL reagent.

### Example 22. Screening COS Supernatants for Nuclease Enzyme Activity

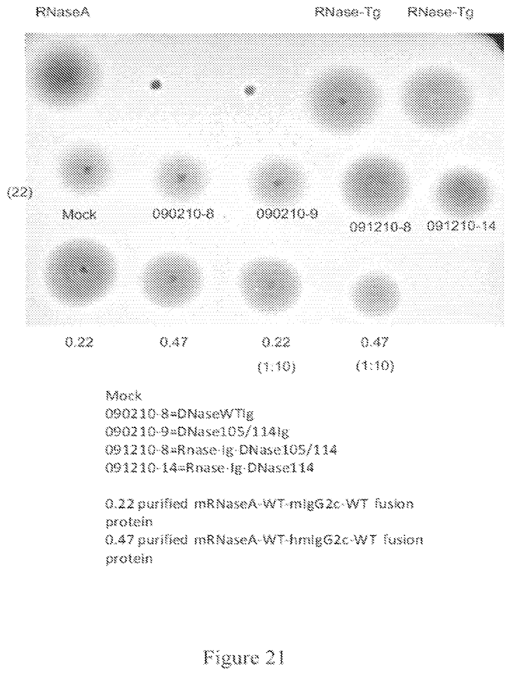

FIG. 21 shows the results of RNase activity assays (SRED) analysis on harvested COS supernatants expressing hDNAse1Ig and hRNase1-Ig-hDNase1 fusion proteins by SRED.

COS supernatants from transient transfections of the hDNase1g single or multispecific nucleases were assayed for nuclease activity as follows. A 2% agarose gel was prepared with distilled water. Poly-C(Sigma) was dissolved in distilled water at 3 mg/ml. the gel plate was prepared as follows: 1.5 ml reaction buffer (0.2M Tris-HCl pH7.0, 40 mM EDTA and 0.1 mg/ml Ethidium bromide), 1 ml Poly-C and 0.5 ml water were place in the tube and maintained at 50 C for 5 min. 3 ml of the agarose (kept at 50 C) was added to the tube. The mixture was immediately poured onto glass plate. Sampling wells were punched in the gel. Approximately 2 ul of each sample was loaded and the gel was incubated at 37 C for 4 hours in the moist chamber. Then the gel was incubated in a buffer (20 mM sodium acetate pH5.2, 20 mg/ml Ethidium bromide) on ice for 30 min. Gels were photographed on a UV transilluminator using a Kodak digital camera DC290 system equipped with ethidium bromide filters and analyzed using Kodak Molecular Imaging software.

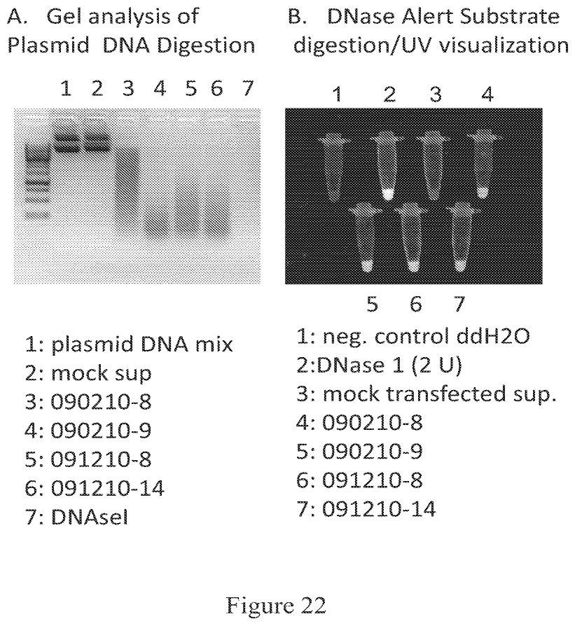

FIG. 22 shows a composite figure displaying results of DNase nuclease activity assays performed on COS supernatants from transfected cells. Culture supernatants were harvested 72 hours after transfecting the following clones of DNase1 wild type and mutant-Ig fusion proteins: (1) 090210-8=hDNAse1-WT-hIgG1 WT (SEQ ID NO:154); (2) 090210-9=hDNase1-G105R; A114F-hIgG1 WT (SEQ ID NO:159); (3) 091210-8=hRNase1-WT-hIgG1-WT-DNase1-G105R; A114F(SEQ ID NO:151), and (4) 091210-14=hRNase-WT-hIgG1-WT-DNase1-A114F(SEQ ID NO:152).

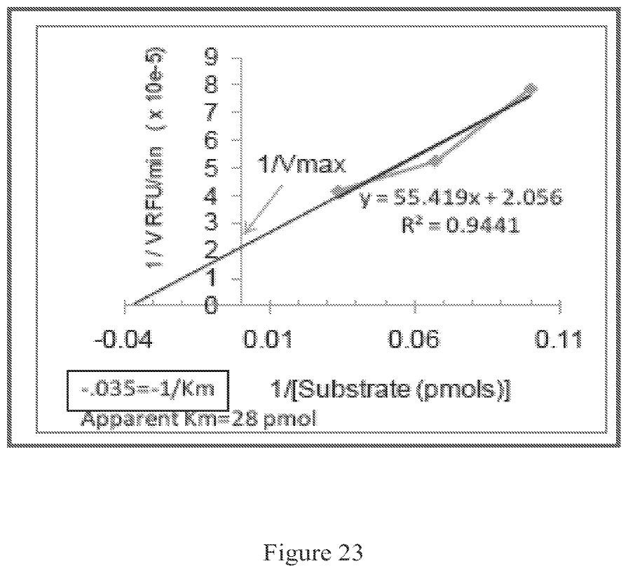

The pH of the supernatants was adjusted to pH 8.0 with bicarbonate buffer to facilitate binding of expressed-Ig fusion proteins to protein A agarose beads. Panel A. of FIG. 23 shows gel electrophoresis analysis of plasmid DNA digestion: Protein A agarose slurry (50 ul per sample) was washed in PBS, and incubated overnight at 4° C. with 100 ul culture supernatant to immunoprecipitate-Ig fusion proteins. Immunoprecipitates were washed 4-5 times in 750 ul PBS, centrifuging at approximately 3500 rpm followed by aspiration of PBS. Final protein A precipitates were resuspended in 50 ul reaction buffer containing 20 mM Tris ph7.5. 2 mM CaCl2 and 2 mM MgCl2 containing 1.5 ug plasmid DNA (pDG expression vector). Reactions were incubated for 30 minutes at 37° C., heated at 65° C. for 5 min, and the DNA present in reactions analyzed by agarose gel electrophoresis on 1.5% TBE-agarose gels.

Panel B shows the results of a nuclease activity assay performed on the same culture supernatants using the DNase Alert Kit (IDT/Ambion). Reaction tubes containing lyophilized DNase Alert Substrate (50 pmoles) were resuspended with 5 ul nuclease free ddH2O supplied with the kit, 5 ul 10× DNase alert buffer, and 40 ul protein A slurry immunoprecipitated as follows: For these immunoprecipitations, 50 ul protein A agarose beads were incubated overnight with 50 ul culture supernatant. Samples were then washed 5 times with 0.75 ml PBS. Final protein A precipitates were resuspended in 80 ul nuclease free ddH2O, and 40 ul of the slurry (one half the precipitate) was transferred to the reaction tubes. Negative controls with mock transfected IP and ddH20 were also set up. A positive control was also set up containing DNase1 provided with the kit (2 units). Reactions were incubated 1 hour at 37° C., and exposed to short wave length UV transillumination to visualize fluorescence. Relative amounts of DNA digestion are indicated by degree of fluorescence.

### Example 22: Examination of Mac-2 Positive Cells in DTg Mice

Early lupus mortality is usually due to nephritis or infection resulting from immunosuppression to treat nephritis. Therefore, an extremely important outcome for any new therapy is improvement in nephritis. While human studies are limited to quantitation of proteinuria and creatinine, in mice one can get an accurate assessment of inflammation and damage to the kidneys by histology and immunocytochemistry. We report that TLR7.1×RNase double transgenic (DTg) mice showed lower anti-RNA antibodies, less B cell activation, fewer immune deposits and less PAS positive staining glomeruli. We have further compared macrophage infiltration of the kidneys using the anti-Mac-2 (galectin3) antibody (Iyoda et al. Nephrol Dial Transplant 22:3451, 2007). Frozen sections from kidneys obtained from single or double Tg were examined for numbers of Mac-2+ macrophages as well as glomerular size as described (Iyoda et al). Twenty randomly selected glomeruli (from the outer to inner side of the kidney) were counted for positive cells. There are many fewer mac-2 positive staining cells in the glomeruli of DTg as compared to single Tg mice (data not shown). The results of counting 20 glomeruli per mouse in a pilot study of n=4-5 in each group, revealed mean+/−SE of 3.8+/−1.1 and 1.4+/−0.2 for single versus DTg respectively, p=0.05. In addition, we quantified glomerular tuft size and observed a significant reduction in glomerular tuft size in the DTg mice (179.4+/−41 versus 128+/−16.8 um2 in single versus DTg respectively, p=0.037).

### Example 23: Km of Purified Murine RNaseA-Ig Fusion Protein

To further define the functional characteristics of the bivalent RNase-Ig fusion protein (SEQ ID NO: 150), we performed determinations of the Michaelis constant, Km. As shown in FIG. 23, the enzyme has a high affinity with a provisional Km of 280 nM (as a comparison, RNase A has a Km of 34 nM using polyC as substrate (delCardayre et al, Prot Eng 8:261, 1995)). FIG. 23 shows enzyme kinetics that were assayed using the Rnase Alert Substrate (Ambion/IDT) and fluorescence was quantified with a Spectramax M2 microplate Reader. Data was analyzed using Softmax Pro software (Molecular Devices). Reaction rates at different substrate concentrations were measured and the data shown as a Lineweaver-Burk plot. The apparent Km, corrected for volume is 280 nM.

### Example 24. Analysis of 564Igi to Mice for Anti-RNA Antibodies

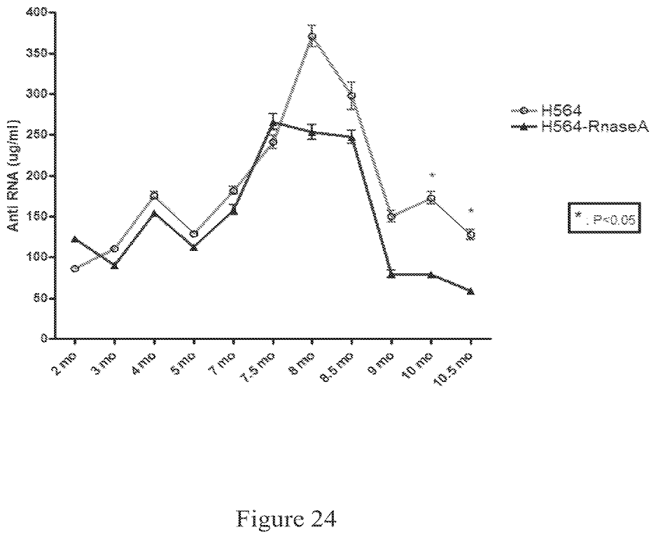

564 Igi Tg mice: Dr. Imanishi-Kara inserted the rearranged VDJ genes from the H564 hybridoma into the endogenous Igh and Igk loci to create the 564Igi mouse on a B6 background. Sera from these mice stained the cytoplasm and nucleoli of fixed cells indicating a predominant anti-RNA specificity. Consistent with this finding and of special relevance to this patent application, antibody production was inhibited when these mice were made TRL7 deficient indicating that the stimulus for antibody production is indeed RNA. This mouse strain develops late onset glomerulonephritis. We analyzed the expression of anti-RNA antibodies in mice transgenic for H564 and also double transgenic mice coexpressing 564Ig and RNase transgenes. FIG. 24 compares the levels of anti-RNA antibodies in mouse sera at successive intervals as these transgenic mice aged.

See Gavalchin, J., R. A. Seder, and S. K. Datta. 1987. The NZB×SWR model of lupus nephritis. I. Cross-reactive idiotypes of monoclonal anti-DNA antibodies in relation to antigenic specificity, charge, and allotype. Identification of interconnected idiotype families inherited from the normal SWR and the autoimmune NZB parents. J. Immunol. 138:128-137; and Berland, R., L. Fernandez, E. Kari, J. H. Han, I. Lomakin, S. Akira, H. H. Wortis, J. F. Kearney, A.A. Ucci, and T. Imanishi-Kari. 2006. Toll-like receptor 7-dependent loss of B cell tolerance in pathogenic autoantibody knockin mice. Immunity 25:429-440.

### Example 25: In Vitro Assessment of Hybrid Nuclease Molecule Biological Activity

One or more hybrid nuclease molecules are purified, e.g., by affinity or ion exchange chromatography as previously described in the examples above. In some instances the hybrid nuclease molecule is a polypeptide. In some instances, the hybrid nuclease molecule includes one or more sequences from Table 2. In some instances the molecule is SEQ ID NO:161, 162, or 163. In some instances the molecule includes SEQ ID NO:145 and SEQ ID NO: 149. In some instances the molecule is SEQ ID NO: 151, 152, 153, 154, 155, 156, 157, 158, 159, 160, 161, 162, 163, 166, 167, 169, 170, 171, 173, 175, 177, 179, 181, 187, 189, 191, 193, 195, 197, 199, 201, 203, 205, or 207. The hybrid nuclease molecule can be any of those disclosed herein and any that can be constructed from the sequences disclosed herein (see Table 2), e.g., by taking a nuclease domain and linking it to an Fc domain; or, e.g., taking a nuclease domain and linking it to an Fc domain with a linker domain. Various linker domains (e.g., those described herein) can be used to link the Fc domains and/or nuclease domains. For example, linker domains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids in length can be used. Molecules are assayed for the specific nuclease activity in vitro using qualitative assays to verify that they possess the desired nuclease function. Specific activities are generally then determined by fluorescence based kinetic assays utilizing substrates such as the RNase or DNase Alert Kit reagents, and a fluorescence plate reader set to take readings as a function of time. In addition, protein solutions are generally checked for endotoxin contamination using a commercially available kits, such as the Pyrotell Limulus Amebocyte Lysate (LAL) kit, 0.06 EU/ml detection limit from Cape Cod, Inc. (E. Palmouth, MA). Molecules are then assayed using a variety of in vitro assays for biological activity.

One series of in vitro assays will measure the effect of the molecules on cytokine production by human PBMC in response to various stimuli, in the presence or absence of the molecules in the cultures. Normal or patient human PBMC (approximately 1×10e6 cells) are cultured for 24, 48, or 96 hours depending on the assay. PBMC are cultured in the presence of stimuli such as TLR ligands, costimulatory antibodies, immune complexes, and normal or autoimmune sera. The effects of the molecules on cytokine production is measured using commercially available reagents, such as the antibody pair kits from Biolegend (San Diego, CA) for IL-6, IL-8, IL-10, IL-4, IFN-gamma, TNF-alpha. Culture supernatants from in vitro cultures are harvested at 24, 48 hours or later time points to determine the effects of the molecules on cytokine production. IFN-alpha production is measured using, e.g., anti-human IFN-alpha antibodies and standard curve reagents available from PBL interferon source (Piscataway, NJ). A similar set of assays is performed using human lymphocyte subpopulations (isolated monocytes, B cells, pDCs, T cells, etc.); purified using, e.g., commercially available magnetic bead based isolation kits available from Miltenyi Biotech (Auburn, CA).

In addition, the effect of the molecules on expression of lymphocyte activation receptors such as CD5, CD23, CD69, CD80, CD86, and CD25 is assessed at various time points after stimulation. PBMC or isolated cell subpopulations are subjected to multi-color flow cytometry to determine how these molecules affect the expression of different receptors associated with immune cell activation.

Another set of assays will measure the effects of these molecules on the proliferation of different lymphocyte subpopulations in vitro. These assays will utilize, e.g., CFDA-SE staining (Invitrogen, Carlsbad, CA) of human PBMCs prior to stimulation. CFSE at 5 mM is diluted 1:3000 in PBS/0.5% BSA with 10e7-10e8 PBMCS or purified cell subsets and labeling reactions incubated for 3-4 minutes at 37 C prior to washing several times in RPMI/10% FBS to remove remaining CFSE. CFSE labeled cells are then incubated in co-culture reactions with various stimuli (TLR ligands, costimulatory antibodies, etc.) and the molecules for 4 days prior to analysis of cell proliferation by flow cytometry using dye-conjugated cell subpopulation specific antibodies.

The effect of these molecules on in vitro maturation of monocytes into DCs and macrophages is also assessed using both normal and patient PBMC samples.

The effectiveness of a hybrid nuclease molecule is demonstrated by comparing the results of an assay from cells treated with a hybrid nuclease molecule disclosed herein to the results of the assay from cells treated with control formulations. After treatment, the levels of the various markers (e.g., cytokines, cell-surface receptors, proliferation) described above are generally improved in an effective molecule-treated group relative to the marker levels existing prior to the treatment, or relative to the levels measured in a control group.

### Example 26: Administration of a Hybrid Nuclease Molecule to a Mammal in Need Thereof

Mammals (e.g., mice, rats, rodents, humans, guinea pigs) are used in the study. Mammals are administered (e.g., intravenously) one or more hybrid nuclease molecules comprising one or more sequences from Table 2 or a control. In some instances the molecule is SEQ ID NO: 161, 162, or 163. In some instances the molecule includes SEQ ID NO: 145 and SEQ ID NO:149. In some instances the molecule is SEQ ID NO: 151, 152, 153, 154, 155, 156, 157, 158, 159, 160, 161, 162, 163, 166, 167, 169, 170, 171, 173, 175, 177, 179, 181, 187, 189, 191, 193, 195, 197, 199, 201, 203, 205, or 207. The hybrid nuclease molecule can be any of those disclosed herein and any that can be constructed from the sequences disclosed herein (see Table 2), e.g., by taking a nuclease domain and linking it to an Fc domain; or, e.g., taking a nuclease domain and linking it to an Fc domain with a linker domain. Various linker domains (e.g., those described herein) can be used to link the Fc domains and/or nuclease domains. For example, linker domains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids in length can be used. In some instances the hybrid nuclease molecule is formulated a pharmaceutically acceptable carrier. In some instances the molecule is formulated as described in the pharmaceutical compositions section above. The hybrid nuclease molecule targets RNase and/or DNase.

Multiple rounds of doses are used where deemed useful. Effects on IFN-alpha levels, IFN-alpha response gene levels, autoantibody titers, kidney function and pathology, and/or circulating immune complex levels are monitored in the mammals. Similar studies are performed with different treatment protocols and administration routes (e.g., intramuscular administration, etc.). The effectiveness of a hybrid nuclease molecule is demonstrated by comparing the IFN-alpha levels, IFN-alpha response gene levels, autoantibody titers, kidney function and pathology, and/or circulating immune complex levels in mammals treated with a hybrid nuclease molecule disclosed herein to mammals treated with control formulations.

In an example, a human subject in need of treatment is selected or identified. The subject can be in need of, e.g., reducing a cause or symptom of SLE. The identification of the subject can occur in a clinical setting, or elsewhere, e.g., in the subject's home through the subject's own use of a self-testing kit.

At time zero, a suitable first dose of a hybrid nuclease molecule is administered to the subject. The hybrid nuclease molecule is formulated as described herein. After a period of time following the first dose, e.g., 7 days, 14 days, and 21 days, the subject's condition is evaluated, e.g., by measuring IFN-alpha levels, IFN-alpha response gene levels, autoantibody titers, kidney function and pathology, and/or circulating immune complex levels. Other relevant criteria can also be measured. The number and strength of doses are adjusted according to the subject's needs.

After treatment, the subject's IFN-alpha levels, IFN-alpha response gene levels, autoantibody titers, kidney function and pathology, and/or circulating immune complex levels are lowered and/or improved relative to the levels existing prior to the treatment, or relative to the levels measured in a similarly afflicted but untreated/control subject.

In another example, a rodent subject in need of treatment is selected or identified. The identification of the subject can occur in a laboratory setting or elsewhere.

At time zero, a suitable first dose of a hybrid nuclease molecule is administered to the subject. The hybrid nuclease molecule is formulated as described herein. After a period of time following the first dose, e.g., 7 days, 14 days, and 21 days, the subject's condition is evaluated, e.g., by measuring IFN-alpha levels, IFN-alpha response gene levels, autoantibody titers, kidney function and pathology, and/or circulating immune complex levels. Other relevant criteria can also be measured. The number and strength of doses are adjusted according to the subject's needs.

After treatment, the subject's IFN-alpha levels, IFN-alpha response gene levels, autoantibody titers, kidney function and pathology, and/or circulating immune complex levels are lowered and/or improved relative to the levels existing prior to the treatment, or relative to the levels measured in a similarly afflicted but untreated/control subject.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### TABLES

TABLE 1

| SEQ ID | | |
|---|---|---|
| NO: | Name | Sequence |
| | | Primer Listing for RNase and DNase -Ig Fusion |
| | | Gene Constructs |
| | | human primers: |
| 30 | mahIgG1CH2M | tgtccaccgtgtccagcacctgaactcctgggtggatcgtcagtcttcc |
| 31 | huIgG1-H1 | agatctcgagcccaaatcttctgacaaaactcacacatgtccaccgtgt |
| 32 | hIgG1-5scc | gaagatctcgagcccaaatcttctgacaaaactcacacatgt |
| 33 | hIgG1SSSH | gttagatctcgagcccaaatcttctgacaaaactcacacatct |
| 34 | mah1gG1S | tctagattatcatttacccggagacagagagaggctcttctgcgtgtag |
| | | tg |
| 35 | P331S | aaggtctccaacaaagccctcccagcctccatcgagaaaacaatctcc |
| 36 | P331AS | gttttctcgatggaggctgggagggctttgttggagacc |
| 37 | 5′hrnase | AAG CTT GCC ACC ATG GCT CTG GAG AAG TCT CTT GTC |
| | | CGG CTC C |
| 38 | 3′hrnasebx | ctcgagatctgtagagtcctccacagaagcatcaaagtgg |
| 39 | 5′hrnaseage | accggtaaggaatcccgggccaagaaattcc |
| 40 | 3′hRNaseRV | gatatcccttccctgggcaaggaatcccgggccaagaaattccag |
| 41 | 3′hRNase- | gtttctagattattaggtagagtcctccacagaagcatcaaagtg |
| | stop | |
| 42 | hdnase1L3- | GGT AAG CTT GCC ACC ATG TCA CGG GAG CTG GCC CCA |
| | 5NL | CTG CTG CTT |
| 43 | hdnase1L3- | CTC GAG ATC TGA GGA GCG TTT GCT CTT TGT TTT CTT |
| | 3bx | CCT TAG |
| 44 | hDNase1L3- | accggtatgaggatctgctccttcaacgtcaggtcctttgg |
| | 5age | |
| 45 | 5′hDNase1- | GTT ACC GGT CTG AAG ATC GCA GCC TTC AAC ATC CAG |
| | age | |
| 46 | 5′hDNase1- | GTT CTC GAG ATC TTT CAG CAT CAC CTC CAC TGG ATA |
| | bx | GTG |
| 47 | 3′hDNase1- | GTT GAT ATC CTG AAG ATC GCA GCC TTC AAC ATC CAG |
| | RV | |
| 48 | 3′hDNase1- | GTT TCT AGA TTA TCA CTT CAG CAT CAC CTC CAC TGG |
| | stop | ATA GTG |
| 49 | hDNase1 | GAT GGC TGC GAG CCC TGC AGG AAC GAC ACC TTC AAC |
| | S105-114 | CGA GAG CCA TTC ATT GTC AGG TTC |
| 50 | hDNase1- | GAA CCT GAC AAT GAA TGG CTC TCG GTT GAA GGT GTC |
| | as114-105 | GTT CCT GCA GGG CTC GCA GCC ATC |
| 51 | hDNase1- | GGA GAA GAA CCT GAC AAT GAA TGG CTC TCG GTT GAA |
| | as114 | GGT |
| 52 | hDNase1- | ACC TTC AAC CGA GAG CCA TTC ATT GTC AGG TTC TTC |
| | s114 | TCC |
| 53 | hTrex1- | accggtatgggccctggagctcgcagacagggcag |
| | 5′age | |
| 54 | hTrex1-3′bx | ctcgagatctttggtcctagcagaggctgtgacc |
| 55 | hTrex1-5′ | accggtctcgagatgggccctggagctcgcagacagg |
| | AX | |
| 56 | hTrex1- | ctcgagtttggtcctagcagaggctgtgacc |
| | 3′xho#2 | |
| | | Murine Primers: |
| 57 | mTrex1- | accggtatgggctcacagaccctgccccatggtcaca |
| | 5′age | |
| 58 | mTrex1-3′bx | ctcgagatctgttgttccagtggtagccggagtgccgtacatg |
| 59 | mdnase1L3- | GTT AAG CTT GCC ACC ATG TCC CTG CAC CCA GCT TCC |
| | 5NL | CCA CGC CTG |
| 60 | mdnase1L3- | CTC GAG ATC TGA GGA GCG ATT GCC TTT TTT TCT CTT |
| | 3bx | TTT GAG AG |
| 61 | mrib1-NL | gTT AAg CTT gCC ACC ATg ggT CTg gAg AAg TCC CTC |
| | | ATT CTg |
| 62 | mrib3NH2 | ggC TCg AgC ACA gTA gCA TCA AAg tGG ACT ggT ACg |
| | | TAg g |
| 63 | muIgG2aCH2 | cctccatgcaaatgcccagcacctaacctcttgggtggatcatccgtct |
| | | tcatcttcc |
| 64 | mIgG2a-5 | agatctcgagcccagaggtcccacaatcaagccctctcctccatgcaaa |
| | | tgcc |
| 65 | mIgG2a-5scc | gaagatctcgagcccagaggtcccacaatcaagccctctcctcca |
| 66 | muIgG2aSSSH | atcaagccctctcctccatctaaatccccagcacctaac |
| 67 | mIgG2aKP5 | agtggcaaggagttcaaatgctcggtcaagaagaaagacctcccagcgt |
| | | ccatcgag |
| 68 | mIgG2aKP3 | ggttctctcgatggacgctgggaggtctttgttgttgaccgagcatttg |
| | | aactcc |
| 69 | mIgG2a3S | gtttctagattatcatttacccggagtccgagagaagctcttagtcgt |
| | | Other Primers for different tail mutations and |
| | | for multispecific fusion genes: |
| 70 | hIgG1-3ns- | gctagctccgtcgactttacccggagacagagagagg |
| | ns | |
| 71 | K322S | gactggctgaatggcaaggagtacaagtgctcggtctccaacaaagccc |
| | | tc |
| 72 | K322AS | gagggctttgttggagaccgagcacttgtaagacttgccattcagccag |
| | | tc |
| 73 | hIgG1N297S | ccgcgggaggagcagtacagcagcacgtaccgtgtggtcagcgtc |
| 74 | hIgG1N297S3 | gacgctgaccacacggtacgtgctgctgtactgctcctcccgcgg |
| 75 | mIgG2aNS | gatatctctagatttacccggagtccgagagaagctcttagtcgt |
| 76 | mIgG2a3ns- | gatatctccggagtcgactttacccggagtccgagagaagctcttag |
| | sal | |
| 77 | mIgG2N297S5 | cacaaacccatagagaggattacagcagtactctccgggtggtc |
| 78 | mIgG2N297S3 | gaccacccggagagtactgctgtaatcctctctatgggtttgag |
| 79 | | |
| 80 | g4s4clnk3 | GAT ATC ACC GGT AGA ACC ACC TCC ACC ACT CCC ACC |
| | | TCC TCC AGT GCC TCC |
| 81 | g4s4clnk5 | GTC GAC TCC GGA GGA GGT GGC TCA GGT GGT GGA GGC |
| | | AGT GGA GGA GGT GG |
| 82 | Nlnkgly5 | aaagtcgacggagctagcagccccgtgaacgtgagcagccccagcgtg |
| 83 | Nlnkgly3 | cccatgatatcctgcacgctggggctgctc |
| 84 | hdnase1age | ACC GGT ATG AGG ATC TGC TCC TTC AAC GTC AGG TCC |
| | | TTT GG |
| 85 | hdnase1L3- | AGA TCT TTA TCA GGA GCG TTT GCT CTT TGT TTT CTT |
| | 3S | CCT TAG |
| 86 | mdnase1L3- | TCT AGA TTA TCA GGA GCG ATT GCC TTT TTT TCT CTT |
| | 3S | TTT GAG AG |
| 87 | mdnase1L3- | ACC GGT CTA AGG CTC TGC TCC TTC AAT GTG AGG TCC |
| | age | TTT GGA |
| 88 | mrib-L5′ | gAT ACC ACC ggT Agg gAA TCT gCA gCA CAg AAg TTT |
| | | CAg |
| 89 | mrib5X | AAA TCT AgA CCT CAA CCA ggT Agg gAA TCT gCA gCA |
| | | CAg AAg TTT CAg |
| 90 | mrib3X | TCT AgA CTA TCA CAC AgT AgC ATC AAA gTg gAC Tgg |
| | | TAC gTA |
| 91 | hRNaseG88D- | agactgccgcctgacaaacgactccaggtaccc |
| | S | |
| 92 | hRNAseG88D- | gggtacctggagtcgtttgtcaggcggcagtct |
| | AS | |
| 93 | g4s5-5-1 | GGC TCA GGT GGT GGA GGA TCT GGA GGA GGT GGC TCA |
| | | GGT GGT GGA GGA TCT G |
| 94 | g4s5-2s | GTT AGA TCT CTC CGG AGG AGG TGG CTC AGG TGG TGG |
| | | AGG ATC TGG A |
| 95 | g4s5-asxho | CTC GAG ACT CCC ACC TCC TCC AGA TCC TCC ACC ACC |
| | | TGA GCC ACC T |
| 96 | g4s4-5′ | AAA GAT CTC TCC GGA GGA GGT GGC TCA GGT GGT GGA |
| | | GGA TCT GGA GGA GG |
| 97 | g4s4-3′ | CTC GAG ACC GGT AGA ACC ACC TCC ACC ACT CCC ACC |
| | | TCC TCC AGA TCC TC |
| 98 | g4s5-5 | GTT AGA TCT CTC CGG AGG AGG TGG CTC A |
| 99 | g4s5-3 | ACC GGT CTC GAG ACT CCC ACC TCC TCC AGA TC |

TABLE 2

| SEQ | | |
|---|---|---|
| ID NO: | DESCRIPTION | SEQUENCE (NUCLEOTIDE SEQUENCES ARE 5′-3′) |
| 100 | g4s4lnk | agatctctccggaggaggtggctcaggtggtggaggatctggaggaggtgggag |
| | | tggtggaggtggttctaccggtctcgag |
| 101 | G4S5-1 | agatctctccggaggaggtggctcaggtggtggaggatctggaggaggtggctc |
| | | aggtggtggaggatctggaggaggtgggagtaccggtctcgag |
| 102 | G4S5-2 | agatctctccggaggaggtggctcaggtggtggaggatctggaggaggtggctc |
| | | aggtggtggaggatctggaggaggtgggagtctcgag |
| 103 | 3′hRNase | gtcgacggagctagcagccccgtgaacgtgagcagccccagcgtgcaggatatc |
| | G88D | ccttccctgggcaaggaatcccgggccaagaaattccagcggcagcatatggac |
| | | tcagacagttcccccagcagcagctccacctactgtaaccaaatgatgaggcgc |
| | | cggaatatgacacaggggcggtgcaaaccagtgaacacctttgtgcacgagccc |
| | | ctggtagatgtccagaatgtctgtttccaggaaaaggtcacctgcaagaacggg |
| | | cagggcaactgctacaagagcaactccagcatgcacatcacagactgccgcctg |
| | | acaaacgactccaggtaccccaactgtgcataccggaccagcccgaaggagaga |
| | | cacatcattgtggcctgtgaagggagcccatatgtgccagtccactttgatgct |
| | | tctgtggaggactctacctaataatctaga |
| 104 | hDNase1- | gatatcctgaagatcgcagccttcaacatccagacatttggggagaccaagatg |
| | 3′-G105R; | tccaatgccaccctcgtcagctacattgtgcagatcctgagccgctatgacatc |
| | A114F | gccctggtccaggaggtcagagacagccacctgactgccgtggggaagctgctg |
| | | gacaacctcaatcaggatgcaccagacacctatcactacgtggtcagtgagcca |
| | | ctgggacggaacagctataaggagcgctacctgttcgtgtacaggcctgaccag |
| | | gtgtctgcggtggacagctactactacgatgatggctgcgagccctgcaggaac |
| | | gacaccttcaaccgagagccattcattgtcaggttcttctcccggttcacagag |
| | | gtcagggagtttgccattgttcccctgcatgcggccccgggggacgcagtagcc |
| | | gagatcgacgctctctatgacgtctacctggatgtccaagagaaatggggcttg |
| | | gaggacgtcatgttgatgggcgacttcaatgcgggctgcagctatgtgagaccc |
| | | tcccagtggtcatccatccgcctgtggacaagccccaccttccagtggctgatc |
| | | cccgacagcgctgacaccacagctacacccacgcactgtgcctatgacaggatc |
| | | gtggttgcagggatgctgctccgaggcgccgttgttcccgactcggctcttccc |
| | | tttaacttccaggctgcctatggcctgagtgaccaactggcccaagccatcagt |
| | | gaccactatccagtggaggtgatgctgaagtgataatctaga |
| 105 | hDNase1- | gatatcctgaagatcgcagccttcaacatccagacatttggggagaccaagatg |
| | 3′-WT | tccaatgccaccctcgtcagctacattgtgcagatcctgagccgctatgacatc |
| | | gccctggtccaggaggtcagagacagccacctgactgccgtggggaagctgctg |
| | | gacaacctcaatcaggatgcaccagacacctatcactacgtggtcagtgagcca |
| | | ctgggacggaacagctataaggagcgctacctgttcgtgtacaggcctgaccag |
| | | gtgtctgcggtggacagctactactacgatgatggctgcgagccctgcgggaac |
| | | gacaccttcaaccgagagccagccattgtcaggttcttctcccggttcacagag |
| | | gtcagggagtttgccattgttcccctgcatgcggccccgggggacgcagtagcc |
| | | gagatcgacgctctctatgacgtctacctggatgtccaagagaaatggggcttg |
| | | gaggacgtcatgttgatgggcgacttcaatgcgggctgcagctatgtgagaccc |
| | | tcccagtggtcatccatccgcctgtggacaagccccaccttccagtggctgatc |
| | | cccgacagcgctgacaccacagctacacccacgcactgtgcctatgacaggatc |
| | | gtggttgcagggatgctgctccgaggcgccgttgttcccgactcggctcttccc |
| | | tttaacttccaggctgcctatggcctgagtgaccaactggcccaagccatcagt |
| | | gaccactatccagtggaggtgatgctgaaatgataatctaga |
| 106 | hDNase1- | gatatcctgaagatcgcagccttcaacatccagacatttggggagaccaagatg |
| | 3′A114F | tccaatgccaccctcgtcagctacattgtgcagatcctgagccgctatgacatc |
| | | gccctggtccaggaggtcagagacagccacctgactgccgtggggaagctgctg |
| | | gacaacctcaatcaggatgcaccagacacctatcactacgtggtcagtgagcca |
| | | ctgggacggaacagctataaggagcgctacctgttcgtgtacaggcctgaccag |
| | | gtgtctgcggtggacagctactactacgatgatggctgcgagccctgcgggaac |
| | | gacaccttcaaccgagagccattcattgtcaggttcttctcccggttcacagag |
| | | gtcagggagtttgccattgttcccctgcatgcggccccgggggacgcagtagcc |
| | | gagatcgacgctctctatgacgtctacctggatgtccaagagaaatggggctta |
| | | gaggacgtcatgttgatgggcgacttcaatgcgggctgcagctatgtgagaccc |
| | | tcccagtggtcatccatccgcctgtggacaagccccaccttccagtggctgatc |
| | | cccgacagcgctgacaccacagctacacccacgcactgtgcctatgacaggatc |
| | | gtggttgcagggatgctgctccgaggcgccgttgttcccgactcggctcttccc |
| | | tttaacttccaggctgcctatggcctgagtgaccaactggcccaagccatcagt |
| | | gaccactatccagtggaggtgatgctgaagtgataatctaga |
| 107 | hDNase1- | accggtctgaagatcgcagccttcaacatccagacatttggggagaccaagatg |
| | 5′-G105R; | tccaatgccaccctcgtcagctacattgtgcagatcctgagccgctatgacatc |
| | A114F | gccctggtccaggaggtcagagacagccacctgactgccgtggggaagctgctg |
| | | gacaacctcaatcaggatgcaccagacacctatcactacgtggtcagtgagcca |
| | | ctgggacggaacagctataaggagcgctacctgttcgtgtacaggcctgaccag |
| | | gtgtctgcggtggacagctactactacgatgatggctgcgagccctgcaggaac |
| | | gacaccttcaaccgagagccattcattgtcaggttcttctcccggttcacagag |
| | | gtcagggagtttgccattgttcccctgcatgcggccccgggggacgcagtagcc |
| | | gagatcgacgctctctatgacgtctacctggatgtccaagagaaatggggcttg |
| | | gaggacgtcatgttgatgggcgacttcaatgcgggctgcagctatgtgagaccc |
| | | tcccagtggtcatccatccgcctgtggacaagccccaccttccagtggctgatc |
| | | cccgacagcgctgacaccacagctacacccacgcactgtgcctatgacaggatc |
| | | gtggttgcagggatgctgctccgaggcgccgttgttcccgactcggctcttccc |
| | | tttaacttccaggctgcctatggcctgagtgaccaactggcccaagccatcagt |
| | | gaccactatccagtggaggtgatgctgaaagatctcgag |
| 108 | hDNase1- | accggtctgaagatcgcagccttcaacatccagacatttggggagaccaagatg |
| | 5′-WT | tccaatgccaccctcgtcagctacattgtgcagatcctgagccgctatgacatc |
| | | gccctggtccaggaggtcagagacagccacctgactgccgtggggaagctgctg |
| | | gacaacctcaatcaggatgcaccagacacctatcactacgtggtcagtgagcca |
| | | ctgggacggaacagctataaggagcgctacctgttcgtgtacaggcctgaccag |
| | | gtgtctgcggtggacagctactactacgatgatggctgcgagccctgcgggaac |
| | | gacaccttcaaccgagagccagccattgtcaggttcttctcccggttcacagag |
| | | gtcagggagtttgccattgttcccctgcatgcggccccgggggacgcagtagcc |
| | | gagatcgacgctctctatgacgtctacctggatgtccaagagaaatggggcttg |
| | | gaggacgtcatgttgatgggcgacttcaatgcgggctgcagctatgtgagaccc |
| | | tcccagtggtcatccatccgcctgtggacaagccccaccttccagtggctgatc |
| | | cccgacagcgctgacaccacagctacacccacgcactgtgcctatgacaggatc |
| | | gtggttgcagggatgctgctccgaggcgccgttgttcccgactcggctcttccc |
| | | tttaacttccaggctgcctatggcctgagtgaccaactggcccaagccatcagt |
| | | gaccactatccagtggaggtgatgctgaaagatctcgag |
| 109 | hDNase1- | accggtctgaagatcgcagccttcaacatccagacatttggggagaccaagatg |
| | 5′-A114F | tccaatgccaccctcgtcagctacattgtgcagatcctgagccgctatgacatc |
| | | gccctggtccaggaggtcagagacagccacctgactgccgtggggaagctgctg |
| | | gacaacctcaatcaggatgcaccagacacctatcactacgtggtcagtgagcca |
| | | ctgggacggaacagctataaggagcgctacctgttcgtgtacaggcctgaccag |
| | | gtgtctgcggtggacagctactactacgatgatggctgcgagccctgcgggaac |
| | | gacaccttcaaccgagagccattcattgtcaggttcttctcccggttcacagag |
| | | gtcagggagtttgccattgttcccctgcatgcggccccgggggacgcagtagcc |
| | | gagatcgacgctctctatgacgtctacctggatgtccaagagaaatggggctta |
| | | gaggacgtcatgttgatgggcgacttcaatgcgggctgcagctatgtgagaccc |
| | | tcccagtggtcatccatccgcctgtggacaagccccaccttccagtggctgatc |
| | | cccgacagcgctgacaccacagctacacccacgcactgtgcctatgacaggatc |
| | | gtggttgcagggatgctgctccgaggcgccgttgttcccgactcggctcttccc |
| | | tttaacttccaggctgcctatggcctgagtgaccaactggcccaagccatcagt |
| | | gaccactatccagtggaggtgatgctgaaagatctcgag |
| 110 | hIgG1WT | agatctcgagcccaaatcttctgacaaaactcacacatgtccaccgtgcccagc |
| | | acctgaactcctggggggaccgtcagtcttcctcttccccccaaaacccaagga |
| | | caccctcatgatctcccggacccctgaggtcacatgcgtggtggtggacgtgag |
| | | ccacgaagaccctgaggtcaagttcaactggtacgtggacggcgtggaggtgca |
| | | taatgccaagacaaagccgcgggaggagcagtacaacagcacgtaccgtgtggt |
| | | cagcgtcctcaccgtcctgcaccaggactggctgaatggcaaggagtacaagtg |
| | | caaggtctccaacaaagccctcccagcccccatcgagaaaaccatctccaaagc |
| | | caaagggcagccccgagaaccacaggtgtacaccctgcccccatcccgggatga |
| | | gctgaccaagaaccaggtcagcctgacctgcctggtcaaaggcttctatcccag |
| | | cgacatcgccgtggagtgggagagcaatgggcagccggagaacaactacaagac |
| | | cacgcctcccgtgctggactccgacggctccttcttcctctacagcaagctcac |
| | | cgtggacaagagcaggtggcagcaggggaacgtcttctcatgctccgtgatgca |
| | | tgaggctctgcacaaccactacacgcagaagagcctctctctgtctccgggtaa |
| | | atgataatctaga |
| 111 | hDNase1 + | gttaagcttgccaccatggaaaccccagcgcagcttctcttcctcctgctactc |
| | VK3LP | tggctcccagataccaccggtctgaagatcgcagccttcaacatccagacattt |
| | | ggggagaccaagatgtccaatgccaccctcgtcagctacattgtgcagatcctg |
| | | agccgctatgacatcgccctggtccaggaggtcagagacagccacctgactgcc |
| | | gtggggaagctgctggacaacctcaatcaggatgcaccagacacctatcactac |
| | | gtggtcagtgagccactgggacggaacagctataaggagcgctacctgttcgtg |
| | | tacaggcctgaccaggtgtctgcggtggacagctactactacgatgatggctgc |
| | | gagccctgcgggaacgacaccttcaaccgagagccagccattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaagtga |
| 112 | hDNase1L3 | atgtcacgggagctggccccactgctgcttctcctcctctccatccacagcgcc |
| | | ctggccatgaggatctgctccttcaacgtcaggtcctttggggaaagcaagcag |
| | | gaagacaagaatgccatggatgtcattgtgaaggtcatcaaacgctgtgacatc |
| | | atactcgtgatggaaatcaaggacagcaacaacaggatctgccccatactgatg |
| | | gagaagctgaacagaaattcaaggagaggcataacatacaactatgtgattagc |
| | | tctcggcttggaagaaacacatataaagaacaatatgcctttctctacaaggaa |
| | | aagctggtgtctgtgaagaggagttatcactaccatgactatcaggatggagac |
| | | gcagatgtgttttccagggagccctttgtggtctggttccaatctccccacact |
| | | gctgtcaaagacttcgtgattatccccctgcacaccaccccagagacatccgtt |
| | | aaggagatcgatgagttggttgaggtctacacggacgtgaaacaccgctggaag |
| | | gcggagaatttcattttcatgggtgacttcaatgccggctgcagctacgtcccc |
| | | aagaaggcctggaagaacatccgcttgaggactgaccccaggtttgtttggctg |
| | | atcggggaccaagaggacaccacggtgaagaagagcaccaactgtgcatatgac |
| | | aggattgtgcttagaggacaagaaatcgtcagttctgttgttcccaagtcaaac |
| | | agtgtttttgacttccagaaagcttacaagctgactgaagaggaggccctggat |
| | | gtcagcgaccactttccagttgaatttaaactacagtcttcaagggccttcacc |
| | | aacagcaaaaaatctgtcactctaaggaagaaaacaaagagcaaacgctcctag |
| 113 | human | atgggtctggagaagtctcttgtccggctccttctgcttgtcctgatactgctg |
| | pancreatic | gtgctgggctgggtccagccttccctgggcaaggaatcccgggccaagaaattc |
| | ribonuclease | cagcggcagcatatggactcagacagttcccccagcagcagctccacctactgt |
| | | aaccaaatgatgaggcgccggaatatgacacaggggcggtgcaaaccagtgaac |
| | | acctttgtgcacgagcccctggtagatgtccagaatgtctgtttccaggaaaag |
| | | gtcacctgcaagaacgggcagggcaactgctacaagagcaactccagcatgcac |
| | | atcacagactgccgcctgacaaacggctccaggtaccccaactgtgcataccgg |
| | | accagcccgaaggagagacacatcattgtggcctgtgaagggagcccatatgtg |
| | | ccagtccactttgatgctactgtgtag |
| 114 | huVK3LP + | gttaagcttgccaccatggaaaccccagcgcagcttctcttcctcctgctactc |
| | mrib1 + | tggctcccagataccaccggtagggaatctgcagcacagaagtttcagcggcag |
| | mIgG2A-C-2S | cacatggatccagatggttcctccatcaacagccccacctactgcaaccaaatg |
| | | atgaaacgccgggatatgacaaatgggtcatgcaagcccgtgaacaccttcgtg |
| | | catgagcccttggcagatgtccaggccgtctgctcccaggaaaatgtcacctgc |
| | | aagaacaggaagagcaactgctacaagagcagctctgccctgcacatcactgac |
| | | tgccacctgaagggcaactccaagtatcccaactgtgactacaagaccactcaa |
| | | taccagaagcacatcattgtggcctgtgaagggaacccctacgtaccagtccac |
| | | tttgatgctactgtgctcgagcccagaggtctcacaatcaagccctctcctcca |
| | | tgcaaatgcccagcacctaacctcttgggtggatcatccgtcttcatcttccct |
| | | ccaaagatcaaggatgtactcatgatctccctgagccccatggtcacatgtgtg |
| | | gtggtggatgtgagcgaggatgacccagacgtccagatcagctggtttgtgaac |
| | | aacgtggaagtacacacagctcagacacaaacccatagagaggattacaacagt |
| | | actctccgggtggtcagtgccctccccatccagcaccaggactggatgagtggc |
| | | aaggagttcaaatgctcggtcaacaacaaagacctcccagcgtccatcgagaga |
| | | accatctcaaaacccagagggccagtaagagctccacaggtatatgtcttgcct |
| | | ccaccagcagaagagatgactaagaaagagttcagtctgacctgcatgatcaca |
| | | ggcttcttacctgccgaaattgctgtggactggaccagcaatgggcgtacagag |
| | | caaaactacaagaacaccgcaacagtcctggactctgatggttcttacttcatg |
| | | tacagcaagctcagagtacaaaagagcacttgggaaagaggaagtcttttcgcc |
| | | tgctcagtggtccacgagggtctgcacaatcaccttacgactaagagcttctct |
| | | cggactccgggtaaatgataatctagaa |
| 115 | huVK3LP- | aagcttgccgccatggaaaccccagcgcagcttctcttcctcctgctactctgg |
| | hRNaseWT- | ctcccagataccaccggtaaggaatcccgggccaagaaattccagcggcagcat |
| | hIgGWt- | atggactcagacagttcccccagcagcagctccacctactgtaaccaaatgatg |
| | NLG- | aggcgccggaatatgacacaggggcggtgcaaaccagtgaacacctttgtgcac |
| | hDNAse1- | gagcccctggtagatgtccagaatgtctgtttccaggaaaaggtcacctgcaag |
| | 105-114 | aacgggcagggcaactgctacaagagcaactccagcatgcacatcacagactgc |
| | | cgcctgacaaacggctccaggtaccccaactgtgcataccggaccagcccgaag |
| | | gagagacacatcattgtggcctgtgaagggagcccatatgtgccagtccacttt |
| | | gatgcttctgtggaggactctacagatctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgagggtctgcacaaccactacacgcagaag |
| | | agcctctctctgtctccgggtaaagtcgacggtgctagcagccatgtgaatgtg |
| | | agcagccctagcgtgcaggatatcctgaagatcgcagccttcaacatccagaca |
| | | tttggggagaccaagatgtccaatgccaccctcgtcagctacattgtgcagatc |
| | | ctgagccgctatgacatcgccctggtccaggaggtcagagacagccacctgact |
| | | gccgtggggaagctgctggacaacctcaatcaggatgcaccagacacctatcac |
| | | tacgtggtcagtgagccactgggacggaacagctataaggagcgctacctgttc |
| | | gtgtacaggcctgaccaggtgtctgcggtggacagctactactacgatgatggc |
| | | tgcgagccctgcgggaacgacaccttcaaccgagagccagccattgtcaggttc |
| | | ttctcccggttcacagaggtcagggagtttgccattgttcccctgcatgcggcc |
| | | ccgggggacgcagtagccgagatcgacgctctctatgacgtctacctggatgtc |
| | | caagagaaatggggctcggaggacgtcatgttgatgggcgacttcaatgcgggc |
| | | tgcagctatgtgagaccctcccagtggtcatccatccgcctgtggacaagcccc |
| | | accttccagtggctgatccccgacagcgctgacaccacagctacacccacgcac |
| | | tgtgcctatgacaggatcgtggttgcagggatgctgctccgaggcgccgttgtt |
| | | cccgactcggctcttccctttaacttccagnctgcctatggcctgagtgaccaa |
| | | ctggcccaagccatcagtgaccactatccagtggaggtgatgctgaagtgataa |
| | | tctaga |
| 116 | huVK3LP- | aagcttgccgccatggaaaccccagcgcagcttctcttcctcctgctactctgg |
| | hRNaseWT- | ctcccagataccaccggtaaggaatcccgggccaagaaattccagcggcagcat |
| | hIgGWT- | atggactcagacagttcccccagcagcagctccacctactgtaaccaaatgatg |
| | NLG- | aggcgccggaatatgacacaggggcggtgcaaaccagtgaacacctttgtgcac |
| | hDNase1- | gagcccctggtagatgtccagaatgtctgtttccaggaaaaggtcacctgcaag |
| | 114F | aacgggcagggcaactgctacaagagcaactccagcatgcacatcacagactgc |
| | | cgcctgacaaacggctccaggtaccccaactgtgcataccggaccagcccgaag |
| | | gagagacacatcattgtggcctgtgaagggagcccatatgtgccagtccacttt |
| | | gatgcttctgtggaggactctacagatctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgagggtctgcacaaccactacacgcagaag |
| | | agcctctctctgtctccgggtaaagtcgacggtgctagcagccatgtgaatgtg |
| | | agcagccctagcgtgcaggatatcctgaagatcgcagccttcaacatccagaca |
| | | tttggggagaccaagatgtccaatgccaccctcgtcagctacattgtgcagatc |
| | | ctgagccgctatgacatcgccctggtccaggaggtcagagacagccacctgact |
| | | gccgtggggaagctgctggacaacctcaatcaggatgcaccagacacctatcac |
| | | tacgtggtcagtgagccactgggacggaacagctataaggagcgctacctgttc |
| | | gtgtacaggcctgaccaggtgtctgcggtggacagctactactacgatgatggc |
| | | tgcgagccctgcgggaacgacaccttcaaccgagagccattcattgtcaggttc |
| | | ttctcccggttcacagaggtcagggagtttgccattgttcccctgcatgcggcc |
| | | ccgggggacgcagtagccgagatcgacgctctctatgacgtctacctggatgtc |
| | | caagagaaatggggcttagaggacgtcatgttgatgggcgacttcaatgcgggc |
| | | tgcagctatgtgagaccctcccagtggtcatccatccgcctgtggacaagcccc |
| | | accttccagtggctgatccccgacagcgctgacaccacagctacacccacgcac |
| | | tgtgcctatgacaggatcgtggttgcagggatgctgctccgaggcgccgttgtt |
| | | cccgactcggctcttccctttaacttccaggctgcctatggcctgagtgaccaa |
| | | ctggcccaagccatcagtgaccactatccagtggaggtgatgctgaagtgataa |
| | | tctaga |
| 117 | huVK3LP- | aagcttgccgccatggaaaccccagcgcagcttctcttcctcctgctactctgg |
| | hRNaseWT- | ctcccagataccaccggtaaggaatcccgggccaagaaattccagcggcagcat |
| | hIgGWT- | atggactcagacagttcccccagcagcagctccacctactgtaaccaaatgatg |
| | NLG- | aggcgccggaatatgacacaggggcggtgcaaaccagtgaacacctttgtgcac |
| | hDNase1-WT | gagcccctggtagatgtccagaatgtctgtttccaggaaaaggtcacctgcaag |
| | | aacgggcagggcaactgctacaagagcaactccagcatgcacatcacagactgc |
| | | cgcctgacaaacggctccaggtaccccaactgtgcataccggaccagcccgaag |
| | | gagagacacatcattgtggcctgtgaagggagcccatatgtgccagtccacttt |
| | | gatgcttctgtggaggactctacagatctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgagggtctgcacaaccactacacgcagaag |
| | | agcctctctctgtctccgggtaaagtcgacggtgctagcagccatgtgaatgtg |
| | | agcagccctagcgtgcaggatatcctgaagatcgcagccttcaacatccagaca |
| | | tttggggagaccaagatgtccaatgccaccctcgtcagctacattgtgcagatc |
| | | ctgagccgctatgacatcgccctggtccaggaggtcagagacagccacctgact |
| | | gccgtggggaagctgctggacaacctcaatcaggatgcaccagacacctatcac |
| | | tacgtggtcagtgagccactgggacggaacagctataaggagcgctacctgttc |
| | | gtgtacaggcctgaccaggtgtctgcggtggacagctactactacgatgatggc |
| | | tgcgagccctgcgggaacgacaccttcaaccgagagccagccattgtcaggttc |
| | | ttctcccggttcacagaggtcagggagtttgccattgttcccctgcatgcggcc |
| | | ccgggggacgcagtagccgagatcgacgctctctatgacgtctacctggatgtc |
| | | caagagaaatggggcttggaggacgtcatgttgatgggcgacttcaatgcgggc |
| | | tgcagctatgtgagaccctcccagtggtcatccatccgcctgtggacaagcccc |
| | | accttccagtggctgatccccgacagcgctgacaccacagctacacccacgcac |
| | | tgtgcctatgacaggatcgtggttgcagggatgctgctccgaggcgccgttgtt |
| | | cccgactcggctcttccctttaacttccaggctgcctatggcctgagtgaccaa |
| | | ctggcccaagccatcagtgaccactatccagtggaggtgatgctgaaatgataa |
| | | tctaga |
| 118 | hVK3LP- | gttaagcttgccaccatggaaaccccagcgcagcttctcttcctcctgctactc |
| | hDNase1(WT)- | tggctcccagataccaccggtctgaagatcgcagccttcaacatccagacattt |
| | hIgG1WT | ggggagaccaagatgtccaatgccaccctcgtcagctacattgtgcagatcctg |
| | | agccgctatgacatcgccctggtccaggaggtcagagacagccacctgactgcc |
| | | gtggggaagctgctggacaacctcaatcaggatgcaccagacacctatcactac |
| | | gtggtcagtgagccactgggacggaacagctataaggagcgctacctgttcgtg |
| | | tacaggcctgaccaggtgtctgcggtggacagctactactacgatgatggctgc |
| | | gagccctgcgggaacgacaccttcaaccgagagccagccattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaatgataatct |
| | | aga |
| 119 | hVK3LP- | gttaagcttgccaccatggaaaccccagcgcagcttctcttcctcctgctactc |
| | hDNase1- | tggctcccagataccaccggtctgaagatcgcagccttcaacatccagacattt |
| | A114F- | ggggagaccaagatgtccaatgccaccctcgtcagctacattgtgcagatcctg |
| | hIgG1WT | agccgctatgacatcgccctggtccaggaggtcagagacagccacctgactgcc |
| | | gtggggaagctgctggacaacctcaatcaggatgcaccagacacctatcactac |
| | | gtggtcagtgagccactgggacggaacagctataaggagcgctacctgttcgtg |
| | | tacaggcctgaccaggtgtctgcggtggacagctactactacgatgatggctgc |
| | | gagccctgcgggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttagaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaatgataatct |
| | | aga |
| 120 | hVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)4- | gtggggaagctgctggacaacctcaatcaggatgcaccagacacctatcactac |
| | hIgG1WT | gtggtcagtgagccactgggacggaacagctataaggagcgctacctgttcgtg |
| | | tacaggcctgaccaggtgtctgcggtggacagctactactacgatgatggctgc |
| | | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtgggagtggtggaggt |
| | | ggttctaccggtctcgagcccaaatcttctgacaaaactcacacatgtccaccg |
| | | tgcccagcacctgaactcctggggggaccgtcagtcttcctcttccccccaaaa |
| | | cccaaggacaccctcatgatctcccggacccctgaggtcacatgcgtggtggtg |
| | | gacgtgagccacgaagaccctgaggtcaagttcaactggtacgtggacggcgtg |
| | | gaggtgcataatgccaagacaaagccgcgggaggagcagtacaacagcacgtac |
| | | cgtgtggtcagcgtcctcaccgtcctgcaccaggactggctgaatggcaaggag |
| | | tacaagtgcaaggtctccaacaaagccctcccagcccccatcgagaaaaccatc |
| | | tccaaagccaaagggcagccccgagaaccacaggtgtacaccctgcccccatcc |
| | | cgggatgagctgaccaagaaccaggtcagcctgacctgcctggtcaaaggcttc |
| | | tatcccagcgacatcgccgtggagtgggagagcaatgggcagccggagaacaac |
| | | tacaagaccacgcctcccgtgctggactccgacggctccttcttcctctacagc |
| | | aagctcaccgtggacaagagcaggtggcagcaggggaacgtcttctcatgctcc |
| | | gtgatgcatgaggctctgcacaaccactacacgcagaagagcctctctctgtct |
| | | ccgggtaaatgataatctaga |
| 121 | hVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)5-1- | gtggggaagctgctggacaacctcaatcaggatgcaccagacacctatcactac |
| | hIgG1WT | gtggtcagtgagccactgggacggaacagctataaggagcgctacctgttcgtg |
| | | tacaggcctgaccaggtgtctgcggtggacagctactactacgatgatggctgc |
| | | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtggctcaggtggtgga |
| | | ggatctggaggaggtgggagtaccggtctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgaggctctgcacaaccactacacgcagaag |
| | | agcctctctctgtctccgggtaaatgataatctaga |
| 122 | hVK3LP- | gttaagcttgccaccatggaaaccccagcgcagcttctcttcctcctgctactc |
| | hDNAse1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)5-2- | gtggggaagctgctggacaacctcaatcaggatgcaccagacacctatcactac |
| | hIgG1-WT | gtggtcagtgagccactgggacggaacagctataaggagcgctacctgttcgtg |
| | | tacaggcctgaccaggtgtctgcggtggacagctactactacgatgatggctgc |
| | | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtggctcaggtggtgga |
| | | ggatctggaggaggtgggagtctcgagcccaaatcttctgacaaaactcacaca |
| | | tgtccaccgtgcccagcacctgaactcctggggggaccgtcagtcttcctcttc |
| | | cccccaaaacccaaggacaccctcatgatctcccggacccctgaggtcacatgc |
| | | gtggtggtggacgtgagccacgaagaccctgaggtcaagttcaactggtacgtg |
| | | gacggcgtggaggtgcataatgccaagacaaagccgcgggaggagcagtacaac |
| | | agcacgtaccgtgtggtcagcgtcctcaccgtcctgcaccaggactggctgaat |
| | | ggcaaggagtacaagtgcaaggtctccaacaaagccctcccagcccccatcgag |
| | | aaaaccatctccaaagccaaagggcagccccgagaaccacaggtgtacaccctg |
| | | cccccatcccgggatgagctgaccaagaaccaggtcagcctgacctgcctggtc |
| | | aaaggcttctatcccagcgacatcgccgtggagtgggagagcaatgggcagccg |
| | | gagaacaactacaagaccacgcctcccgtgctggactccgacggctccttcttc |
| | | ctctacagcaagctcaccgtggacaagagcaggtggcagcaggggaacgtcttc |
| | | tcatgctccgtgatgcatgaggctctgcacaaccactacacgcagaagagcctc |
| | | tctctgtctccgggtaaatgataatctaga |
| 123 | hVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | hIgG1WT | gtggggaagctgctggacaacctcaatcaggatgcaccagacacctatcactac |
| | | gtggtcagtgagccactgggacggaacagctataaggagcgctacctgttcgtg |
| | | tacaggcctgaccaggtgtctgcggtggacagctactactacgatgatggctgc |
| | | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaatgataatct |
| | | aga |
| 124 | hVK3LP- | gttaagcttgccaccatggaaaccccagcgcagcttctcttcctcctgctactc |
| | hRNase(MT)- | tggctcccagataccaccggtaaggaatcccgggccaagaaattccagcggcag |
| | hIgG1WT | catatggactcagacagttcccccagcagcagctccacctactgtaaccaaatg |
| | | atgaggcgccggaatatgacacaggggcggtgcaaaccagtgaacacctttgtg |
| | | cacgagcccctggtagatgtccagaatgtctgtttccaggaaaaggtcacctgc |
| | | aagaacgggcagggcaactgctacaagagcaactccagcatgcacatcacagac |
| | | tgccgcctgacaaacgactccaggtaccccaactgtgcataccggaccagcccg |
| | | aaggagagacacatcattgtggcctgtgaagggagcccatatgtgccagtccac |
| | | tttgatgcttctgtggaggactctacagatctcgagcccaaatcttctgacaaa |
| | | actcacacatgtccaccgtgcccagcacctgaactcctggggggaccgtcagtc |
| | | ttcctcttccccccaaaacccaaggacaccctcatgatctcccggacccctgag |
| | | gtcacatgcgtggtggtggacgtgagccacgaagaccctgaggtcaagttcaac |
| | | tggtacgtggacggcgtggaggtgcataatgccaagacaaagccgcgggaggag |
| | | cagtacaacagcacgtaccgtgtggtcagcgtcctcaccgtcctgcaccaggac |
| | | tggctgaatggcaaggagtacaagtgcaaggtctccaacaaagccctcccagcc |
| | | cccatcgagaaaaccatctccaaagccaaagggcagccccgagaaccacaggtg |
| | | tacaccctgcccccatcccgggatgagctgaccaagaaccaggtcagcctgacc |
| | | tgcctggtcaaaggcttctatcccagcgacatcgccgtggagtgggagagcaat |
| | | gggcagccggagaacaactacaagaccacgcctcccgtgctggactccgacggc |
| | | tccttcttcctctacagcaagctcaccgtggacaagagcaggtggcagcagggg |
| | | aacgtcttctcatgctccgtgatgcatgaggctctgcacaaccactacacgcag |
| | | aagagcctctctctgtctccgggtaaatgataatctaga |
| 125 | hVK3LP- | gttaagcttgccaccatggaaaccccagcgcagcttctcttcctcctgctactc |
| | hRNase(WT)- | tggctcccagataccaccggtaaggaatcccgggccaagaaattccagcggcag |
| | (G4S)4lnk- | catatggactcagacagttcccccagcagcagctccacctactgtaaccaaatg |
| | hIgG1WT | atgaggcgccggaatatgacacaggggcggtgcaaaccagtgaacacctttgtg |
| | | cacgagcccctggtagatgtccagaatgtctgtttccaggaaaaggtcacctgc |
| | | aagaacgggcagggcaactgctacaagagcaactccagcatgcacatcacagac |
| | | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacagatctctccggaggaggtggctcaggt |
| | | ggtggaggatctggaggaggtgggagtggtggaggtggttctaccggtctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaatgataatct |
| | | aga |
| 126 | hVK3LP- | gttaagcttgccaccatggaaaccccagcgcagcttctcttcctcctgctactc |
| | hRNase(WT)- | tggctcccagataccaccggtaaggaatcccgggccaagaaattccagcggcag |
| | (G4S)5- | catatggactcagacagttcccccagcagcagctccacctactgtaaccaaatg |
| | 2-lnk- | atgaggcgccggaatatgacacaggggcggtgcaaaccagtgaacacctttgtg |
| | hIgG1WT | cacgagcccctggtagatgtccagaatgtctgtttccaggaaaaggtcacctgc |
| | | aagaacgggcagggcaactgctacaagagcaactccagcatgcacatcacagac |
| | | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacagatctctccggaggaggtggctcaggt |
| | | ggtggaggatctggaggaggtggctcaggtggtggaggatctggaggaggtggg |
| | | agtctcgagcccaaatcttctgacaaaactcacacatgtccaccgtgcccagca |
| | | cctgaactcctggggggaccgtcagtcttcctcttccccccaaaacccaaggac |
| | | accctcatgatctcccggacccctgaggtcacatgcgtggtggtggacgtgagc |
| | | cacgaagaccctgaggtcaagttcaactggtacgtggacggcgtggaggtgcat |
| | | aatgccaagacaaagccgcgggaggagcagtacaacagcacgtaccgtgtggtc |
| | | agcgtcctcaccgtcctgcaccaggactggctgaatggcaaggagtacaagtgc |
| | | aaggtctccaacaaagccctcccagcccccatcgagaaaaccatctccaaagcc |
| | | aaagggcagccccgagaaccacaggtgtacaccctgcccccatcccgggatgag |
| | | ctgaccaagaaccaggtcagcctgacctgcctggtcaaaggcttctatcccagc |
| | | gacatcgccgtggagtgggagagcaatgggcagccggagaacaactacaagacc |
| | | acgcctcccgtgctggactccgacggctccttcttcctctacagcaagctcacc |
| | | gtggacaagagcaggtggcagcaggggaacgtcttctcatgctccgtgatgcat |
| | | gaggctctgcacaaccactacacgcagaagagcctctctctgtctccgggtaaa |
| | | tgataatctaga |
| 127 | hVK3LP- | gttaagcttgccaccatggaaaccccagcgcagcttctcttcctcctgctactc |
| | hRNase(WT)- | tggctcccagataccaccggtaaggaatcccgggccaagaaattccagcggcag |
| | hIgG1WT | catatggactcagacagttcccccagcagcagctccacctactgtaaccaaatg |
| | | atgaggcgccggaatatgacacaggggcggtgcaaaccagtgaacacctttgtg |
| | | cacgagcccctggtagatgtccagaatgtctgtttccaggaaaaggtcacctgc |
| | | aagaacgggcagggcaactgctacaagagcaactccagcatgcacatcacagac |
| | | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacagatctcgagcccaaatcttctgacaaa |
| | | actcacacatgtccaccgtgcccagcacctgaactcctggggggaccgtcagtc |
| | | ttcctcttccccccaaaacccaaggacaccctcatgatctcccggacccctgag |
| | | gtcacatgcgtggtggtggacgtgagccacgaagaccctgaggtcaagttcaac |
| | | tggtacgtggacggcgtggaggtgcataatgccaagacaaagccgcgggaggag |
| | | cagtacaacagcacgtaccgtgtggtcagcgtcctcaccgtcctgcaccaggac |
| | | tggctgaatggcaaggagtacaagtgcaaggtctccaacaaagccctcccagcc |
| | | cccatcgagaaaaccatctccaaagccaaagggcagccccgagaaccacaggtg |
| | | tacaccctgcccccatcccgggatgagctgaccaagaaccaggtcagcctgacc |
| | | tgcctggtcaaaggcttctatcccagcgacatcgccgtggagtgggagagcaat |
| | | gggcagccggagaacaactacaagaccacgcctcccgtgctggactccgacggc |
| | | tccttcttcctctacagcaagctcaccgtggacaagagcaggtggcagcagggg |
| | | aacgtcttctcatgctccgtgatgcatgaggctctgcacaaccactacacgcag |
| | | aagagcctctctctgtctccgggtaaatgataatctaga |
| 128 | murine | atgggctcacagaccctgccccatggtcacatgcagaccctcatcttcttagac |
| | Trex1(FL) | ctggaagccactggcctgccttcgtctcggcccgaagtcacagagctgtgcctg |
| | transcript | ctggctgtccacagacgtgctctggagaacacttccatttctcagggacatcca |
| | variant 1 | cctccagtgcccagaccgccccgtgtggtggacaagctctctctgtgcattgct |
| | | ccagggaaagcctgtagccctggggccagtgagatcacaggtctgagcaaagct |
| | | gagctggaagtacaggggcgtcaacgcttcgatgacaacctggccatcctgctc |
| | | cgagccttcctgcagcgccagccacagccttgctgccttgtggcacacaacggt |
| | | gaccgctatgactttcctctgctccagacagagcttgctaggctgagcactccc |
| | | agtcccctagatggtaccttctgtgtggacagcatcgctgccctaaaggccttg |
| | | gaacaagctagcagcccctcagggaatggttcgaggaaaagctacagcctgggc |
| | | agcatctacacccgcctgtactggcaagcaccgacagactcacatactgctgaa |
| | | ggtgatgttctaaccctgctcagcatctgtcagtggaagccacaggccctactg |
| | | cagtgggtggacgaacatgcccggccctttagcaccgtcaagcccatgtacggc |
| | | actccggctaccactggaacaaccaacctaaggccacatgctgccacagctact |
| | | acacccctggccacagccaatggaagtcccagcaatggcaggagcaggcgacct |
| | | aagagtcctcctccagagaaggtcccagaagccccatcacaggaggggctgctg |
| | | gccccactgagcctgctgaccctcctgaccttggcaatagccactctgtatgga |
| | | ctcttcctggcctcacctgggcagtaa |
| 129 | mTREX1minec | atgggctcacagaccctgccccatggtcacatgcagaccctcatcttcttagac |
| | | ctggaagccactggcctgccttcgtctcggcccgaagtcacagagctgtgcctg |
| | | ctggctgtccacagacgtgctctggagaacacttccatttctcagggacatcca |
| | | cctccagtgcccagaccgccccgtgtggtggacaagctctctctgtgcattgct |
| | | ccagggaaagcctgtagccctggggccagtgagatcacaggtctgagcaaagct |
| | | gagctggaagtacaggggcgtcaacgcttcgatgacaacctggccatcctgctc |
| | | cgagccttcctgcagcgccagccacagccttgctgccttgtggcacacaacggt |
| | | gaccgctatgactttcctctgctccagacagagcttgctaggctgagcactccc |
| | | agtcccctagatggtaccttctgtgtggacagcatcgctgccctaaaggccttg |
| | | gaacaagctagcagcccctcagggaatggttcgaggaaaagctacagcctgggc |
| | | agcatctacacccgcctgtactggcaagcaccgacagactcacatactgctgaa |
| | | ggtgatgttctaaccctgctcagcatctgtcagtggaagccacaggccctactg |
| | | cagtgggtggacgaacatgcccggccctttagcaccgtcaagcccatgtacggc |
| | | actccggctaccactggaacaacagatctcgag |
| 130 | murine | aagcttgccaccatggaaaccccagcgcagcttctcttcctcctgctactctgg |
| | Trex1- | ctcccagataccaccggtatgggctcacagaccctgccccatggtcacatgcag |
| | (G4S)4- | accctcatcttcttagacctggaagccactggcctgccttcgtctcggcccgaa |
| | mIgG2a-c | gtcacagagctgtgcctgctggctgtccacagacgtgctctggagaacacttcc |
| | | atttctcagggacatccacctccagtgcccagaccgccccgtgtggtggacaag |
| | | ctctctctgtgcattgctccagggaaagcctgtagccctggggccagtgagatc |
| | | acaggtctgagcaaagctgagctggaagtacaggggcgtcaacgcttcgatgac |
| | | aacctggccatcctgctccgagccttcctgcagcgccagccacagccttgctgc |
| | | cttgtggcacacaacggtgaccgctatgactttcctctgctccagacagagctt |
| | | gctaggctgagcactcccagtcccctagatggtaccttctgtgtggacagcatc |
| | | gctgccctaaaggccttggaacaagctagcagcccctcagggaatggttcgagg |
| | | aaaagctacagcctgggcagcatctacacccgcctgtactggcaagcaccgaca |
| | | gactcacatactgctgaaggtgatgttctaaccctgctcagcatctgtcagtgg |
| | | aagccacaggccctactgcagtgggtggacgaacatgcccggccctttagcacc |
| | | gtcaagcccatgtacggcactccggctaccactggaacaacagatctctccgga |
| | | ggaggtggctcaggtggtggaggatctggaggaggtggctcagggagtggtgga |
| | | ggtggttctaccggtctcgagcccagaggtcccacaatcaagccctctcctcca |
| | | tgcaaatgcccagcacctaacctcttgggtggatcatccgtcttcatcttccct |
| | | ccaaagatcaaggatgtactcatgatctccctgagccccatggtcacatgtgtg |
| | | gtggtggatgtgagcgaggatgacccagacgtccagatcagctggtttgtgaac |
| | | aacgtggaagtacacacagctcagacacaaacccatagagaggattacaacagt |
| | | actctccgggtggtcagtgccctccccatccagcaccaggactggatgagtggc |
| | | aaggagttcaaatgctcggtcaacaacaaagacctcccagcgtccatcgagaga |
| | | accatctcaaaacccagagggccagtaagagctccacaggtatatgtcttgcct |
| | | ccaccagcagaagagatgactaagaaagagttcagtctgacctgcatgatcaca |
| | | ggcttcttacctgccgaaattgctgtggactggaccagcaatgggcgtacagag |
| | | caaaactacaagaacaccgcaacagtcctggactctgatggttcttacttcatg |
| | | tacagcaagctcagagtacaaaagagcacttgggaaagaggaagtcttttcgcc |
| | | tgctcagtggtccacgagggtctgcacaatcaccttacgactaagagcttctct |
| | | cggactccgggtaaatgataatctaga |
| 131 | murine | aagcttgccaccatggaaaccccagcgcagcttctcttcctcctgctactctgg |
| | Trex1- | ctcccagataccaccggtatgggctcacagaccctgccccatggtcacatgcag |
| | (G4S)5- | accctcatcttcttagacctggaagccactggcctgccttcgtctcggcccgaa |
| | mIgG2a-c | gtcacagagctgtgcctgctggctgtccacagacgtgctctggagaacacttcc |
| | | atttctcagggacatccacctccagtgcccagaccgccccgtgtggtggacaag |
| | | ctctctctgtgcattgctccagggaaagcctgtagccctggggccagtgagatc |
| | | acaggtctgagcaaagctgagctggaagtacaggggcgtcaacgcttcgatgac |
| | | aacctggccatcctgctccgagccttcctgcagcgccagccacagccttgctgc |
| | | cttgtggcacacaacggtgaccgctatgactttcctctgctccagacagagctt |
| | | gctaggctgagcactcccagtcccctagatggtaccttctgtgtggacagcatc |
| | | gctgccctaaaggccttggaacaagctagcagcccctcagggaatggttcgagg |
| | | aaaagctacagcctgggcagcatctacacccgcctgtactggcaagcaccgaca |
| | | gactcacatactgctgaaggtgatgttctaaccctgctcagcatctgtcagtgg |
| | | aagccacaggccctactgcagtgggtggacgaacatgcccggccctttagcacc |
| | | gtcaagcccatgtacggcactccggctaccactggaacaacagatctctccgga |
| | | ggaggtggctcaggtggtggaggatctggaggaggtggctcaggtggtggagga |
| | | tctggaggaggtgggagtctcgagcccagaggtcccacaatcaagccctctcct |
| | | ccatgcaaatgcccagcacctaacctcttgggtggatcatccgtcttcatcttc |
| | | cctccaaagatcaaggatgtactcatgatctccctgagccccatggtcacatgt |
| | | gtggtggtggatgtgagcgaggatgacccagacgtccagatcagctggtttgtg |
| | | aacaacgtggaagtacacacagctcagacacaaacccatagagaggattacaac |
| | | agtactctccgggtggtcagtgccctccccatccagcaccaggactggatgagt |
| | | ggcaaggagttcaaatgctcggtcaacaacaaagacctcccagcgtccatcgag |
| | | agaaccatctcaaaacccagagggccagtaagagctccacaggtatatgtcttg |
| | | cctccaccagcagaagagatgactaagaaagagttcagtctgacctgcatgatc |
| | | acaggcttcttacctgccgaaattgctgtggactggaccagcaatgggcgtaca |
| | | gagcaaaactacaagaacaccgcaacagtcctggactctgatggttcttacttc |
| | | atgtacagcaagctcagagtacaaaagagcacttgggaaagaggaagtcttttc |
| | | gcctgctcagtggtccacgagggtctgcacaatcaccttacgactaagagcttc |
| | | tctcggactccgggtaaatgataatctaga |
| 132 | NLG | gtcgacggcgcggccgccagccccgtgaacgtgagcagccccagcgtgcaggat |
| | linker | atc |
| 133 | murine | aagcttgccaccatggaaaccccagcgcagcttctcttcctcctgctactctgg |
| | Trex1- | ctcccagataccaccggtatgggctcacagaccctgccccatggtcacatgcag |
| | Trex1- | accctcatcttcttagacctggaagccactggcctgccttcgtctcggcccgaa |
| | (G4S)5- | gtcacagagctgtgcctgctggctgtccacagacgtgctctggagaacacttcc |
| | mIgG2a-c | atttctcagggacatccacctccagtgcccagaccgccccgtgtggtggacaag |
| | | ctctctctgtgcattgctccagggaaagcctgtagccctggggccagtgagatc |
| | | acaggtctgagcaaagctgagctggaagtacaggggcgtcaacgcttcgatgac |
| | | aacctggccatcctgctccgagccttcctgcagcgccagccacagccttgctgc |
| | | cttgtggcacacaacggtgaccgctatgactttcctctgctccagacagagctt |
| | | gctaggctgagcactcccagtcccctagatggtaccttctgtgtggacagcatc |
| | | gctgccctaaaggccttggaacaagctagcagcccctcagggaatggttcgagg |
| | | aaaagctacagcctgggcagcatctacacccgcctgtactggcaagcaccgaca |
| | | gactcacatactgctgaaggtgatgttctaaccctgctcagcatctgtcagtgg |
| | | aagccacaggccctactgcagtgggtggacgaacatgcccggccctttagcacc |
| | | gtcaagcccatgtacggcactccggctaccactggaacaacagatctcatgggc |
| | | tcacagaccctgccccatggtcacatgcagaccctcatcttcttagacctggaa |
| | | gccactggcctgccttcgtctcggcccgaagtcacagagctgtgcctgctggct |
| | | gtccacagacgtgctctggagaacacttccatttctcagggacatccacctcca |
| | | gtgcccagaccgccccgtgtggtggacaagctctctctgtgcattgctccaggg |
| | | aaagcctgtagccctggggccagtgagatcacaggtctgagcaaagctgagctg |
| | | gaagtacaggggcgtcaacgcttcgatgacaacctggccatcctgctccgagcc |
| | | ttcctgcagcgccagccacagccttgctgccttgtggcacacaacggtgaccgc |
| | | tatgactttcctctgctccagacagagcttgctaggctgagcactcccagtccc |
| | | ctagatggtaccttctgtgtggacagcatcgctgccctaaaggccttggaacaa |
| | | gctagcagcccctcagggaatggttcgaggaaaagctacagcctgggcagcatc |
| | | tacacccgcctgtactggcaagcaccgacagactcacatactgctgaaggtgat |
| | | gttctaaccctgctcagcatctgtcagtggaagccacaggccctactgcagtgg |
| | | gtggacgaacatgcccggccctttagcaccgtcaagcccatgtacggcactccg |
| | | gctaccactggaacaacagatctctccggaggaggtggctcaggtggtggagga |
| | | tctggaggaggtggctcaggtggtggaggatctggaggaggtgggagtctcgag |
| | | cccagaggtcccacaatcaagccctctcctccatgcaaatgcccagcacctaac |
| | | ctcttgggtggatcatccgtcttcatcttccctccaaagatcaaggatgtactc |
| | | atgatctccctgagccccatggtcacatgtgtggtggtggatgtgagcgaggat |
| | | gacccagacgtccagatcagctggtttgtgaacaacgtggaagtacacacagct |
| | | cagacacaaacccatagagaggattacaacagtactctccgggtggtcagtgcc |
| | | ctccccatccagcaccaggactggatgagtggcaaggagttcaaatgctcggtc |
| | | aacaacaaagacctcccagcgtccatcgagagaaccatctcaaaacccagaggg |
| | | ccagtaagagctccacaggtatatgtcttgcctccaccagcagaagagatgact |
| | | aagaaagagttcagtctgacctgcatgatcacaggcttcttacctgccgaaatt |
| | | gctgtggactggaccagcaatgggcgtacagagcaaaactacaagaacaccgca |
| | | acagtcctggactctgatggttcttacttcatgtacagcaagctcagagtacaa |
| | | aagagcacttgggaaagaggaagtcttttcgcctgctcagtggtccacgagggt |
| | | ctgcacaatcaccttacgactaagagcttctctcggactccgggtaaatgataa |
| | | tctaga |
| 134 | huVK3LP- | aagcttgccaccatggaaaccccagcgcagcttctcttcctcctgctactctgg |
| | huTREX1- | ctcccagataccaccggtatgggccctggagctcgcagacagggcaggattgtg |
| | 72aa- | cagggaaggcctgagatgtgcttctgcccaccccctaccccactccctcccctt |
| | (g4s)4- | cggatcttaacactgggcactcacacacccaccccatgctcctctccaggctca |
| | hIgG1-WT | gcagcaggtacgtacccaaccatgggctcgcaggccctgcccccggggcccatg |
| | | cagaccctcatctttttcgacatggaggccactggcttgcccttctcccagccc |
| | | aaggtcacggagctgtgcctgctggctgtccacagatgtgccctggagagcccc |
| | | cccacctctcaggggccacctcccacagttcctccaccaccgcgtgtggtagac |
| | | aagctctccctgtgtgtggctccggggaaggcctgcagccctgcagccagcgag |
| | | atcacaggtctgagcacagctgtgctggcagcgcatgggcgtcaatgttttgat |
| | | gacaacctggccaacctgctcctagccttcctgcggcgccagccacagccctgg |
| | | tgcctggtggcacacaatggtgaccgctacgacttccccctgctccaagcagag |
| | | ctggctatgctgggcctcaccagtgctctggatggtgccttctgtgtggatagc |
| | | atcactgcgctgaaggccctggagcgagcaagcagcccctcagaacacggccca |
| | | aggaagagctacagcctaggcagcatctacactcgcctgtatgggcagtcccct |
| | | ccagactcgcacacggctgagggtgatgtcctggccctgctcagcatctgtcag |
| | | tggagaccacaggccctgctgcggtgggtggatgctcacgccaggcctttcggc |
| | | accatcaggcccatgtatggggtcacagcctctgctaggaccaaagatctctcc |
| | | ggaggaggtggctcaggtggtggaggatctggaggaggtgggagtggtggaggt |
| | | ggttctaccggtctcgagcccaaatcttctgacaaaactcacacatgtccaccg |
| | | tgcccagcacctgaactcctggggggaccgtcagtcttcctcttccccccaaaa |
| | | cccaaggacaccctcatgatctcccggacccctgaggtcacatgcgtggtggtg |
| | | gacgtgagccacgaagaccctgaggtcaagttcaactggtacgtggacggcgtg |
| | | gaggtgcataatgccaagacaaagccgcgggaggagcagtacaacagcacgtac |
| | | cgtgtggtcagcgtcctcaccgtcctgcaccaggactggctgaatggcaaggag |
| | | tacaagtgcaaggtctccaacaaagccctcccagcccccatcgagaaaaccatc |
| | | tccaaagccaaagggcagccccgagaaccacaggtgtacaccctgcccccatcc |
| | | cgggatgagctgaccaagaaccaggtcagcctgacctgcctggtcaaaggcttc |
| | | tatcccagcgacatcgccgtggagtgggagagcaatgggcagccggagaacaac |
| | | tacaagaccacgcctcccgtgctggactccgacggctccttcttcctctacagc |
| | | aagctcaccgtggacaagagcaggtggcagcaggggaacgtcttctcatgctcc |
| | | gtgatgcatgaggctctgcacaaccactacacgcagaagagcctctctctgtct |
| | | ccgggtaaatgataatctaga |
| 135 | huVK3LP- | aagcttgccaccatggaaaccccagcgcagcttctcttcctcctgctactctgg |
| | huTREX1- | ctcccagataccaccggtatgggccctggagctcgcagacagggcaggattgtg |
| | 72aa- | cagggaaggcctgagatgtgcttctgcccaccccctaccccactccctcccctt |
| | (g4s)5- | cggatcttaacactgggcactcacacacccaccccatgctcctctccaggctca |
| | hIgG1-WT | gcagcaggtacgtacccaaccatgggctcgcaggccctgcccccggggcccatg |
| | | cagaccctcatctttttcgacatggaggccactggcttgcccttctcccagccc |
| | | aaggtcacggagctgtgcctgctggctgtccacagatgtgccctggagagcccc |
| | | cccacctctcaggggccacctcccacagttcctccaccaccgcgtgtggtagac |
| | | aagctctccctgtgtgtggctccggggaaggcctgcagccctgcagccagcgag |
| | | atcacaggtctgagcacagctgtgctggcagcgcatgggcgtcaatgttttgat |
| | | gacaacctggccaacctgctcctagccttcctgcggcgccagccacagccctgg |
| | | tgcctggtggcacacaatggtgaccgctacgacttccccctgctccaagcagag |
| | | ctggctatgctgggcctcaccagtgctctggatggtgccttctgtgtggatagc |
| | | atcactgcgctgaaggccctggagcgagcaagcagcccctcagaacacggccca |
| | | aggaagagctacagcctaggcagcatctacactcgcctgtatgggcagtcccct |
| | | ccagactcgcacacggctgagggtgatgtcctggccctgctcagcatctgtcag |
| | | tggagaccacaggccctgctgcggtgggtggatgctcacgccaggcctttcggc |
| | | accatcaggcccatgtatggggtcacagcctctgctaggaccaaagatctctcc |
| | | ggaggaggtggctcaggtggtggaggatctggaggaggtggctcaggtggtgga |
| | | ggatctggaggaggtgggagtctcgagcccaaatcttctgacaaaactcacaca |
| | | tgtccaccgtgcccagcacctgaactcctggggggaccgtcagtcttcctcttc |
| | | cccccaaaacccaaggacaccctcatgatctcccggacccctgaggtcacatgc |
| | | gtggtggtggacgtgagccacgaagaccctgaggtcaagttcaactggtacgtg |
| | | gacggcgtggaggtgcataatgccaagacaaagccgcgggaggagcagtacaac |
| | | agcacgtaccgtgtggtcagcgtcctcaccgtcctgcaccaggactggctgaat |
| | | ggcaaggagtacaagtgcaaggtctccaacaaagccctcccagcccccatcgag |
| | | aaaaccatctccaaagccaaagggcagccccgagaaccacaggtgtacaccctg |
| | | cccccatcccgggatgagctgaccaagaaccaggtcagcctgacctgcctggtc |
| | | aaaggcttctatcccagcgacatcgccgtggagtgggagagcaatgggcagccg |
| | | gagaacaactacaagaccacgcctcccgtgctggactccgacggctccttcttc |
| | | ctctacagcaagctcaccgtggacaagagcaggtggcagcaggggaacgtcttc |
| | | tcatgctccgtgatgcatgaggctctgcacaaccactacacgcagaagagcctc |
| | | tctctgtctccgggtaaatgataatctaga |
| 136 | g4s4lnk | dlsggggsggggsggggsggggstgle |
| 137 | G4S5-1 | dlsggggsggggsggggsggggsggggstgle |
| 138 | G4S5-2 | dlsggggsggggsggggsggggsggggsle |
| 139 | hDNase1- | dilkiaafniqtfgetkmsnatlvsyivqilsrydialvqevrdshltavgkll |
| | 3′-G105R; | dnlnqdapdtyhyvvseplgrnsykerylfvyrpdqvsavdsyyyddgcepcrn |
| | A114F | dtfnrepfivrffsrftevrefaivplhaapgdavaeidalydvyldvqekwgl |
| | | edvmlmgdfnagcsyvrpsqwssirlwtsptfqwlipdsadttatpthcaydri |
| | | vvagmllrgavvpdsalpfnfqaayglsdqlaqaisdhypvevmlk* |
| 140 | hDNase1- | dilkiaafniqtfgetkmsnatlvsyivqilsrydialvqevrdshltavgkll |
| | 3′-WT | dnlnqdapdtyhyvvseplgrnsykerylfvyrpdqvsavdsyyyddgcepcgn |
| | | dtfnrepaivrffsrftevrefaivplhaapgdavaeidalydvyldvqekwgl |
| | | edvmimgdfnagcsyvrpsqwssirlwtsptfqwlipdsadttatpthcaydri |
| | | vvagmllrgavvpdsalpfnfqaayglsdqlaqaisdhypvevmlk* |
| 141 | hDNase1- | dilkiaafniqtfgetkmsnatlvsyivqilsrydialvqevrdshltavgkll |
| | 3′A114F | dnlnqdapdtyhyvvseplgrnsykerylfvyrpdqvsavdsyyyddgcepcgn |
| | | dtfnrepfivrffsrftevrefaivplhaapgdavaeidalydvyldvqekwgl |
| | | edvmlmgdfnagcsyvrpsqwssirlwtsptfqwlipdsadttatpthcaydri |
| | | vvagmllrgavvpdsalpfnfqaayglsdqlaqaisdhypvevmlk* |
| 142 | hDNase1- | tglkiaafniqtfgetkmsnatlvsyivqilsrydialvqevrdshltavgkll |
| | 5′-G105R; | dnlnqdapdtyhyvvseplgrnsykerylfvyrpdqvsavdsyyyddgcepcrn |
| | A114F | dtfnrepfivrffsrftevrefaivplhaapgdavaeidalydvyldvqekwgl |
| | | edvmlmgdfnagcsyvrpsqwssirlwtsptfqwlipdsadttatpthcaydri |
| | | vvagmllrgavvpdsalpfnfqaayglsdqlaqaisdhypvevmlkdle |
| 143 | hDNase1- | tglkiaafniqtfgetkmsnatlvsyivqilsrydialvqevrdshltavgkll |
| | 5′-WT | dnlnqdapdtyhyvvseplgrnsykerylfvyrpdqvsavdsyyyddgcepcgn |
| | | dtfnrepaivrffsrftevrefaivplhaapgdavaeidalydvyldvqekwgl |
| | | edvmlmgdfnagcsyvrpsqwssirlwtsptfqwlipdsadttatpthcaydri |
| | | vvagmllrgavvpdsalpfnfqaayglsdqlaqaisdhypvevmlkdle |
| 144 | hDNase1- | tglkiaafniqtfgetkmsnatlvsyivqilsrydialvqevrdshltavgkll |
| | 5′-A114F | dnlnqdapdtyhyvvseplgrnsykerylfvyrpdqvsavdsyyyddgcepcgn |
| | | dtfnrepfivrffsrftevrefaivplhaapgdavaeidalydvyldvqekwgl |
| | | edvmlmgdfnagcsyvrpsqwssirlwtsptfqwlipdsadttatpthcaydri |
| | | vvagmllrgavvpdsalpfnfqaayglsdqlaqaisdhypvevmlkdle |
| 145 | hIgG1WT | dlepkssdkthtcppcpapellggpsvflfppkpkdtlmisrtpevtcvvvdvs |
| | | hedpevkfnwyvdgvevhnaktkpreeqynstyrvvsvltvlhqdwlngkeykc |
| | | kvsnkalpapiektiskakgqprepqvytlppsrdeltknqvsltclvkgfyps |
| | | diavewesngqpennykttppvldsdgsfflyskitvdksrwqqgnvfscsvmh |
| | | ealhnhytqkslslspgk |
| 146 | hRNase- | vdgasspvnvsspsvqdipslgkesrakkfqrqhmdsdsspsssstycnqmmrr |
| | G88D-3′ | rnmtqgrckpvntfvheplvdvqnvcfqekvtckngqgncyksnssmhitdcrl |
| | | tndsrypncayrtspkerhiivacegspyvpvhfdasvedst* |
| 147 | human | metpaqllfllllwlpdttglkiaafniqtfgetkmsnatlvsyivqilsrydi |
| | DNase1 + | alvqevrdshltavgklldnlnqdapdtyhyvvseplgrnsykerylfvyrpdq |
| | VK3LP | vsavdsyyyddgcepcgndtfnrepaivrffsrftevrefaivplhaapgdava |
| | | eidalydvyldvqekwgledvmlmgdfnagcsyvrpsqwssirlwtsptfqwli |
| | | pdsadttatpthcaydrivvagmllrgavvpdsalpfnfqaayglsdqlaqais |
| | | dhypvevmlk* |
| 148 | DNase1L3 | msrelapllllllsihsalamricsfnvrsfgeskqedknamdvivkvikrcdi |
| | | ilvmeikdsnnricpilmeklnrnsrrgitynyvissrlgrntykeqyaflyke |
| | | klvsvkrsyhyhdyqdgdadvfsrepfvvwfqsphtavkdfviiplhttpetsv |
| | | keidelvevytdvkhrwkaenfifmgdfnagcsyvpkkawknirlrtdprfvwl |
| | | igdqedttvkkstncaydrivlrgqeivssvvpksnsvfdfqkayklteeeald |
| | | vsdhfpvefklqssraftnskksvtlrkktkskrs* |
| 149 | human | Mglekslvrllllvlillvlgwvqpslgkesrakkfqrqhmdsdsspsssstyc |
| | pancreatic | nqmmrrrnmtqgrckpvntfvheplvdvqnvcfqekvtckngqgncyksnssmh |
| | ribonuclease | itdcrltngsrypncayrtspkerhiivacegspyvpvhfdatv* |
| 150 | huVK3LP + | metpaqllfllllwlpdttgresaaqkfqrqhmdpdgssinsptycnqmmkrrd |
| | mrib1 + | mtngsckpvntfvhepladvqavcsqenvtcknrksncyksssalhitdchlkg |
| | mIgG2A- | nskypncdykttqyqkhiivacegnpyvpvhfdatvleprgltikpsppckcpa |
| | C + 2S | pnllggssvfifppkikdvlmislspmvtcvvvdvseddpdvqiswfvnnvevh |
| | | taqtqthredynstlrvvsalpiqhqdwmsgkefkcsvnnkdlpasiertiskp |
| | | rgpvrapqvyvlpppaeemtkkefsltcmitgflpaeiavdwtsngrteqnykn |
| | | tatvldsdgsyfmysklrvqkstwergslfacsvvheglhnhlttksfsrtp |
| | | gk* |
| 151 | huVK3LP- | metpaqllfllllwlpdttgkesrakkfqrqhmdsdsspsssstycnqmmrrrn |
| | hRNaseWT- | mtqgrckpvntfvheplvdvqnvcfqekvtckngqgncyksnssmhitdcrltn |
| | hIgGW-NLG- | gsrypncayrtspkerhiivacegspyvpvhfdasvedstdlepkssdkthtcp |
| | hDNase1- | pcpapellggpsvflfppkpkdtlmisrtpevtcvvvdvshedpevkfnwyvdg |
| | 105-114 | vevhnaktkpreeqynstyrvvsvltvlhqdwlngkeykckvsnkalpapiekt |
| | | iskakgqprepqvytlppsrdeltknqvsltclvkgfypsdiavewesngqpen |
| | | nykttppvldsdgsfflyskitvdksrwqqgnvfscsvmheglhnhytqkslsl |
| | | spgkvdgasshvnvsspsvqdilkiaafniqtfgetkmsnatlvsyivqilsry |
| | | dialvqevrdshltavgklldnlnqdapdtyhyvvseplgrnsykerylfvyrp |
| | | dqvsavdsyyyddgcepcgndtfnrepaivrffsrftevrefaivplhaapgda |
| | | vaeidalydvyldvqekwgsedvmlmgdfnagcsyvrpsqwssirlwtsptfqw |
| | | lipdsadttatpthcaydrivvagmllrgavvpdsalpfnfqxayglsdqlaqa |
| | | isdhypvevmlk** |
| 152 | huVK3LP- | metpaqllfllllwlpdttgkesrakkfqrqhmdsdsspsssstycnqmmrrrn |
| | hRNaseWT- | mtqgrckpvntfvheplvdvqnvcfqekvtckngqgncyksnssmhitdcrltn |
| | hIgGWT-NLG- | gsrypncayrtspkerhiivacegspyvpvhfdasvedstdlepkssdkthtcp |
| | hDNase1- | pcpapellggpsvflfppkpkdtlmisrtpevtcvvvdvshedpevkfnwyvdg |
| | 114F | vevhnaktkpreeqynstyrvvsvltvlhqdwlngkeykckvsnkalpapiekt |
| | | iskakgqprepqvytlppsrdeltknqvsltclvkgfypsdiavewesngqpen |
| | | nykttppvldsdgsfflyskitvdksrwqqgnvfscsvmheglhnhytqkslsl |
| | | spgkvdgasshvnvsspsvqdilkiaafniqtfgetkmsnatlvsyivqilsry |
| | | dialvqevrdshltavgklldnlnqdapdtyhyvvseplgrnsykerylfvyrp |
| | | dqvsavdsyyyddgcepcgndtfnrepfivrffsrftevrefaivplhaapgda |
| | | vaeidalydvyldvqekwgledvmlmgdfnagcsyvrpsqwssirlwtsptfqw |
| | | lipdsadttatpthcaydrivvagmllrgavvpdsalpfnfqaayglsdqlaqa |
| | | isdhypvevmlk* |
| 153 | huVK3LP- | metpaqllfllllwlpdttgkesrakkfqrqhmdsdsspsssstycnqmmrrrn |
| | hRNaseWT- | mtqgrckpvntfvheplvdvqnvcfqekvtckngqgncyksnssmhitdcrltn |
| | hIgGWT-NLG- | gsrypncayrtspkerhiivacegspyvpvhfdasvedstdlepkssdkthtcp |
| | hDNAse1-WT | pcpapellggpsvflfppkpkdtlmisrtpevtcvvvdvshedpevkfnwyvdg |
| | | vevhnaktkpreeqynstyrvvsvltvlhqdwlngkeykckvsnkalpapiekt |
| | | iskakgqprepqvytlppsrdeltknqvsltclvkgfypsdiavewesngqpen |
| | | nykttppvldsdgsfflyskltvdksrwqqgnvfscsvmheglhnhytqkslsl |
| | | spgkvdgasshvnvsspsvqdilkiaafniqtfgetkmsnatlvsyivqilsry |
| | | dialvqevrdshltavgklldnlnqdapdtyhyvvseplgrnsykerylfvyrp |
| | | dqvsavdsyyyddgcepcgndtfnrepaivrffsrftevrefaivplhaapgda |
| | | vaeidalydvyldvqekwgledvmlmgdfnagcsyvrpsqwssirlwtsptfqw |
| | | lipdsadttatpthcaydrivvagmllrgavvpdsalpfnfqaayglsdqlaqa |
| | | isdhypvevmlk* |
| 154 | hVK3LP- | metpaqllfllllwlpdttglkiaafniqtfgetkmsnatlvsyivqilsrydi |
| | hDNase1(WT)- | alvqevrdshltavgklldnlnqdapdtyhyvvseplgrnsykerylfvyrpdq |
| | hIgG1WT | vsavdsyyyddgcepcgndtfnrepaivrffsrftevrefaivplhaapgdava |
| | | eidalydvyldvqekwgledvmlmgdfnagcsyvrpsqwssirlwtsptfqwli |
| | | pdsadttatpthcaydrivvagmllrgavvpdsalpfnfqaayglsdqlaqais |
| | | dhypvevmlkdlepkssdkthtcppcpapellggpsvflfppkpkdtlmisrtp |
| | | evtcvvvdvshedpevkfnwyvdgvevhnaktkpreeqynstyrvvsvltvlhq |
| | | dwlngkeykckvsnkalpapiektiskakgqprepqvytlppsrdeltknqvsl |
| | | tclvkgfypsdiavewesngqpennykttppvldsdgsfflyskitvdksrwqq |
| | | gnvfscsvmhealhnhytqkslslspgk* |
| 155 | hVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | A114F- | vsavdsyyyddgcepcgndtfnrepfivrffsrftevrefaivplhaapgdava |
| | hIgG1WT | eidalydvyldvqekwgledvmlmgdfnagcsyvrpsqwssirlwtsptfqwli |
| | | pdsadttatpthcaydrivvagmllrgavvpdsalpfnfqaayglsdqlaqais |
| | | dhypvevmlkdlepkssdkthtcppcpapellggpsvflfppkpkdtlmisrtp |
| | | evtcvvvdvshedpevkfnwyvdgvevhnaktkpreeqynstyrvvsvltvlhq |
| | | dwlngkeykckvsnkalpapiektiskakgqprepqvytlppsrdeltknqvsl |
| | | tclvkgfypsdiavewesngqpennykttppvldsdgsfflyskitvdksrwqq |
| | | gnvfscsvmhealhnhytqkslslspgk* |
| 156 | hVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | (G4S)4- | pdsadttatpthcaydrivvagmllrgavvpdsalpfnfqaayglsdqlaqais |
| | hIgG1 WT | dhypvevmlkdlsggggsggggsggggsggggstglepkssdkthtcppcpape |
| | | llggpsvflfppkpkdtlmisrtpevtcvvvdvshedpevkfnwyvdgvevhna |
| | | ktkpreeqynstyrvvsvltvlhqdwlngkeykckvsnkalpapiektiskakg |
| | | qprepqvytlppsrdeltknqvsltclvkgfypsdiavewesngqpennykttp |
| | | pvidsdgsfflyskltvdksrwqqgnvfscsvmhealhnhytqkslslspgk* |
| 157 | hVK3LP- | metpaqllfllllwlpdttglkiaafniqtfgetkmsnatlvsyivqilsrydi |
| | hDNase1G105R; | alvqevrdshltavgklldnlnqdapdtyhyvvseplgrnsykerylfvyrpdq |
| | A114F | vsavdsyyyddgcepcrndtfnrepfivrffsrftevrefaivplhaapgdava |
| | (G4s)5- | eidalydvyldvqekwgledvmlmgdfnagcsyvrpsqwssirlwtsptfqwli |
| | hIgG1 WT | pdsadttatpthcaydrivvagmllrgavvpdsalpfnfqaayglsdqlaqais |
| | | dhypvevmlkdlsggggsggggsggggsggggsggggstglepkssdkthtcpp |
| | | cpapellggpsvflfppkpkdtlmisrtpevtcvvvdvshedpevkfnwyvdgv |
| | | evhnaktkpreeqynstyrvvsvltvlhqdwlngkeykckvsnkalpapiekti |
| | | skakgqprepqvytippsrdeltknqvsltcivkgfypsdiavewesngqpenn |
| | | ykttppvldsdgsfflyskltvdksrwqqgnvfscsvmhealhnhytqkslsls |
| | | pgk* |
| 158 | hVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | (G4S)5-2- | pdsadttatpthcaydrivvagmllrgavvpdsalpfnfqaayglsdqlaqais |
| | hIgG1 WT | dhypvevmlkdlsggggsggggsggggsggggsggggslepkssdkthtcppcp |
| | | apellggpsvflfppkpkdtlmisrtpevtcvvvdvshedpevkfnwyvdgvev |
| | | hnaktkpreeqynstyrvvsvltvlhqdwlngkeykckvsnkalpapiektisk |
| | | akgqprepqvytlppsrdeltknqvsltclvkgfypsdiavewesngqpennyk |
| | | ttppvldsdgsfflyskltvdksrwqqgnvfscsvmhealhnhytqkslslspg |
| | | k* |
| 159 | hVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | hIgG1 WT | pdsadttatpthcaydrivvagmllrgavvpdsalpfnfqaayglsdqlaqais |
| | | dhypvevmlkdlepkssdkthtcppcpapellggpsvflfppkpkdtlmisrtp |
| | | evtcvvvdvshedpevkfnwyvdgvevhnaktkpreeqynstyrvvsvltvlhq |
| | | dwlngkeykckvsnkalpapiektiskakgqprepqvytlppsrdeltknqvsl |
| | | tcivkgfypsdiavewesngqpennykttppvldsdgsfflyskitvdksrwqq |
| | | gnvfscsvmhealhnhytqkslslspgk* |
| 160 | hVK3LP- | metpaqllfllllwlpdttgkesrakkfqrqhmdsdsspsssstycnqmmrrrn |
| | hRNase1(MT)- | mtqgrckpvntfvheplvdvqnvcfqekvtckngqgncyksnssmhitdcrltn |
| | hIgG1WT | dsrypncayrtspkerhiivacegspyvpvhfdasvedstdlepkssdkthtcp |
| | | pcpapellggpsvflfppkpkdtlmisrtpevtcvvvdvshedpevkfnwyvdg |
| | | vevhnaktkpreeqynstyrvvsvltvlhqdwlngkeykckvsnkalpapiekt |
| | | iskakgqprepqvytlppsrdeltknqvsltcivkgfypsdiavewesngqpen |
| | | nykttppvldsdgsfflyskltvdksrwqqgnvfscsvmhealhnhytqkslsl |
| | | spgk* |
| 161 | hVK3Lp- | metpaqllfllllwlpdttgkesrakkfqrqhmdsdsspsssstycnqmmrrrn |
| | hRNase1(WT)- | mtqgrckpvntfvheplvdvqnvcfqekvtckngqgncyksnssmhitdcrltn |
| | (G4S)4lnk- | gsrypncayrtspkerhiivacegspyvpvhfdasvedstdlsggggsggggsg |
| | hIgG1WT | gggsggggstglepkssdkthtcppcpapellggpsvflfppkpkdtlmisrtp |
| | | evtcvvvdvshedpevkfnwyvdgvevhnaktkpreeqynstyrvvsvltvlhq |
| | | dwlngkeykckvsnkalpapiektiskakgqprepqvytippsrdeltknqvsl |
| | | tcivkgfypsdiavewesngqpennykttppvldsdgsfflyskitvdksrwqq |
| | | gnvfscsvmhealhnhytqkslslspgk* |
| 162 | hVK3LP- | metpaqllfllllwlpdttgkesrakkfqrqhmdsdsspsssstycnqmmrrrn |
| | hRNase(WT)- | mtqgrckpvntfvheplvdvqnvcfqekvtckngqgncyksnssmhitdcrltn |
| | (G4S)5- | gsrypncayrtspkerhiivacegspyvpvhfdasvedstdlsggggsggggsg |
| | 2lnk- | gggsggggsggggslepkssdkthtcppcpapellggpsvflfppkpkdtlmis |
| | hIgG1WT | rtpevtcvvvdvshedpevkfnwyvdgvevhnaktkpreeqynstyrvvsvltv |
| | | ihqdwlngkeykckvsnkalpapiektiskakgqprepqvytlppsrdeltknq |
| | | vsltclvkgfypsdiavewesngqpennykttppvldsdgsfflyskitvdksr |
| | | wqqgnvfscsvmhealhnhytqkslslspgk* |
| 163 | hVK3LP- | metpaqllfllllwlpdttgkesrakkfqrqhmdsdsspsssstycnqmmrrrn |
| | hRNase(WT)- | mtqgrckpvntfvheplvdvqnvcfqekvtckngqgncyksnssmhitdcrltn |
| | hIgG1 WT | gsrypncayrtspkerhiivacegspyvpvhfdasvedstdlepkssdkthtcp |
| | | pcpapellggpsvflfppkpkdtlmisrtpevtcvvvdvshedpevkfnwyvdg |
| | | vevhnaktkpreeqynstyrvvsvltvlhqdwlngkeykckvsnkalpapiekt |
| | | iskakgqprepqvytlppsrdeltknqvsltcivkgfypsdiavewesngqpen |
| | | nykttppvldsdgsfflyskltvdksrwqqgnvfscsvmhealhnhytqkslsl |
| | | spgk* |
| 164 | murine | mgsqtlphghmqtlifidleatglpssrpevtelcllavhrralentsisqghp |
| | Trex1(FL)- | ppvprpprvvdklslciapgkacspgaseitglskaelevqgrqrfddnlaill |
| | transcript | rafiqrqpqpcclvahngdrydfpllqtelarlstpspldgtfcvdsiaalkal |
| | variant 1 | eqasspsgngsrksyslgsiytrlywqaptdshtaegdvltllsicqwkpqall |
| | | qwvdeharpfstvkpmygtpattgttnlrphaatattplatangspsngrsrrp |
| | | kspppekvpeapsqegllaplslltlltlaiatlyglflaspgq* |
| 165 | mouse | mgsqtlphghmqtlifidleatglpssrpevtelcllavhrralentsisqghp |
| | Trex1minec | ppvprpprvvdklslciapgkacspgaseitglskaelevqgrqrfddnlaill |
| | | raflqrqpqpcclvahngdrydfpliqtelaristpspldgtfcvdsiaalkal |
| | | eqasspsgngsrksyslgsiytrlywqaptdshtaegdvltllsicqwkpqall |
| | | qwvdeharpfstvkpmygtpattgttdle |
| 166 | murine | metpaqllfllllwlpdttgmgsqtlphghmqtlifidleatglpssrpevtel |
| | Trex1- | cllavhrralentsisqghpppvprpprvvdklslciapgkacspgaseitgls |
| | (G4S)4- | kaelevqgrqrfddnlaillraflqrqpqpcclvahngdrydfpllqtelaris |
| | mIgG2a-c | tpspldgtfcvdsiaalkaleqasspsgngsrksyslgsiytrlywqaptdsht |
| | | aegdvltllsicqwkpqallqwvdeharpfstvkpmygtpattgttdlsggggs |
| | | ggggsggggsggggsleprgptikpsppckcpapnllggssvfifppkikdvlm |
| | | islspmvtcvvvdvseddpdvqiswfvnnvevhtaqtqthredynstlrvvsal |
| | | piqhqdwmsgkefkcsvnnkdlpasiertiskprgpvrapqvyvlpppaeemtk |
| | | kefsltcmitgflpaeiavdwtsngrteqnykntatvldsdgsyfmysklrvqk |
| | | stwergsifacsvvheglhnhlttksfsrtpgk* |
| 167 | murine | metpaqllfllllwlpdttgmgsqtlphghmqtlifldleatglpssrpevtel |
| | Trex1- | cllavhrralentsisqghpppvprpprvvdklslciapgkacspgaseitgls |
| | (G4S)5- | kaelevqgrqrfddnlaillraflqrqpqpcclvahngdrydfpllqtelaris |
| | mIgG2a-c | tpspldgtfcvdsiaalkaleqasspsgngsrksyslgsiytrlywqaptdsht |
| | | aegdvltllsicqwkpqallqwvdeharpfstvkpmygtpattgttdlsggggs |
| | | ggggsggggsggggsggggsleprgptikpsppckcpapnllggssvfifppki |
| | | kdvlmislspmvtcvvvdvseddpdvqiswfvnnvevhtaqtqthredynstlr |
| | | vvsalpiqhqdwmsgkefkcsvnnkdlpasiertiskprgpvrapqvyvlpppa |
| | | eemtkkefsltcmitgflpaeiavdwtsngrteqnykntatvldsdgsyfmysk |
| | | irvqkstwergslfacsvvheglhnhlttksfsrtpgk* |
| 168 | NLGlnk | vdgaaaspvnvsspsvqdi |
| 169 | Murine | metpaqllfllllwlpdttgmgsqtlphghmqtlifldleatglpssrpevtel |
| | Trex- | cllavhrralentsisqghpppvprpprvvdklslciapgkacspgaseitgls |
| | Trex1- | kaelevqgrqrfddnlaillraflqrqpqpcclvahngdrydfpllqtelaris |
| | (G4s)5- | tpspldgtfcvdsiaalkaleqasspsgngsrksyslgsiytrlywqaptdsht |
| | mIgG2a-c | aegdvltllsicqwkpqallqwvdeharpfstvkpmygtpattgttdlmgsqtl |
| | | phghmqtlifidleatglpssrpevtelcllavhrralentsisqghpppvprp |
| | | prvvdklslciapgkacspgaseitglskaelevqgrqrfddnlaillrafiqr |
| | | qpqpcclvahngdrydfpllqtelaristpspldgtfcvdsiaalkaleqassp |
| | | sgngsrksyslgsiytrlywqaptdshtaegdvltllsicqwkpqallqwvdeh |
| | | arpfstvkpmygtpattgttdlsggggsggggsggggsggggsggggsleprgp |
| | | tikpsppckcpapnllggssvfifppkikdvimisispmvtcvvvdvseddpdv |
| | | qiswfvnnvevhtaqtqthredynstlrvvsalpiqhqdwmsgkefkcsvnnkd |
| | | lpasiertiskprgpvrapqvyvlpppaeemtkkefsltcmitgflpaeiavdw |
| | | tsngrteqnykntatvldsdgsyfmysklrvqkstwergslfacsvvheglhnh |
| | | ittksfsrtpgk* |
| 170 | huVK3LP- | metpaqllfllllwlpdttgmgpgarrqgrivqgrpemcfcppptplpplrilt |
| | huTREX1- | lgthtptpcsspgsaagtyptmgsqalppgpmqtliffdmeatglpfsqpkvte |
| | 72aa- | lcllavhrcalespptsqgppptvpppprvvdklslcvapgkacspaaseitgl |
| | (g4s)4- | stavlaahgrqcfddnlanlllaflrrqpqpwclvahngdrydfpllqaelaml |
| | hIgG1 WT | gltsaldgafcvdsitalkalerasspsehgprksyslgsiytrlygqsppdsh |
| | | taegdvlallsicqwrpqallrwvdaharpfgtirpmygvtasartkdlsgggg |
| | | sggggsggggsggggslepkssdkthtcppcpapellggpsvflfppkpkdtlm |
| | | isrtpevtcvvvdvshedpevkfnwyvdgvevhnaktkpreeqynstyrvvsvl |
| | | tvlhqdwlngkeykckvsnkalpapiektiskakgqprepqvytlppsrdeltk |
| | | nqvsltclvkgfypsdiavewesngqpennykttppvldsdgsfflyskitvdk |
| | | srwqqgnvfscsvmhealhnhytqkslslspgk* |
| 171 | huVK3LP- | metpaqllfllllwlpdttgmgpgarrqgrivqgrpemcfcppptplpplrilt |
| | huTREX1- | lgthtptpcsspgsaagtyptmgsqalppgpmqtliffdmeatglpfsqpkvte |
| | 72aa- | lcllavhrcalespptsqgppptvpppprvvdklslcvapgkacspaaseitgl |
| | (g4s)5- | stavlaahgrqcfddnlanlllafirrqpqpwclvahngdrydfpllqaelaml |
| | hIgG1 WT | gltsaldgafcvdsitalkalerasspsehgprksyslgsiytrlygqsppdsh |
| | | taegdvlallsicqwrpqallrwvdaharpfgtirpmygvtasartkdlsgggg |
| | | sggggsggggsggggsggggslepkssdkthtcppcpapellggpsvflfppkp |
| | | kdtlmisrtpevtcvvvdvshedpevkfnwyvdgvevhnaktkpreeqynstyr |
| | | vvsvltvlhqdwlngkeykckvsnkalpapiektiskakgqprepqvytlppsr |
| | | deltknqvsltclvkgfypsdiavewesngqpennykttppvldsdgsfflysk |
| | | itvdksrwqqgnvfscsvmhealhnhytqkslslspgk* |
| 172 | huVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)4- | gtggggaagctgctggacaacctcaatcaggatgcaccagacacctatcactac |
| | hIgG1- | gtggtcagtgagccactgggacggaacagctataaggagcgctacctgttcgtg |
| | WT-NLG- | tacaggcctgaccaggtgtctgcggtggacagctactactacgatgatggctgc |
| | hRNase1-WT | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtgggagtggtggaggt |
| | | ggttctaccggtctcgagcccaaatcttctgacaaaactcacacatgtccaccg |
| | | tgcccagcacctgaactcctggggggaccgtcagtcttcctcttccccccaaaa |
| | | cccaaggacaccctcatgatctcccggacccctgaggtcacatgcgtggtggtg |
| | | gacgtgagccacgaagaccctgaggtcaagttcaactggtacgtggacggcgtg |
| | | gaggtgcataatgccaagacaaagccgcgggaggagcagtacaacagcacgtac |
| | | cgtgtggtcagcgtcctcaccgtcctgcaccaggactggctgaatggcaaggag |
| | | tacaagtgcaaggtctccaacaaagccctcccagcccccatcgagaaaaccatc |
| | | tccaaagccaaagggcagccccgagaaccacaggtgtacaccctgcccccatcc |
| | | cgggatgagctgaccaagaaccaggtcagcctgacctgcctggtcaaaggcttc |
| | | tatcccagcgacatcgccgtggagtgggagagcaatgggcagccggagaacaac |
| | | tacaagaccacgcctcccgtgctggactccgacggctccttcttcctctacagc |
| | | aagctcaccgtggacaagagcaggtggcagcaggggaacgtcttctcatgctcc |
| | | gtgatgcatgaggctctgcacaaccactacacgcagaagagcctctctctgtct |
| | | ccgggtaaagtcgacggagctagcagccccgtgaacgtgagcagccccagcgtg |
| | | caggatatcccttccctgggcaaggaatcccgggccaagaaattccagcggcag |
| | | catatggactcagacagttcccccagcagcagctccacctactgtaaccaaatg |
| | | atgaggcgccggaatatgacacaggggcggtgcaaaccagtgaacacctttgtg |
| | | cacgagcccctggtagatgtccagaatgtctgtttccaggaaaaggtcacctgc |
| | | aagaacgggcagggcaactgctacaagagcaactccagcatgcacatcacagac |
| | | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacctaataatctaga |
| 173 | huVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | (G4S)4- | pdsadttatpthcaydrivvagmllrgavvpdsalpfnfqaayglsdqlaqais |
| | hIgG1- | dhypvevmlkdlsggggsggggsggggsggggstglepkssdkthtcppcpape |
| | WT-NLG- | llggpsvflfppkpkdtlmisrtpevtcvvvdvshedpevkfnwyvdgvevhna |
| | hRNase1-WT | ktkpreeqynstyrvvsvltvlhqdwlngkeykckvsnkalpapiektiskakg |
| | | qprepqvytlppsrdeltknqvsltclvkgfypsdiavewesngqpennykttp |
| | | pvldsdgsfflyskltvdksrwqqgnvfscsvmhealhnhytqkslslspgkvd |
| | | gasspvnvsspsvqdipslgkesrakkfqrqhmdsdsspsssstycnqmmrrrn |
| | | mtqgrckpvntfvheplvdvqnvcfqekvtckngqgncyksnssmhitdcrltn |
| | | gsrypncayrtspkerhiivacegspyvpvhfdasvedst* |
| 174 | huVK3LP- | gttaagcttgccaccatggaaaccccagcgcagcttctcttcctcctgctactc |
| | hRNAseG88D- | tggctcccagataccaccggtccttccctgggcaaggaatcccgggccaagaaa |
| | hIgG1- | ttccagcggcagcatatggactcagacagttcccccagcagcagctccacctac |
| | P238S; | tgtaaccaaatgatgaggcgccggaatatgacacaggggcggtgcaaaccagtg |
| | K322S; | aacacctttgtgcacgagcccctggtagatgtccagaatgtctgtttccaggaa |
| | P331S | aaggtcacctgcaagaacgggcagggcaactgctacaagagcaactccagcatg |
| | | cacatcacagactgccgcctgacaaacgactccaggtaccccaactgtgcatac |
| | | cggaccagcccgaaggagagacacatcattgtggcctgtgaagggagcccatat |
| | | gtgccagtccactttgatgcttctgtggaggactctacagatctcgagcccaaa |
| | | tcttctgacaaaactcacacatgtccaccgtgtccagcacctgaactcctgggt |
| | | ggatcgtcagtcttcctcttccccccaaaacccaaggacactctcatgatctcc |
| | | cggacccctgaggtcacgtgcgtggtggtggacgtgagccaggaagaccccgag |
| | | gtccagttcaactggtacgtggacggcatggaggtgcataatgccaagacaaag |
| | | ccacgggaggagcagttcaacagcacgttccgtgtggtcagcgtcctcaccgtc |
| | | gtgcaccaggactggctgaacggcaaggagtacaagtgcaaggtctccaacaaa |
| | | gccctcccagcctccatcgagaaaacaatctccaaaaccaaagggcagccccga |
| | | gaaccacaggtgtacaccctgcccccatcccgggaggagatgaccaagaaccag |
| | | gtcagcctgacctgcctggtcaaaggcttctatcccagcgacatcgccgtggag |
| | | tgggagagcaatgggcagccggagaacaactacaacaccacgcctcccgtgctg |
| | | gactccgacggctccttctccctctacagcaagctcaccgtggacaagagcagg |
| | | tggcagcaggggaacgtcttctcatgctccgtgatgcatgaggctctgcacaac |
| | | cactacacgcagaagagcctctctctgtctccgggtaaatgataatctaga |
| 175 | huVK3LP- | metpaqllfllllwlpdttgpslgkesrakkfqrqhmdsdsspsssstycnqmm |
| | hRNAseG88D- | rrrnmtqgrckpvntfvheplvdvqnvcfqekvtckngqgncyksnssmhitdc |
| | hIgG1- | rltndsrypncayrtspkerhiivacegspyvpvhfdasvedstdlepkssdkt |
| | P238S; | htcppcpapellggssvfifppkpkdtlmisrtpevtcvvvdvsqedpevqfnw |
| | K322S; | yvdgmevhnaktkpreeqfnstfrvvsvltvvhqdwlngkeykckvsnkalpas |
| | P331S | iektisktkgqprepqvytlppsreemtknqvsltclvkgfypsdiavewesng |
| | | qpennynttppvldsdgsfslyskltvdksrwqqgnvfscsvmhealhnhytqk |
| | | slslspgk* |
| 176 | huVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)5- | gtggggaagctgctggacaacctcaatcaggatgcaccagacacctatcactac |
| | 1-hIgG1- | gtggtcagtgagccactgggacggaacagctataaggagcgctacctgttcgtg |
| | WT-NLG- | tacaggcctgaccaggtgtctgcggtggacagctactactacgatgatggctgc |
| | hRNase1-WT | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtggctcaggtggtgga |
| | | ggatctggaggaggtgggagtaccggtctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgaggctctgcacaaccactacacgcagaag |
| | | agcctctctctgtctccgggtaaagtcgacggagctagcagccccgtgaacgtg |
| | | agcagccccagcgtgcaggatatcccttccctgggcaaggaatcccgggccaag |
| | | aaattccagcggcagcatatggactcagacagttcccccagcagcagctccacc |
| | | tactgtaaccaaatgatgaggcgccggaatatgacacaggggcggtgcaaacca |
| | | gtgaacacctttgtgcacgagcccctggtagatgtccagaatgtctgtttccag |
| | | gaaaaggtcacctgcaagaacgggcagggcaactgctacaagagcaactccagc |
| | | atgcacatcacagactgccgcctgacaaacggctccaggtaccccaactgtgca |
| | | taccggaccagcccgaaggagagacacatcattgtggcctgtgaagggagccca |
| | | tatgtgccagtccactttgatgcttctgtggaggactctacctaataatctaga |
| 177 | huVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eldalydvyldvqekwgledvmlmgdfnagcsyvrpsqwssirlwtsptfqwli |
| | (G4S)5- | pdsadttatpthcaydrivvagmllrgavvpdsalpfnfqaayglsdqlaqais |
| | 1-hIgG1- | dhypvevmlkdlsggggsggggsggggsggggsggggstglepkssdkthtcpp |
| | WT-NLG- | cpapellggpsvflfppkpkdtlmisrtpevtcvvvdvshedpevkfnwyvdgv |
| | hRNase1-WT | evhnaktkpreeqynstyrvvsvltvlhqdwlngkeykckvsnkalpapiekti |
| | | skakgqprepqvytlppsrdeltknqvsltclvkgfypsdiavewesngqpenn |
| | | ykttppvldsdgsfflyskltvdksrwqqgnvfscsvmhealhnhytqkslsls |
| | | pgkvdgasspvnvsspsvqdipslgkesrakkfqrqhmdsdsspsssstycnqm |
| | | mrrrnmtqgrckpvntfvheplvdvqnvcfqekvtckngqgncyksnssmhitd |
| | | crltngsrypncayrtspkerhiivacegspyvpvhfdasvedst* |
| 178 | huVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)5- | gtggggaagctgctggacaacctcaatcaggatgcaccagacacctatcactac |
| | 2-hIgG1- | gtggtcagtgagccactgggacggaacagctataaggagcgctacctgttcgtg |
| | WT-NLG- | tacaggcctgaccaggtgtctgcggtggacagctactactacgatgatggctgc |
| | hRNase1-WT | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtggctcaggtggtgga |
| | | ggatctggaggaggtgggagtctcgagcccaaatcttctgacaaaactcacaca |
| | | tgtccaccgtgcccagcacctgaactcctggggggaccgtcagtcttcctcttc |
| | | cccccaaaacccaaggacaccctcatgatctcccggacccctgaggtcacatgc |
| | | gtggtggtggacgtgagccacgaagaccctgaggtcaagttcaactggtacgtg |
| | | gacggcgtggaggtgcataatgccaagacaaagccgcgggaggagcagtacaac |
| | | agcacgtaccgtgtggtcagcgtcctcaccgtcctgcaccaggactggctgaat |
| | | ggcaaggagtacaagtgcaaggtctccaacaaagccctcccagcccccatcgag |
| | | aaaaccatctccaaagccaaagggcagccccgagaaccacaggtgtacaccctg |
| | | cccccatcccgggatgagctgaccaagaaccaggtcagcctgacctgcctggtc |
| | | aaaggcttctatcccagcgacatcgccgtggagtgggagagcaatgggcagccg |
| | | gagaacaactacaagaccacgcctcccgtgctggactccgacggctccttcttc |
| | | ctctacagcaagctcaccgtggacaagagcaggtggcagcaggggaacgtcttc |
| | | tcatgctccgtgatgcatgaggctctgcacaaccactacacgcagaagagcctc |
| | | tctctgtctccgggtaaagtcgacggagctagcagccccgtgaacgtgagcagc |
| | | cccagcgtgcaggatatcccttccctgggcaaggaatcccgggccaagaaattc |
| | | cagcggcagcatatggactcagacagttcccccagcagcagctccacctactgt |
| | | aaccaaatgatgaggcgccggaatatgacacaggggcggtgcaaaccagtgaac |
| | | acctttgtgcacgagcccctggtagatgtccagaatgtctgtttccaggaaaag |
| | | gtcacctgcaagaacgggcagggcaactgctacaagagcaactccagcatgcac |
| | | atcacagactgccgcctgacaaacggctccaggtaccccaactgtgcataccgg |
| | | accagcccgaaggagagacacatcattgtggcctgtgaagggagcccatatgtg |
| | | ccagtccactttgatgcttctgtggaggactctacctaataatctaga |
| 179 | huVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | (G4S)5- | pdsadttatpthcaydrivvagmllrgavvpdsalpfnfqaayglsdqlaqais |
| | 2-hIgG1- | dhypvevmlkdlsggggsggggsggggsggggsggggslepkssdkthtcppcp |
| | WT-NLG- | apellggpsvflfppkpkdtlmisrtpevtcvvvdvshedpevkfnwyvdgvev |
| | hRNase1-WT | hnaktkpreeqynstyrvvsvltvlhqdwlngkeykckvsnkalpapiektisk |
| | | akgqprepqvytlppsrdeltknqvsltclvkgfypsdiavewesngqpennyk |
| | | ttppvldsdgsfflyskltvdksrwqqgnvfscsvmhealhnhytqkslslspg |
| | | kvdgasspvnvsspsvqdipslgkesrakkfqrqhmdsdsspsssstycnqmmr |
| | | rrnmtqgrckpvntfvheplvdvqnvcfqekvtckngqgncyksnssmhitdcr |
| | | ltngsrypncayrtspkerhiivacegspyvpvhfdasvedst |
| 180 | huVK3LP- | gttaagcttgccaccatggaaaccccagcgcagcttctcttcctcctgctactc |
| | hDNase1 | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | hIgG1- | gtggggaagctgctggacaacctcaatcaggatgcaccagacacctatcactac |
| | WT-NLG- | gtggtcagtgagccactgggacggaacagctataaggagcgctacctgttcgtg |
| | hRNasse1-WT | tacaggcctgaccaggtgtctgcggtggacagctactactacgatgatggctgc |
| | | gagccctgcaggaacgacaccttcaaccgagagccattcattgtcaggttcttc |
| | | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaagtcgacgga |
| | | gctagcagccccgtgaacgtgagcagccccagcgtgcaggatatcccttccctg |
| | | ggcaaggaatcccgggccaagaaattccagcggcagcatatggactcagacagt |
| | | tcccccagcagcagctccacctactgtaaccaaatgatgaggcgccggaatatg |
| | | acacaggggcggtgcaaaccagtgaacacctttgtgcacgagcccctggtagat |
| | | gtccagaatgtctgtttccaggaaaaggtcacctgcaagaacgggcagggcaac |
| | | tgctacaagagcaactccagcatgcacatcacagactgccgcctgacaaacggc |
| | | tccaggtaccccaactgtgcataccggaccagcccgaaggagagacacatcatt |
| | | gtggcctgtgaagggagcccatatgtgccagtccactttgatgcttctgtggag |
| | | gactctacctaataatctaga |
| 181 | huVK3LP- | metpaqllfllllwlpdttglkiaafniqtfgetkmsnatlvsyivqilsrydi |
| | hDNase1 | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | hIgG1- | pdsadttatpthcaydrivvagmllrgavvpdsalpfnfqaayglsdqlaqais |
| | WT-NLG- | dhypvevmlkdlepkssdkthtcppcpapellggpsvflfppkpkdtlmisrtp |
| | hRNasse1-WT | evtcvvvdvshedpevkfnwyvdgvevhnaktkpreeqynstyrvvsvltvlhq |
| | | dwlngkeykckvsnkalpapiektiskakgqprepqvytlppsrdeltknqvsl |
| | | tclvkgfypsdiavewesngqpennykttppvldsdgsfflyskitvdksrwqq |
| | | gnvfscsvmhealhnhytqkslslspgkvdgasspvnvsspsvqdipslgkesr |
| | | akkfqrqhmdsdsspsssstycnqmmrrrnmtqgrckpvntfvheplvdvqnvc |
| | | fqekvtckngqgncyksnssmhitdcrltngsrypncayrtspkerhiivaceg |
| | | spyvpvhfdasvedst* |
| 182 | huVK3LP- | gaccaagcttgccaccatggaaaccccagcgcagcttctcttcctcctgctact |
| | mDNase1L3- | ctggctcccagataccaccggtctaaggctctgctccttcaatgtgaggtcctt |
| | mIgG2A- | tggagcgagcaagaaggaaaaccatgaagccatggatatcattgtgaagatcat |
| | C(mut) | caaacgctgtgaccttatactgttgatggaaatcaaggacagcagcaacaacat |
| | | ctgtcccatgctgatggagaagctgaatggaaattcacgaagaagcacaacata |
| | | caactatgtgattagttctcgacttggaagaaacacgtacaaagagcagtatgc |
| | | cttcgtctacaaggagaagctggtgtctgtgaagacaaaataccactaccatga |
| | | ctatcaggatggagacacagacgtgttttccagggagccctttgtggtttggtt |
| | | ccattccccctttactgctgtcaaggacttcgtgattgtccccttgcacacaac |
| | | tcccgagacctccgttaaagagatagatgagctggtcgatgtctacacggatgt |
| | | gagaagccagtggaagacagagaatttcatcttcatgggtgatttcaacgccgg |
| | | ctgtagctatgtccccaagaaggcctggcagaacattcgtttgaggacggaccc |
| | | caagtttgtttggctgattggggaccaagaggacactacggtcaagaagagtac |
| | | cagctgtgcctatgacaggattgtgctttgtggacaagagatagtcaactccgt |
| | | ggttccccgttccagtggcgtctttgactttcagaaagcttatgacttgtctga |
| | | agaggaggccctggatgtcagtgatcactttccagttgagtttaagctacagtc |
| | | ttcaagggccttcaccaacaacagaaaatctgtttctctcaaaaagagaaaaaa |
| | | aggcaatcgctcctcagatctcgagcccagaggtctcacaatcaagccctctcc |
| | | tccatgcaaatgcccagcacctaacctcttgggtggatcatccgtcttcatctt |
| | | ccctccaaagatcaaggatgtactcatgatctccctgagccccatggtcacatg |
| | | tgtggtggtggatgtgagcgaggatgacccagacgtccagatcagctggtttgt |
| | | gaacaacgtggaagtacacacagctcagacacaaacccatagagaggattacaa |
| | | cagtactctccgggtggtcagtgccctccccatccagcaccaggactggatgag |
| | | tggcaaggagttcaaatgctcggtcaacaacaaagacctcccagcgtccatcga |
| | | gagaaccatctcaaaacccagagggccagtaagagctccacaggtatatgtctt |
| | | gcctccaccagcagaagagatgactaagaaagagttcagtctgacctgcatgat |
| | | cacaggcttcttacctgccgaaattgctgtggactggaccagcaatgggcgtac |
| | | agagcaaaactacaagaacaccgcaacagtcctggactctgatggttcttactt |
| | | catgtacagcaagctcagagtacaaaagagcacttgggaaagaggaagtctttt |
| | | cgcctgctcagtggtccacgagggtctgcacaatcaccttacgactaagagctt |
| | | ctctcggactccgggtaaatgataatctagaa |
| 183 | huVK3LP- | metpaqllfllllwlpdttglrlcsfnvrsfgaskkenheamdiivkiikrcdl |
| | mDNase1L3- | illmeikdssnnicpmlmeklngnsrrsttynyvissrlgrntykeqyafvyke |
| | mIgG2A- | klvsvktkyhyhdyqdgdtdvfsrepfvvwfhspftavkdfvivplhttpetsv |
| | C(mut) | keldelvdvytdvrsqwktenfifmgdfnagcsyvpkkawqnirlrtdpkfvwl |
| | | igdqedttvkkstscaydrivlcgqeivnsvvprssgvfdfqkaydlseeeald |
| | | vsdhfpvefklqssraftnnrksvslkkrkkgnrssdleprgltikpsppckcp |
| | | apnllggssvfifppkikdvimislspmvtcvvvdvseddpdvqiswfvnnvev |
| | | htaqtqthredynstlrvvsalpiqhqdwmsgkefkcsvnnkdlpasiertisk |
| | | prgpvrapqvyvlpppaeemtkkefsltcmitgflpaeiavdwtsngrteqnyk |
| | | ntatvldsdgsyfmysklrvqkstwergslfacsvvheglhnhlttksfsrtpg |
| | | k* |
| 184 | mDNase1L3- | gagaccagcttgccccatgtccctgcacccagcttccccacgcctggcctccct |
| | NL- | gctgctcttcatccttgccctccatgacaccctggccctaaggctctgctcctt |
| | mIgG2A_C(mut) | caatgtgaggtcctttggagcgagcaagaaggaaaaccatgaagccatggatat |
| | | cattgtgaagatcatcaaacgctgtgaccttatactgttgatggaaatcaagga |
| | | cagcagcaacaacatctgtcccatgctgatggagaagctgaatggaaattcacg |
| | | aagaagcacaacatacaactatgtgattagttctcgacttggaagaaacacgta |
| | | caaagagcagtatgccttcgtctacaaggagaagctggtgtctgtgaagacaaa |
| | | ataccactaccatgactatcaggatggagacacagacgtgttttccagggagcc |
| | | ctttgtggtttggttccattccccctttactgctgtcaaggacttcgtgattgt |
| | | ccccttgcacacaactcccgagacctccgttaaagagatagatgagctggtcga |
| | | tgtctacacggatgtgagaagccagtggaagacagagaatttcatcttcatggg |
| | | tgatttcaacgccggctgtagctatgtccccaagaaggcctggcagaacattcg |
| | | tttgaggacggaccccaagtttgtttggctgattggggaccaagaggacactac |
| | | ggtcaagaagagtaccagctgtgcctatgacaggattgtgctttgtggacaaga |
| | | gatagtcaactccgtggttccccgttccagtggcgtctttgactttcagaaagc |
| | | ttatgacttgtctgangaggangccctggatgtcagtgatcactttccagttga |
| | | gtttaagctacagtcttcaagggccttcaccaacaacagaaaatctgtttctct |
| | | caaaaagagaaaaaaaggcaatcgctcctcagatctcgagcccagaggtctcac |
| | | aatcaagccctctcctccatgcaaatgcccagcacctaacctcttgggtggatc |
| | | atccgtcttcatcttccctccaaagatcaaggatgtactcatgatctccctgag |
| | | ccccatggtcacatgtgtggtggtggatgtgagcgaggatgacccagacgtcca |
| | | gatcagctggtttgtgaacaacgtggaagtacacacagctcagacacaaaccca |
| | | tagagaggattacaacagtactctccgggtggtcagtgccctccccatccagca |
| | | ccaggactggatgagtggcaaggagttcaaatgctcggtcaacaacaaagacct |
| | | cccagcgtccatcgagagaaccatctcaaaacccagagggccagtaagagctcc |
| | | acaggtatatgtcttgcctccaccagcagaagagatgactaagaaagagttcag |
| | | tctgacctgcatgatcacaggcttcttacctgccgaaattgctgtggactggac |
| | | cagcaatgggcgtacagagcaaaactacaagaacaccgcaacagtcctggactc |
| | | tgatggttcttacttcatgtacagcaagctcagagtacaaaagagcacttggga |
| | | aagaggaagtcttttcgcctgctcagtggtccacgagggtctgcacaatcacct |
| | | tacgactaagagcttctctcggactccgggtaaatgataatctagaa |
| 185 | mDNase1L3- | mslhpasprlaslllfilalhdtlalrlcsfnvrsfgaskkenheamdiivkii |
| | NL- | krcdlillmeikdssnnicpmlmeklngnsrrsttynyvissrlgrntykeqya |
| | mIgG2A_C(mut) | fvykeklvsvktkyhyhdyqdgdtdvfsrepfvvwfhspftavkdfvivplhtt |
| | | petsvkeidelvdvytdvrsqwktenfifmgdfnagcsyvpkkawqnirlrtdp |
| | | kfvwligdqedttvkkstscaydrivlcgqeivnsvvprssgvfdfqkaydlsx |
| | | exaldvsdhfpvefklqssraftnnrksvslkkrkkgnrssdleprgltikpsp |
| | | pckcpapnllggssvfifppkikdvlmislspmvtcvvvdvseddpdvqiswfv |
| | | nnvevhtaqtqthredynstlrvvsalpiqhqdwmsgkefkcsvnnkdlpasie |
| | | rtiskprgpvrapqvyvlpppaeemtkkefsltcmitgflpaeiavdwtsngrt |
| | | eqnykntatvldsdgsyfmysklrvqkstwergslfacsvvheglhnhlttksf |
| | | srtpgk* |
| 186 | huVK3LP- | gttaagcttgccaccatggaaaccccagcgcagcttctcttcctcctgctactc |
| | hDNase1L3- | tggctcccagataccaccggtatgaggatctgctccttcaacgtcaggtccttt |
| | hIgG1WT- | ggggaaagcaagcaggaagacaagaatgccatggatgtcattgtgaaggtcatc |
| | NLG- | aaacgctgtgacatcatactcgtgatggaaatcaaggacagcaacaacaggatc |
| | hRNase1-WT | tgccccatactgatggagaagctgaacagaaattcaaggagaggcataacatac |
| | | aactatgtgattagctctcggcttggaagaaacacatataaagaacaatatgcc |
| | | tttctctacaaggaaaagctggtgtctgtgaagaggagttatcactaccatgac |
| | | tatcaggatggagacgcagatgtgttttccagggagccctttgtggtctggttc |
| | | caatctccccacactgctgtcaaagacttcgtgattatccccctgcacaccacc |
| | | ccagagacatccgttaaggagatcgatgagttggttgaggtctacacggacgtg |
| | | aaacaccgctggaaggcggagaatttcattttcatgggtgacttcaatgccggc |
| | | tgcagctacgtccccaagaaggcctggaagaacatccgcttgaggactgacccc |
| | | aggtttgtttggctgatcggggaccaagaggacaccacggtgaagaagagcacc |
| | | aactgtgcatatgacaggattgtgcttagaggacaagaaatcgtcagttctgtt |
| | | gttcccaagtcaaacagtgtttttgacttccagaaagcttacaagctgactgaa |
| | | gaggaggccctggatgtcagcgaccactttccagttgaatttaaactacagtct |
| | | tcaagggccttcaccaacagcaaaaaatctgtcactctaaggaagaaaacaaag |
| | | agcaaacgctcagatctcgagcccaaatcttctgacaaaactcacacatgtcca |
| | | ccgtgcccagcacctgaactcctggggggaccgtcagtcttcctcttcccccca |
| | | aaacccaaggacaccctcatgatctcccggacccctgaggtcacatgcgtggtg |
| | | gtggacgtgagccacgaagaccctgaggtcaagttcaactggtacgtggacggc |
| | | gtggaggtgcataatgccaagacaaagccgcgggaggagcagtacaacagcacg |
| | | taccgtgtggtcagcgtcctcaccgtcctgcaccaggactggctgaatggcaag |
| | | gagtacaagtgcaaggtctccaacaaagccctcccagcccccatcgagaaaacc |
| | | atctccaaagccaaagggcagccccgagaaccacaggtgtacaccctgccccca |
| | | tcccgggatgagctgaccaagaaccaggtcagcctgacctgcctggtcaaaggc |
| | | ttctatcccagcgacatcgccgtggagtgggagagcaatgggcagccggagaac |
| | | aactacaagaccacgcctcccgtgctggactccgacggctccttcttcctctac |
| | | agcaagctcaccgtggacaagagcaggtggcagcaggggaacgtcttctcatgc |
| | | tccgtgatgcatgaggctctgcacaaccactacacgcagaagagcctctctctg |
| | | tctccgggtaaagtcgacggagctagcagccccgtgaacgtgagcagccccagc |
| | | gtgcaggatatcccttccctgggcaaggaatcccgggccaagaaattccagcgg |
| | | cagcatatggactcagacagttcccccagcagcagctccacctactgtaaccaa |
| | | atgatgaggcgccggaatatgacacaggggcggtgcaaaccagtgaacaccttt |
| | | gtgcacgagcccctggtagatgtccagaatgtctgtttccaggaaaaggtcacc |
| | | tgcaagaacgggcagggcaactgctacaagagcaactccagcatgcacatcaca |
| | | gactgccgcctgacaaacggctccaggtaccccaactgtgcataccggaccagc |
| | | ccgaaggagagacacatcattgtggcctgtgaagggagcccatatgtgccagtc |
| | | cactttgatgcttctgtggaggactctacctaataatctaga |
| 187 | huVK3LP- | metpaqllfllllwlpdttgmricsfnvrsfgeskqedknamdvivkvikrcdi |
| | hDNase1L3- | ilvmeikdsnnricpilmeklnrnsrrgitynyvissrlgrntykeqyaflyke |
| | hIgG1- | klvsvkrsyhyhdyqdgdadvfsrepfvvwfqsphtavkdfviiplhttpetsv |
| | WT-NLG- | keidelvevytdvkhrwkaenfifmgdfnagcsyvpkkawknirlrtdprfvwl |
| | hRNase1-WT | lgdqedttvkkstncaydrivlrgqeivssvvpksnsvfdfqkayklteeeald |
| | | vsdhfpvefklqssraftnskksvtlrkktkskrsdlepkssdkthtcppcpap |
| | | ellggpsvflfppkpkdtlmisrtpevtcvvvdvshedpevkfnwyvdgvevhn |
| | | aktkpreeqynstyrvvsvltvlhqdwlngkeykckvsnkalpapiektiskak |
| | | gqprepqvytlppsrdeltknqvsltclvkgfypsdiavewesngqpennyktt |
| | | ppvldsdgsfflyskltvdksrwqqgnvfscsvmhealhnhytqkslslspgkv |
| | | dgasspvnvsspsvqdipslgkesrakkfqrqhmdsdsspsssstycnqmmrrr |
| | | nmtqgrckpvntfvheplvdvqnvcfqekvtckngqgncyksnssmhitdcrlt |
| | | ngsrypncayrtspkerhiivacegspyvpvhfdasvedst* |
| 188 | huVK3LP- | aagcttgccgccatggaaaccccagcgcagcttctcttcctcctgctactctgg |
| | hRNase1- | ctcccagataccaccggtaaggaatcccgggccaagaaattccagcggcagcat |
| | WT-hIgG1- | atggactcagacagttcccccagcagcagctccacctactgtaaccaaatgatg |
| | WT-NLG- | aggcgccggaatatgacacaggggcggtgcaaaccagtgaacacctttgtgcac |
| | hRNase1-WT | gagcccctggtagatgtccagaatgtctgtttccaggaaaaggtcacctgcaag |
| | | aacgggcagggcaactgctacaagagcaactccagcatgcacatcacagactgc |
| | | cgcctgacaaacggctccaggtaccccaactgtgcataccggaccagcccgaag |
| | | gagagacacatcattgtggcctgtgaagggagcccatatgtgccagtccacttt |
| | | gatgcttctgtggaggactctacagatctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgagggtctgcacaaccactacacgcagaag |
| | | agcctctctctgtctccgggtaaagtcgacggtgctagcagccatgtgaatgtg |
| | | agcagccctagcgtgcaggatatcccttccctgggcaaggaatcccgggccaag |
| | | aaattccagcggcagcatatggactcagacagttcccccagcagcagctccacc |
| | | tactgtaaccaaatgatgaggcgccggaatatgacacaggggcggtgcaaacca |
| | | gtgaacacctttgtgcacgagcccctggtagatgtccagaatgtctgtttccag |
| | | gaaaaggtcacctgcaagaacgggcagggcaactgctacaagagcaactccagc |
| | | atgcacatcacagactgccgcctgacaaacggctccaggtaccccaactgtgca |
| | | taccggaccagcccgaaggagagacacatcattgtggcctgtgaagggagccca |
| | | tatgtgccagtccactttgatgcttctgtggaggactctacctaataatctaga |
| 189 | huVK3LP- | metpaqllfllllwlpdttgkesrakkfqrqhmdsdsspsssstycnqmmrrrn |
| | hRNase1- | mtqgrckpvntfvheplvdvqnvcfqekvtckngqgncyksnssmhitdcrltn |
| | WT-hIgG1- | gsrypncayrtspkerhiivacegspyvpvhfdasvedstdlepkssdkthtcp |
| | WT-NLG- | pcpapellggpsvflfppkpkdtlmisrtpevtcvvvdvshedpevkfnwyvdg |
| | hRNase1-WT | vevhnaktkpreeqynstyrvvsvltvlhqdwlngkeykckvsnkalpapiekt |
| | | iskakgqprepqvytlppsrdeltknqvsltclvkgfypsdiavewesngqpen |
| | | nykttppvldsdgsfflyskltvdksrwqqgnvfscsvmheglhnhytqkslsl |
| | | spgkvdgasshvnvsspsvqdipslgkesrakkfqrqhmdsdsspsssstycnq |
| | | mmrrrnmtqgrckpvntfvheplvdvqnvcfqekvtckngqgncyksnssmhit |
| | | dcrltngsrypncayrtspkerhiivacegspyvpvhfdasvedst* |
| 190 | huVK3LP- | aagcttgccgccatggaaaccccagcgcagcttctcttcctcctgctactctgg |
| | hRNase1- | ctcccagataccaccggtaaggaatcccgggccaagaaattccagcggcagcat |
| | WT-(G4S)4- | atggactcagacagttcccccagcagcagctccacctactgtaaccaaatgatg |
| | hIgG1- | aggcgccggaatatgacacaggggcggtgcaaaccagtgaacacctttgtgcac |
| | WT-NLG- | gagcccctggtagatgtccagaatgtctgtttccaggaaaaggtcacctgcaag |
| | hRNase1-WT | aacgggcagggcaactgctacaagagcaactccagcatgcacatcacagactgc |
| | | cgcctgacaaacggctccaggtaccccaactgtgcataccggaccagcccgaag |
| | | gagagacacatcattgtggcctgtgaagggagcccatatgtgccagtccacttt |
| | | gatgcttctgtggaggactctacagatctctccggaggaggtggctcaggtggt |
| | | ggaggatctggaggaggtgggagtggtggaggtggttctaccggtctcgagccc |
| | | aaatcttctgacaaaactcacacatgtccaccgtgcccagcacctgaactcctg |
| | | gggggaccgtcagtcttcctcttccccccaaaacccaaggacaccctcatgatc |
| | | tcccggacccctgaggtcacatgcgtggtggtggacgtgagccacgaagaccct |
| | | gaggtcaagttcaactggtacgtggacggcgtggaggtgcataatgccaagaca |
| | | aagccgcgggaggagcagtacaacagcacgtaccgtgtggtcagcgtcctcacc |
| | | gtcctgcaccaggactggctgaatggcaaggagtacaagtgcaaggtctccaac |
| | | aaagccctcccagcccccatcgagaaaaccatctccaaagccaaagggcagccc |
| | | cgagaaccacaggtgtacaccctgcccccatcccgggatgagctgaccaagaac |
| | | caggtcagcctgacctgcctggtcaaaggcttctatcccagcgacatcgccgtg |
| | | gagtgggagagcaatgggcagccggagaacaactacaagaccacgcctcccgtg |
| | | ctggactccgacggctccttcttcctctacagcaagctcaccgtggacaagagc |
| | | aggtggcagcaggggaacgtcttctcatgctccgtgatgcatgagggtctgcac |
| | | aaccactacacgcagaagagcctctctctgtctccgggtaaagtcgacggtgct |
| | | agcagccatgtgaatgtgagcagccctagcgtgcaggatatcccttccctgggc |
| | | aaggaatcccgggccaagaaattccagcggcagcatatggactcagacagttcc |
| | | cccagcagcagctccacctactgtaaccaaatgatgaggcgccggaatatgaca |
| | | caggggcggtgcaaaccagtgaacacctttgtgcacgagcccctggtagatgtc |
| | | cagaatgtctgtttccaggaaaaggtcacctgcaagaacgggcagggcaactgc |
| | | tacaagagcaactccagcatgcacatcacagactgccgcctgacaaacggctcc |
| | | aggtaccccaactgtgcataccggaccagcccgaaggagagacacatcattgtg |
| | | gcctgtgaagggagcccatatgtgccagtccactttgatgcttctgtggaggac |
| | | tctacctaataatctaga |
| 191 | huVK3LP- | metpaqllfllllwlpdttgkesrakkfqrqhmdsdsspsssstycnqmmrrrn |
| | hRNase1- | mtqgrckpvntfvheplvdvqnvcfqekvtckngqgncyksnssmhitdcrltn |
| | WT-(G4S)4- | gsrypncayrtspkerhiivacegspyvpvhfdasvedstdlsggggsggggsg |
| | hIgG1- | gggsggggstglepkssdkthtcppcpapellggpsvflfppkpkdtlmisrtp |
| | WT-NLG- | evtcvvvdvshedpevkfnwyvdgvevhnaktkpreeqynstyrvvsvltvlhq |
| | hRNase1-WT | dwlngkeykckvsnkalpapiektiskakgqprepqvytlppsrdeltknqvsl |
| | | tclvkgfypsdiavewesngqpennykttppvldsdgsfflyskitvdksrwqq |
| | | gnvfscsvmheglhnhytqkslslspgkvdgasshvnvsspsvqdipslgkesr |
| | | akkfqrqhmdsdsspsssstycnqmmrrrnmtqgrckpvntfvheplvdvqnvc |
| | | fqekvtckngqgncyksnssmhitdcrltngsrypncayrtspkerhiivaceg |
| | | spyvpvhfdasvedst* |
| 192 | huVK3LP- | aagcttgccaccatggaaaccccagcgcagcttctcttcctcctgctactctgg |
| | hTREX1- | ctcccagataccaccggtatgggccctggagctcgcagacagggcaggattgtg |
| | 72AA- | cagggaaggcctgagatgtgcttctgcccaccccctaccccactccctcccctt |
| | (G4S)4- | cggatcttaacactgggcactcacacacccaccccatgctcctctccaggctca |
| | hIgG1- | gcagcaggtacgtacccaaccatgggctcgcaggccctgcccccggggcccatg |
| | WT-NLG- | cagaccctcatctttttcgacatggaggccactggcttgcccttctcccagccc |
| | hRNase1-WT | aaggtcacggagctgtgcctgctggctgtccacagatgtgccctggagagcccc |
| | | cccacctctcaggggccacctcccacagttcctccaccaccgcgtgtggtagac |
| | | aagctctccctgtgtgtggctccggggaaggcctgcagccctgcagccagcgag |
| | | atcacaggtctgagcacagctgtgctggcagcgcatgggcgtcaatgttttgat |
| | | gacaacctggccaacctgctcctagccttcctgcggcgccagccacagccctgg |
| | | tgcctggtggcacacaatggtgaccgctacgacttccccctgctccaagcagag |
| | | ctggctatgctgggcctcaccagtgctctggatggtgccttctgtgtggatagc |
| | | atcactgcgctgaaggccctggagcgagcaagcagcccctcagaacacggccca |
| | | aggaagagctacagcctaggcagcatctacactcgcctgtatgggcagtcccct |
| | | ccagactcgcacacggctgagggtgatgtcctggccctgctcagcatctgtcag |
| | | tggagaccacaggccctgctgcggtgggtggatgctcacgccaggcctttcggc |
| | | accatcaggcccatgtatggggtcacagcctctgctaggaccaaagatctctcc |
| | | ggaggaggtggctcaggtggtggaggatctggaggaggtgggagtggtggaggt |
| | | ggttctaccggtctcgagcccaaatcttctgacaaaactcacacatgtccaccg |
| | | tgcccagcacctgaactcctggggggaccgtcagtcttcctcttccccccaaaa |
| | | cccaaggacaccctcatgatctcccggacccctgaggtcacatgcgtggtggtg |
| | | gacgtgagccacgaagaccctgaggtcaagttcaactggtacgtggacggcgtg |
| | | gaggtgcataatgccaagacaaagccgcgggaggagcagtacaacagcacgtac |
| | | cgtgtggtcagcgtcctcaccgtcctgcaccaggactggctgaatggcaaggag |
| | | tacaagtgcaaggtctccaacaaagccctcccagcccccatcgagaaaaccatc |
| | | tccaaagccaaagggcagccccgagaaccacaggtgtacaccctgcccccatcc |
| | | cgggatgagctgaccaagaaccaggtcagcctgacctgcctggtcaaaggcttc |
| | | tatcccagcgacatcgccgtggagtgggagagcaatgggcagccggagaacaac |
| | | tacaagaccacgcctcccgtgctggactccgacggctccttcttcctctacagc |
| | | aagctcaccgtggacaagagcaggtggcagcaggggaacgtcttctcatgctcc |
| | | gtgatgcatgaggctctgcacaaccactacacgcagaagagcctctctctgtct |
| | | ccgggtaaagtcgacggagctagcagccccgtgaacgtgagcagccccagcgtg |
| | | caggatatcccttccctgggcaaggaatcccgggccaagaaattccagcggcag |
| | | catatggactcagacagttcccccagcagcagctccacctactgtaaccaaatg |
| | | atgaggcgccggaatatgacacaggggcggtgcaaaccagtgaacacctttgtg |
| | | cacgagcccctggtagatgtccagaatgtctgtttccaggaaaaggtcacctgc |
| | | aagaacgggcagggcaactgctacaagagcaactccagcatgcacatcacagac |
| | | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacctaataatctaga |
| 193 | huVK3LP- | metpaqllfllllwlpdttgmgpgarrqgrivqgrpemcfcppptplpplrilt |
| | hTREX1- | lgthtptpcsspgsaagtyptmgsqalppgpmqtliffdmeatglpfsqpkvte |
| | 72AA- | lcllavhrcalespptsqgppptvpppprvvdklslcvapgkacspaaseitgl |
| | (G4S)4- | stavlaahgrqcfddnlanlllafirrqpqpwclvahngdrydfpllqaelaml |
| | hIgG1- | gltsaldgafcvdsitalkalerasspsehgprksyslgsiytrlygqsppdsh |
| | WT-NLG- | taegdvlallsicqwrpqallrwvdaharpfgtirpmygvtasartkdlsgggg |
| | hRNase1-WT | sggggsggggsggggstglepkssdkthtcppcpapellggpsvflfppkpkdt |
| | | lmisrtpevtcvvvdvshedpevkfnwyvdgvevhnaktkpreeqynstyrvvs |
| | | vltvlhqdwlngkeykckvsnkalpapiektiskakgqprepqvytlppsrdel |
| | | tknqvsltclvkgfypsdiavewesngqpennykttppvldsdgsfflyskitv |
| | | dksrwqqgnvfscsvmhealhnhytqkslslspgkvdgasspvnvsspsvqdip |
| | | slgkesrakkfqrqhmdsdsspsssstycnqmmrrrnmtqgrckpvntfvhepl |
| | | vdvqnvcfqekvtckngqgncyksnssmhitdcrltngsrypncayrtspkerh |
| | | iivacegspyvpvhfdasvedst* |
| 194 | huVK3LP- | aagcttgccgccatggaaaccccagcgcagcttctcttcctcctgctactctgg |
| | hRNase1- | ctcccagataccaccggtaaggaatcccgggccaagaaattccagcggcagcat |
| | WT-hIgG1- | atggactcagacagttcccccagcagcagctccacctactgtaaccaaatgatg |
| | WT-NLG- | aggcgccggaatatgacacaggggcggtgcaaaccagtgaacacctttgtgcac |
| | hTREX1- | gagcccctggtagatgtccagaatgtctgtttccaggaaaaggtcacctgcaag |
| | 72AA | aacgggcagggcaactgctacaagagcaactccagcatgcacatcacagactgc |
| | | cgcctgacaaacggctccaggtaccccaactgtgcataccggaccagcccgaag |
| | | gagagacacatcattgtggcctgtgaagggagcccatatgtgccagtccacttt |
| | | gatgcttctgtggaggactctacagatctcgagcccaaatcttctgacaaaact |
| | | cacacatgtccaccgtgcccagcacctgaactcctggggggaccgtcagtcttc |
| | | ctcttccccccaaaacccaaggacaccctcatgatctcccggacccctgaggtc |
| | | acatgcgtggtggtggacgtgagccacgaagaccctgaggtcaagttcaactgg |
| | | tacgtggacggcgtggaggtgcataatgccaagacaaagccgcgggaggagcag |
| | | tacaacagcacgtaccgtgtggtcagcgtcctcaccgtcctgcaccaggactgg |
| | | ctgaatggcaaggagtacaagtgcaaggtctccaacaaagccctcccagccccc |
| | | atcgagaaaaccatctccaaagccaaagggcagccccgagaaccacaggtgtac |
| | | accctgcccccatcccgggatgagctgaccaagaaccaggtcagcctgacctgc |
| | | ctggtcaaaggcttctatcccagcgacatcgccgtggagtgggagagcaatggg |
| | | cagccggagaacaactacaagaccacgcctcccgtgctggactccgacggctcc |
| | | ttcttcctctacagcaagctcaccgtggacaagagcaggtggcagcaggggaac |
| | | gtcttctcatgctccgtgatgcatgagggtctgcacaaccactacacgcagaag |
| | | agcctctctctgtctccgggtaaagtcgacggtgctagcagccatgtgaatgtg |
| | | agcagccctagcgtgcaggatatcatgggccctggagctcgcagacagggcagg |
| | | attgtgcagggaaggcctgagatgtgcttctgcccaccccctaccccactccct |
| | | ccccttcggatcttaacactgggcactcacacacccaccccatgctcctctcca |
| | | ggctcagcagcaggtacgtacccaaccatgggctcgcaggccctgcccccgggg |
| | | cccatgcagaccctcatctttttcgacatggaggccactggcttgcccttctcc |
| | | cagcccaaggtcacggagctgtgcctgctggctgtccacagatgtgccctggag |
| | | agcccccccacctctcaggggccacctcccacagttcctccaccaccgcgtgtg |
| | | gtagacaagctctccctgtgtgtggctccggggaaggcctgcagccctgcagcc |
| | | agcgagatcacaggtctgagcacagctgtgctggcagcgcatgggcgtcaatgt |
| | | tttgatgacaacctggccaacctgctcctagccttcctgcggcgccagccacag |
| | | ccctggtgcctggtggcacacaatggtgaccgctacgacttccccctgctccaa |
| | | gcagagctggctatgctgggcctcaccagtgctctggatggtgccttctgtgtg |
| | | gatagcatcactgcgctgaaggccctggagcgagcaagcagcccctcagaacac |
| | | ggcccaaggaagagctacagcctaggcagcatctacactcgcctgtatgggcag |
| | | tcccctccagactcgcacacggctgagggtgatgtcctggccctgctcagcatc |
| | | tgtcagtggagaccacaggccctgctgcggtgggtggatgctcacgccaggcct |
| | | ttcggcaccatcaggcccatgtatggggtcacagcctctgctaggaccaaatga |
| | | taatctaga |
| 195 | huVK3LP- | metpaqllfllllwlpdttgkesrakkfqrqhmdsdsspsssstycnqmmrrrn |
| | hRNase1- | mtqgrckpvntfvheplvdvqnvcfqekvtckngqgncyksnssmhitdcrltn |
| | WT-hIgG1- | gsrypncayrtspkerhiivacegspyvpvhfdasvedstdlepkssdkthtcp |
| | WT-NLG- | pcpapellggpsvflfppkpkdtlmisrtpevtcvvvdvshedpevkfnwyvdg |
| | hTREX1- | vevhnaktkpreeqynstyrvvsvltvlhqdwlngkeykckvsnkalpapiekt |
| | 72AA | lskakgqprepqvytlppsrdeltknqvsltclvkgfypsdiavewesngqpen |
| | | nykttppvldsdgsfflyskltvdksrwqqgnvfscsvmheglhnhytqkslsl |
| | | spgkvdgasshvnvsspsvqdimgpgarrqgrivqgrpemcfcppptplpplri |
| | | ltlgthtptpcsspgsaagtyptmgsqalppgpmqtliffdmeatglpfsqpkv |
| | | telcllavhrcalespptsqgppptvpppprvvdklslcvapgkacspaaseit |
| | | glstavlaahgrqcfddnlanlllafirrqpqpwclvahngdrydfpllqaela |
| | | mlgltsaldgafcvdsitalkalerasspsehgprksyslgsiytrlygqsppd |
| | | shtaegdvlallsicqwrpqallrwvdaharpfgtirpmygvtasartk* |
| 196 | huVK3LP- | gttaagcttgccaccatggaaaccccagcgcagcttctcttcctcctgctactc |
| | hDNase1L3- | tggctcccagataccaccggtatgaggatctgctccttcaacgtcaggtccttt |
| | (G4S)4- | ggggaaagcaagcaggaagacaagaatgccatggatgtcattgtgaaggtcatc |
| | hIgG1Wt- | aaacgctgtgacatcatactcgtgatggaaatcaaggacagcaacaacaggatc |
| | NLG- | tgccccatactgatggagaagctgaacagaaattcaaggagaggcataacatac |
| | hRNase1-WT | aactatgtgattagctctcggcttggaagaaacacatataaagaacaatatgcc |
| | | tttctctacaaggaaaagctggtgtctgtgaagaggagttatcactaccatgac |
| | | tatcaggatggagacgcagatgtgttttccagggagccctttgtggtctggttc |
| | | caatctccccacactgctgtcaaagacttcgtgattatccccctgcacaccacc |
| | | ccagagacatccgttaaggagatcgatgagttggttgaggtctacacggacgtg |
| | | aaacaccgctggaaggcggagaatttcattttcatgggtgacttcaatgccggc |
| | | tgcagctacgtccccaagaaggcctggaagaacatccgcttgaggactgacccc |
| | | aggtttgtttggctgatcggggaccaagaggacaccacggtgaagaagagcacc |
| | | aactgtgcatatgacaggattgtgcttagaggacaagaaatcgtcagttctgtt |
| | | gttcccaagtcaaacagtgtttttgacttccagaaagcttacaagctgactgaa |
| | | gaggaggccctggatgtcagcgaccactttccagttgaatttaaactacagtct |
| | | tcaagggccttcaccaacagcaaaaaatctgtcactctaaggaagaaaacaaag |
| | | agcaaacgctcagatctctccggaggaggtggctcaggtggtggaggatctgga |
| | | ggaggtgggagtggtggaggtggttctaccggtctcgagcccaaatcttctgac |
| | | aaaactcacacatgtccaccgtgcccagcacctgaactcctggggggaccgtca |
| | | gtcttcctcttccccccaaaacccaaggacaccctcatgatctcccggacccct |
| | | gaggtcacatgcgtggtggtggacgtgagccacgaagaccctgaggtcaagttc |
| | | aactggtacgtggacggcgtggaggtgcataatgccaagacaaagccgcgggag |
| | | gagcagtacaacagcacgtaccgtgtggtcagcgtcctcaccgtcctgcaccag |
| | | gactggctgaatggcaaggagtacaagtgcaaggtctccaacaaagccctccca |
| | | gcccccatcgagaaaaccatctccaaagccaaagggcagccccgagaaccacag |
| | | gtgtacaccctgcccccatcccgggatgagctgaccaagaaccaggtcagcctg |
| | | acctgcctggtcaaaggcttctatcccagcgacatcgccgtggagtgggagagc |
| | | aatgggcagccggagaacaactacaagaccacgcctcccgtgctggactccgac |
| | | ggctccttcttcctctacagcaagctcaccgtggacaagagcaggtggcagcag |
| | | gggaacgtcttctcatgctccgtgatgcatgagggtctgcacaaccactacacg |
| | | cagaagagcctctctctgtctccgggtaaagtcgacggtgctagcagccatgtg |
| | | aatgtgagcagccctagcgtgcaggatatcccttccctgggcaaggaatcccgg |
| | | gccaagaaattccagcggcagcatatggactcagacagttcccccagcagcagc |
| | | tccacctactgtaaccaaatgatgaggcgccggaatatgacacaggggcggtgc |
| | | aaaccagtgaacacctttgtgcacgagcccctggtagatgtccagaatgtctgt |
| | | ttccaggaaaaggtcacctgcaagaacgggcagggcaactgctacaagagcaac |
| | | tccagcatgcacatcacagactgccgcctgacaaacggctccaggtaccccaac |
| | | tgtgcataccggaccagcccgaaggagagacacatcattgtggcctgtgaaggg |
| | | agcccatatgtgccagtccactttgatgcttctgtggaggactctacctaataa |
| | | tctaga |
| 197 | huVK3LP- | metpaqllfllllwlpdttgmricsfnvrsfgeskqedknamdvivkvikrcdi |
| | hDNase1L3- | llvmeikdsnnricpilmeklnrnsrrgitynyvissrlgrntykeqyaflyke |
| | (G4S)4- | klvsvkrsyhyhdyqdgdadvfsrepfvvwfqsphtavkdfviiplhttpetsv |
| | hIgG1Wt- | keidelvevytdvkhrwkaenfifmgdfnagcsyvpkkawknirlrtdprfvwl |
| | NLG- | lgdqedttvkkstncaydrivlrgqeivssvvpksnsvfdfqkayklteeeald |
| | hRNase1-WT | vsdhfpvefklqssraftnskksvtlrkktkskrsdlsggggsggggsggggsg |
| | | gggstglepkssdkthtcppcpapellggpsvflfppkpkdtlmisrtpevtcv |
| | | vvdvshedpevkfnwyvdgvevhnaktkpreeqynstyrvvsvltvlhqdwlng |
| | | keykckvsnkalpapiektiskakgqprepqvytlppsrdeltknqvsltclvk |
| | | gfypsdiavewesngqpennykttppvldsdgsfflyskitvdksrwqqgnvfs |
| | | csvmheglhnhytqkslslspgkvdgasshvnvsspsvqdipslgkesrakkfq |
| | | rqhmdsdsspsssstycnqmmrrrnmtqgrckpvntfvheplvdvqnvcfqekv |
| | | tckngqgncyksnssmhitdcrltngsrypncayrtspkerhiivacegspyvp |
| | | vhfdasvedst* |
| 198 | huVK3LP- | gttaagcttgccaccatggaaaccccagcgcagcttctcttcctcctgctactc |
| | hRNase1- | tggctcccagataccaccggtaaggaatcccgggccaagaaattccagcggcag |
| | WT-(G4S)4- | catatggactcagacagttcccccagcagcagctccacctactgtaaccaaatg |
| | hIgG1- | atgaggcgccggaatatgacacaggggcggtgcaaaccagtgaacacctttgtg |
| | WT-NLG- | cacgagcccctggtagatgtccagaatgtctgtttccaggaaaaggtcacctgc |
| | hDNase1L3 | aagaacgggcagggcaactgctacaagagcaactccagcatgcacatcacagac |
| | | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacagatctctccggaggaggtggctcaggt |
| | | ggtggaggatctggaggaggtgggagtggtggaggtggttctaccggtctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaagtcgacggt |
| | | gctagcagccatgtgaatgtgagcagccctagcgtgcaggatatcatgaggatc |
| | | tgctccttcaacgtcaggtcctttggggaaagcaagcaggaagacaagaatgcc |
| | | atggatgtcattgtgaaggtcatcaaacgctgtgacatcatactcgtgatggaa |
| | | atcaaggacagcaacaacaggatctgccccatactgatggagaagctgaacaga |
| | | aattcaaggagaggcataacatacaactatgtgattagctctcggcttggaaga |
| | | aacacatataaagaacaatatgcctttctctacaaggaaaagctggtgtctgtg |
| | | aagaggagttatcactaccatgactatcaggatggagacgcagatgtgttttcc |
| | | agggagccctttgtggtctggttccaatctccccacactgctgtcaaagacttc |
| | | gtgattatccccctgcacaccaccccagagacatccgttaaggagatcgatgag |
| | | ttggttgaggtctacacggacgtgaaacaccgctggaaggcggagaatttcatt |
| | | ttcatgggtgacttcaatgccggctgcagctacgtccccaagaaggcctggaag |
| | | aacatccgcttgaggactgaccccaggtttgtttggctgatcggggaccaagag |
| | | gacaccacggtgaagaagagcaccaactgtgcatatgacaggattgtgcttaga |
| | | ggacaagaaatcgtcagttctgttgttcccaagtcaaacagtgtttttgacttc |
| | | cagaaagcttacaagctgactgaagaggaggccctggatgtcagcgaccacttt |
| | | ccagttgaatttaaactacagtcttcaagggccttcaccaacagcaaaaaatct |
| | | gtcactctaaggaagaaaacaaagagcaaacgctcctaatgatctaga |
| 199 | huVK3LP- | metpaqllfllllwlpdttgkesrakkfqrqhmdsdsspsssstycnqmmrrrn |
| | hRNase1- | mtqgrckpvntfvheplvdvqnvcfqekvtckngqgncyksnssmhitdcrltn |
| | WT-(G4S)4- | gsrypncayrtspkerhiivacegspyvpvhfdasvedstdlsggggsggggsg |
| | hIgG1- | gggsggggstglepkssdkthtcppcpapellggpsvflfppkpkdtlmisrtp |
| | WT-NLG- | evtcvvvdvshedpevkfnwyvdgvevhnaktkpreeqynstyrvvsvltvlhq |
| | hDNase1L3 | dwlngkeykckvsnkalpapiektiskakgqprepqvytlppsrdeltknqvsl |
| | | tclvkgfypsdiavewesngqpennykttppvldsdgsfflyskitvdksrwqq |
| | | gnvfscsvmhealhnhytqkslslspgkvdgasshvnvsspsvqdimricsfnv |
| | | rsfgeskqedknamdvivkvikrcdiilvmeikdsnnricpilmeklnrnsrrg |
| | | itynyvissrlgrntykeqyaflykeklvsvkrsyhyhdyqdgdadvfsrepfv |
| | | vwfqsphtavkdfviiplhttpetsvkeidelvevytdvkhrwkaenfifmgdf |
| | | nagcsyvpkkawknirlrtdprfvwligdqedttvkkstncaydrivlrgqeiv |
| | | ssvvpksnsvfdfqkayklteeealdvsdhfpvefklqssraftnskksvtlrk |
| | | ktkskrs* |
| 200 | huVK3LP- | gttaagcttgccaccatggaaaccccagcgcagcttctcttcctcctgctactc |
| | hDNase1- | tggctcccagataccaccggtctgaagatcgcagccttcaacatccagacattt |
| | G105R; | ggggagaccaagatgtccaatgccaccctcgtcagctacattgtgcagatcctg |
| | A114F- | agccgctatgacatcgccctggtccaggaggtcagagacagccacctgactgcc |
| | (G4S)4- | gtggggaagctgctggacaacctcaatcaggatgcaccagacacctatcactac |
| | hIgG1- | gtggtcagtgagccactgggacggaacagctataaggagcgctacctgttcgtg |
| | WT-NLG- | tacaggcctgaccaggtgtctgcggtggacagctactactacgatgatggctgc |
| | hTREX1- | gagccctgcaggaacgacaccttcaaccgagagccattcattgtcaggttcttc |
| | 72AA | tcccggttcacagaggtcagggagtttgccattgttcccctgcatgcggccccg |
| | | ggggacgcagtagccgagatcgacgctctctatgacgtctacctggatgtccaa |
| | | gagaaatggggcttggaggacgtcatgttgatgggcgacttcaatgcgggctgc |
| | | agctatgtgagaccctcccagtggtcatccatccgcctgtggacaagccccacc |
| | | ttccagtggctgatccccgacagcgctgacaccacagctacacccacgcactgt |
| | | gcctatgacaggatcgtggttgcagggatgctgctccgaggcgccgttgttccc |
| | | gactcggctcttccctttaacttccaggctgcctatggcctgagtgaccaactg |
| | | gcccaagccatcagtgaccactatccagtggaggtgatgctgaaagatctctcc |
| | | ggaggaggtggctcaggtggtggaggatctggaggaggtgggagtggtggaggt |
| | | ggttctaccggtctcgagcccaaatcttctgacaaaactcacacatgtccaccg |
| | | tgcccagcacctgaactcctggggggaccgtcagtcttcctcttccccccaaaa |
| | | cccaaggacaccctcatgatctcccggacccctgaggtcacatgcgtggtggtg |
| | | gacgtgagccacgaagaccctgaggtcaagttcaactggtacgtggacggcgtg |
| | | gaggtgcataatgccaagacaaagccgcgggaggagcagtacaacagcacgtac |
| | | cgtgtggtcagcgtcctcaccgtcctgcaccaggactggctgaatggcaaggag |
| | | tacaagtgcaaggtctccaacaaagccctcccagcccccatcgagaaaaccatc |
| | | tccaaagccaaagggcagccccgagaaccacaggtgtacaccctgcccccatcc |
| | | cgggatgagctgaccaagaaccaggtcagcctgacctgcctggtcaaaggcttc |
| | | tatcccagcgacatcgccgtggagtgggagagcaatgggcagccggagaacaac |
| | | tacaagaccacgcctcccgtgctggactccgacggctccttcttcctctacagc |
| | | aagctcaccgtggacaagagcaggtggcagcaggggaacgtcttctcatgctcc |
| | | gtgatgcatgaggctctgcacaaccactacacgcagaagagcctctctctgtct |
| | | ccgggtaaagtcgacggtgctagcagccatgtgaatgtgagcagccctagcgtg |
| | | caggatatcatgggccctggagctcgcagacagggcaggattgtgcagggaagg |
| | | cctgagatgtgcttctgcccaccccctaccccactccctccccttcggatctta |
| | | acactgggcactcacacacccaccccatgctcctctccaggctcagcagcaggt |
| | | acgtacccaaccatgggctcgcaggccctgcccccggggcccatgcagaccctc |
| | | atctttttcgacatggaggccactggcttgcccttctcccagcccaaggtcacg |
| | | gagctgtgcctgctggctgtccacagatgtgccctggagagcccccccacctct |
| | | caggggccacctcccacagttcctccaccaccgcgtgtggtagacaagctctcc |
| | | ctgtgtgtggctccggggaaggcctgcagccctgcagccagcgagatcacaggt |
| | | ctgagcacagctgtgctggcagcgcatgggcgtcaatgttttgatgacaacctg |
| | | gccaacctgctcctagccttcctgcggcgccagccacagccctggtgcctggtg |
| | | gcacacaatggtgaccgctacgacttccccctgctccaagcagagctggctatg |
| | | ctgggcctcaccagtgctctggatggtgccttctgtgtggatagcatcactgcg |
| | | ctgaaggccctggagcgagcaagcagcccctcagaacacggcccaaggaagagc |
| | | tacagcctaggcagcatctacactcgcctgtatgggcagtcccctccagactcg |
| | | cacacggctgagggtgatgtcctggccctgctcagcatctgtcagtggagacca |
| | | caggccctgctgcggtgggtggatgctcacgccaggcctttcggcaccatcagg |
| | | cccatgtatggggtcacagcctctgctaggaccaaatgataatctaga |
| 201 | huVK3LP- | metpaqllfllllwlpdttglkiaafniqtfgetkmsnatlvsyivqilsrydi |
| | hDNase1- | alvqevrdshltavgklldnlnqdapdtyhyvvseplgrnsykerylfvyrpdq |
| | G105R; | vsavdsyyyddgcepcrndtfnrepfivrffsrftevrefaivplhaapgdava |
| | A114F- | eidalydvyldvqekwgledvmlmgdfnagcsyvrpsqwssirlwtsptfqwli |
| | (G4S)4- | pdsadttatpthcaydrivvagmllrgavvpdsalpfnfqaayglsdqlaqais |
| | hIgG1- | dhypvevmlkdlsggggsggggsggggsggggstglepkssdkthtcppcpape |
| | WT-NLG- | llggpsvflfppkpkdtlmisrtpevtcvvvdvshedpevkfnwyvdgvevhna |
| | hTREX1- | ktkpreeqynstyrvvsvltvlhqdwlngkeykckvsnkalpapiektiskakg |
| | 72AA | qprepqvytlppsrdeltknqvsltclvkgfypsdiavewesngqpennykttp |
| | | pvidsdgsfflyskltvdksrwqqgnvfscsvmhealhnhytqkslslspgkvd |
| | | gasshvnvsspsvqdimgpgarrqgrivqgrpemcfcppptplpplriltlgth |
| | | tptpcsspgsaagtyptmgsqalppgpmqtliffdmeatgipfsqpkvtelcll |
| | | avhrcalespptsqgppptvpppprvvdklslcvapgkacspaaseitglstav |
| | | laahgrqcfddnlanlllaflrrqpqpwclvahngdrydfpllqaelamlglts |
| | | aldgafcvdsitalkalerasspsehgprksyslgsiytrlygqsppdshtaeg |
| | | dvlallsicqwrpqallrwvdaharpfgtirpmygvtasartk* |
| 202 | huVK3LP- | aagcttgccaccatggaaaccccagcgcagcttctcttcctcctgctactctgg |
| | hTREX1- | ctcccagataccaccggtatgggccctggagctcgcagacagggcaggattgtg |
| | 72AA | cagggaaggcctgagatgtgcttctgcccaccccctaccccactccctcccctt |
| | (G4S)4- | cggatcttaacactgggcactcacacacccaccccatgctcctctccaggctca |
| | hIgG1- | gcagcaggtacgtacccaaccatgggctcgcaggccctgcccccggggcccatg |
| | WT-NLG- | cagaccctcatctttttcgacatggaggccactggcttgcccttctcccagccc |
| | hDNase1- | aaggtcacggagctgtgcctgctggctgtccacagatgtgccctggagagcccc |
| | G105R; | cccacctctcaggggccacctcccacagttcctccaccaccgcgtgtggtagac |
| | A114F | aagctctccctgtgtgtggctccggggaaggcctgcagccctgcagccagcgag |
| | | atcacaggtctgagcacagctgtgctggcagcgcatgggcgtcaatgttttgat |
| | | gacaacctggccaacctgctcctagccttcctgcggcgccagccacagccctgg |
| | | tgcctggtggcacacaatggtgaccgctacgacttccccctgctccaagcagag |
| | | ctggctatgctgggcctcaccagtgctctggatggtgccttctgtgtggatagc |
| | | atcactgcgctgaaggccctggagcgagcaagcagcccctcagaacacggccca |
| | | aggaagagctacagcctaggcagcatctacactcgcctgtatgggcagtcccct |
| | | ccagactcgcacacggctgagggtgatgtcctggccctgctcagcatctgtcag |
| | | tggagaccacaggccctgctgcggtgggtggatgctcacgccaggcctttcggc |
| | | accatcaggcccatgtatggggtcacagcctctgctaggaccaaagatctctcc |
| | | ggaggaggtggctcaggtggtggaggatctggaggaggtgggagtggtggaggt |
| | | ggttctaccggtctcgagcccaaatcttctgacaaaactcacacatgtccaccg |
| | | tgcccagcacctgaactcctggggggaccgtcagtcttcctcttccccccaaaa |
| | | cccaaggacaccctcatgatctcccggacccctgaggtcacatgcgtggtggtg |
| | | gacgtgagccacgaagaccctgaggtcaagttcaactggtacgtggacggcgtg |
| | | gaggtgcataatgccaagacaaagccgcgggaggagcagtacaacagcacgtac |
| | | cgtgtggtcagcgtcctcaccgtcctgcaccaggactggctgaatggcaaggag |
| | | tacaagtgcaaggtctccaacaaagccctcccagcccccatcgagaaaaccatc |
| | | tccaaagccaaagggcagccccgagaaccacaggtgtacaccctgcccccatcc |
| | | cgggatgagctgaccaagaaccaggtcagcctgacctgcctggtcaaaggcttc |
| | | tatcccagcgacatcgccgtggagtgggagagcaatgggcagccggagaacaac |
| | | tacaagaccacgcctcccgtgctggactccgacggctccttcttcctctacagc |
| | | aagctcaccgtggacaagagcaggtggcagcaggggaacgtcttctcatgctcc |
| | | gtgatgcatgaggctctgcacaaccactacacgcagaagagcctctctctgtct |
| | | ccgggtaaagtcgacggtgctagcagccatgtgaatgtgagcagccctagcgtg |
| | | caggatatcctgaagatcgcagccttcaacatccagacatttggggagaccaag |
| | | atgtccaatgccaccctcgtcagctacattgtgcagatcctgagccgctatgac |
| | | atcgccctggtccaggaggtcagagacagccacctgactgccgtggggaagctg |
| | | ctggacaacctcaatcaggatgcaccagacacctatcactacgtggtcagtgag |
| | | ccactgggacggaacagctataaggagcgctacctgttcgtgtacaggcctgac |
| | | caggtgtctgcggtggacagctactactacgatgatggctgcgagccctgcggg |
| | | aacgacaccttcaaccgagagccagccattgtcaggttcttctcccggttcaca |
| | | gaggtcagggagtttgccattgttcccctgcatgcggccccgggggacgcagta |
| | | gccgagatcgacgctctctatgacgtctacctggatgtccaagagaaatggggc |
| | | tcggaggacgtcatgttgatgggcgacttcaatgcgggctgcagctatgtgaga |
| | | ccctcccagtggtcatccatccgcctgtggacaagccccaccttccagtggctg |
| | | atccccgacagcgctgacaccacagctacacccacgcactgtgcctatgacagg |
| | | atcgtggttgcagggatgctgctccgaggcgccgttgttcccgactcggctctt |
| | | ccctttaacttccagnctgcctatggcctgagtgaccaactggcccaagccatc |
| | | agtgaccactatccagtggaggtgatgctgaagtgataatctaga |
| 203 | huVK3LP- | metpaqllfllllwlpdttgmgpgarrqgrivqgrpemcfcppptplpplrilt |
| | hTREX1- | lgthtptpcsspgsaagtyptmgsqalppgpmqtliffdmeatglpfsqpkvte |
| | 72AA | lcllavhrcalespptsqgppptvpppprvvdklslcvapgkacspaaseitgl |
| | (G4S)4- | stavlaahgrqcfddnlanlllaflrrqpqpwclvahngdrydfpllqaelaml |
| | hIgG1- | gltsaldgafcvdsitalkalerasspsehgprksyslgsiytrlygqsppdsh |
| | WT-NLG- | taegdvlallsicqwrpqallrwvdaharpfgtirpmygvtasartkdlsgggg |
| | hDNase1- | sggggsggggsggggstglepkssdkthtcppcpapellggpsvflfppkpkdt |
| | G105R; | lmisrtpevtcvvvdvshedpevkfnwyvdgvevhnaktkpreeqynstyrvvs |
| | A114F | vltvlhqdwlngkeykckvsnkalpapiektiskakgqprepqvytlppsrdel |
| | | tknqvsltclvkgfypsdiavewesngqpennykttppvldsdgsfflyskltv |
| | | dksrwqqgnvfscsvmhealhnhytqkslslspgkvdgasshvnvsspsvqdil |
| | | kiaafniqtfgetkmsnatlvsyivqilsrydialvqevrdshltavgklldnl |
| | | nqdapdtyhyvvseplgrnsykerylfvyrpdqvsavdsyyyddgcepcgndtf |
| | | nrepaivrffsrftevrefaivplhaapgdavaeidalydvyldvqekwgsedv |
| | | mlmgdfnagcsyvrpsqwssirlwtsptfqwlipdsadttatpthcaydrivva |
| | | gmllrgavvpdsalpfnfqxayglsdqlaqaisdhypvevmlk* |
| 204 | huVK3LP- | gttaagcttgccaccatggaaaccccagcgcagcttctcttcctcctgctactc |
| | hRNase1- | tggctcccagataccaccggtaaggaatcccgggccaagaaattccagcggcag |
| | WT-(G4S)4- | catatggactcagacagttcccccagcagcagctccacctactgtaaccaaatg |
| | hIgG1- | atgaggcgccggaatatgacacaggggcggtgcaaaccagtgaacacctttgtg |
| | WT-NLG- | cacgagcccctggtagatgtccagaatgtctgtttccaggaaaaggtcacctgc |
| | hTREX1- | aagaacgggcagggcaactgctacaagagcaactccagcatgcacatcacagac |
| | 72AA | tgccgcctgacaaacggctccaggtaccccaactgtgcataccggaccagcccg |
| | | aaggagagacacatcattgtggcctgtgaagggagcccatatgtgccagtccac |
| | | tttgatgcttctgtggaggactctacagatctctccggaggaggtggctcaggt |
| | | ggtggaggatctggaggaggtgggagtggtggaggtggttctaccggtctcgag |
| | | cccaaatcttctgacaaaactcacacatgtccaccgtgcccagcacctgaactc |
| | | ctggggggaccgtcagtcttcctcttccccccaaaacccaaggacaccctcatg |
| | | atctcccggacccctgaggtcacatgcgtggtggtggacgtgagccacgaagac |
| | | cctgaggtcaagttcaactggtacgtggacggcgtggaggtgcataatgccaag |
| | | acaaagccgcgggaggagcagtacaacagcacgtaccgtgtggtcagcgtcctc |
| | | accgtcctgcaccaggactggctgaatggcaaggagtacaagtgcaaggtctcc |
| | | aacaaagccctcccagcccccatcgagaaaaccatctccaaagccaaagggcag |
| | | ccccgagaaccacaggtgtacaccctgcccccatcccgggatgagctgaccaag |
| | | aaccaggtcagcctgacctgcctggtcaaaggcttctatcccagcgacatcgcc |
| | | gtggagtgggagagcaatgggcagccggagaacaactacaagaccacgcctccc |
| | | gtgctggactccgacggctccttcttcctctacagcaagctcaccgtggacaag |
| | | agcaggtggcagcaggggaacgtcttctcatgctccgtgatgcatgaggctctg |
| | | cacaaccactacacgcagaagagcctctctctgtctccgggtaaagtcgacggt |
| | | gctagcagccatgtgaatgtgagcagccctagcgtgcaggatatcatgggccct |
| | | ggagctcgcagacagggcaggattgtgcagggaaggcctgagatgtgcttctgc |
| | | ccaccccctaccccactccctccccttcggatcttaacactgggcactcacaca |
| | | cccaccccatgctcctctccaggctcagcagcaggtacgtacccaaccatgggc |
| | | tcgcaggccctgcccccggggcccatgcagaccctcatctttttcgacatggag |
| | | gccactggcttgcccttctcccagcccaaggtcacggagctgtgcctgctggct |
| | | gtccacagatgtgccctggagagcccccccacctctcaggggccacctcccaca |
| | | gttcctccaccaccgcgtgtggtagacaagctctccctgtgtgtggctccgggg |
| | | aaggcctgcagccctgcagccagcgagatcacaggtctgagcacagctgtgctg |
| | | gcagcgcatgggcgtcaatgttttgatgacaacctggccaacctgctcctagcc |
| | | ttcctgcggcgccagccacagccctggtgcctggtggcacacaatggtgaccgc |
| | | tacgacttccccctgctccaagcagagctggctatgctgggcctcaccagtgct |
| | | ctggatggtgccttctgtgtggatagcatcactgcgctgaaggccctggagcga |
| | | gcaagcagcccctcagaacacggcccaaggaagagctacagcctaggcagcatc |
| | | tacactcgcctgtatgggcagtcccctccagactcgcacacggctgagggtgat |
| | | gtcctggccctgctcagcatctgtcagtggagaccacaggccctgctgcggtgg |
| | | gtggatgctcacgccaggcctttcggcaccatcaggcccatgtatggggtcaca |
| | | gcctctgctaggaccaaatgataatctaga |
| 205 | huVK3LP- | metpaqllfllllwlpdttgkesrakkfqrqhmdsdsspsssstycnqmmrrrn |
| | hRNase1- | mtqgrckpvntfvheplvdvqnvcfqekvtckngqgncyksnssmhitdcrltn |
| | WT-(G4S)4- | gsrypncayrtspkerhiivacegspyvpvhfdasvedstdlsggggsggggsg |
| | hIgG1- | gggsggggstglepkssdkthtcppcpapellggpsvflfppkpkdtlmisrtp |
| | WT-NLG- | evtcvvvdvshedpevkfnwyvdgvevhnaktkpreeqynstyrvvsvltvlhq |
| | hTREX1- | dwlngkeykckvsnkalpapiektiskakgqprepqvytlppsrdeltknqvsl |
| | 72AA | tcivkgfypsdiavewesngqpennykttppvldsdgsfflyskitvdksrwqq |
| | | gnvfscsvmhealhnhytqkslslspgkvdgasshvnvsspsvqdimgpgarrq |
| | | grivqgrpemcfcppptplpplriltlgthtptpcsspgsaagtyptmgsqalp |
| | | pgpmqtliffdmeatglpfsqpkvtelcllavhrcalespptsqgppptvpppp |
| | | rvvdklslcvapgkacspaaseitglstavlaahgrqcfddnlanlllafirrq |
| | | pqpwclvahngdrydfpllqaelamlgltsaldgafcvdsitalkalerassps |
| | | ehgprksyslgsiytrlygqsppdshtaegdvlallsicqwrpqallrwvdaha |
| | | rpfgtirpmygvtasartk* |
| 206 | huVK3LP- | gttaagcttgccaccatggaaaccccagcgcagcttctcttcctcctgctactc |
| | hDNase1L3- | tggctcccagataccaccggtatgaggatctgctccttcaacgtcaggtccttt |
| | hIgG1- | ggggaaagcaagcaggaagacaagaatgccatggatgtcattgtgaaggtcatc |
| | WT-NLG- | aaacgctgtgacatcatactcgtgatggaaatcaaggacagcaacaacaggatc |
| | hTREX1- | tgccccatactgatggagaagctgaacagaaattcaaggagaggcataacatac |
| | 72AA | aactatgtgattagctctcggcttggaagaaacacatataaagaacaatatgcc |
| | | tttctctacaaggaaaagctggtgtctgtgaagaggagttatcactaccatgac |
| | | tatcaggatggagacgcagatgtgttttccagggagccctttgtggtctggttc |
| | | caatctccccacactgctgtcaaagacttcgtgattatccccctgcacaccacc |
| | | ccagagacatccgttaaggagatcgatgagttggttgaggtctacacggacgtg |
| | | aaacaccgctggaaggcggagaatttcattttcatgggtgacttcaatgccggc |
| | | tgcagctacgtccccaagaaggcctggaagaacatccgcttgaggactgacccc |
| | | aggtttgtttggctgatcggggaccaagaggacaccacggtgaagaagagcacc |
| | | aactgtgcatatgacaggattgtgcttagaggacaagaaatcgtcagttctgtt |
| | | gttcccaagtcaaacagtgtttttgacttccagaaagcttacaagctgactgaa |
| | | gaggaggccctggatgtcagcgaccactttccagttgaatttaaactacagtct |
| | | tcaagggccttcaccaacagcaaaaaatctgtcactctaaggaagaaaacaaag |
| | | agcaaacgctcagatctcgagcccaaatcttctgacaaaactcacacatgtcca |
| | | ccgtgcccagcacctgaactcctggggggaccgtcagtcttcctcttcccccca |
| | | aaacccaaggacaccctcatgatctcccggacccctgaggtcacatgcgtggtg |
| | | gtggacgtgagccacgaagaccctgaggtcaagttcaactggtacgtggacggc |
| | | gtggaggtgcataatgccaagacaaagccgcgggaggagcagtacaacagcacg |
| | | taccgtgtggtcagcgtcctcaccgtcctgcaccaggactggctgaatggcaag |
| | | gagtacaagtgcaaggtctccaacaaagccctcccagcccccatcgagaaaacc |
| | | atctccaaagccaaagggcagccccgagaaccacaggtgtacaccctgccccca |
| | | tcccgggatgagctgaccaagaaccaggtcagcctgacctgcctggtcaaaggc |
| | | ttctatcccagcgacatcgccgtggagtgggagagcaatgggcagccggagaac |
| | | aactacaagaccacgcctcccgtgctggactccgacggctccttcttcctctac |
| | | agcaagctcaccgtggacaagagcaggtggcagcaggggaacgtcttctcatgc |
| | | tccgtgatgcatgaggctctgcacaaccactacacgcagaagagcctctctctg |
| | | tctccgggtaaagtcgacggagctagcagccccgtgaacgtgagcagccccagc |
| | | gtgcaggatatcatgggccctggagctcgcagacagggcaggattgtgcaggga |
| | | aggcctgagatgtgcttctgcccaccccctaccccactccctccccttcggatc |
| | | ttaacactgggcactcacacacccaccccatgctcctctccaggctcagcagca |
| | | ggtacgtacccaaccatgggctcgcaggccctgcccccggggcccatgcagacc |
| | | ctcatctttttcgacatggaggccactggcttgcccttctcccagcccaaggtc |
| | | acggagctgtgcctgctggctgtccacagatgtgccctggagagcccccccacc |
| | | tctcaggggccacctcccacagttcctccaccaccgcgtgtggtagacaagctc |
| | | tccctgtgtgtggctccggggaaggcctgcagccctgcagccagcgagatcaca |
| | | ggtctgagcacagctgtgctggcagcgcatgggcgtcaatgttttgatgacaac |
| | | ctggccaacctgctcctagccttcctgcggcgccagccacagccctggtgcctg |
| | | gtggcacacaatggtgaccgctacgacttccccctgctccaagcagagctggct |
| | | atgctgggcctcaccagtgctctggatggtgccttctgtgtggatagcatcact |
| | | gcgctgaaggccctggagcgagcaagcagcccctcagaacacggcccaaggaag |
| | | agctacagcctaggcagcatctacactcgcctgtatgggcagtcccctccagac |
| | | tcgcacacggctgagggtgatgtcctggccctgctcagcatctgtcagtggaga |
| | | ccacaggccctgctgcggtgggtggatgctcacgccaggcctttcggcaccatc |
| | | aggcccatgtatggggtcacagcctctgctaggaccaaatgataatctaga |
| 207 | huVK3LP- | metpaqllfllllwlpdttgmricsfnvrsfgeskqedknamdvivkvikrcdi |
| | hDNase1L3- | ilvmeikdsnnricpilmeklnrnsrrgitynyvissrlgrntykeqyaflyke |
| | hIgG1- | klvsvkrsyhyhdyqdgdadvfsrepfvvwfqsphtavkdfviiplhttpetsv |
| | WT-NLG- | keidelvevytdvkhrwkaenfifmgdfnagcsyvpkkawknirlrtdprfvwl |
| | hTREX1- | igdqedttvkkstncaydrivlrgqeivssvvpksnsvfdfqkayklteeeald |
| | 72AA | vsdhfpvefklqssraftnskksvtlrkktkskrsdlepkssdkthtcppcpap |
| | | ellggpsvflfppkpkdtlmisrtpevtcvvvdvshedpevkfnwyvdgvevhn |
| | | aktkpreeqynstyrvvsvltvlhqdwlngkeykckvsnkalpapiektiskak |
| | | gqprepqvytlppsrdeltknqvsltclvkgfypsdiavewesngqpennyktt |
| | | ppvldsdgsfflyskltvdksrwqqgnvfscsvmhealhnhytqkslslspgkv |
| | | dgasspvnvsspsvqdimgpgarrqgrivqgrpemcfcppptplpplriltlgt |
| | | htptpcsspgsaagtyptmgsqalppgpmqtliffdmeatglpfsqpkvtelcl |
| | | lavhrcalespptsqgppptvpppprvvdklslcvapgkacspaaseitglsta |
| | | vlaahgrqcfddnlanlllaflrrqpqpwclvahngdrydfpllqaelamlglt |
| | | saldgafcvdsitalkalerasspsehgprksyslgsiytrlygqsppdshtae |
| | | gdvlallsicqwrpqallrwvdaharpfgtirpmygvtasartk* |

1. A polypeptide comprising a first nuclease domain, a second nuclease domain, and a human immunoglobulin Fc domain or mutant immunoglobulin Fc domain, wherein the first and second nuclease domains are operably linked, with or without a linker, to each other in series to form a tandem array, wherein the tandem array of nuclease domains is operably linked, with or without a linker, to either the C-terminus or the N-terminus of the human immunoglobulin Fc domain or mutant immunoglobulin Fc domain, wherein the first and second nuclease domains are selected from a human RNase 1 and a human DNase 1.

2. The polypeptide of claim 1, wherein the first and second nuclease domains are selected from:
(a) a human RNase, such as a human pancreatic RNase 1; and
(b) human DNase selected from the group consisting of a Type 1 human DNase, a human DNase 1L3, or human TREX1.

3. The polypeptide of claim 1, wherein:
(a) the mutant immunoglobulin Fc domain comprises a hinge domain, a CH2 domain and a CH3 domain;
(b) the mutant immunoglobulin Fc domain comprises a modified hinge domain comprising at least one amino acid substitution, wherein the modified hinge domain comprises a substitution of one or more of the three hinge cysteines with serine, such as SCC or SSS; or
(c) the mutant immunoglobulin Fc domain comprises a modified CH2 domain comprising at least one substitution, wherein the substitution is selected from the group consisting of P238S, P331S, N297S or a combination thereof.

4. The polypeptide of claim 1, wherein:
the first or second nuclease domain is operatively coupled to the mutant immunoglobulin Fc domain via a linker domain;
wherein the first or second nuclease domain is operatively coupled to the N-terminus of the mutant immunoglobulin Fc domain; or
wherein the first or second nuclease domain is operatively coupled to the C-terminus of the mutant immunoglobulin Fc domain.

5. A composition comprising the polypeptide of claim 1 and a pharmaceutically acceptable carrier.

6. A nucleic acid molecule encoding the polypeptide according to claim 1.

7. A recombinant expression vector comprising the nucleic acid molecule according to claim 6.

8. A host cell transformed with the recombinant expression vector according to claim 7.

9. A method of making the polypeptide of claim 1, comprising: providing a host cell comprising a nucleic acid sequence that encodes the polypeptide; and maintaining the host cell under conditions in which the polypeptide is expressed.

10. The polypeptide of claim 1 for use in a method for treating or preventing a condition associated with an abnormal immune response, wherein the condition is an autoimmune disease, selected from the group consisting of insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbs-ve, cryptogenic cirrhosis, ulcerative colitis, Sjogren's syndrome, scleroderma, Wegener's granulomatosis, polymyositis, dermatomyositis, discoid LE, systemic lupus erythematosus (SLE), and connective tissue disease.

11. A dimeric polypeptide comprising the polypeptide of claim 1.

12. The dimeric polypeptide of claim 11, wherein the polypeptide is a homodimer.

13. A composition comprising the homodimer of claim 12 and a pharmaceutically acceptable carrier.

14. A method of treating systemic lupus erythematosus comprising administering to a subject the polypeptide of claim 1.

15. A method of treating Sjogren's syndrome comprising administering to a subject the polypeptide of claim 1.
